(19) United States
(12) Patent Application Publication
(10) Pub. No.: US 2024/0425870 A1
(43) Pub. Date: Dec. 26, 2024

(54) PLANT REGULATORY ELEMENTS AND USES THEREOF

(71) Applicant: Monsanto Technology LLC, St. Louis, MO (US)

(72) Inventor: Stanislaw Flasinski, Ballwin, MO (US)

(21) Appl. No.: 18/773,700

(22) Filed: Jul. 16, 2024

Related U.S. Application Data
(62) Division of application No. 17/821,123, filed on Aug. 19, 2022, now Pat. No. 12,071,628.
Application No. 17/821,123 is a division of application No. 16/900,717, filed on Jun. 12, 2020, now Pat. No. 11,466,282.
Application No. 16/900,717 is a division of application No. 15/798,319, filed on Oct. 30, 2017, now Pat. No. 10,752,910.
Application No. 15/798,319 is a division of application No. 14/686,602, filed on Apr. 14, 2015, now Pat. No. 9,834,777.
Application No. 14/686,602 is a division of application No. 13/428,994, filed on Mar. 23, 2012, now Pat. No. 9,062,316.
(60) Provisional application No. 61/467,875, filed on Mar. 25, 2011.

(51) Int. Cl. C12N 15/82; C12N 15/113

(52) U.S. Cl. CPC C12N 15/8216; C12N 15/113; C12N 15/8222

(57) ABSTRACT

The present invention provides novel DNA molecules and constructs, including their nucleotide sequences, useful for modulating gene expression in plants and plant cells. The invention also provides transgenic plants, plant cells, plant parts, seeds, and commodity products comprising the DNA molecules operably linked to heterologous transcribable polynucleotides, along with methods of their use.

### CROSS-REFERENCE TO RELATED APPLICATIONS

This application is a divisional of U.S. patent application Ser. No. 17/821,123, filed Aug. 19, 2022, which is a divisional of U.S. patent application Ser. No. 16/900,717, filed Jun. 12, 2020, now U.S. Pat. No. 11,466,282, which is a divisional of U.S. patent application Ser. No. 15/798,319, filed Oct. 30, 2017, now U.S. Pat. No. 10,752,910, which is a divisional of U.S. patent application Ser. No. 14/686,602, filed Apr. 14, 2015, now U.S. Pat. No. 9,834,777, which is a divisional of U.S. patent application Ser. No. 13/428,994, filed Mar. 23, 2012, now U.S. Pat. No. 9,062,316, which claims the benefit of U.S. Provisional Application No. 61/467,875, filed Mar. 25, 2011, each of which is herein incorporated by reference in its entirety.

### INCORPORATION OF SEQUENCE LISTING

The sequence listing that is contained in the file named “MONS282USD8_ST26.xml”, which is 421 KB (as measured in Microsoft Windows®) and was created on Jul. 12, 2024, is filed herewith by electronic submission and is incorporated by reference herein.

### FIELD OF THE INVENTION

The invention relates to the field of plant molecular biology and plant genetic engineering, and DNA molecules useful for modulating gene expression in plants.

### BACKGROUND

Regulatory elements are genetic elements that regulate gene activity by modulating the transcription of an operably linked transcribable polynucleotide molecule. Such elements include promoters, leaders, introns, and 3′ untranslated regions and are useful in the field of plant molecular biology and plant genetic engineering.

### SUMMARY OF THE INVENTION

The present invention provides novel gene regulatory elements for use in plants. The present invention also provides DNA constructs comprising the regulatory elements. The present invention also provides transgenic plant cells, plants, and seeds comprising the regulatory elements. The sequences may be provided operably linked to a transcribable polynucleotide molecule. In one embodiment, the transcribable polynucleotide molecule may be heterologous with respect to a regulatory sequence provided herein. A regulatory element sequence provided by the invention thus may, in particular embodiments, be defined as operably linked to a heterologous transcribable polynucleotide molecule. The present invention also provides methods of making and using the regulatory elements, the DNA constructs comprising the regulatory elements, and the transgenic plant cells, plants, and seeds comprising the regulatory elements operably linked to a transcribable polynucleotide molecule.

Thus, in one aspect, the present invention provides a DNA molecule comprising a DNA sequence selected from the group consisting of: a) a sequence with at least about 85 percent sequence identity to any of SEQ ID NOs: 1-158 and 180-183; b) a sequence comprising any of SEQ ID NOs: 1-158 and 180-183; and c) a fragment of any of SEQ ID NOs: 1-158 and 180-183, wherein the fragment has gene-regulatory activity; wherein the sequence is operably linked to a heterologous transcribable polynucleotide molecule. In specific embodiments, the DNA molecule comprises at least about 90 percent, at least about 95 percent, at least about 98 percent, or at least about 99 percent sequence identity to the DNA sequence of any of SEQ ID NOs: 1-158 and 180-183. In certain embodiments of the DNA molecule, the DNA sequence comprises a regulatory element. In some embodiments the regulatory element comprises a promoter. In particular embodiments, the heterologous transcribable polynucleotide molecule comprises a gene of agronomic interest, such as a gene capable of providing herbicide resistance in plants, or a gene capable of providing plant pest resistance in plants.

The invention also provides a transgenic plant cell comprising a heterologous DNA construct provided by the invention, including a sequence of any of SEQ ID NOs: 1-158 and 180-183, or a fragment or variant thereof, wherein said sequence is operably linked to a heterologous transcribable polynucleotide molecule. In certain embodiments, the transgenic plant cell is a monocotyledonous plant cell. In other embodiments, the transgenic plant cell is a dicotyledonous plant cell.

Further provided by the invention is a transgenic plant, or part thereof, comprising a DNA molecule as provided herein, including a DNA sequence selected from the group consisting of: a) a sequence with at least 85 percent sequence identity to any of SEQ ID NOs: 1-158 and 180-183; b) a sequence comprising any of SEQ ID NOs: 1-158 and 180-183; and c) a fragment of any of SEQ ID NOs: 1-158 and 180-183, wherein the fragment has gene-regulatory activity; wherein the sequence is operably linked to a heterologous transcribable polynucleotide molecule. In specific embodiments, the transgenic plant may be a progeny plant of any generation that comprises the DNA molecule, relative to a starting transgenic plant comprising the DNA molecule. Still further provided is a transgenic seed comprising a DNA molecule according to the invention.

In yet another aspect, the invention provides a method of producing a commodity product comprising obtaining a transgenic plant or part thereof according to the invention and producing the commodity product therefrom. In one embodiment, a commodity product of the invention is protein concentrate, protein isolate, grain, starch, seeds, meal, flour, biomass, or seed oil. In another aspect, the invention provides a commodity produced using the above method. For instance, in one embodiment the invention provides a commodity product comprising a DNA molecule as provided herein, including a DNA sequence selected from the group consisting of: a) a sequence with at least 85 percent sequence identity to any of SEQ ID NOs: 1-158 and 180-183; b) a sequence comprising any of SEQ ID NOs: 1-158 and 180-183; and c) a fragment of any of SEQ ID NOs: 1-158 and 180-183, wherein the fragment has gene-regulatory activity; wherein the sequence is operably linked to a heterologous transcribable polynucleotide molecule.

In still yet another aspect, the invention provides a method of expressing a transcribable polynucleotide molecule that comprises obtaining a transgenic plant according to the invention, such as a plant comprising a DNA molecule as described herein, and cultivating plant, wherein a transcribable polynucleotide in the DNA molecule is expressed.

Throughout this specification and the claims, unless the context requires otherwise, the word “comprise” and its variations, such as “comprises” and “comprising,” will be understood to imply the inclusion of a stated composition, step, and/or value, or group thereof, but not the exclusion of any other composition, step, and/or value, or group thereof.

### BRIEF DESCRIPTION OF THE FIGURES

FIGS. 1a-1h depict alignment of promoter size variants corresponding to promoter elements isolated from the grass species Andropogon gerardii. In particular, FIGS. 1a-1h show alignment of the 2603 bp promoter sequence P-ANDge.Ubq1-1:1:11 (SEQ ID NO: 2), found in the transcriptional regulatory expression element group EXP-ANDge.Ubq1:1:9 (SEQ ID NO: 1), with promoter sequences derived via deletion analysis of P-ANDge.Ubq1-1:1:11. Deletion, for instance of the 5′ end of P-ANDge.Ubq1-1:1:11, produced the promoter P-ANDge.Ubq1-1:1:9 (SEQ ID NO: 6), a 2114 bp sequence which is found within EXP-ANDge.Ubq1:1:7 (SEQ ID NO: 5). Other promoter sequences in FIG. 1 include P-ANDge.Ubq1-1:1:10 (SEQ ID NO: 9), a 1644 bp sequence comprised within EXP-ANDge.Ubq1:1:8 (SEQ ID NO: 8); P-ANDge.Ubq1-1:1:12 (SEQ ID NO: 11), a 1472 bp sequence comprised within EXP-ANDge.Ubq1:1:10 (SEQ ID NO: 10); P-ANDge.Ubq1-1:1:8 (SEQ ID NO: 13), a 1114 bp sequence comprised within EXP-ANDge.Ubq1:1:6 (SEQ ID NO: 12); P-ANDge.Ubq1-1:1:13 (SEQ ID NO: 15), a 771 bp sequence comprised within EXP-ANDge.Ubq1:1:11 (SEQ ID NO: 14); and P-ANDge.Ubq1-1:1:14 (SEQ ID NO: 17), a 482 bp sequence comprised within EXP-ANDge.Ubq1:1:12 (SEQ ID NO: 16). Asterisks indicate bases which are identical between the aligned sequences.

FIGS. 2a-2g depict alignment of promoter variants isolated from the grass Saccharum ravennae (Erianthus ravennae). In particular, FIGS. 2a-2g show an alignment of the 2536 bp promoter sequence P-ERIra.Ubq1-1:1:10 (SEQ ID NO: 19) (found, for instance, within the transcriptional regulatory expression element group EXP-ERIra.Ubq1 (SEQ ID NO: 18)) with promoter sequences derived from deletion analysis of P-ERIra.Ubq1-1:1:10: a 2014 bp promoter sequence P-ERIra.Ubq1-1:1:9 (SEQ ID NO: 23); a 1525 bp promoter sequence P-ERIra.Ubq1-1:1:11 (SEQ ID NO: 26); a 1044 bp promoter sequence P-ERIra.Ubq1-1:1:8 (SEQ ID NO: 28); a 796 bp sequence P-ERIra.Ubq1-1:1:12 (SEQ ID NO: 30); and a 511 bp sequence P-ERIra.Ubq1-1:1:13 (SEQ ID NO: 32). Asterisks indicate bases which are identical between the aligned sequences.

FIGS. 3a-3c depict alignment of promoter size variants corresponding to promoter elements isolated from the grass species Setaria viridis. In particular, FIGS. 3a-3c show an alignment of a 1493 bp promoter sequence, P-Sv.Ubq1-1:1:1 (SEQ ID NO: 34) with promoters derived from deletion analysis of the 5′ end of P-Sv.Ubq1-1:1:1: a 1035 bp sized promoter P-Sv.Ubq1-1:1:2 (SEQ ID NO: 38); and a 681 bp promoter sequence P-Sv.Ubq1-1:1:3 (SEQ ID NO: 40). Asterisks indicate bases which are identical between the aligned sequences.

FIGS. 4a-4e depict alignment of transcriptional regulatory expression element group variants derived from the grass Zea mays subsp. mexicana. In particular, FIGS. 4a-4e compare a 2005 bp transcriptional regulatory expression element group termed EXP-Zm.UbgM1:1:2 (SEQ ID NO: 49) with allelic variant EXP-Zm.UbgM1:1:5 (SEQ ID NO: 53), as well as with size variants EXP-Zm.UbgM1:1:1 (SEQ ID NO: 41), which is 1922 bps in length, and EXP-Zm.UbgM1:1:4 (SEQ ID NO: 45), which is 1971 bps in length. Asterisks indicate bases which are identical between the aligned sequences.

FIGS. 5a-5b depict alignment of promoter size variants isolated from the grass Sorghum bicolor. In particular, FIGS. 5a-5b shows alignment of the 791 bp sized promoter element, P-Sb.Ubg6-1:1:2 (SEQ ID NO: 60) comprised within the transcriptional regulatory expression element group EXP-Sb.Ubg6 (SEQ ID NO: 59), with 855 bp promoter element P-Sb.Ubg6-1:1:1 (SEQ ID NO: 64) comprised within EXP-Sb.Ubg6:1:1 (SEQ ID NO: 63). Asterisks indicate bases which are identical between the aligned sequences.

FIGS. 6a-6c depict alignment of promoter size variants corresponding to promoter elements isolated from the grass Setaria italica. In particular, FIGS. 6a-6c show an alignment of the 1492 bp promoter variant P-SETit.Ubq1-1:1:1 (SEQ ID NO: 70) with 1492 bp promoter variant P-SETit.Ubq1-1:1:4 (SEQ ID NO: 74), 1034 bp promoter element P-SETit.Ubq1-1:1:2 (SEQ ID NO: 76), and 680 bp promoter element P-SETit.Ubq1-1:1:3 (SEQ ID NO: 78). Asterisks indicate bases which are identical between the aligned sequences.

FIGS. 7a-7b depict alignment of promoter size variants and an enhancer element corresponding to promoter elements isolated from the grass species Coix lachryma-jobi. In particular, FIGS. 7a and 7b show an alignment of the 837 bp promoter variant, P-Cl.Ubq1-1:1:1 (SEQ ID NO: 80) found within transcriptional regulatory expression element group EXP-Cl.Ubq1:1:1 (SEQ ID NO: 79), with an enhancer fragment derived from P-Cl.Ubq1-1:1:1, termed E-Cl.Ubq1:1:1 (SEQ ID NO: 89) that is 798 bp in length, as well as with three 5′ end deletion variants of P-Cl.Ubq1-1:1:1: a 742 bp element P-Cl.Ubq1-1:1:4 (SEQ ID NO: 84); a 401 bp element P-Cl.Ubq1-1:1:3 (SEQ ID NO: 86); and a 54 bp minimal promoter element P-Cl.Ubq1-1:1:5 (SEQ ID NO: 88). Asterisks indicate bases which are identical between the aligned sequences.

FIG. 8 depicts transgene cassette configurations of the present invention.

### BRIEF DESCRIPTION OF THE SEQUENCES

SEQ ID NOS: 1, 5, 8, 10, 12, 14, 16, 18, 22, 25, 27, 29, 31, 33, 37, 39, 41, 45, 49, 53, 55, 59, 63, 65, 69, 73, 75, 77, 79, 83, 85, 87, 90, 93, 95, 97, 98, 99, 100, 102, 104, 106, 108, 110, 112, 114, 115, 116, 117, 119, 121, 123, 124, 125, 126, 128, 130, 132, 133, 134, 136, 137, 139, 141, 143, 145, 147, 149, 151, 153, 155, 157, 180, 181 and 183 are sequences of transcriptional regulatory expression element groups or EXP sequences comprising a promoter sequence operably linked 5′ to a leader sequence which is operably linked 5′ to an intron sequence.

SEQ ID NOS: 2, 6, 9, 11, 13, 15, 17, 19, 23, 26, 28, 30, 32, 34, 38, 40, 42, 46, 50, 56, 60, 64, 66, 70, 74, 76, 78, 80, 84, 86, 88, 91, 96 and 135 are promoter sequences.

SEQ ID NOS: 3, 20, 35, 43, 47, 51, 57, 61, 67, 71 and 81 are leader sequences.

SEQ ID NOS: 4, 7, 21, 24, 36, 44, 48, 52, 54, 58, 62, 68, 72, 82, 92, 94, 101, 103, 105, 107, 109, 111, 113, 118, 120, 122, 127, 129, 131, 138, 140, 142, 144, 146, 148, 150, 152, 154, 156, 158 and 182 are intron sequences.

SEQ ID NO: 89 is the sequence of an enhancer.

### DETAILED DESCRIPTION OF THE INVENTION

The invention disclosed herein provides polynucleotide molecules having beneficial gene regulatory activity from plant species. The design, construction, and use of these polynucleotide molecules are provided by the invention. The nucleotide sequences of these polynucleotide molecules are provided among SEQ ID NOs: 1-158 and 180-183. These polynucleotide molecules are, for instance, capable of affecting the expression of an operably linked transcribable polynucleotide molecule in plant tissues, and therefore selectively regulating gene expression, or activity of an encoded gene product, in transgenic plants. The present invention also provides methods of modifying, producing, and using the same. The invention also provides compositions, transformed host cells, transgenic plants, and seeds containing the promoters and/or other disclosed nucleotide sequences, and methods for preparing and using the same.

The following definitions and methods are provided to better define the present invention and to guide those of ordinary skill in the art in the practice of the present invention. Unless otherwise noted, terms are to be understood according to conventional usage by those of ordinary skill in the relevant art.

### DNA Molecules

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

Any number of methods well known to those skilled in the art can be used to isolate and manipulate a DNA molecule, or fragment thereof, disclosed in the present invention. For example, PCR (polymerase chain reaction) technology can be used to amplify a particular starting DNA molecule and/or to produce variants of the original molecule. DNA molecules, or fragment thereof, can also be obtained by other techniques such as by directly synthesizing the fragment by chemical means, as is commonly practiced by using an automated oligonucleotide synthesizer.

As used herein, the term “sequence identity” refers to the extent to which two optimally aligned polynucleotide sequences or two optimally aligned polypeptide sequences are identical. An optimal sequence alignment is created by manually aligning two sequences, e.g. a reference sequence and another sequence, to maximize the number of nucleotide matches in the sequence alignment with appropriate internal nucleotide insertions, deletions, or gaps. As used herein, the term “reference sequence” refers to a sequence provided as the polynucleotide sequences of SEQ ID NOs: 1-158 and 180-183.

As used herein, the term “percent sequence identity” or “percent identity” or “% identity” is the identity fraction times 100. The “identity fraction” for a sequence optimally aligned with a reference sequence is the number of nucleotide matches in the optimal alignment, divided by the total number of nucleotides in the reference sequence, e.g. the total number of nucleotides in the full length of the entire reference sequence. Thus, one embodiment of the invention is a DNA molecule comprising a sequence that when optimally aligned to a reference sequence, provided herein as SEQ ID NOs: 1-158 and 180-183, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments such sequences may be defined as having gene-regulatory activity.

### Regulatory Elements

A regulatory element is a DNA molecule having gene regulatory activity, i.e. one that has the ability to affect the transcription and/or translation of an operably linked transcribable polynucleotide molecule. The term “gene regulatory activity” thus refers to the ability to affect the expression pattern of an operably linked transcribable polynucleotide molecule by affecting the transcription and/or translation of that operably linked transcribable polynucleotide molecule. As used herein, a transcriptional regulatory expression element group or “EXP” sequence may be comprised of expression elements, such as enhancers, promoters, leaders and introns, operably linked. Thus a transcriptional regulatory expression element group may be comprised, for instance, of a promoter operably linked 5′ to a leader sequence, which is in turn operably linked 5′ to an intron sequence. The intron sequence may be comprised of a sequence beginning at the point of the first intron/exon splice junction of the native sequence and further may be comprised of a small leader fragment comprising the second intron/exon splice junction so as to provide for proper intron/exon processing to facilitate transcription and proper processing of the resulting transcript. Leaders and introns may positively affect transcription of an operably linked transcribable polynucleotide molecule as well as translation of the resulting transcribed RNA. The pre-processed RNA molecule comprises leaders and introns, which may affect the post-transcriptional processing of the transcribed RNA and/or the export of the transcribed RNA molecule from the cell nucleus into the cytoplasm. Following post-transcriptional processing of the transcribed RNA molecule, the leader sequence may be retained as part of the final messenger RNA and may positively affect the translation of the messenger RNA molecule.

Regulatory elements such as promoters, leaders, introns, and transcription termination regions (or 3′ UTRs) are DNA molecules that have gene regulatory activity and play an integral part in the overall expression of genes in living cells. The term “regulatory element” refers to a DNA molecule having gene regulatory activity, i.e. one that has the ability to affect the transcription and/or translation of an operably linked transcribable polynucleotide molecule. Isolated regulatory elements, such as promoters and leaders that function in plants are therefore useful for modifying plant phenotypes through the methods of genetic engineering.

Regulatory elements may be characterized by their expression pattern effects (qualitatively and/or quantitatively), e.g. positive or negative effects and/or constitutive or other effects such as by their temporal, spatial, developmental, tissue, environmental, physiological, pathological, cell cycle, and/or chemically responsive expression pattern, and any combination thereof, as well as by quantitative or qualitative indications. A promoter is useful as a regulatory element for modulating the expression of an operably linked transcribable polynucleotide molecule.

As used herein, a “gene expression pattern” is any pattern of transcription of an operably linked DNA molecule into a transcribed RNA molecule. The transcribed RNA molecule may be translated to produce a protein molecule or may provide an antisense or other regulatory RNA molecule, such as a dsRNA, a tRNA, an rRNA, a miRNA, and the like.

As used herein, the term “protein expression” is any pattern of translation of a transcribed RNA molecule into a protein molecule. Protein expression may be characterized by its temporal, spatial, developmental, or morphological qualities as well as by quantitative or qualitative indications.

As used herein, the term “promoter” refers generally to a DNA molecule that is involved in recognition and binding of RNA polymerase II and other proteins (trans-acting transcription factors) to initiate transcription. A promoter may be initially isolated from the 5′ untranslated region (5′ UTR) of a genomic copy of a gene. Alternately, promoters may be synthetically produced or manipulated DNA molecules. Promoters may also be chimeric, that is a promoter produced through the fusion of two or more heterologous DNA molecules. Promoters useful in practicing the present invention include SEQ ID NOS: 2, 6, 9, 11, 13, 15, 17, 19, 23, 26, 28, 30, 32, 34, 38, 40, 42, 46, 50, 56, 60, 64, 66, 70, 74, 76, 78, 80, 84, 86, 88, 91, 96 and 135, or fragments or variants thereof. In specific embodiments of the invention, such molecules and any variants or derivatives thereof as described herein, are further defined as comprising promoter activity, i.e., are capable of acting as a promoter in a host cell, such as in a transgenic plant. In still further specific embodiments, a fragment may be defined as exhibiting promoter activity possessed by the starting promoter molecule from which it is derived, or a fragment may comprise a “minimal promoter” which provides a basal level of transcription and is comprised of a TATA box or equivalent sequence for recognition and binding of the RNA polymerase II complex for initiation of transcription.

In one embodiment, fragments are provided of a promoter sequence disclosed herein. Promoter fragments may comprise promoter activity, as described above, and may be useful alone or in combination with other promoters and promoter fragments, such as in constructing chimeric promoters. In specific embodiments, fragments of a promoter are provided comprising at least about 50, 95, 150, 250, 500, 750, or at least about 1000 contiguous nucleotides, or longer, of a polynucleotide molecule having promoter activity disclosed herein.

Compositions derived from any of the promoters presented as SEQ ID NOS: 2, 6, 9, 11, 13, 15, 17, 19, 23, 26, 28, 30, 32, 34, 38, 40, 42, 46, 50, 56, 60, 64, 66, 70, 74, 76, 78, 80, 84, 86, 88, 91, 96 and 135, such as internal or 5′ deletions, for example, can be produced using methods known in the art to improve or alter expression, including by removing elements that have either positive or negative effects on expression; duplicating elements that have positive or negative effects on expression; and/or duplicating or removing elements that have tissue or cell specific effects on expression. Compositions derived from any of the promoters presented as SEQ ID NOS: 2, 6, 9, 11, 13, 15, 17, 19, 23, 26, 28, 30, 32, 34, 38, 40, 42, 46, 50, 56, 60, 64, 66, 70, 74, 76, 78, 80, 84, 86, 88, 91, 96 and 135 comprised of 3′ deletions in which the TATA box element or equivalent sequence thereof and downstream sequence is removed can be used, for example, to make enhancer elements. Further deletions can be made to remove any elements that have positive or negative; tissue specific; cell specific; or timing specific (such as, but not limited to, circadian rhythms) effects on expression. Any of the promoters presented as SEQ ID NOS: 2, 6, 9, 11, 13, 15, 17, 19, 23, 26, 28, 30, 32, 34, 38, 40, 42, 46, 50, 56, 60, 64, 66, 70, 74, 76, 78, 80, 84, 86, 88, 91, 96 and 135 and fragments or enhancers derived there from can be used to make chimeric transcriptional regulatory element compositions comprised of any of the promoters presented as SEQ ID NOS: 2, 6, 9, 11, 13, 15, 17, 19, 23, 26, 28, 30, 32, 34, 38, 40, 42, 46, 50, 56, 60, 64, 66, 70, 74, 76, 78, 80, 84, 86, 88, 91, 96 and 135 and the fragments or enhancers derived therefrom operably linked to other enhancers and promoters. The efficacy of the modifications, duplications or deletions described herein on the desired expression aspects of a particular transgene may be tested empirically in stable and transient plant assays, such as those described in the working examples herein, so as to validate the results, which may vary depending upon the changes made and the goal of the change in the starting molecule.

As used herein, the term “leader” refers to a DNA molecule isolated from the untranslated 5′ region (5′ UTR) of a genomic copy of a gene and defined generally as a nucleotide segment between the transcription start site (TSS) and the protein coding sequence start site. Alternately, leaders may be synthetically produced or manipulated DNA elements. A leader can be used as a 5′ regulatory element for modulating expression of an operably linked transcribable polynucleotide molecule. Leader molecules may be used with a heterologous promoter or with their native promoter. Promoter molecules of the present invention may thus be operably linked to their native leader or may be operably linked to a heterologous leader. Leaders useful in practicing the present invention include SEQ ID NOS: 3, 20, 35, 43, 47, 51, 57, 61, 67, 71 and 81 or fragments or variants thereof. In specific embodiments, such sequences may be provided defined as being capable of acting as a leader in a host cell, including, for example, a transgenic plant cell. In one embodiment such sequences are decoded as comprising leader activity.

The leader sequences (5′ UTR) presented as SEQ ID NOS: 3, 20, 35, 43, 47, 51, 57, 61, 67, 71 and 81 may be comprised of regulatory elements or may adopt secondary structures that can have an effect on transcription or translation of a transgene. The leader sequences presented as SEQ ID NOS: 3, 20, 35, 43, 47, 51, 57, 61, 67, 71 and 81 can be used in accordance with the invention to make chimeric regulatory elements that affect transcription or translation of a transgene. In addition, the leader sequences presented as SEQ ID NOS: 3, 20, 35, 43, 47, 51, 57, 61, 67, 71 and 81 can be used to make chimeric leader sequences that affect transcription or translation of a transgene.

The introduction of a foreign gene into a new plant host does not always result in a high expression of the incoming gene. Furthermore, if dealing with complex traits, it is sometimes necessary to modulate several genes with spatially or temporarily different expression pattern. Introns can principally provide such modulation. However multiple use of the same intron in one plant has shown to exhibit disadvantages. In those cases it is necessary to have a collection of basic control elements for the construction of appropriate recombinant DNA elements. As the available collection of introns known in the art with expression enhancing properties is limited, alternatives are needed.

Compositions derived from any of the introns presented as SEQ ID NOS: 4, 7, 21, 24, 36, 44, 48, 52, 54, 58, 62, 68, 72, 82, 92, 94, 101, 103, 105, 107, 109, 111, 113, 118, 120, 122, 127, 129, 131, 138, 140, 142, 144, 146, 148, 150, 152, 154, 156, 158 and 182 can be comprised of internal deletions or duplications of cis regulatory elements; and/or alterations of the 5′ and 3′ sequences comprising the intron/exon splice junctions can be used to improve expression or specificity of expression when operably linked to a promoter+leader or chimeric promoter+leader and coding sequence. Alterations of the 5′ and 3′ regions comprising the intron/exon splice junction can also be made to reduce the potential for introduction of false start and stop codons being produced in the resulting transcript after processing and splicing of the messenger RNA. The introns can be tested empirically as described in the working examples to determine the intron's effect on expression of a transgene.

In accordance with the invention a promoter or promoter fragment may be analyzed for the presence of known promoter elements, i.e. DNA sequence characteristics, such as a TATA-box and other known transcription factor binding site motifs. Identification of such known promoter elements may be used by one of skill in the art to design variants of the promoter having a similar expression pattern to the original promoter.

As used herein, the term “enhancer” or “enhancer element” refers to a cis-acting transcriptional regulatory element, a.k.a. cis-element, which confers an aspect of the overall expression pattern, but is usually insufficient alone to drive transcription, of an operably linked polynucleotide sequence. Unlike promoters, enhancer elements do not usually include a transcription start site (TSS) or TATA box or equivalent sequence. A promoter may naturally comprise one or more enhancer elements that affect the transcription of an operably linked polynucleotide sequence. An isolated enhancer element may also be fused to a promoter to produce a chimeric promoter cis-element, which confers an aspect of the overall modulation of gene expression. A promoter or promoter fragment may comprise one or more enhancer elements that effect the transcription of operably linked genes. Many promoter enhancer elements are believed to bind DNA-binding proteins and/or affect DNA topology, producing local conformations that selectively allow or restrict access of RNA polymerase to the DNA template or that facilitate selective opening of the double helix at the site of transcriptional initiation. An enhancer element may function to bind transcription factors that regulate transcription. Some enhancer elements bind more than one transcription factor, and transcription factors may interact with different affinities with more than one enhancer domain. Enhancer elements can be identified by a number of techniques, including deletion analysis, i.e. deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis using known cis-element motifs or enhancer elements as a target sequence or target motif with conventional DNA sequence comparison methods, such as BLAST. The fine structure of an enhancer domain can be further studied by mutagenesis (or substitution) of one or more nucleotides or by other conventional methods. Enhancer elements can be obtained by chemical synthesis or by isolation from regulatory elements that include such elements, and they can be synthesized with additional flanking nucleotides that contain useful restriction enzyme sites to facilitate subsequence manipulation. Thus, the design, construction, and use of enhancer elements according to the methods disclosed herein for modulating the expression of operably linked transcribable polynucleotide molecules are encompassed by the present invention.

In plants, the inclusion of some introns in gene constructs leads to increased mRNA and protein accumulation relative to constructs lacking the intron.

This effect has been termed “intron mediated enhancement” (IME) of gene expression (Mascarenhas et al., (1990) Plant Mol. Biol. 15:913-920). Introns known to stimulate expression in plants have been identified in maize genes (e.g. tubA1, Adh1, Sh1, Ubi1 (Jeon et al. (2000) Plant Physiol. 123:1005-1014; Callis et al. (1987) Genes Dev. 1:1183-1200; Vasil et al. (1989) Plant Physiol. 91:1575-1579; Christiansen et al. (1992) Plant Mol. Biol. 18:675-689) and in rice genes (e.g. salt, tpi: McElroy et al., Plant Cell 2:163-171 (1990); Xu et al., Plant Physiol. 106:459-467 (1994)). Similarly, introns from dicotyledonous plant genes like those from petunia (e.g. rbcS), potato (e.g. st-ls1) and from Arabidopsis thaliana (e.g. ubq3 and pat1) have been found to elevate gene expression rates (Dean et al. (1989) Plant Cell 1:201-208; Leon et al. (1991) Plant Physiol. 95:968-972; Norris et al. (1993) Plant Mol Biol 21:895-906; Rose and Last (1997) Plant J. 11:455-464). It has been shown that deletions or mutations within the splice sites of an intron reduce gene expression, indicating that splicing might be needed for IME (Mascarenhas et al. (1990) Plant Mol Biol. 15:913-920; Clancy and Hannah (2002) Plant Physiol. 130:918-929). However, that splicing per se is not required for a certain IME in dicotyledonous plants has been shown by point mutations within the splice sites of the pat1 gene from A. thaliana (Rose and Beliakoff (2000) Plant Physiol. 122:535-542).

Enhancement of gene expression by introns is not a general phenomenon because some intron insertions into recombinant expression cassettes fail to enhance expression (e.g. introns from dicot genes (rbcS gene from pea, phaseolin gene from bean and the stls-1 gene from Solanum tuberosum) and introns from maize genes (adh1 gene the ninth intron, hsp81 gene the first intron)) (Chee et al. (1986) Gene 41:47-57; Kuhlemeier et al. (1988) Mol Gen Genet 212:405-411; Mascarenhas et al. (1990) Plant Mol. Biol. 15:913-920; Sinibaldi and Mettler (1992) In WE Cohn, K Moldave, eds, Progress in Nucleic Acid Research and Molecular Biology, Vol 42. Academic Press, New York, pp 229-257; Vancanneyt et al. 1990 Mol. Gen. Genet. 220:245-250). Therefore, not each intron can be employed in order to manipulate the gene expression level of non-endogenous genes or endogenous genes in transgenic plants. What characteristics or specific sequence features must be present in an intron sequence in order to enhance the expression rate of a given gene is not known in the prior art and therefore from the prior art it is not possible to predict whether a given plant intron, when used heterologously, will cause enhancement of expression at the DNA level or at the transcript level (IME).

As used herein, the term “chimeric” refers to a single DNA molecule produced by fusing a first DNA molecule to a second DNA molecule, where neither first nor second DNA molecule would normally be found in that configuration, i.e. fused to the other. The chimeric DNA molecule is thus a new DNA molecule not otherwise normally found in nature. As used herein, the term “chimeric promoter” refers to a promoter produced through such manipulation of DNA molecules. A chimeric promoter may combine two or more DNA fragments; an example would be the fusion of a promoter to an enhancer element. Thus, the design, construction, and use of chimeric promoters according to the methods disclosed herein for modulating the expression of operably linked transcribable polynucleotide molecules are encompassed by the present invention.

As used herein, the term “variant” refers to a second DNA molecule that is in composition similar, but not identical to, a first DNA molecule and yet the second DNA molecule still maintains the general functionality, i.e. same or similar expression pattern, of the first DNA molecule. A variant may be a shorter or truncated version of the first DNA molecule and/or an altered version of the sequence of the first DNA molecule, such as one with different restriction enzyme sites and/or internal deletions, substitutions, and/or insertions. A “variant” can also encompass a regulatory element having a nucleotide sequence comprising a substitution, deletion and/or insertion of one or more nucleotides of a reference sequence, wherein the derivative regulatory element has more or less or equivalent transcriptional or translational activity than the corresponding parent regulatory molecule. The regulatory element “variants” will also encompass variants arising from mutations that naturally occur in bacterial and plant cell transformation. In the present invention, a polynucleotide sequence provided as SEQ ID NOs: 1-158 and 180-183 may be used to create variants that are in composition similar, but not identical to, the polynucleotide sequence of the original regulatory element, while still maintaining the general functionality, i.e. same or similar expression pattern, of the original regulatory element. Production of such variants of the present invention is well within the ordinary skill of the art in light of the disclosure and is encompassed within the scope of the present invention. Chimeric regulatory element “variants” comprise the same constituent elements as a reference sequence but the constituent elements comprising the chimeric regulatory element may be operatively linked by various methods known in the art such as, restriction enzyme digestion and ligation, ligation independent cloning, modular assembly of PCR products during amplification, or direct chemical synthesis of the regulatory element as well as other methods known in the art. The resulting chimeric regulatory element “variant” can be comprised of the same, or variants of the same, constituent elements of the reference sequence but differ in the sequence or sequences that comprise the linking sequence or sequences which allow the constituent parts to be operatively linked. In the present invention, a polynucleotide sequence provided as SEQ ID NOs: 1-158 and 180-183 provide a reference sequence wherein the constituent elements that comprise the reference sequence may be joined by methods known in the art and may comprise substitutions, deletions and/or insertions of one or more nucleotides or mutations that naturally occur in bacterial and plant cell transformation.

### Constructs

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. As used herein, the term “vector” means any recombinant polynucleotide construct that may be used for the purpose of transformation, i.e. the introduction of heterologous DNA into a host cell. The term includes an expression cassette isolated from any of the aforementioned molecules.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. A leader, for example, is operably linked to coding sequence when it is capable of serving as a leader for the polypeptide encoded by the coding sequence.

The constructs of the present invention may be provided, in one embodiment, as double Ti plasmid border DNA constructs that have the right border (RB or AGRtu.RB) and left border (LB or AGRtu.LB) regions of the Ti plasmid isolated from Agrobacterium tumefaciens comprising a T-DNA, that along with transfer molecules provided by the A. tumefaciens cells, permit the integration of the T-DNA into the genome of a plant cell (see, for example, U.S. Pat. No. 6,603,061). The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often A. tumefaciens ABI, C58, or LBA4404; however, other strains known to those skilled in the art of plant transformation can function in the present invention.

Methods are known in the art for assembling and introducing constructs into a cell in such a manner that the transcribable polynucleotide molecule is transcribed into a functional mRNA molecule that is translated and expressed as a protein product. For the practice of the present invention, conventional compositions and methods for preparing and using constructs and host cells are well known to one skilled in the art, see, for example, Molecular Cloning: A Laboratory Manual, 3^{rd} edition Volumes 1, 2, and 3 (2000) J. Sambrook, D. W. Russell, and N. Irwin, Cold Spring Harbor Laboratory Press. Methods for making recombinant vectors particularly suited to plant transformation include, without limitation, those described in U.S. Pat. Nos. 4,971,908; 4,940,835; 4,769,061; and 4,757,011 in their entirety. These types of vectors have also been reviewed in the scientific literature (see, for example, Rodriguez, et al., Vectors: A Survey of Molecular Cloning Vectors and Their Uses, Butterworths, Boston, (1988) and Glick, et al., Methods in Plant Molecular Biology and Biotechnology, CRC Press, Boca Raton, FL. (1993)). Typical vectors useful for expression of nucleic acids in higher plants are well known in the art and include vectors derived from the tumor-inducing (Ti) plasmid of Agrobacterium tumefaciens (Rogers, et al., Methods in Enzymology 153: 253-277 (1987)). Other recombinant vectors useful for plant transformation, including the pCaMVCN transfer control vector, have also been described in the scientific literature (see, for example, Fromm, et al., Proc. Natl. Acad. Sci. USA 82: 5824-5828 (1985)).

Various regulatory elements may be included in a construct including any of those provided herein. Any such regulatory elements may be provided in combination with other regulatory elements. Such combinations can be designed or modified to produce desirable regulatory features. In one embodiment, constructs of the present invention comprise at least one regulatory element operably linked to a transcribable polynucleotide molecule operably linked to a 3′UTR.

Constructs of the present invention may include any promoter or leader provided herein or known in the art. For example, a promoter of the present invention may be operably linked to a heterologous non-translated 5′ leader such as one derived from a heat shock protein gene (see, for example, U.S. Pat. Nos. 5,659,122 and 5,362,865). Alternatively, a leader of the present invention may be operably linked to a heterologous promoter such as the Cauliflower Mosaic Virus 35S transcript promoter (see, U.S. Pat. No. 5,352,605).

As used herein, the term “intron” refers to a DNA molecule that may be isolated or identified from the genomic copy of a gene and may be defined generally as a region spliced out during mRNA processing prior to translation. Alternately, an intron may be a synthetically produced or manipulated DNA element. An intron may contain enhancer elements that effect the transcription of operably linked genes. An intron may be used as a regulatory element for modulating expression of an operably linked transcribable polynucleotide molecule. A DNA construct may comprise an intron, and the intron may or may not be heterologous with respect to the transcribable polynucleotide molecule sequence. Examples of introns in the art include the rice actin intron (U.S. Pat. No. 5,641,876) and the corn HSP70 intron (U.S. Pat. No. 5,859,347). Introns useful in practicing the present invention include SEQ ID NOS: 4, 7, 21, 24, 36, 44, 48, 52, 54, 58, 62, 68, 72, 82, 92, 94, 101, 103, 105, 107, 109, 111, 113, 118, 120, 122, 127, 129, 131, 138, 140, 142, 144, 146, 148, 150, 152, 154, 156, 158 and 182. Further, when modifying intron/exon boundary sequences, it may be preferable to avoid using the nucleotide sequence AT or the nucleotide A just prior to the 5′ end of the splice site (GT) and the nucleotide G or the nucleotide sequence TG, respectively just after 3′ end of the splice site (AG) to eliminate the potential of unwanted start codons from being formed during processing of the messenger RNA into the final transcript. The sequence around the 5′ or 3′ end splice junction sites of the intron can thus be modified in this manner.

As used herein, the term “3′ transcription termination molecule” or “3′ UTR” refers to a DNA molecule that is used during transcription to produce the 3′ untranslated region (3′ UTR) of an mRNA molecule. The 3′ untranslated region of an mRNA molecule may be generated by specific cleavage and 3′ polyadenylation, a.k.a. polyA tail. A 3′ UTR may be operably linked to and located downstream of a transcribable polynucleotide molecule and may include polynucleotides that provide a polyadenylation signal and other regulatory signals capable of affecting transcription, mRNA processing, or gene expression. PolyA tails are thought to function in mRNA stability and in initiation of translation. Examples of 3′ transcription termination molecules in the art are the nopaline synthase 3′ region (see, Fraley, et al., Proc. Natl. Acad. Sci. USA, 80: 4803-4807 (1983)); wheat hsp17 3′ region; pea rubisco small subunit 3′ region; cotton E6 3′ region (U.S. Pat. No. 6,096,950); 3′ regions disclosed in WO0011200A2; and the coixin 3′ UTR (U.S. Pat. No. 6,635,806).

3′ UTRs typically find beneficial use for the recombinant expression of specific genes. In animal systems, a machinery of 3′ UTRs has been well defined (e.g. Zhao et al., Microbiol Mol Biol Rev 63:405-445 (1999); Proudfoot, Nature 322:562-565 (1986); Kim et al., Biotechnology Progress 19:1620-1622 (2003); Yonaha and Proudfoot, EMBO J. 19:3770-3777 (2000); Cramer et al., FEBS Letters 498:179-182 (2001); Kuerstem and Goodwin, Nature Reviews Genetics 4:626-637 (2003)). Effective termination of RNA transcription is required to prevent unwanted transcription of trait-unrelated (downstream) sequences, which may interfere with trait performance. Arrangement of multiple gene expression cassettes in local proximity to one another (e.g. within one T-DNA) may cause suppression of gene expression of one or more genes in said construct in comparison to independent insertions (Padidam and Cao, BioTechniques 31:328-334 (2001). This may interfere with achieving adequate levels of expression, for instance in cases were strong gene expression from all cassettes is desired.

In plants, clearly defined polyadenylation signal sequences are not known. Hasegawa et al., Plant J. 33:1063-1072, (2003)) were not able to identify conserved polyadenylation signal sequences in both in vitro and in vivo systems in Nicotiana sylvestris and to determine the actual length of the primary (non-polyadenylated) transcript. A weak 3′ UTR has the potential to generate read-through, which may affect the expression of the genes located in the neighboring expression cassettes (Padidam and Cao, BioTechniques 31:328-334 (2001)). Appropriate control of transcription termination can prevent read-through into sequences (e.g. other expression cassettes) localized downstream and can further allow efficient recycling of RNA polymerase, to improve gene expression. Efficient termination of transcription (release of RNA Polymerase II from the DNA) is pre-requisite for re-initiation of transcription and thereby directly affects the overall transcript level. Subsequent to transcription termination, the mature mRNA is released from the site of synthesis and template to the cytoplasm. Eukaryotic mRNAs are accumulated as poly(A) forms in vivo, so that it is difficult to detect transcriptional termination sites by conventional methods. However, prediction of functional and efficient 3′ UTRs by bioinformatics methods is difficult in that there are no conserved sequences which would allow easy prediction of an effective 3′ UTR.

From a practical standpoint, it is typically beneficial that a 3′ UTR used in a transgene cassette possesses the following characteristics. The 3′ UTR should be able to efficiently and effectively terminate transcription of the transgene and prevent read-through of the transcript into any neighboring DNA sequence which can be comprised of another transgene cassette as in the case of multiple cassettes residing in one T-DNA, or the neighboring chromosomal DNA into which the T-DNA has inserted. The 3′ UTR should not cause a reduction in the transcriptional activity imparted by the promoter, leader and introns that are used to drive expression of the transgene. In plant biotechnology, the 3′ UTR is often used for priming of amplification reactions of reverse transcribed RNA extracted from the transformed plant and used to (1) assess the transcriptional activity or expression of the transgene cassette once integrated into the plant chromosome; (2) assess the copy number of insertions within the plant DNA; and (3) assess zygosity of the resulting seed after breeding. The 3′ UTR is also used in amplification reactions of DNA extracted from the transformed plant to characterize the intactness of the inserted cassette.

3′ UTRs useful in providing expression of a transgene in plants may be identified based upon the expression of expressed sequence tags (ESTs) in cDNA libraries made from messenger RNA isolated from seed, flower and other tissues derived from Big bluestem (Andropogon gerardii), Plume grass (Saccharum ravennae (Erianthus ravennae)), Green bristlegrass (Setaria viridis), Teosinte (Zea mays subsp. mexicana), Foxtail millet (Setaria italica), or Coix (Coix lacryma-jobi). Libraries of cDNA are made from tissues isolated from selected plant species using methods known to those skilled in the art from flower tissue, seed, leaf and root. The resulting cDNAs are sequenced using various sequencing methods known in the art. The resulting ESTs are assembled into clusters using bioinformatics software such as clc_ref_assemble_complete version 2.01.37139 (CLC bio USA, Cambridge, Massachusetts 02142). Transcript abundance of each cluster is determined by counting the number of cDNA reads for each cluster. The identified 3′ UTRs may be comprised of sequence derived from cDNA sequence as well as sequence derived from genomic DNA. The cDNA sequence is used to design primers, which are then used with GenomeWalker™ (Clontech Laboratories, Inc, Mountain View, CA) libraries constructed following the manufacturer's protocol to clone the 3′ region of the corresponding genomic DNA sequence to provide a longer termination sequence. Analysis of relative transcript abundance either by direct counts or normalized counts of observed sequence reads for each tissue library can be used to infer properties about patters of expression. For example, some 3′ UTRs may be found in transcripts seen in higher abundance in root tissue as opposed to leaf. This is suggestive that the transcript is highly expressed in root and that the properties of root expression may be attributable to the transcriptional regulation of the promoter, the lead, the introns or the 3′ UTR. Empirical testing of 3′ UTRs identified by the properties of expression within specific organs, tissues or cell types can result in the identification of 3′ UTRs that enhance expression in those specific organs, tissues or cell types.

Constructs and vectors may also include a transit peptide coding sequence that expresses a linked peptide that is useful for targeting of a protein product, particularly to a chloroplast, leucoplast, or other plastid organelle; mitochondria; peroxisome; vacuole; or an extracellular location. For descriptions of the use of chloroplast transit peptides, see U.S. Pat. Nos. 5,188,642 and 5,728,925. Many chloroplast-localized proteins are expressed from nuclear genes as precursors and are targeted to the chloroplast by a chloroplast transit peptide (CTP). Examples of such isolated chloroplast proteins include, but are not limited to, those associated with the small subunit (SSU) of ribulose-1,5,-bisphosphate carboxylase, ferredoxin, ferredoxin oxidoreductase, the light-harvesting complex protein I and protein II, thioredoxin F, enolpyruvyl shikimate phosphate synthase (EPSPS), and transit peptides described in U.S. Pat. No. 7,193,133. It has been demonstrated in vivo and in vitro that non-chloroplast proteins may be targeted to the chloroplast by use of protein fusions with a heterologous CTP and that the CTP is sufficient to target a protein to the chloroplast. Incorporation of a suitable chloroplast transit peptide such as the Arabidopsis thaliana EPSPS CTP (CTP2) (See, Klee et al., Mol. Gen. Genet. 210:437-442 (1987)) or the Petunia hybrida EPSPS CTP (CTP4) (See, della-Cioppa et al., Proc. Natl. Acad. Sci. USA 83:6873-6877 (1986)) has been show to target heterologous EPSPS protein sequences to chloroplasts in transgenic plants (See, U.S. Pat. Nos. 5,627,061; 5,633,435; and 5,312,910 and EP 0218571; EP 189707; EP 508909; and EP 924299).

### Transcribable Polynucleotide Molecules

As used herein, the term “transcribable polynucleotide molecule” refers to any DNA molecule capable of being transcribed into a RNA molecule, including, but not limited to, those having protein coding sequences and those producing RNA molecules having sequences useful for gene suppression. A “transgene” refers to a transcribable polynucleotide molecule heterologous to a host cell at least with respect to its location in the genome and/or a transcribable polynucleotide molecule artificially incorporated into a host cell's genome in the current or any prior generation of the cell.

A promoter of the present invention may be operably linked to a transcribable polynucleotide molecule that is heterologous with respect to the promoter molecule. As used herein, the term “heterologous” refers to the combination of two or more polynucleotide molecules when such a combination is not normally found in nature. For example, the two molecules may be derived from different species and/or the two molecules may be derived from different genes, e.g. different genes from the same species or the same genes from different species. A promoter is thus heterologous with respect to an operably linked transcribable polynucleotide molecule if such a combination is not normally found in nature, i.e. that transcribable polynucleotide molecule is not naturally occurring operably linked in combination with that promoter molecule.

The transcribable polynucleotide molecule may generally be any DNA molecule for which expression of a RNA transcript is desired. Such expression of an RNA transcript may result in translation of the resulting mRNA molecule and thus protein expression. Alternatively, for example, a transcribable polynucleotide molecule may be designed to ultimately cause decreased expression of a specific gene or protein. In one embodiment, this may be accomplished by using a transcribable polynucleotide molecule that is oriented in the antisense direction. One of ordinary skill in the art is familiar with using such antisense technology. Briefly, as the antisense transcribable polynucleotide molecule is transcribed, the RNA product hybridizes to and sequesters a complimentary RNA molecule inside the cell. This duplex RNA molecule cannot be translated into a protein by the cell's translational machinery and is degraded in the cell. Any gene may be negatively regulated in this manner.

Thus, one embodiment of the invention is a regulatory element of the present invention, such as those provided as SEQ ID NOs: 1-158 and 180-183, operably linked to a transcribable polynucleotide molecule so as to modulate transcription of the transcribable polynucleotide molecule at a desired level or in a desired pattern when the construct is integrated in the genome of a plant cell. In one embodiment, the transcribable polynucleotide molecule comprises a protein-coding region of a gene, and the promoter affects the transcription of an RNA molecule that is translated and expressed as a protein product. In another embodiment, the transcribable polynucleotide molecule comprises an antisense region of a gene, and the promoter affects the transcription of an antisense RNA molecule, double stranded RNA or other similar inhibitory RNA molecule in order to inhibit expression of a specific RNA molecule of interest in a target host cell.

### Genes of Agronomic Interest

Transcribable polynucleotide molecules may be genes of agronomic interest. As used herein, the term “gene of agronomic interest” refers to a transcribable polynucleotide molecule that when expressed in a particular plant tissue, cell, or cell type confers a desirable characteristic, such as associated with plant morphology, physiology, growth, development, yield, product, nutritional profile, disease or pest resistance, and/or environmental or chemical tolerance. Genes of agronomic interest include, but are not limited to, those encoding a yield protein, a stress resistance protein, a developmental control protein, a tissue differentiation protein, a meristem protein, an environmentally responsive protein, a senescence protein, a hormone responsive protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, a pesticidal protein, or any other agent such as an antisense or RNAi molecule targeting a particular gene for suppression. The product of a gene of agronomic interest may act within the plant in order to cause an effect upon the plant physiology or metabolism or may be act as a pesticidal agent in the diet of a pest that feeds on the plant.

In one embodiment of the invention, a promoter of the present invention is incorporated into a construct such that the promoter is operably linked to a transcribable polynucleotide molecule that is a gene of agronomic interest. The expression of the gene of agronomic interest is desirable in order to confer an agronomically beneficial trait. A beneficial agronomic trait may be, for example, but is not limited to, herbicide tolerance, insect control, modified yield, fungal disease resistance, virus resistance, nematode resistance, bacterial disease resistance, plant growth and development, starch production, modified oils production, high oil production, modified fatty acid content, high protein production, fruit ripening, enhanced animal and human nutrition, biopolymers, environmental stress resistance, pharmaceutical peptides and secretable peptides, improved processing traits, improved digestibility, enzyme production, flavor, nitrogen fixation, hybrid seed production, fiber production, and biofuel production. Examples of genes of agronomic interest known in the art include those for herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; and 5,763,241), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6,171,640), biopolymers (U.S. Pat. Nos. RE37,543; 6,228,623; and U.S. Pat. Nos. 5,958,745, and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700).

Alternatively, a gene of agronomic interest can affect the above mentioned plant characteristic or phenotype by encoding a RNA molecule that causes the targeted modulation of gene expression of an endogenous gene, for example via antisense (see e.g. U.S. Pat. No. 5,107,065); inhibitory RNA (“RNAi”, including modulation of gene expression via miRNA-, siRNA-, trans-acting siRNA-, and phased sRNA-mediated mechanisms, e.g. as described in published applications US 2006/0200878 and US 2008/0066206, and in U.S. patent application Ser. No. 11/974,469); or cosuppression-mediated mechanisms. The RNA could also be a catalytic RNA molecule (e.g. a ribozyme or a riboswitch; see e.g. US 2006/0200878) engineered to cleave a desired endogenous mRNA product. Thus, any transcribable polynucleotide molecule that encodes a transcribed RNA molecule that affects an agronomically important phenotype or morphology change of interest may be useful for the practice of the present invention. Methods are known in the art for constructing and introducing constructs into a cell in such a manner that the transcribable polynucleotide molecule is transcribed into a molecule that is capable of causing gene suppression. For example, posttranscriptional gene suppression using a construct with an anti-sense oriented transcribable polynucleotide molecule to regulate gene expression in plant cells is disclosed in U.S. Pat. Nos. 5,107,065 and 5,759,829, and posttranscriptional gene suppression using a construct with a sense-oriented transcribable polynucleotide molecule to regulate gene expression in plants is disclosed in U.S. Pat. Nos. 5,283,184 and 5,231,020. Expression of a transcribable polynucleotide in a plant cell can also be used to suppress plant pests feeding on the plant cell, for example, compositions isolated from coleopteran pests (U.S. Patent Publication No. US20070124836) and compositions isolated from nematode pests (U.S. Patent Publication No. US20070250947). Plant pests include, but are not limited to arthropod pests, nematode pests, and fungal or microbial pests. Exemplary transcribable polynucleotide molecules for incorporation into constructs of the present invention include, for example, DNA molecules or genes from a species other than the target species or genes that originate with or are present in the same species, but are incorporated into recipient cells by genetic engineering methods rather than classical reproduction or breeding techniques. The type of polynucleotide molecule can include, but is not limited to, a polynucleotide molecule that is already present in the plant cell, a polynucleotide molecule from another plant, a polynucleotide molecule from a different organism, or a polynucleotide molecule generated externally, such as a polynucleotide molecule containing an antisense message of a gene, or a polynucleotide molecule encoding an artificial, synthetic, or otherwise modified version of a transgene.

### Selectable Markers

As used herein the term “marker” refers to any transcribable polynucleotide molecule whose expression, or lack thereof, can be screened for or scored in some way. Marker genes for use in the practice of the present invention include, but are not limited to transcribable polynucleotide molecules encoding β-glucuronidase (GUS described in U.S. Pat. No. 5,599,670), green fluorescent protein and variants thereof (GFP described in U.S. Pat. Nos. 5,491,084 and 6,146,826), proteins that confer antibiotic resistance, or proteins that confer herbicide tolerance. Useful antibiotic resistance markers, including those encoding proteins conferring resistance to kanamycin (nptII), hygromycin B (aph IV), streptomycin or spectinomycin (aad, spec/strep) and gentamycin (aac3 and aacC4) are known in the art. Herbicides for which transgenic plant tolerance has been demonstrated and the method of the present invention can be applied, include, but are not limited to: amino-methyl-phosphonic acid, glyphosate, glufosinate, sulfonylureas, imidazolinones, bromoxynil, dalapon, dicamba, cyclohexanedione, protoporphyrinogen oxidase inhibitors, and isoxasflutole herbicides. Transcribable polynucleotide molecules encoding proteins involved in herbicide tolerance are known in the art, and include, but are not limited to, a transcribable polynucleotide molecule encoding 5-enolpyruvylshikimate-3-phosphate synthase (EPSPS for glyphosate tolerance described in U.S. Pat. Nos. 5,627,061; 5,633,435; 6,040,497; and 5,094,945); a transcribable polynucleotide molecule encoding a glyphosate oxidoreductase and a glyphosate-N-acetyl transferase (GOX described in U.S. Pat. No. 5,463,175; GAT described in U.S. Patent publication No. 20030083480, and dicamba monooxygenase U.S. Patent publication No. 20030135879); a transcribable polynucleotide molecule encoding bromoxynil nitrilase (Bxn for Bromoxynil tolerance described in U.S. Pat. No. 4,810,648); a transcribable polynucleotide molecule encoding phytoene desaturase (crtl) described in Misawa, et al., Plant Journal 4:833-840 (1993) and Misawa, et al., Plant Journal 6:481-489 (1994) for norflurazon tolerance; a transcribable polynucleotide molecule encoding acetohydroxyacid synthase (AHAS, aka ALS) described in Sathasiivan, et al., Nucl. Acids Res. 18:2188-2193 (1990) for tolerance to sulfonylurea herbicides; and the bar gene described in DeBlock, et al., EMBO Journal 6:2513-2519 (1987) for glufosinate and bialaphos tolerance. The promoter molecules of the present invention can express linked transcribable polynucleotide molecules that encode for phosphinothricin acetyltransferase, glyphosate resistant EPSPS, aminoglycoside phosphotransferase, hydroxyphenyl pyruvate dehydrogenase, hygromycin phosphotransferase, neomycin phosphotransferase, dalapon dehalogenase, bromoxynil resistant nitrilase, anthranilate synthase, aryloxyalkanoate dioxygenases, acetyl CoA carboxylase, glyphosate oxidoreductase, and glyphosate-N-acetyl transferase.

Included within the term “selectable markers” are also genes which encode a secretable marker whose secretion can be detected as a means of identifying or selecting for transformed cells. Examples include markers that encode a secretable antigen that can be identified by antibody interaction, or even secretable enzymes which can be detected catalytically. Selectable secreted marker proteins fall into a number of classes, including small, diffusible proteins which are detectable, (e.g. by ELISA), small active enzymes which are detectable in extracellular solution (e.g, alpha-amylase, beta-lactamase, phosphinothricin transferase), or proteins which are inserted or trapped in the cell wall (such as proteins which include a leader sequence such as that found in the expression unit of extension or tobacco pathogenesis related proteins also known as tobacco PR-S). Other possible selectable marker genes will be apparent to those of skill in the art and are encompassed by the present invention.

### Cell Transformation

The invention is also directed to a method of producing transformed cells and plants which comprise a promoter operably linked to a transcribable polynucleotide molecule.

The term “transformation” refers to the introduction of nucleic acid into a recipient host. As used herein, the term “host” refers to bacteria, fungi, or plant, including any cells, tissue, organs, or progeny of the bacteria, fungi, or plant. Plant tissues and cells of particular interest include protoplasts, calli, roots, tubers, seeds, stems, leaves, seedlings, embryos, and pollen.

As used herein, the term “transformed” refers to a cell, tissue, organ, or organism into which a foreign polynucleotide molecule, such as a construct, has been introduced. The introduced polynucleotide molecule may be integrated into the genomic DNA of the recipient cell, tissue, organ, or organism such that the introduced polynucleotide molecule is inherited by subsequent progeny. A “transgenic” or “transformed” cell or organism also includes progeny of the cell or organism and progeny produced from a breeding program employing such a transgenic organism as a parent in a cross and exhibiting an altered phenotype resulting from the presence of a foreign polynucleotide molecule. The term “transgenic” refers to a bacteria, fungi, or plant containing one or more heterologous polynucleic acid molecules.

There are many methods for introducing polynucleic acid molecules into plant cells. The method generally comprises the steps of selecting a suitable host cell, transforming the host cell with a recombinant vector, and obtaining the transformed host cell. Suitable methods include bacterial infection (e.g. Agrobacterium), binary bacterial artificial chromosome vectors, direct delivery of DNA (e.g. via PEG-mediated transformation, desiccation/inhibition-mediated DNA uptake, electroporation, agitation with silicon carbide fibers, and acceleration of DNA coated particles, etc. (reviewed in Potrykus, et al., Ann. Rev. Plant Physiol. Plant Mol. Biol. 42: 205 (1991)).

Technology for introduction of a DNA molecule into cells is well known to those of skill in the art. Methods and materials for transforming plant cells by introducing a plant DNA construct into a plant genome in the practice of this invention can include any of the well-known and demonstrated methods. Any transformation methods may be utilized to transform a host cell with one or more promoters and/or constructs of the present. Host cells may be any cell or organism such as a plant cell, algae cell, algae, fungal cell, fungi, bacterial cell, or insect cell. Preferred hosts and transformed cells include cells from: plants, Aspergillus, yeasts, insects, bacteria and algae.

Regenerated transgenic plants can be self-pollinated to provide homozygous transgenic plants. Alternatively, pollen obtained from the regenerated transgenic plants may be crossed with non-transgenic plants, preferably inbred lines of agronomically important species. Descriptions of breeding methods that are commonly used for different traits and crops can be found in one of several reference books, see, for example, Allard, Principles of Plant Breeding, John Wiley & Sons, NY, U. of CA, Davis, CA, 50-98 (1960); Simmonds, Principles of crop improvement, Longman, Inc., NY, 369-399 (1979); Sneep and Hendriksen, Plant breeding perspectives, Wageningen (ed), Center for Agricultural Publishing and Documentation (1979); Fehr, Soybeans: Improvement, Production and Uses, 2nd Edition, Monograph, 16:249 (1987); Fehr, Principles of variety development, Theory and Technique, (Vol. 1) and Crop Species Soybean (Vol 2), Iowa State Univ., Macmillan Pub. Co., NY, 360-376 (1987). Conversely, pollen from non-transgenic plants may be used to pollinate the regenerated transgenic plants.

The transformed plants may be analyzed for the presence of the genes of interest and the expression level and/or profile conferred by the regulatory elements of the present invention. Those of skill in the art are aware of the numerous methods available for the analysis of transformed plants. For example, methods for plant analysis include, but are not limited to Southern blots or northern blots, PCR-based approaches, biochemical analyses, phenotypic screening methods, field evaluations, and immunodiagnostic assays. The expression of a transcribable polynucleotide molecule can be measured using TaqMan® (Applied Biosystems, Foster City, CA) reagents and methods as described by the manufacturer and PCR cycle times determined using the TaqMan® Testing Matrix. Alternatively, the Invader® (Third Wave Technologies, Madison, WI) reagents and methods as described by the manufacturer can be used transgene expression.

The seeds of the plants of this invention can be harvested from fertile transgenic plants and be used to grow progeny generations of transformed plants of this invention including hybrid plant lines comprising the construct of this invention and expressing a gene of agronomic interest.

The present invention also provides for parts of the plants of the present invention. Plant parts, without limitation, include leaves, stems, roots, tubers, seeds, endosperm, ovule, and pollen. The invention also includes and provides transformed plant cells which comprise a nucleic acid molecule of the present invention.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. The progeny from these plants are evaluated, among other things, for gene expression. The gene expression may be detected by several common methods such as western blotting, northern blotting, immuno-precipitation, and ELISA.

### Commodity Products

The present invention provides a commodity product comprising DNA molecules according to the invention. As used herein, a “commodity product” refers to any composition or product which is comprised of material derived from a plant, seed, plant cell or plant part comprising a DNA molecule of the invention. Commodity products may be sold to consumers and may be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animals consumption, oil, meal, flour, flakes, bran, fiber, milk, cheese, paper, cream, wine, and any other food for human consumption; and biomasses and fuel products. Viable commodity products include but are not limited to seeds and plant cells. Plants comprising a DNA molecule according to the invention can thus be used to manufacture any commodity product typically acquired from plants or parts thereof.

Having now generally described the invention, the same will be more readily understood through reference to the following examples which are provided by way of illustration, and are not intended to be limiting of the present invention, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1: Identification and Cloning of Regulatory Elements

Novel ubiquitin transcriptional regulatory elements, or transcriptional regulatory expression element group (EXP) sequences were identified and isolated from genomic DNA of the monocot species Big bluestem (Andropogon gerardii), Plume Grass (Saccharum ravennae (Erianthus ravennae)), Green bristlegrass (Setaria viridis), Teosinte (Zea mays subsp. mexicana), Foxtail millet (Setaria italica), and Coix (Coix lacryma-jobi).

Ubiquitin 1 transcript sequences were identified from each of the above species. The 5′ untranslated region (5′ UTR) of each of the Ubiquitin 1 transcripts was used to design primers to amplify the corresponding transcriptional regulatory elements for the identified Ubiquitin gene, which comprises a promoter, leader (5′ UTR) and first intron operably linked. The primers were used with GenomeWalker™ (Clontech Laboratories, Inc, Mountain View, CA) libraries constructed following the manufacturer's protocol to clone the 5′ region of the corresponding genomic DNA sequence. Ubiquitin transcriptional regulatory elements were also isolated from the monocot Sorghum bicolor using public sequences that are homologs to the Ubiquitin 4, 6 and 7 genes of Zea mays.

Using the identified sequences, a bioinformatic analysis was conducted to identify regulatory elements within the amplified DNA. Using the results of this analysis, regulatory elements were defined within the DNA sequences and primers designed to amplify the regulatory elements. The corresponding DNA molecule for each regulatory element was amplified using standard polymerase chain reaction conditions with primers containing unique restriction enzyme sites and genomic DNA isolated from A. gerardii, S. ravennae, S. viridis, Z. mays subsp. mexicana, S. italica, C. lacryma-jobi, and S. bicolor. The resulting DNA fragments were ligated into base plant expression vectors and sequenced. An analysis of the regulatory element TSS and intron/exon splice junctions was then done using transformed plant protoplasts. Briefly, the protoplasts were transformed with the plant expression vectors comprising the cloned DNA fragments operably linked to a heterologous transcribable polynucleotide molecule and the 5′ RACE System for Rapid Amplification of cDNA Ends, Version 2.0 (Invitrogen, Carlsbad, California 92008) was used to confirm the regulatory element TSS and intron/exon splice junctions by analyzing the sequence of the mRNA transcripts produced thereby.

Sequences of the identified transcriptional regulatory expression element groups (“EXP's”) are provided herein as SEQ ID NOS: 1, 5, 8, 10, 12, 14, 16, 18, 22, 25, 27, 29, 31, 33, 37, 39, 41, 45, 49, 53, 55, 59, 63, 65, 69, 73, 75, 77, 79, 83, 85, 87, 90, 93, 95, 97, 98, 99, 100, 102, 104, 106, 108, 110, 112, 114, 115, 116, 117, 119, 121, 123, 124, 125, 126, 128, 130, 132, 133, 134, 136, 137, 139, 141, 143, 145, 147, 149, 151, 153, 155, 157, 180, 181 and 183, as listed in Table 1 below. Promoter sequences are provided herein as SEQ ID NOS: 2, 6, 9, 11, 13, 15, 17, 19, 23, 26, 28, 30, 32, 34, 38, 40, 42, 46, 50, 56, 60, 64, 66, 70, 74, 76, 78, 80, 84, 86, 88, 91, 96 and 135. Leader sequences are provided herein as SEQ ID NOS: 3, 20, 35, 43, 47, 51, 57, 61, 67,71 and 81. Intron sequences are provided herein as SEQ ID NOS: 4,7, 21, 24, 36, 44, 48, 52, 54, 58, 62, 68, 72, 82, 92, 94, 101, 103, 105, 107, 109, 111, 113, 118, 120, 122, 127, 129, 131, 138, 140, 142, 144, 146, 148, 150, 152, 154, 156, 158 and 182. An enhancer sequence is provided as SEQ ID NO: 89.

TABLE 1
Transcriptional regulatory expression element groups (“EXP's”),
promoters, enhancers, leaders and introns isolated from various grass species.

| | SEQ | | | | Plasmid Construct(s) |
|---|---|---|---|---|---|
| | ID | Size | Source | Description and/or regulatory elements of EXP | and Amplicons |
| Annotation | NO: | (bp) | Genus/species | linked in 5′ → 3′ direction (SEQ ID NOs): | comprising EXP |
| EXP-ANDge.Ubq1:1:9 | 1 | 3741 | A. gerardii | EXP: P-ANDge.Ubq1-1:1:11 (SEQ ID NO: 2); | |
| | | | | L-ANDge.Ubq1-1:1:2 (SEQ ID NO: 3); | |
| | | | | I-ANDge.Ubq1-1:1:3 (SEQ ID NO: 4). | |
| P-ANDge.Ubq1-1:1:11 | 2 | 2603 | A. gerardii | promoter | |
| L-ANDge.Ubq1-1:1:2 | 3 | 99 | A. gerardii | leader | |
| I-ANDge.Ubq1-1:1:3 | 4 | 1039 | A. gerardii | intron | |
| EXP-ANDge.Ubq1:1:7 | 5 | 3255 | A. gerardii | EXP: P-ANDge.Ubq1-1:1:9 (SEQ ID NO: 6); | pMON136264, |
| | | | | L-ANDge.Ubq1-1:1:2 (SEQ ID NO: 3); | PCR0145892, |
| | | | | I-ANDge.Ubq1-1:1:4 (SEQ ID NO: 7). | pMON140896, |
| | | | | | PCR41 |
| P-ANDge.Ubq1-1:1:9 | 6 | 2114 | A. gerardii | promoter | |
| I-ANDge.Ubq1-1:1:4 | 7 | 1042 | A. gerardii | intron | |
| EXP-ANDge.Ubq1:1:8 | 8 | 2785 | A. gerardii | EXP: P-ANDge.Ubq1-1:1:10 (SEQ ID NO: 9); | pMON140917, |
| | | | | L-ANDge.Ubq1-1:1:2 (SEQ ID NO: 3); | PCR42 |
| | | | | I-ANDge.Ubq1-1:1:4 (SEQ ID NO: 7). | |
| P-ANDge.Ubq1-1:1:10 | 9 | 1644 | A. gerardii | promoter | |
| EXP-ANDge.Ubq1:1:10 | 10 | 2613 | A. gerardii | EXP: P-ANDge.Ubq1-1:1:12 (SEQ ID NO: 11); | PCR0145815, |
| | | | | L-ANDge.Ubq1-1:1:2 (SEQ ID NO: 3); | PCR43 |
| | | | | I-ANDge.Ubq1-1:1:4 (SEQ ID NO: 7). | |
| P-ANDge.Ubq1-1:1:12 | 11 | 1472 | A. gerardii | promoter | |
| EXP-ANDge.Ubq1:1:6 | 12 | 2255 | A. gerardii | EXP: P-ANDge.Ubq1-1:1:8 (SEQ ID NO: 13); | pMON136259, |
| | | | | L-ANDge.Ubq1-1:1:2 (SEQ ID NO: 3); | PCR0145893, |
| | | | | I-ANDge.Ubq1-1:1:4 (SEQ ID NO: 7). | pMON140898, |
| | | | | | PCR44 |
| P-ANDge.Ubq1-1:1:8 | 13 | 1114 | A. gerardii | promoter | |
| EXP-ANDge.Ubq1:1:11 | 14 | 1912 | A. gerardii | EXP: P-ANDge.Ubq1-1:1:13 (SEQ ID NO: 15); | PCR0145817, |
| | | | | L-ANDge.Ubq1-1:1:2 (SEQ ID NO: 3); | pMON140899, |
| | | | | I-ANDge.Ubq1-1:1:4 (SEQ ID NO: 7). | PCR45 |
| P-ANDge.Ubq1-1:1:13 | 15 | 771 | A. gerardii | promoter | |
| EXP-ANDge.Ubq1:1:12 | 16 | 1623 | A. gerardii | EXP: P-ANDge.Ubq1-1:1:14 (SEQ ID NO: 17); | PCR0145819, |
| | | | | L-ANDge.Ubq1-1:1:2 (SEQ ID NO: 3); | pMON140900, |
| | | | | I-ANDge.Ubq1-1:1:4 (SEQ ID NO: 7). | PCR46 |
| P-ANDge.Ubq1-1:1:14 | 17 | 482 | A. gerardii | promoter | |
| EXP-ERIra.Ubq1 | 18 | 3483 | E. ravennae | EXP: P-ERIra.Ubq1-1:1:10 (SEQ ID NO: 19); | |
| | | | | L-ERIra.Ubq1-1:1:2 (SEQ ID NO: 20); | |
| | | | | I-ERIra.Ubq1-1:1:1 (SEQ ID NO: 21). | |
| P-ERIra.Ubq1-1:1:10 | 19 | 2536 | E. ravennae | promoter | |
| L-ERIra.Ubq1-1:1:2 | 20 | 94 | E. ravennae | leader | |
| I-ERIra.Ubq1-1:1:1 | 21 | 1041 | E. ravennae | intron | |
| EXP-ERIra.Ubq1:1:9 | 22 | 3152 | E. ravennae | EXP: P-ERIra.Ubq1-1:1:9 (SEQ ID NO: 23); | pMON136263, |
| | | | | L-ERIra.Ubq1-1:1:2 (SEQ ID NO: 20); | PCR0145896, |
| | | | | I-ERIra.Ubq1-1:1:2 (SEQ ID NO: 24). | pMON140904, |
| | | | | | PCR50 |
| P-ERIra.Ubq1-1:1:9 | 23 | 2014 | E. ravennae | promoter | |
| I-ERIra.Ubq1-1:1:2 | 24 | 1044 | E. ravennae | intron | |
| EXP-ERIra.Ubq1:1:10 | 25 | 2663 | E. ravennae | EXP: P-ERIra.Ubq1-1:1:11 (SEQ ID NO: 26); | PCR0145820, |
| | | | | L-ERIra.Ubq1-1:1:2 (SEQ ID NO: 20); | pMON140905, |
| | | | | I-ERIra.Ubq1-1:1:2 (SEQ ID NO: 24). | PCR51 |
| P-ERIra.Ubq1-1:1:11 | 26 | 1525 | E. ravennae | promoter | |
| EXP-ERIra.Ubq1:1:8 | 27 | 2182 | E. ravennae | EXP: P-ERIra.Ubq1-1:1:8 (SEQ ID NO: 28); | pMON136258, |
| | | | | L-ERIra.Ubq1-1:1:2 (SEQ ID NO: 20); | PCR0145897, |
| | | | | I-ERIra.Ubq1-1:1:2 (SEQ ID NO: 24). | pMON140906, |
| | | | | | PCR52, |
| | | | | | pMON142864, |
| | | | | | pMON142862 |
| P-ERIra.Ubq1-1:1:8 | 28 | 1044 | E. ravennae | promoter | |
| EXP-ERIra.Ubq1:1:11 | 29 | 1934 | E. ravennae | EXP: P-ERIra.Ubq1-1:1:12 (SEQ ID NO: 30); | PCR0145821, |
| | | | | L-ERIra.Ubq1-1:1:2 (SEQ ID NO: 20); | pMON140907, |
| | | | | I-ERIra.Ubq1-1:1:2 (SEQ ID NO: 24). | PCR53 |
| P-ERIra.Ubq1-1:1:12 | 30 | 796 | E. ravennae | promoter | |
| EXP-ERIra.Ubq1:1:12 | 31 | 1649 | E. ravennae | EXP: P-ERIra.Ubq1-1:1:13 (SEQ ID NO: 32); | PCR0145822, |
| | | | | L-ERIra.Ubq1-1:1:2 (SEQ ID NO: 20); | pMON140908, |
| | | | | I-ERIra.Ubq1-1:1:2 (SEQ ID NO: 24). | PCR54 |
| P-ERIra.Ubq1-1:1:13 | 32 | 511 | E. ravennae | promoter | |
| EXP-Sv.Ubq1:1:2 | 33 | 2631 | S. viridis | EXP: P-Sv.Ubq1-1:1:1 (SEQ ID NO: 34); | pMON140878, |
| | | | | L-Sv.Ubq1-1:1:2 (SEQ ID NO: 35); | PCR0145909, |
| | | | | I-Sv.Ubq1-1:1:1 (SEQ ID NO: 36). | pMON129203, |
| | | | | | pMON131958 |
| P-Sv.Ubq1-1:1:1 | 34 | 1493 | S. viridis | promoter | |
| L-Sv.Ubq1-1:1:2 | 35 | 127 | S. viridis | leader | |
| I-Sv.Ubq1-1:1:1 | 36 | 1011 | S. viridis | intron | |
| EXP-Sv.Ubq1:1:3 | 37 | 2173 | S. viridis | EXP: P-Sv.Ubq1-1:1:2 (SQ ID NO: 38); | PCR0145929, |
| | | | | L-Sv.Ubq1-1:1:2 (SEQ ID NO: 35); | pMON129204 |
| | | | | I-Sv.Ubq1-1:1:1 (SEQ ID NO: 36). | |
| P-Sv.Ubq1-1:1:2 | 38 | 1035 | S. viridis | promoter | |
| EXP-Sv.Ubq1:1:5 | 39 | 1819 | S. viridis | EXP: P-Sv.Ubq1-1:1:3 (SEQ ID NO: 40); | pMON129205, |
| | | | | L-Sv.Ubq1-1:1:2 (SEQ ID NO: 35); | pMON131959 |
| | | | | I-Sv.Ubq1-1:1:1 (SEQ ID NO: 36). | |
| P-Sv.Ubq1-1:1:3 | 40 | 681 | S. viridis | promoter | |
| EXP-Zm.UbqM1:1:1 | 41 | 1922 | Z. mays subsp. | EXP: P-Zm.UbqM1-1:1:1 (SEQ ID NO: 42); | pMON140881, |
| (Allele-1) | | | mexicana | L-Zm.UbqM1-1:1:1 (SEQ ID NO: 43); | PCR0145914, |
| | | | | I-Zm.UbqM1-1:1:5 (SEQ ID NO: 44). | pMON129210, |
| | | | | | pMON131961 |
| P-Zm.UbqM1-1:1:1 | 42 | 850 | Z. mays subsp. | promoter | |
| (Allele-1) | | | mexicana | | |
| L-Zm.UbqM1-1:1:1 | 43 | 78 | Z. mays subsp. | leader | |
| (Allele-1) | | | mexicana | | |
| I-Zm.UbqM1-1:1:5 | 44 | 994 | Z. mays subsp. | intron | |
| (Allele-1) | | | mexicana | | |
| EXP-Zm.UbqM1:1:4 | 45 | 1971 | Z. mays subsp. | EXP: P-Zm.UbqM1-1:1:4 (SEQ ID NO: 46); | pMON140882, |
| (Allele-2) | | | mexicana | L-Zm.UbqM1-1:1:5 (SEQ ID NO: 47); | PCR0145915, |
| | | | | I-Zm.UbqM1-1:1:4 (SEQ ID NO: 48). | pMON129212, |
| | | | | | pMON131963 |
| P-Zm.UbqM1-1:1:4 | 46 | 887 | Z. mays subsp. | promoter | |
| (Allele-2) | | | mexicana | | |
| L-Zm.UbqM1-1:1:5 | 47 | 77 | Z. mays subsp. | leader | |
| (Allele-2) | | | mexicana | | |
| I-Zm.UbqM1-1:1:4 | 48 | 1007 | Z. mays subsp. | intron | |
| (Allele-2) | | | mexicana | | |
| EXP-Zm.UbqM1:1:2 | 49 | 2005 | Z. mays subsp. | EXP: P-Zm.UbqM1-1:1:5 (SEQ ID NO: 50); | PCR0145916, |
| (Allele-3) | | | mexicana | L-Zm.UbqM1-1:1:4 (SEQ ID NO: 51); | pMON129211, |
| | | | | I-Zm.UbqM1-1:1:11 (SEQ ID NO: 52). | pMON131962, |
| | | | | | pMON132047 |
| P-Zm.UbqM1-1:1:5 | 50 | 877 | Z. mays subsp. | promoter | |
| (Allele-3) | | | mexicana | | |
| L-Zm.UbqM1-1:1:4 | 51 | 78 | Z. mays subsp. | leader | |
| (Allele-3) | | | mexicana | | |
| I-Zm.UbqM1-1:1:11 | 52 | 1050 | Z. mays subsp. | intron | |
| (Allele-3) | | | mexicana | | |
| EXP-Zm.UbqM1:1:5 | 53 | 2005 | Z. mays subsp. | EXP: P-Zm.UbqM1-1:1:5 (SEQ ID NO: 50); | |
| (Allele-3) | | | mexicana | L-Zm.UbqM1-1:1:4 (SEQ ID NO: 51); | |
| | | | | I-Zm.UbqM1-1:1:12 (SEQ ID NO: 54). | |
| I-Zm.UbqM1-1:1:12 | 54 | 1050 | Z. mays subsp. | intron | |
| (Allele-3) | | | mexicana | | |
| EXP-Sb.Ubq4:1:1 | 55 | 1632 | S. bicolor | EXP: P-Sb.Ubq4-1:1:1 (SEQ ID NO: 56); | pMON140886, |
| | | | | L-Sb.Ubq4-1:1:1 (SEQ ID NO: 57); | PCR0145921, |
| | | | | I-Sb.Ubq4-1:1:1 (SEQ ID NO: 58). | pMON129219, |
| | | | | | pMON132932 |
| P-Sb.Ubq4-1:1:1 | 56 | 401 | S. bicolor | promoter | |
| L-Sb.Ubq4-1:1:1 | 57 | 154 | S. bicolor | leader | |
| I-Sb.Ubq4-1:1:1 | 58 | 1077 | S. bicolor | intron | |
| EXP-Sb.Ubq6 | 59 | 2000 | S. bicolor | EXP: P-Sb.Ubq6-1:1:2 (SEQ ID NO: 60); | |
| | | | | L-Sb.Ubq6-1:1:1 (SEQ ID NO: 61); | |
| | | | | I-Sb.Ubq6-1:1:1 (SEQ ID NO: 62). | |
| P-Sb.Ubq6-1:1:2 | 60 | 791 | S. bicolor | promoter | |
| L-Sb.Ubq6-1:1:1 | 61 | 136 | S. bicolor | leader | |
| I-Sb.Ubq6-1:1:1 | 62 | 1073 | S. bicolor | intron | |
| EXP-Sb.Ubq6:1:1 | 63 | 2064 | S. bicolor | EXP: P-Sb.Ubq6-1:1:1 (SEQ ID NO: 64); | pMON140887, |
| | | | | L-Sb.Ubq6-1:1:1 (SEQ ID NO: 61); | PCR0145920, |
| | | | | I-Sb.Ubq6-1:1:1 (SEQ ID NO: 62). | pMON129218 |
| P-Sb.Ubq6-1:1:1 | 64 | 855 | S. bicolor | promoter | |
| EXP-Sb.Ubq7:1:1 | 65 | 2000 | S. bicolor | EXP: P-Sb.Ubq7-1:1:1 (SEQ ID NO: 66); | pMON132974 |
| | | | | L-Sb.Ubq7-1:1:1 (SEQ ID NO: 67); | |
| | | | | I-Sb.Ubq7-1:1:1 (SEQ ID NO: 68). | |
| P-Sb.Ubq7-1:1:1 | 66 | 565 | S. bicolor | promoter | |
| L-Sb.Ubq7-1:1:1 | 67 | 77 | S. bicolor | leader | |
| I-Sb.Ubq7-1:1:1 | 68 | 1358 | S. bicolor | intron | |
| EXP-SETit.Ubq1:1:1 | 69 | 2622 | S. italica | EXP: P-SETit.Ubq1-1:1:1 (SEQ ID NO: 70); | pMON140877, |
| | | | | L-SETit.Ubq1-1:1:1 (SEQ ID NO: 71); | PCR0145900, |
| | | | | I-SETit.Ubq1-1:1:1 (SEQ ID NO: 72). | pMON129200 |
| P-SETit.Ubq1-1:1:1 | 70 | 1492 | S. italica | promoter | |
| L-SETit.Ubq1-1:1:1 | 71 | 127 | S. italica | leader | |
| I-SETit.Ubq1-1:1:1 | 72 | 1003 | S. italica | intron | |
| EXP-SETit.Ubq1:1:4 | 73 | 2622 | S. italica | EXP: P-SETit.Ubq1-1:1:4 (SEQ ID NO: 74); | pMON132037 |
| | | | | L-SETit.Ubq1-1:1:1 (SEQ ID NO: 71); | |
| | | | | I-SETit.Ubq1-1:1:1 (SEQ ID NO: 72). | |
| P-SETit.Ubq1-1:1:4 | 74 | 1492 | S. italica | promoter | |
| EXP-SETit.Ubq1:1:2 | 75 | 2164 | S. italica | EXP: P-SETit.Ubq1-1:1:2 (SEQ ID NO: 76); | |
| | | | | L-SETit.Ubq1-1:1:1 (SEQ ID NO: 71); | |
| | | | | I-SETit.Ubq1-1:1:1 (SEQ ID NO: 72). | |
| P-SETit.Ubq1-1:1:2 | 76 | 1034 | S. italica | promoter | |
| EXP-SETit.Ubq1:1:3 | 77 | 1810 | S. italica | EXP: P-SETit.Ubq1-1:1:3 (SEQ ID NO: 78); | PCR0145905, |
| | | | | L-SETit.Ubq1-1:1:1 (SEQ ID NO: 71); | pMON129202, |
| | | | | I-SETit.Ubq1-1:1:1 (SEQ ID NO: 72). | pMON131957 |
| P-SETit.Ubq1-1:1:3 | 78 | 680 | S. italica | promoter | |
| EXP-Cl.Ubq1:1:1 | 79 | 1940 | C. lacryma-jobi | EXP: P-Cl.Ubq1-1:1:1 (SEQ ID NO: 80); | pMON140889, |
| | | | | L-Cl.Ubq1-1:1:1 (SEQ ID NO: 81); | PCR0145922, |
| | | | | I-Cl.Ubq1-1:1:1 (SEQ ID NO: 82). | pMON140913, |
| | | | | | PCR19, |
| | | | | | pMON129221, |
| | | | | | pMON146795, |
| | | | | | pMON146796, |
| | | | | | pMON146797, |
| | | | | | pMON146798, |
| | | | | | pMON146799, |
| | | | | | pMON132047, |
| | | | | | pMON146800, |
| | | | | | pMON146801, |
| | | | | | pMON146802 |
| P-Cl.Ubq1-1:1:1 | 80 | 837 | C. lacryma-jobi | promoter | |
| L-Cl.Ubq1-1:1:1 | 81 | 86 | C. lacryma-jobi | leader | |
| I-Cl.Ubq1-1:1:1 | 82 | 1017 | C. lacryma-jobi | intron | |
| EXP-Cl.Ubq1:1:3 | 83 | 1845 | C. lacryma-jobi | EXP: P-Cl.Ubq1-1:1:4 (SEQ ID NO: 84); | PCR0145945, |
| | | | | L-Cl.Ubq1-1:1:1 (SEQ ID NO: 81); | pMON140914, |
| | | | | I-Cl.Ubq1-1:1:1 (SEQ ID NO: 82). | PCR20 |
| P-Cl.Ubq1-1:1:4 | 84 | 742 | C. lacryma-jobi | promoter | |
| EXP-Cl.Ubq1:1:4 | 85 | 1504 | C. lacryma-jobi | EXP: P-Cl.Ubq1-1:1:3 (SEQ ID NO: 86); | PCR0145946, |
| | | | | L-Cl.Ubq1-1:1:1 (SEQ ID NO: 81); | pMON140915, |
| | | | | I-Cl.Ubq1-1:1:1 (SEQ ID NO: 82). | PCR21 |
| P-Cl.Ubq1-1:1:3 | 86 | 401 | C. lacryma-jobi | promoter | |
| EXP-Cl.Ubq1:1:5 | 87 | 1157 | C. lacryma-jobi | EXP: P-Cl.Ubq1-1:1:5 (SEQ ID NO: 88); | PCR0145947, |
| | | | | L-Cl.Ubq1-1:1:1 (SEQ ID NO: 81); | pMON140916, |
| | | | | I-Cl.Ubq1-1:1:1 (SEQ ID NO: 82). | PCR22 |
| P-Cl.Ubq1-1:1:5 | 88 | 54 | C. lacryma-jobi | promoter | |
| E-Cl.Ubq1-1:1:1 | 89 | 798 | C. lacryma-jobi | enhancer | |
| EXP-Cl.Ubq1:1:12 | 90 | 3393 | C. lacryma-jobi | EXP: P-Cl.Ubq1-1:1:9 (SEQ ID NO: 91); | pMON142729 |
| | | | | L-Cl.Ubq1-1:1:1 (SEQ ID NO: 81); | |
| | | | | I-Cl.Ubq1-1:1:7 (SEQ ID NO: 92) | |
| P-Cl.Ubq1-1:1:9 | 91 | 2287 | C. lacryma-jobi | Promoter | |
| I-Cl.Ubq1-1:1:7 | 92 | 1020 | C. lacryma-jobi | Intron | |
| EXP-Cl.Ubq1:1:16 | 93 | 3393 | C. lacryma-jobi | EXP: P-Cl.Ubq1-1:1:9 (SEQ ID NO: 91); | pMON146750, |
| | | | | L-Cl.Ubq1-1:1:1 (SEQ ID NO: 81); | pMON142748 |
| | | | | I-Cl.Ubq1-1:1:6 (SEQ ID NO: 94) | |
| I-Cl.Ubq1-1:1:6 | 94 | 1020 | C. lacryma-jobi | Intron | |
| EXP-Cl.Ubq1:1:11 | 95 | 2166 | C. lacryma-jobi | EXP: P-Cl.Ubq1-1:1:10 (SEQ ID NO: 96); | pMON142730 |
| | | | | L-Cl.Ubq1-1:1:1 (SEQ ID NO: 81); | |
| | | | | I-Cl.Ubq1-1:1:7 (SEQ ID NO: 92) | |
| P-Cl.Ubq1-1:1:10 | 96 | 1060 | C. lacryma-jobi | Promoter | |
| EXP-Cl.Ubq1:1:17 | 97 | 2166 | C. lacryma-jobi | EXP: P-Cl.Ubq1-1:1:10 (SEQ ID NO: 96); | pMON146751, |
| | | | | L-Cl.Ubq1-1:1:1 (SEQ ID NO: 81); | pMON142749 |
| | | | | I-Cl.Ubq1-1:1:6 (SEQ ID NO: 94) | |
| EXP-Cl.Ubq1:1:10 | 98 | 1943 | C. lacryma-jobi | EXP: P-Cl.Ubq1-1:1:1 (SEQ ID NO: 80); | pMON140889, |
| | | | | L-Cl.Ubq1-1:1:1 (SEQ ID NO: 81); | PCR0145922, |
| | | | | I-Cl.Ubq1-1:1:6 (SEQ ID NO: 94) | pMON140913, |
| | | | | | PCR19, |
| | | | | | pMON129221 |
| EXP-Cl.Ubq1:1:18 | 99 | 1943 | C. lacryma-jobi | EXP: P-Cl.Ubq1-1:1:1 (SEQ ID NO: 80); | pMON146795 |
| | | | | L-Cl.Ubq1-1:1:1 (SEQ ID NO: 81); | |
| | | | | I-Cl.Ubq1-1:1:7 (SEQ ID NO: 92) | |
| EXP-Cl.Ubq1:1:19 | 100 | 1943 | C. lacryma-jobi | EXP: P-Cl.Ubq1-1:1:1 (SEQ ID NO: 80); | pMON146796 |
| | | | | L-Cl.Ubq1-1:1:1 (SEQ ID NO: 81); | |
| | | | | I-Cl.Ubq1-1:1:8 (SEQ ID NO: 101) | |
| I-Cl.Ubq1-1:1:8 | 101 | 1020 | C. lacryma-jobi | Intron | |
| EXP-Cl.Ubq1:1:20 | 102 | 1943 | C. lacryma-jobi | EXP: P-Cl.Ubq1-1:1:1 (SEQ ID NO: 80); | pMON146797 |
| | | | | L-Cl.Ubq1-1:1:1 (SEQ ID NO: 81); | |
| | | | | I-Cl.Ubq1-1:1:9 (SEQ ID NO: 103) | |
| I-Cl.Ubq1-1:1:9 | 103 | 1020 | C. lacryma-jobi | Intron | |
| EXP-Cl.Ubq1:1:21 | 104 | 1943 | C. lacryma-jobi | EXP: P-Cl.Ubq1-1:1:1 (SEQ ID NO: 80); | pMON146798 |
| | | | | L-Cl.Ubq1-1:1:1 (SEQ ID NO: 81); | |
| | | | | I-Cl.Ubq1-1:1:10 (SEQ ID NO: 105) | |
| I-Cl.Ubq1-1:1:10 | 105 | 1020 | C. lacryma-jobi | Intron | |
| EXP-Cl.Ubq1:1:22 | 106 | 1943 | C. lacryma-jobi | EXP: P-Cl.Ubq1-1:1:1 (SEQ ID NO: 80); | pMON146799 |
| | | | | L-Cl.Ubq1-1:1:1 (SEQ ID NO: 81); | |
| | | | | I-Cl.Ubq1-1:1:11 (SEQ ID NO: 107) | |
| I-Cl.Ubq1-1:1:11 | 107 | 1020 | C. lacryma-jobi | Intron | |
| EXP-Cl.Ubq1:1:23 | 108 | 1943 | C. lacryma-jobi | EXP: P-Cl.Ubq1-1:1:1 (SEQ ID NO: 80); | pMON132047, |
| | | | | L-Cl.Ubq1-1:1:1 (SEQ ID NO: 81); | pMON146800 |
| | | | | I-Cl.Ubq1-1:1:12 (SEQ ID NO: 109) | |
| I-Cl.Ubq1-1:1:12 | 109 | 1020 | C. lacryma-jobi | Intron | |
| EXP-Cl.Ubq1:1:24 | 110 | 1943 | C. lacryma-jobi | EXP: P-Cl.Ubq1-1:1:1 (SEQ ID NO: 80); | pMON146801 |
| | | | | L-Cl.Ubq1-1:1:1 (SEQ ID NO: 81); | |
| | | | | I-Cl.Ubq1-1:1:13 (SEQ ID NO: 111) | |
| I-Cl.Ubq1-1:1:13 | 111 | 1020 | C. lacryma-jobi | Intron | |
| EXP-Cl.Ubq1:1:25 | 112 | 1943 | C. lacryma-jobi | EXP: P-Cl.Ubq1-1:1:1 (SEQ ID NO: 80); | pMON146802 |
| | | | | L-Cl.Ubq1-1:1:1 (SEQ ID NO: 81); | |
| | | | | I-Cl.Ubq1-1:1:14 (SEQ ID NO: 113) | |
| I-Cl.Ubq1-1:1:14 | 113 | 1020 | C. lacryma-jobi | Intron | |
| EXP-Cl.Ubq1:1:13 | 114 | 1848 | C. lacryma-jobi | EXP: P-Cl.Ubq1-1:1:4 (SEQ ID NO: 84); | PCR0145945, |
| | | | | L-Cl.Ubq1-1:1:1 (SEQ ID NO: 81); | pMON140914, |
| | | | | I-Cl.Ubq1-1:1:6 (SEQ ID NO: 94) | PCR20 |
| EXP-Cl.Ubq1:1:14 | 115 | 1507 | C. lacryma-jobi | EXP: P-Cl.Ubq1-1:1:3 (SEQ ID NO: 86); | PCR0145946, |
| | | | | L-C1.Ubq1-1:1:1 (SEQ ID NO: 81); | pMON140915, |
| | | | | I-Cl.Ubq1-1:1:6 (SEQ ID NO: 94) | PCR21 |
| EXP-Cl.Ubq1:1:15 | 116 | 1160 | C. lacryma-jobi | EXP: P-Cl.Ubq1-1:1:5 (SEQ ID NO: 88); | PCR0145947, |
| | | | | L-Cl.Ubq1-1:1:1 (SEQ ID NO: 81); | pMON140916, |
| | | | | I-Cl.Ubq1-1:1:6 (SEQ ID NO: 94) | PCR22 |
| EXP-SETit.Ubq1:1:5 | 117 | 2625 | S. italica | EXP: P-SETit.Ubq1-1:1:1 (SEQ ID NO: 70); | pMON140877, |
| | | | | L-SETit.Ubq1-1:1:1 (SEQ ID NO: 71); | PCR0145900, |
| | | | | I-SETit.Ubq1-1:1:2 (SEQ ID NO: 118) | pMON129200 |
| I-SETit.Ubq1-1:1:2 | 118 | 1006 | S. italica | Intron | |
| EXP-SETit.Ubq1:1:10 | 119 | 2625 | S. italica | EXP: P-SETit.Ubq1-1:1:4 (SEQ ID NO: 64); | pMON132037 |
| | | | | L-SETit.Ubq1-1:1:1 (SEQ ID NO: 71); | |
| | | | | I-SETit.Ubq1-1:1:3 (SEQ ID NO: 120) | |
| I-SETit.Ubq1-1:1:3 | 120 | 1006 | S. italica | Intron | |
| EXP-SETit.Ubq1:1:12 | 121 | 2625 | S. italica | EXP: P-SETit.Ubq1-1:1:4 (SEQ ID NO: 64); | |
| | | | | L-SETit.Ubq1-1:1:1 (SEQ ID NO: 71); | |
| | | | | I-SETit.Ubq1-1:1:4 (SEQ ID NO: 122) | |
| I-SETit.Ubq1-1:1:4 | 122 | 1006 | S. italica | Intron | |
| EXP-SETit.Ubq1:1:7 | 123 | 2167 | S. italica | EXP: P-SETit.Ubq1-1:1:2 (SEQ ID NO: 71); | PCR0145928, |
| | | | | L-SETit.Ubq1-1:1:1 (SEQ ID NO: 71); | pMON129201 |
| | | | | I-SETit.Ubq1-1:1:2 (SEQ ID NO: 118) | |
| EXP-SETit.Ubq1:1:6 | 124 | 1813 | S. italica | EXP: P-SETit.Ubq1-1:1:3 (SEQ ID NO: 73); | PCR0145905, |
| | | | | L-SETit.Ubq1-1:1:1 (SEQ ID NO: 71); | pMON129202 |
| | | | | I-SETit.Ubq1-1:1:2 (SEQ ID NO: 118) | |
| EXP-SETit.Ubq1:1:11 | 125 | 1813 | S. italica | EXP: P-SETit.Ubq1-1:1:3 (SEQ ID NO: 73); | pMON131957 |
| | | | | L-SETit.Ubq1-1:1:1 (SEQ ID NO: 71); | |
| | | | | I-SETit.Ubq1-1:1:3 (SEQ ID NO: 120) | |
| EXP-SETit.Ubq1:1:13 | 126 | 1813 | S. italica | EXP: P-SETit.Ubq1-1:1:3 (SEQ ID NO: 73); | |
| | | | | L-SETit.Ubq1-1:1:1 (SEQ ID NO: 71); | |
| | | | | I-SETit.Ubq1-1:1:5 (SEQ ID NO: 127) | |
| I-SETit.Ubq1-1:1:5 | 127 | 1006 | S. italica | Intron | |
| EXP-Sv.Ubq1:1:7 | 128 | 2634 | S. viridis | EXP: P-Sv.Ubq1-1:1:1 (SEQ ID NO: 34); | pMON140878, |
| | | | | L-Sv.Ubq1-1:1:2 (SEQ ID NO: 35); | PCR0145909, |
| | | | | I-Sv.Ubq1-1:1:2 (SEQ ID NO: 129) | pMON129203 |
| I-Sv.Ubq1-1:1:2 | 129 | 1014 | S. viridis | Intron | |
| EXP-Sv.Ubq1:1:11 | 130 | 2634 | S. viridis | EXP: P-Sv.Ubq1-1:1:1 (SEQ ID NO: 34); | pMON131958 |
| | | | | L-Sv.Ubq1-1:1:2 (SEQ ID NO: 35); | |
| | | | | I-Sv.Ubq1-1:1:3 (SEQ ID NO: 131) | |
| I-Sv.Ubq1-1:1:3 | 131 | 1014 | S. viridis | Intron | |
| EXP-Sv.Ubq1:1:8 | 132 | 2176 | S. viridis | EXP: P-Sv.Ubq1-1:1:2 (SEQ ID NO: 38); | PCR0145929, |
| | | | | L-Sv.Ubq1-1:1:2 (SEQ ID NO: 35); | pMON129204 |
| | | | | I-Sv.Ubq1-1:1:2 (SEQ ID NO: 129) | |
| EXP-Sv.Ubq1:1:9 | 133 | 1822 | S. viridis | EXP: P-Sv.Ubq1-1:1:3 (SEQ ID NO: 40); | pMON129205 |
| | | | | L-Sv.Ubq1-1:1:2 (SEQ ID NO: 35); | |
| | | | | I-Sv.Ubq1-1:1:2 (SEQ ID NO: 129) | |
| EXP-Sv.Ubq1:1:10 | 134 | 1822 | S. viridis | EXP: P-Sv.Ubq1-1:1:4 (SEQ ID NO: 135); | PCR0145911 |
| | | | | L-Sv.Ubq1-1:1:2 (SEQ ID NO: 35); | |
| | | | | I-Sv.Ubq1-1:1:2 (SEQ ID NO: 129) | |
| P-Sv.Ubq1-1:1:4 | 135 | 681 | S. viridis | Promoter | |
| EXP-Sv.Ubq1:1:12 | 136 | 1822 | S. viridis | EXP: P-Sv.Ubq1-1:1:3 (SEQ ID NO: 40); | pMON131959 |
| | | | | L-Sv.Ubq1-1:1:2 (SEQ ID NO: 35); | |
| | | | | I-Sv.Ubq1-1:1:3 (SEQ ID NO: 131) | |
| EXP-Zm.UbqM1:1:6 | 137 | 1925 | Z. mays subsp. | EXP: P-Zm.UbqM1-1:1:1 (SEQ ID NO: 42); | pMON140881, |
| (Allele-1) | | | Mexicana | L-Zm.UbqM1-1:1:1 (SEQ ID NO: 43); | PCR0145914, |
| | | | | I-Zm.UbqM1-1:1:13 (SEQ ID NO: 138) | pMON129210 |
| I-Zm.UbqM1-1:1:13 | 138 | 997 | Z. mays subsp. | Intron | |
| (Allele-1) | | | Mexicana | | |
| EXP-Zm.UbqM1:1:10 | 139 | 1925 | Z. mays subsp. | EXP: P-Zm.UbqM1-1:1:1 (SEQ ID NO: 42); | pMON131961 |
| (Allele-1) | | | Mexicana | L-Zm.UbqM1-1:1:1 (SEQ ID NO: 43); | |
| | | | | I-Zm.UbqM1-1:1:17 (SEQ ID NO: 140) | |
| I-Zm.UbqM1-1:1:17 | 140 | 997 | Z. mays subsp. | Intron | |
| (Allele-1) | | | Mexicana | | |
| EXP-Zm.UbqM1:1:7 | 141 | 1974 | Z. mays subsp. | EXP: P-Zm.UbqM1-1:1:4 (SEQ ID NO: 46); | pMON140882, |
| (Allele-2) | | | Mexicana | L-Zm.UbqM1-1:1:5 (SEQ ID NO: 47); | PCR0145915, |
| | | | | I-Zm.UbqM1-1:1:14 (SEQ ID NO: 142) | pMON129212 |
| I-Zm.UbqM1-1:1:14 | 142 | 1010 | Z. mays subsp. | Intron | |
| (Allele-2) | | | Mexicana | | |
| EXP-Zm.UbqM1:1:12 | 143 | 1974 | Z. mays subsp. | EXP: P-Zm.UbqM1-1:1:4 (SEQ ID NO: 46); | pMON131963 |
| (Allele-2) | | | Mexicana | L-Zm.UbqM1-1:1:5 (SEQ ID NO: 47); | |
| | | | | I-Zm.UbqM1-1:1:19 (SEQ ID NO: 144) | |
| I-Zm.UbqM1-1:1:19 | 144 | 1010 | Z. mays subsp. | Intron | |
| (Allele-2) | | | Mexicana | | |
| EXP-Zm.UbqM1:1:8 | 145 | 2008 | Z. mays subsp. | EXP: P-Zm.UbqM1-1:1:5 (SEQ ID NO: 50); | PCR0145916, |
| (Allele-3) | | | Mexicana | L-Zm.UbqM1-1:1:4 (SEQ ID NO: 51); | pMON129211 |
| | | | | I-Zm.UbqM1-1:1:15 (SEQ ID NO: 146) | |
| I-Zm.UbqM1-1:1:15 | 146 | 1053 | Z. mays subsp. | Intron | |
| (Allele-3) | | | Mexicana | | |
| EXP-Zm.UbqM1:1:9 | 147 | 2008 | Z. mays subsp. | EXP: P-Zm.UbqM1-1:1:5 (SEQ ID NO: 50); | |
| (Allele-3) | | | Mexicana | L-Zm.UbqM1-1:1:4 (SEQ ID NO: 51); | |
| | | | | I-Zm.UbqM1-1:1:16 (SEQ ID NO: 148) | |
| I-Zm.UbqM1-1:1:16 | 148 | 1053 | Z. mays subsp. | Intron | |
| (Allele-3) | | | Mexicana | | |
| EXP-Zm.UbqM1:1:11 | 149 | 2008 | Z. mays subsp. | EXP: P-Zm.UbqM1-1:1:5 (SEQ ID NO: 50); | pMON131962, |
| (Allele-3) | | | Mexicana | L-Zm.UbqM1-1:1:4 (SEQ ID NO: 51); | pMON132047 |
| | | | | I-Zm.UbqM1-1:1:18 (SEQ ID NO: 150) | |
| I-Zm.UbqM1-1:1:18 | 150 | 1053 | Z. mays subsp. | Intron | |
| (Allele-3) | | | Mexicana | | |
| EXP-Sb.Ubq4:1:2 | 151 | 1635 | S. bicolor | EXP: P-Sb.Ubq4-1:1:1 (SEQ ID NO: 56); | pMON140886, |
| | | | | L-Sb.Ubq4-1:1:1 (SEQ ID NO: 57); | PCR0145921, |
| | | | | I-Sb.Ubq4-1:1:2 (SEQ ID NO: 152) | pMON129219, |
| | | | | | pMON132932 |
| I-Sb.Ubq4-1:1:2 | 152 | 1080 | S. bicolor | Intron | |
| EXP-Sb.Ubq6:1:2 | 153 | 2067 | S. bicolor | EXP: P-Sb.Ubq6-1:1:1 (SEQ ID NO: 64); | pMON140887, |
| | | | | L-Sb.Ubq6-1:1:1 (SEQ ID NO: 57); | PCR0145920, |
| | | | | I-Sb.Ubq6-1:1:2 (SEQ ID NO: 154) | pMON129218, |
| | | | | | pMON132931 |
| I-Sb.Ubq6-1:1:2 | 154 | 1076 | S. bicolor | Intron | |
| EXP-Sb.Ubq6:1:3 | 155 | 2067 | S. bicolor | EXP: P-Sb.Ubq6-1:1:1 (SEQ ID NO: 64); | pMON132931 |
| | | | | L-Sb.Ubq6-1:1:1 (SEQ ID NO: 57); | |
| | | | | I-Sb.Ubq6-1:1:3 (SEQ ID NO: 1569) | |
| I-Sb.Ubq6-1:1:3 | 156 | 1076 | S. bicolor | Intron | |
| EXP-Sb.Ubq7:1:2 | 157 | 2003 | S. bicolor | EXP: P-Sb.Ubq7-1:1:1 (SEQ ID NO: 66); | pMON132974 |
| | | | | L-Sb.Ubq7-1:1:1 (SEQ ID NO: 67); | |
| | | | | I-Sb.Ubq7-1:1:A (SEQ ID NO: 158) | |
| I-Sb.Ubq7-1:1:2 | 158 | 1361 | S. bicolor | Intron | |
| EXP-SETit.Ubq1:1:E | 180 | 2625 | S. italica | EXP: P-SETit.Ubq1-1:1:4 (SEQ ID NO: 64); | |
| | | | | L-SETit.Ubq1-1:1:1 (SEQ ID NO: 71); | |
| | | | | I-SETit.Ubq1-1:1:5 (SEQ ID NO: 127) | |
| EXP-Zm.UbqM1:1:13 | 181 | 2008 | Z. mays subsp. | EXP: P-Zm.UbqM1-1:1:5 (SEQ ID NO: 50); | |
| (Allele-3) | | | Mexicana | L-Zm.UbqM1-1:1:4 (SEQ ID NO: 51); | |
| | | | | I-Zm.UbqM1-1:1:20 (SEQ ID NO: 182) | |
| I-Zm.UbqM1-1:1:20 | 182 | 1053 | Z. mays subsp. | Intron | |
| (Allele-3) | | | Mexicana | | |
| EXP-SETit.Ubq1:1:9 | 183 | 2625 | S. italica | EXP: P-SETit.Ubq1-1:1:4 (SEQ ID NO: 64); | |
| | | | | L-SETit.Ubq1-1:1:1 (SEQ ID NO: 71); | |
| | | | | I-SETit.Ubq1-1:1:2 (SEQ ID NO: 118) | |

As shown in Table 1, for example, the transcriptional regulatory EXP sequence designated EXP-ANDge.Ubq1:1:9 (SEQ ID NO: 1), with components isolated from A. gerardii, comprises a promoter element, P-ANDge.Ubq1-1:1:11 (SEQ ID NO: 2), operably linked 5′ to a leader element, L-ANDge.Ubq1-1:1:2 (SEQ ID NO: 3), operably linked 5′ to an intron element, I-ANDge.Ubq1-1:1:3 (SEQ ID NO: 4). Other EXP's are linked similarly, as outlined in Table 1.

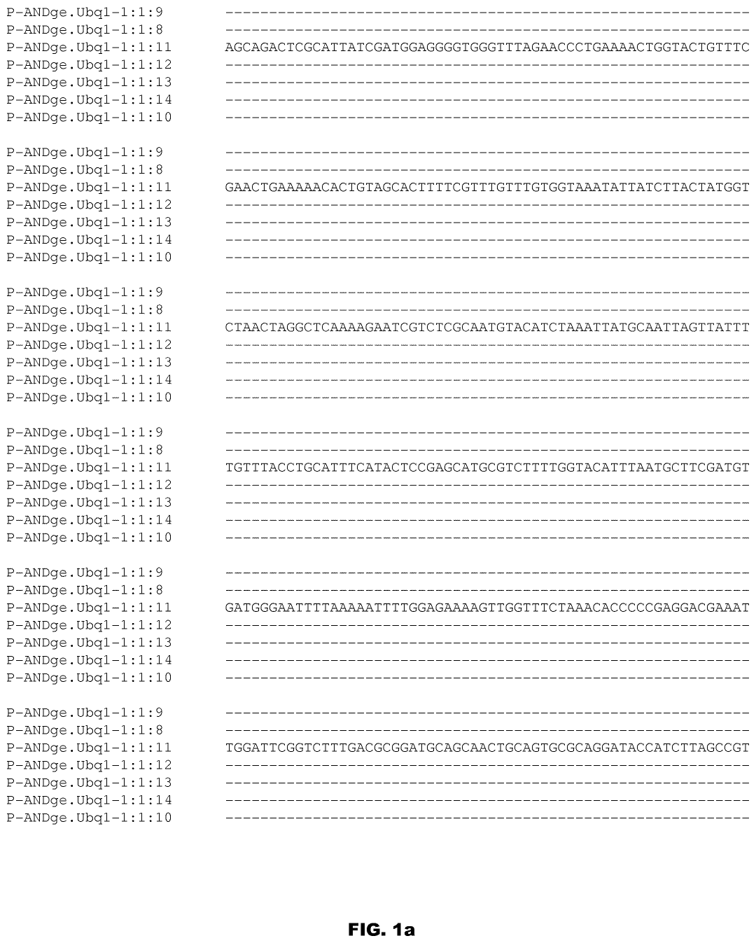

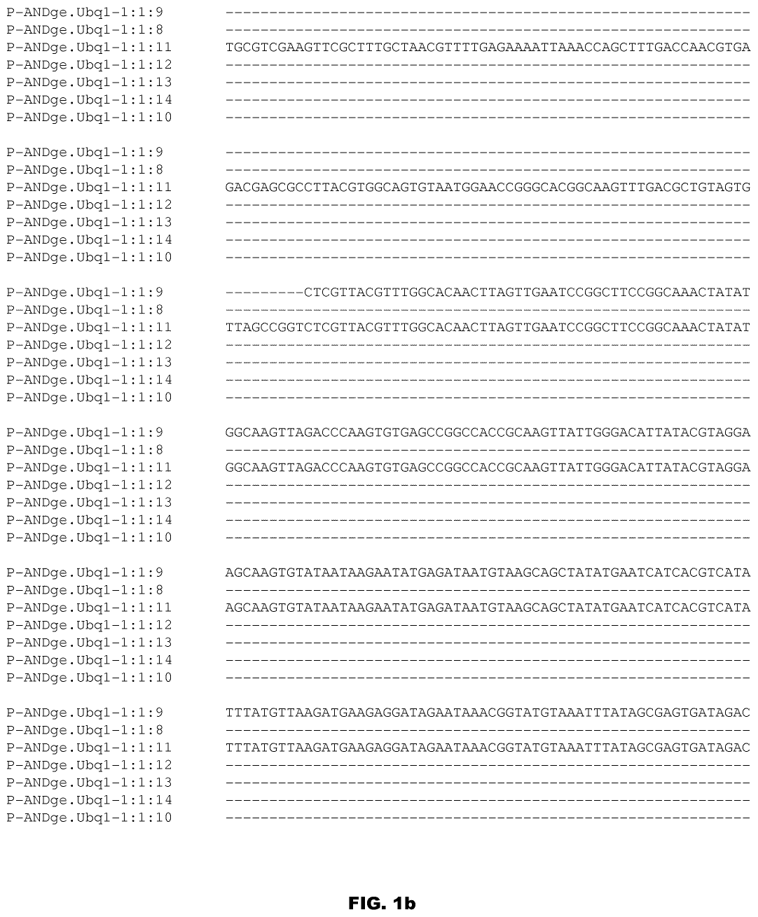

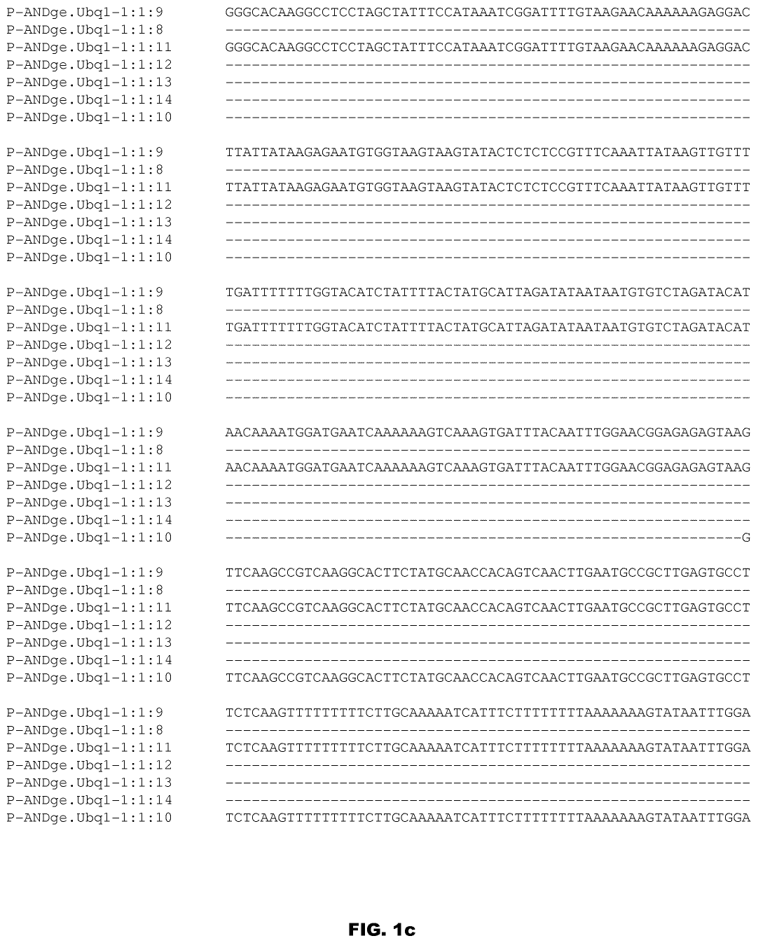

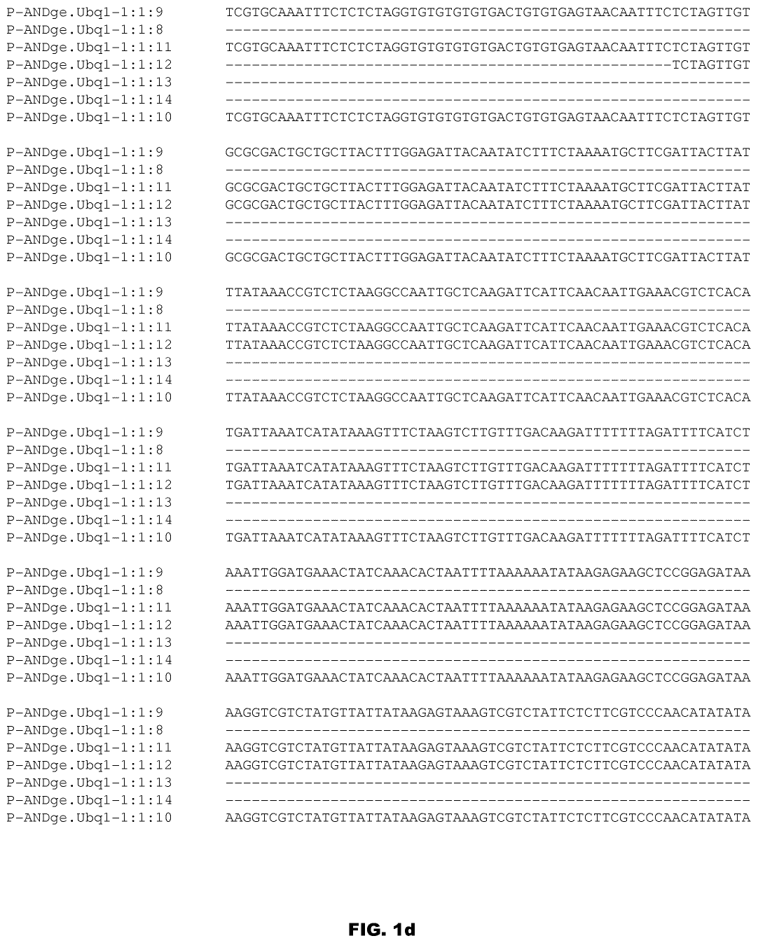

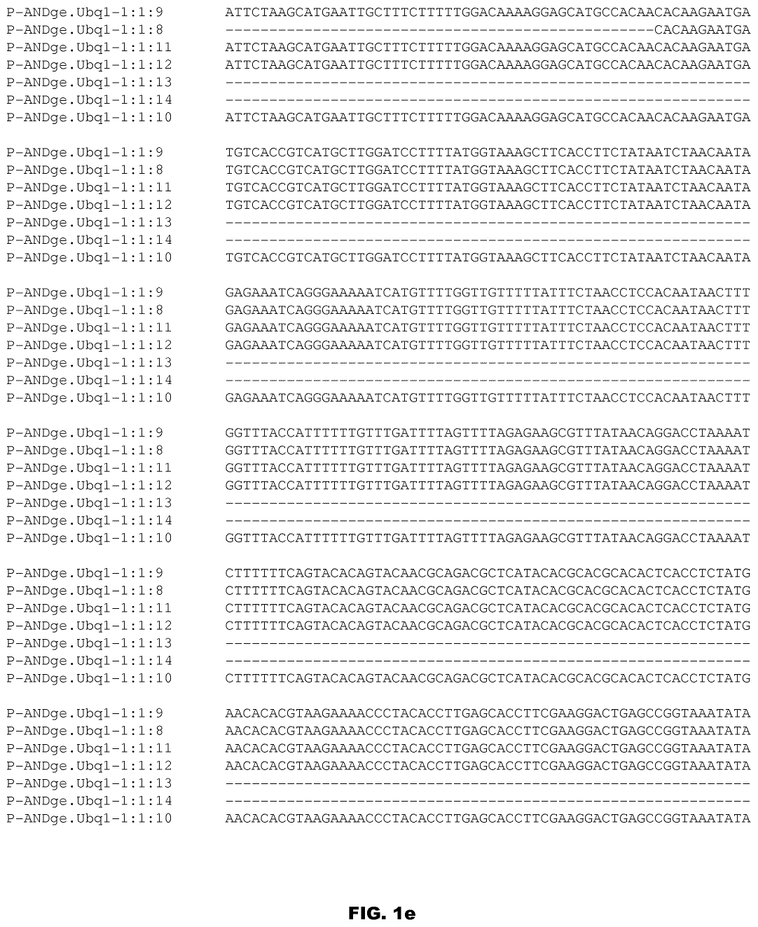

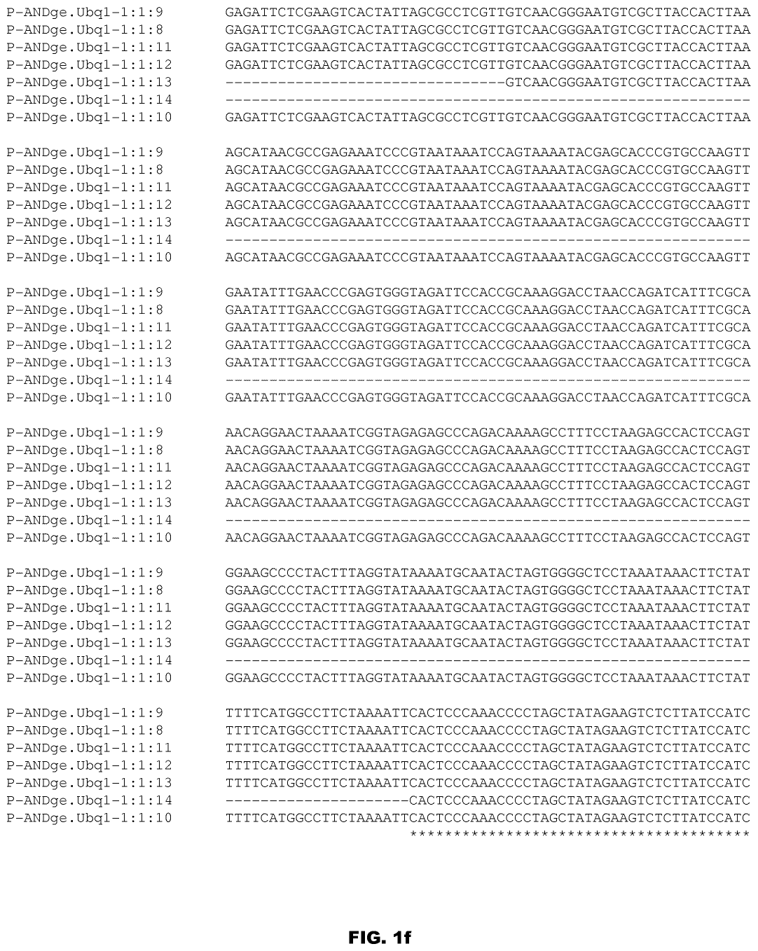

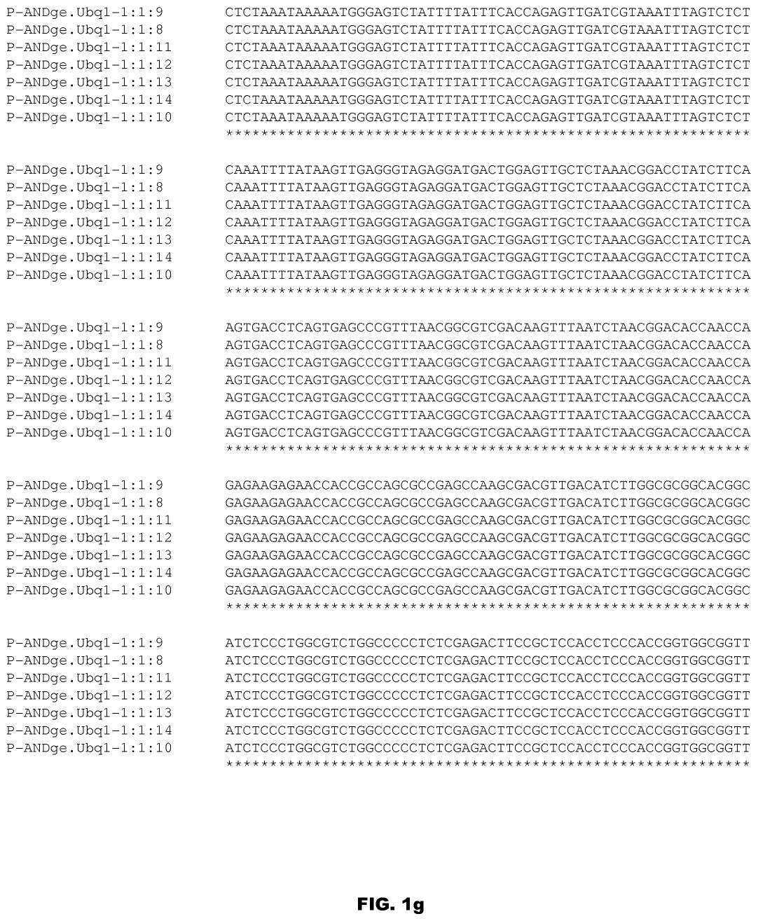

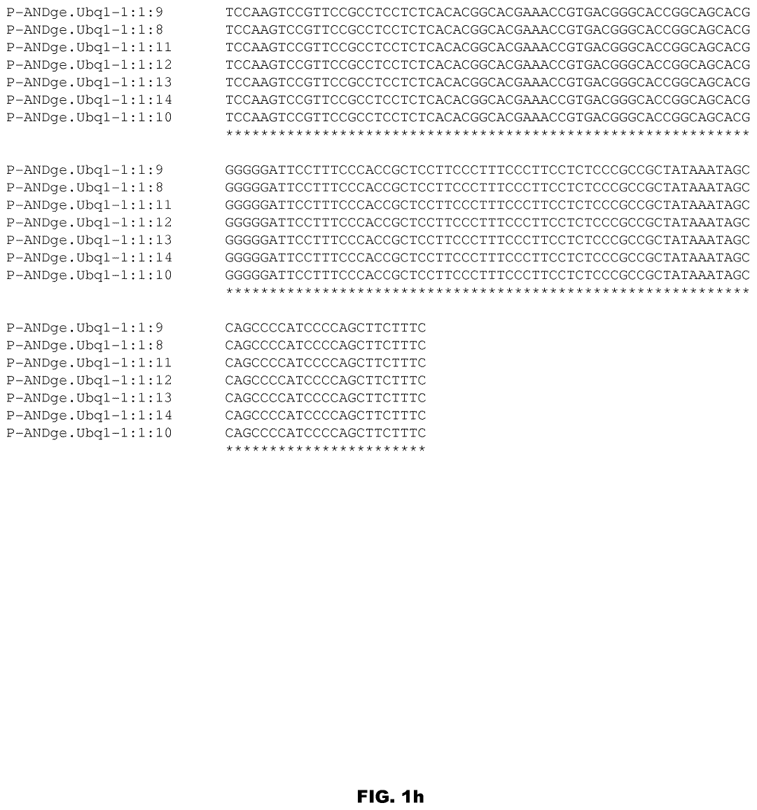

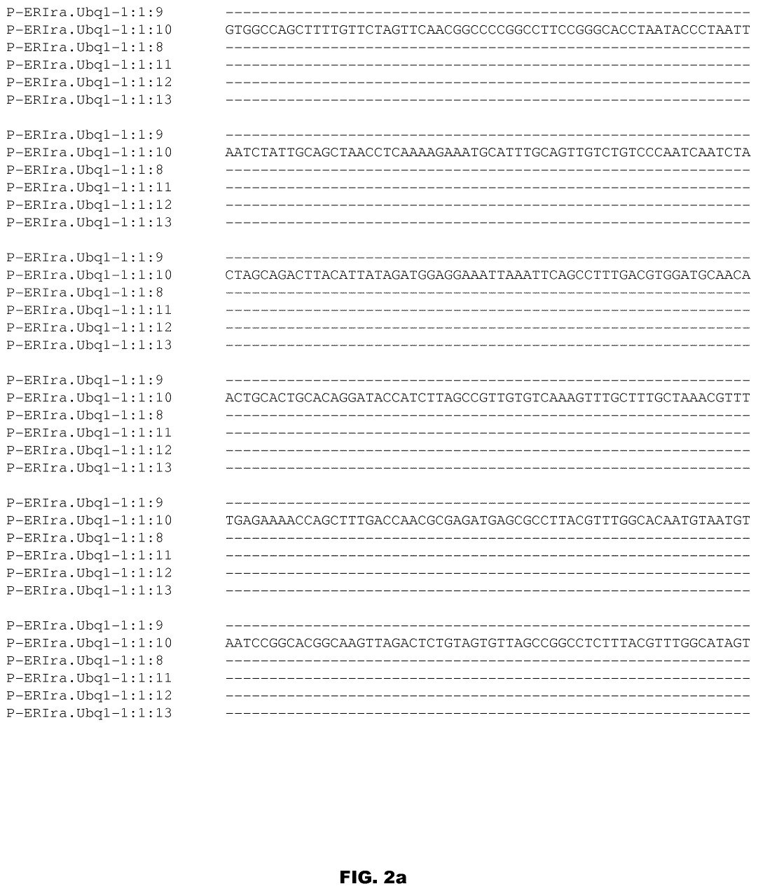

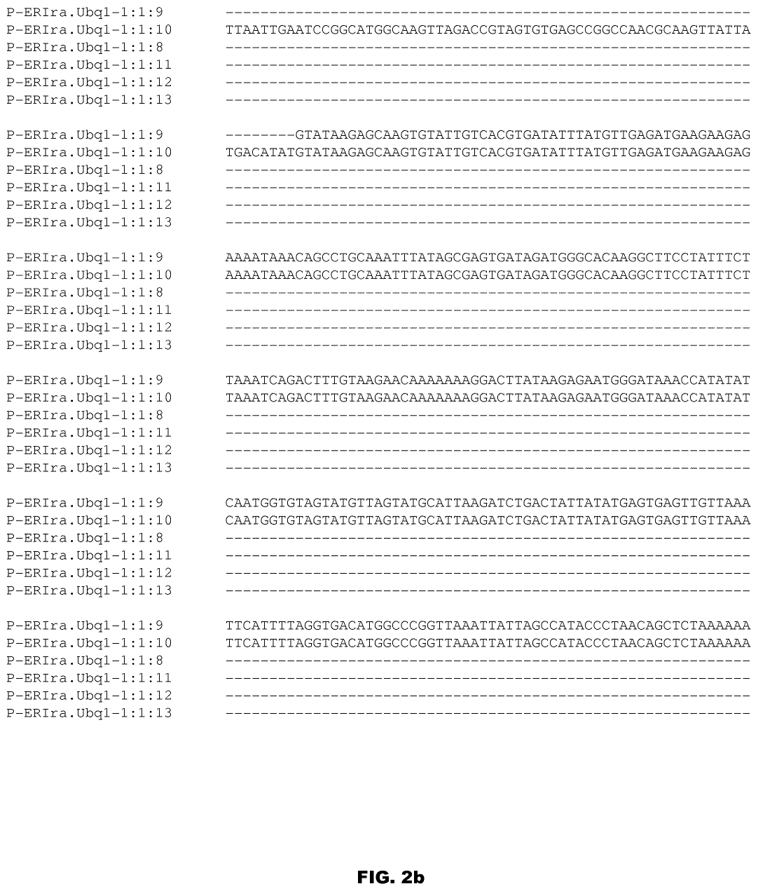

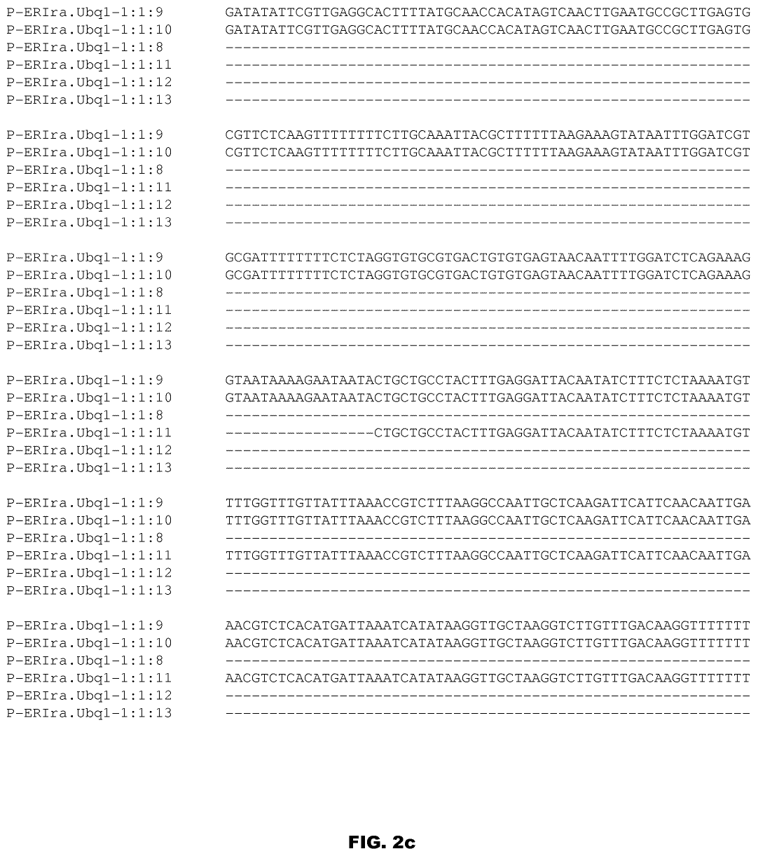

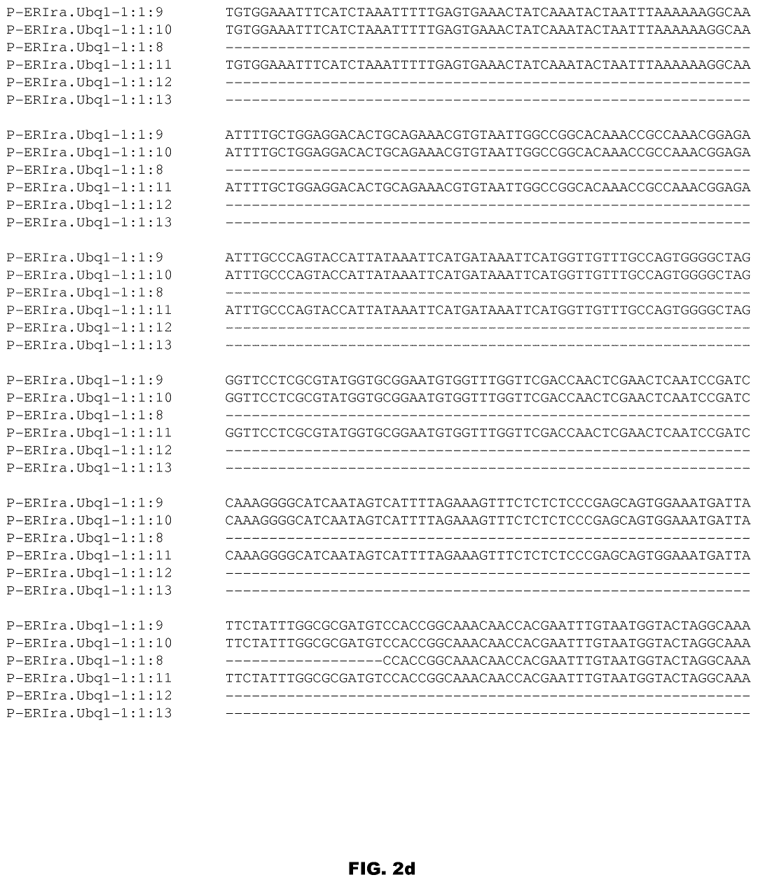

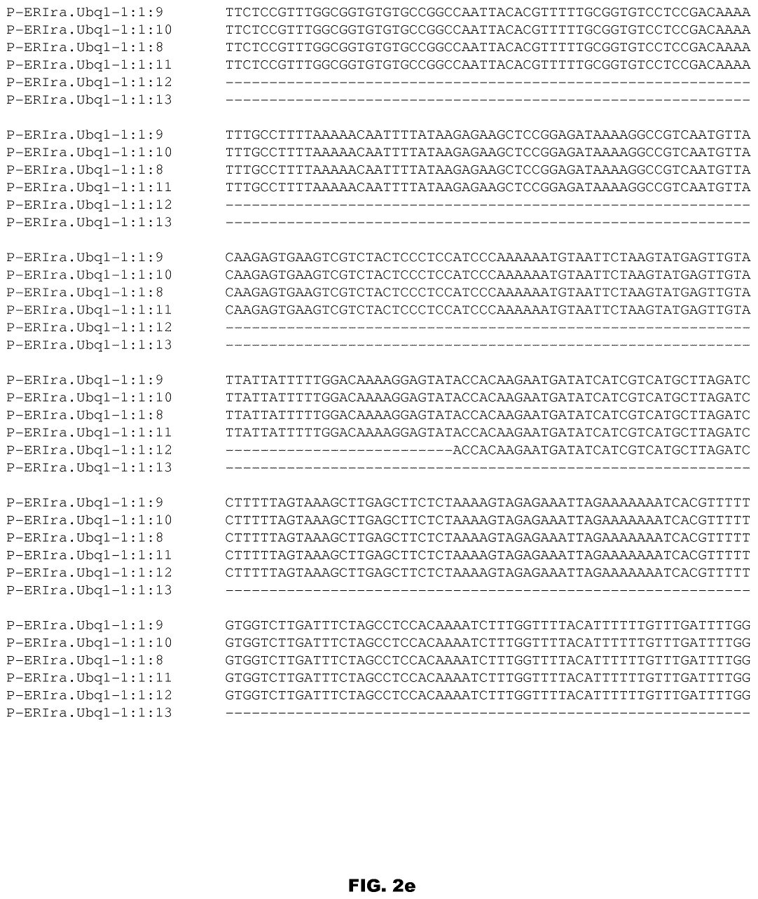

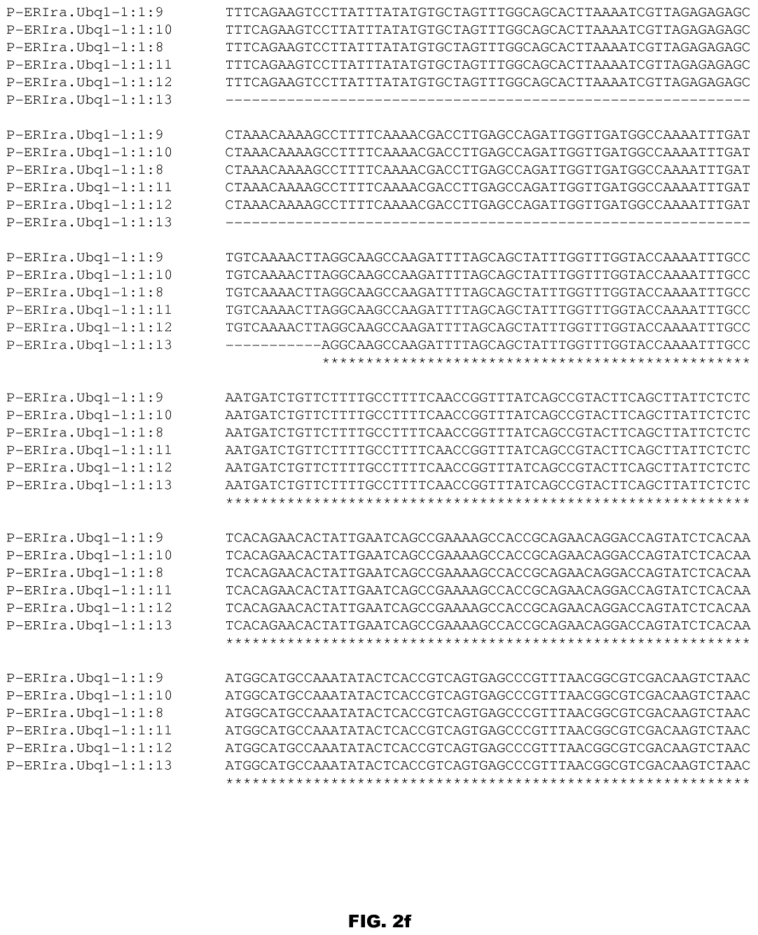

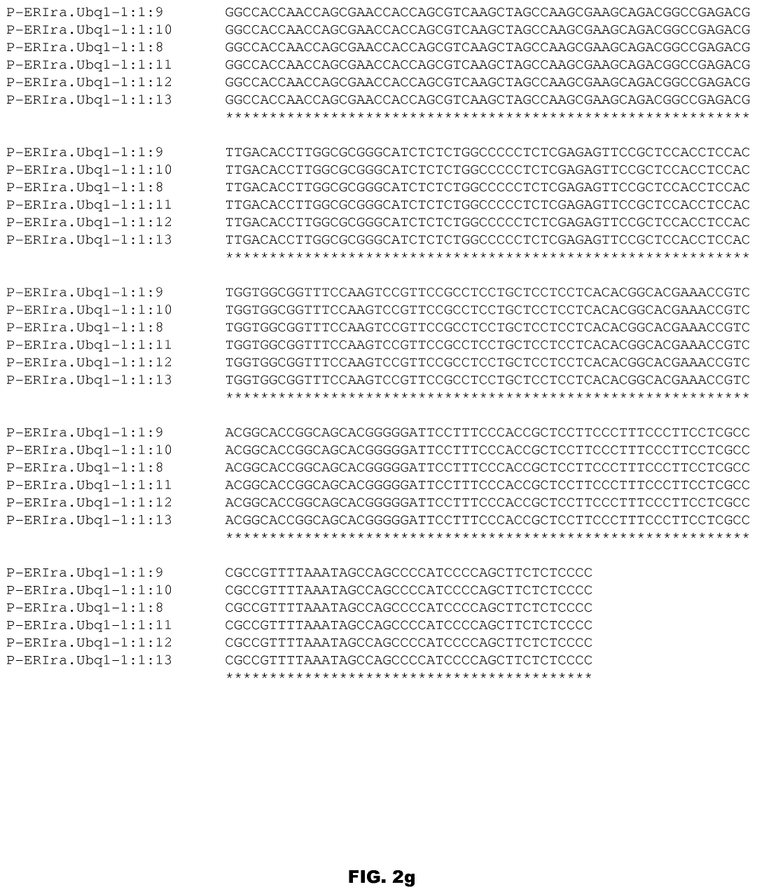

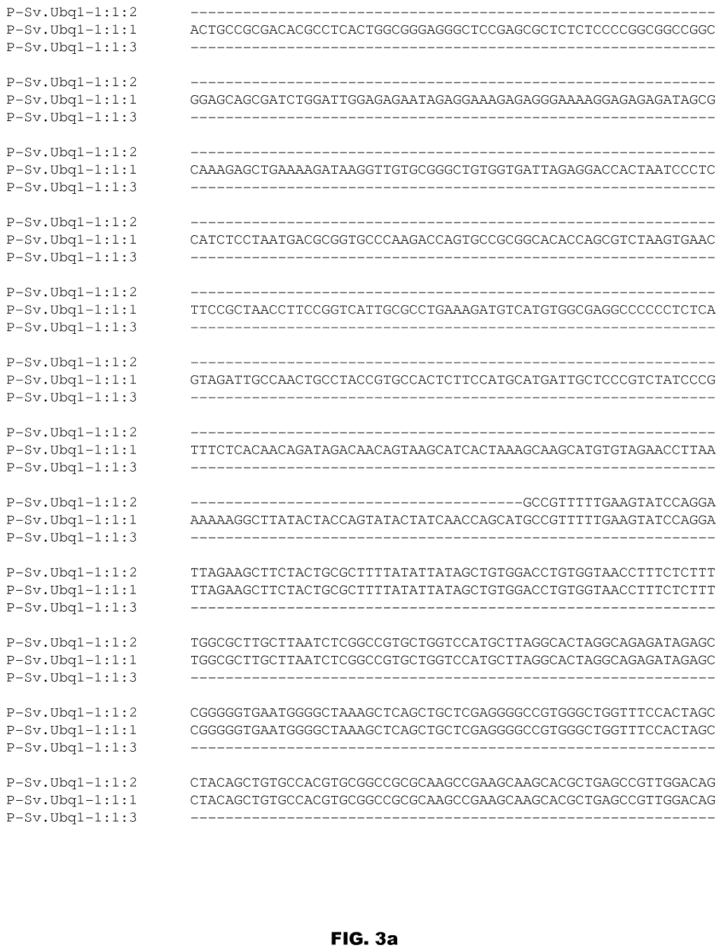

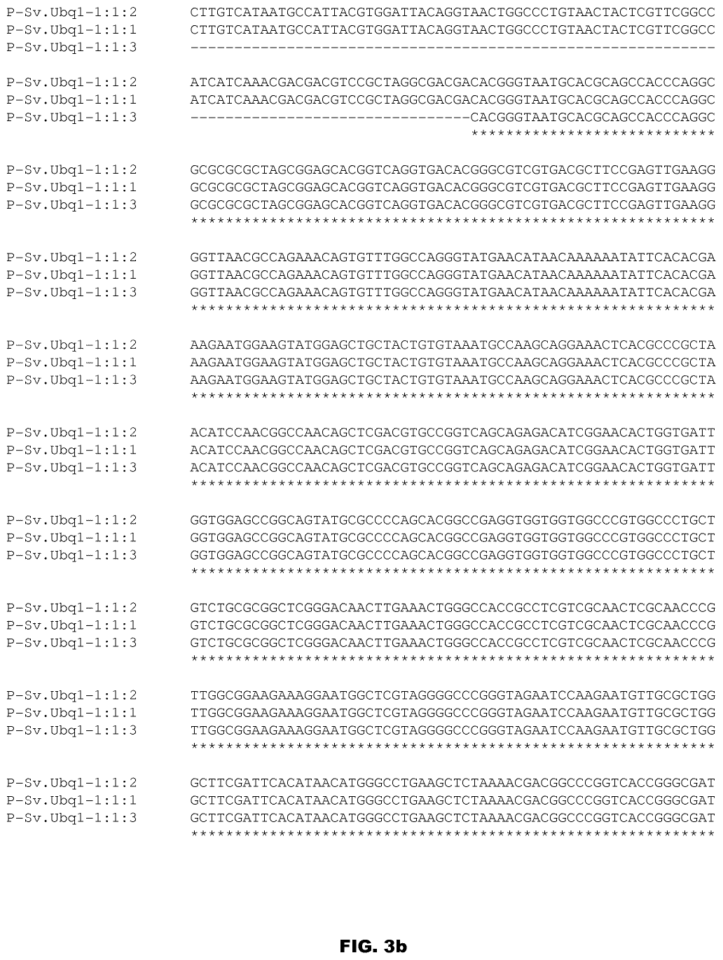

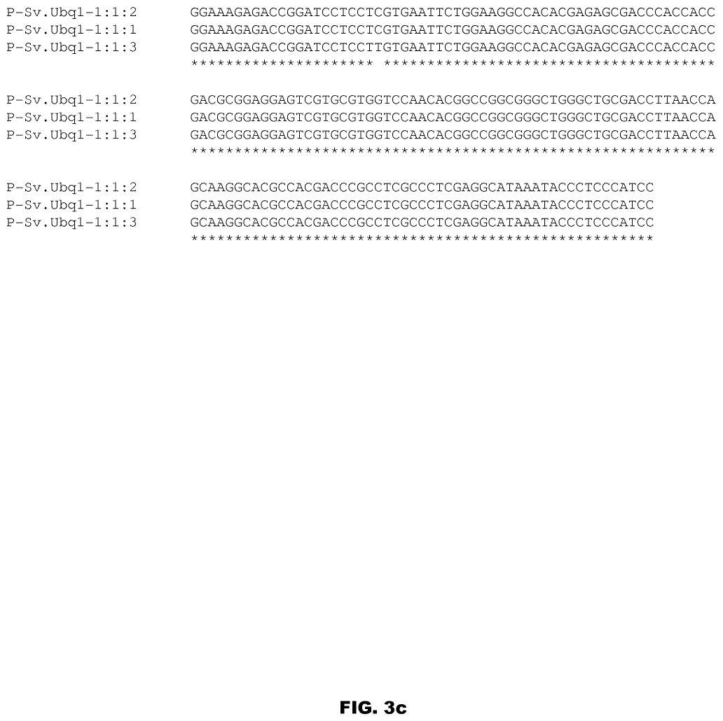

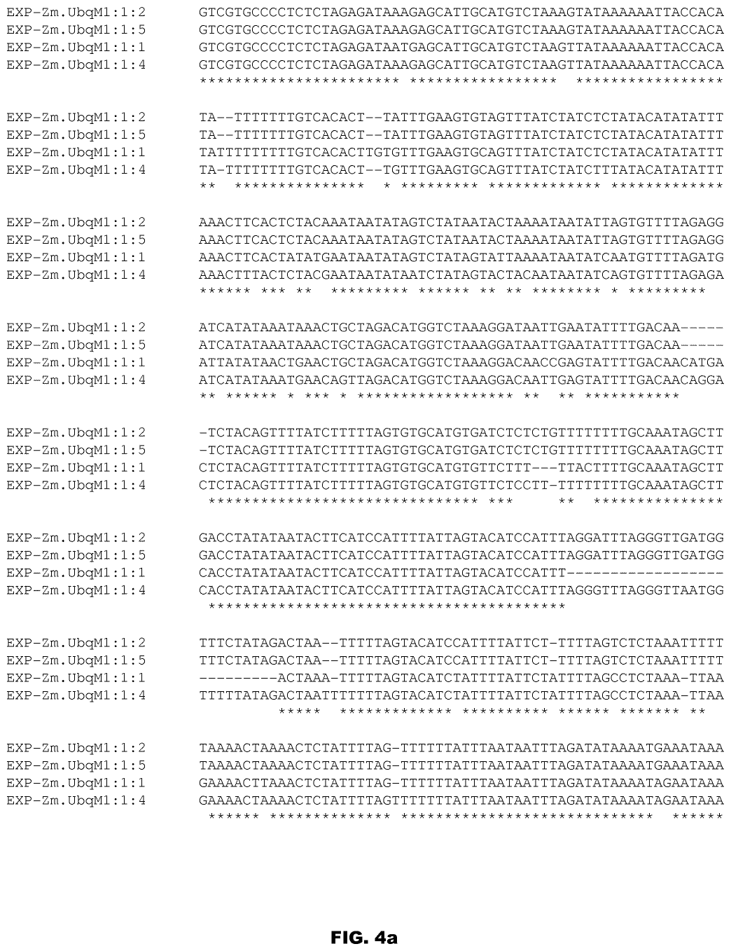

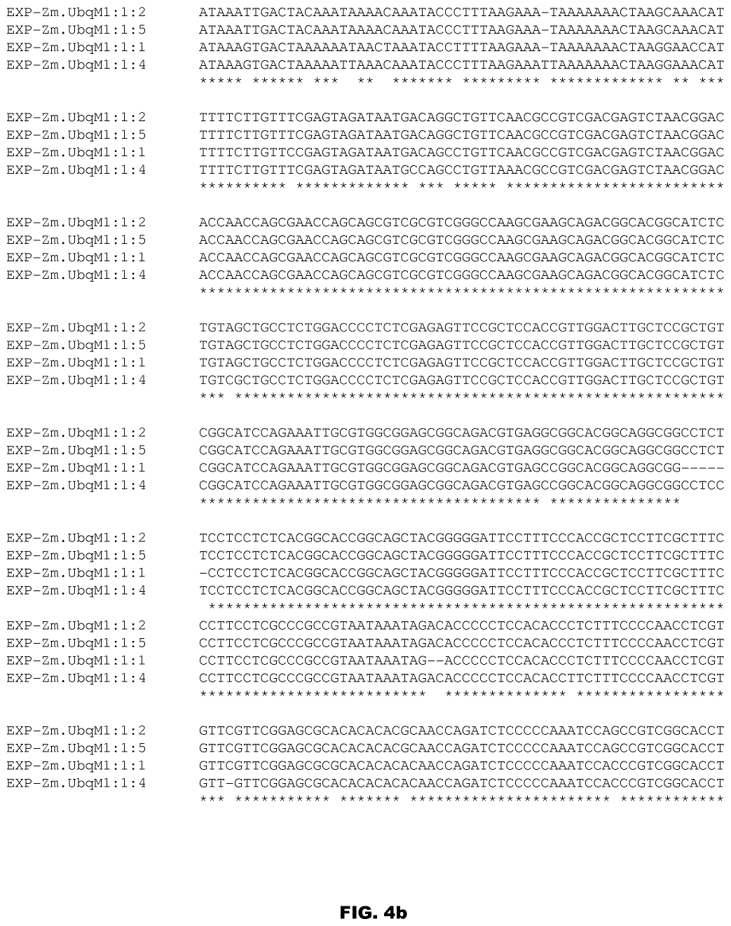

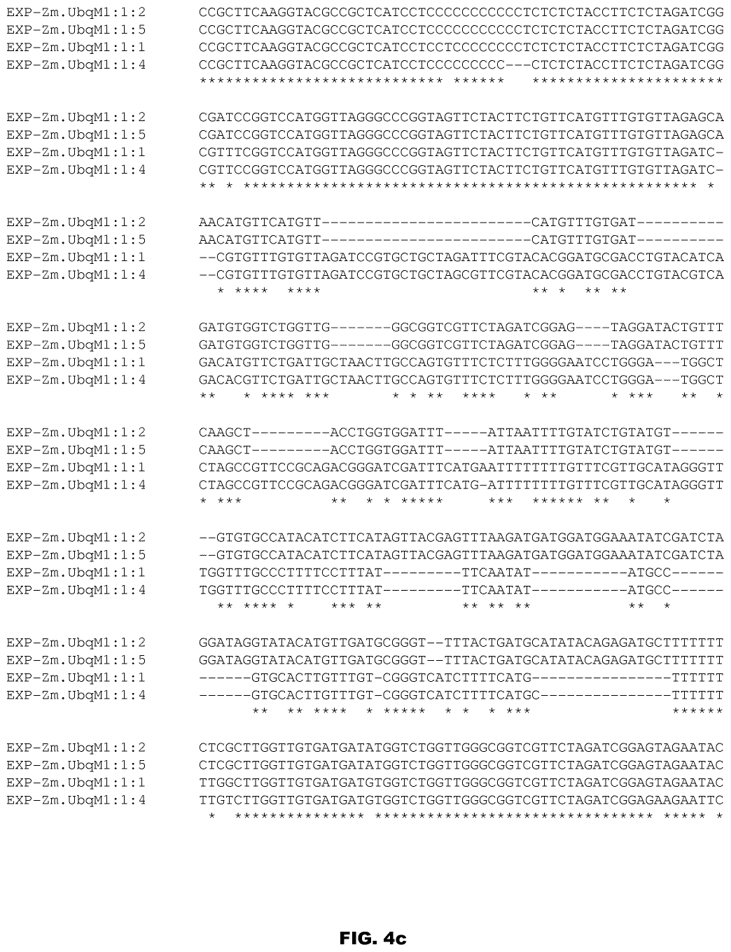

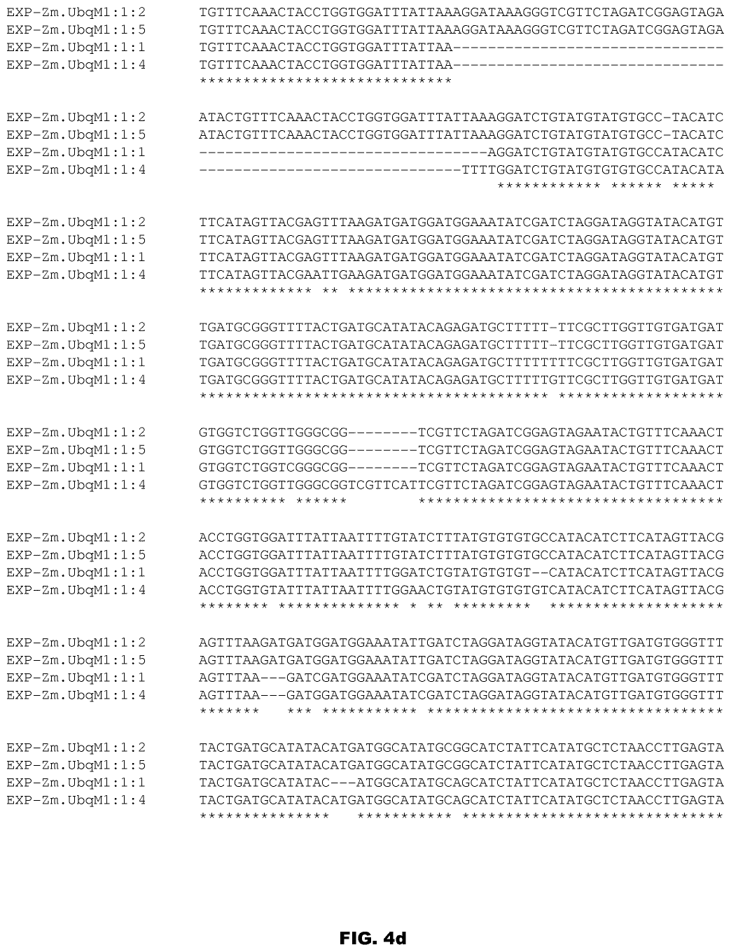

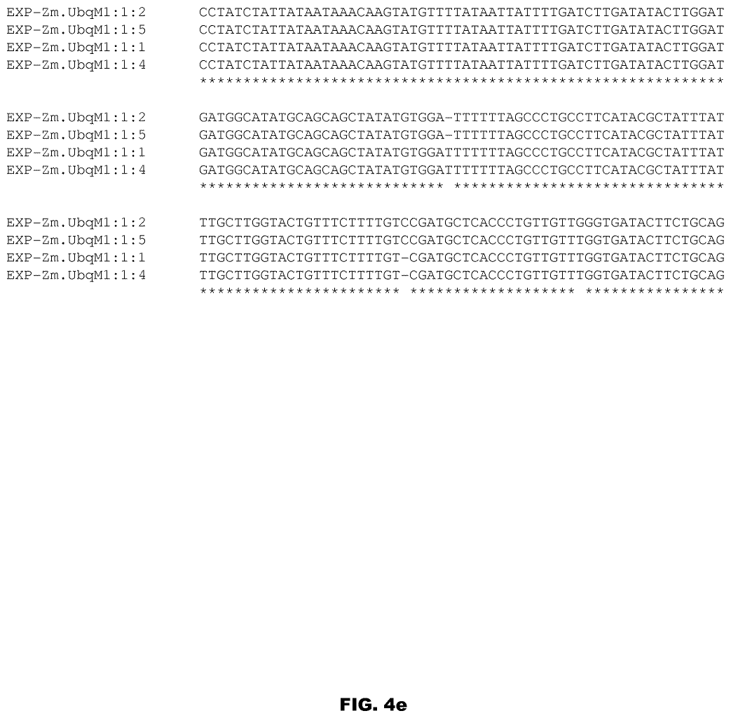

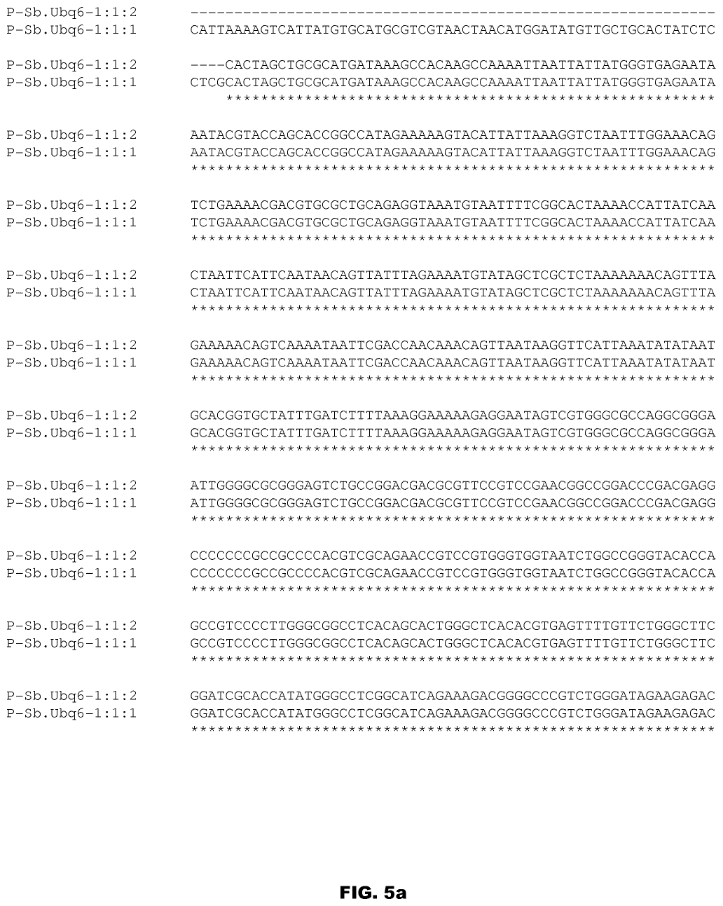

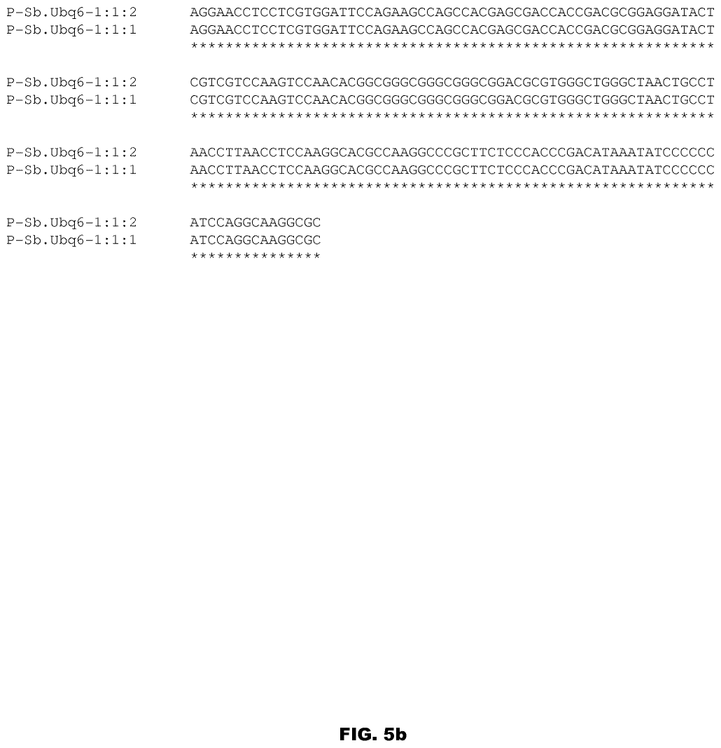

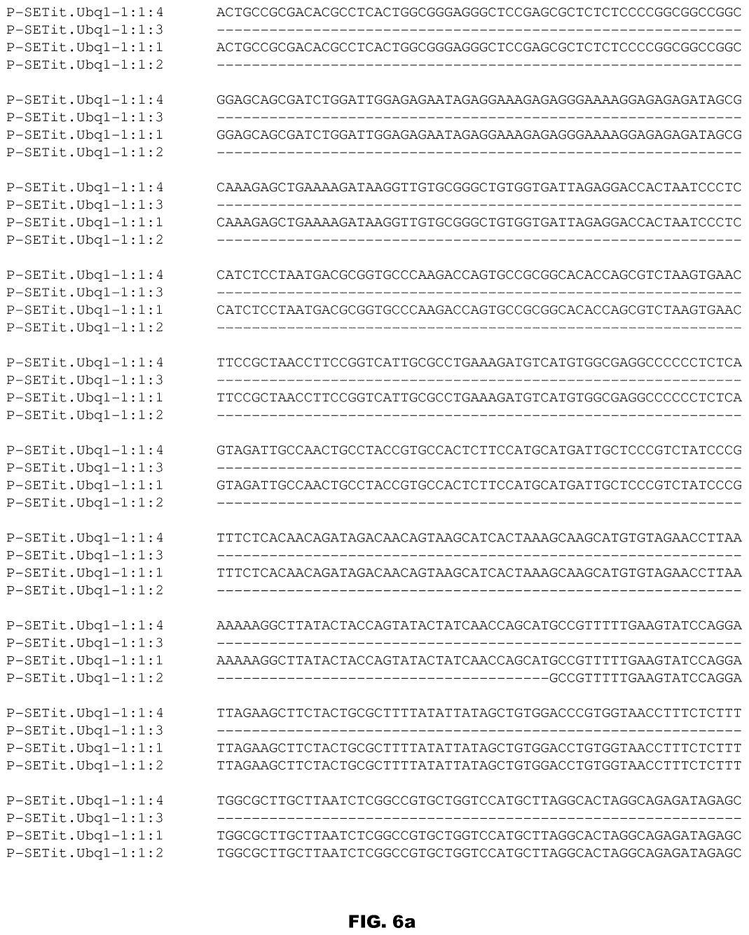

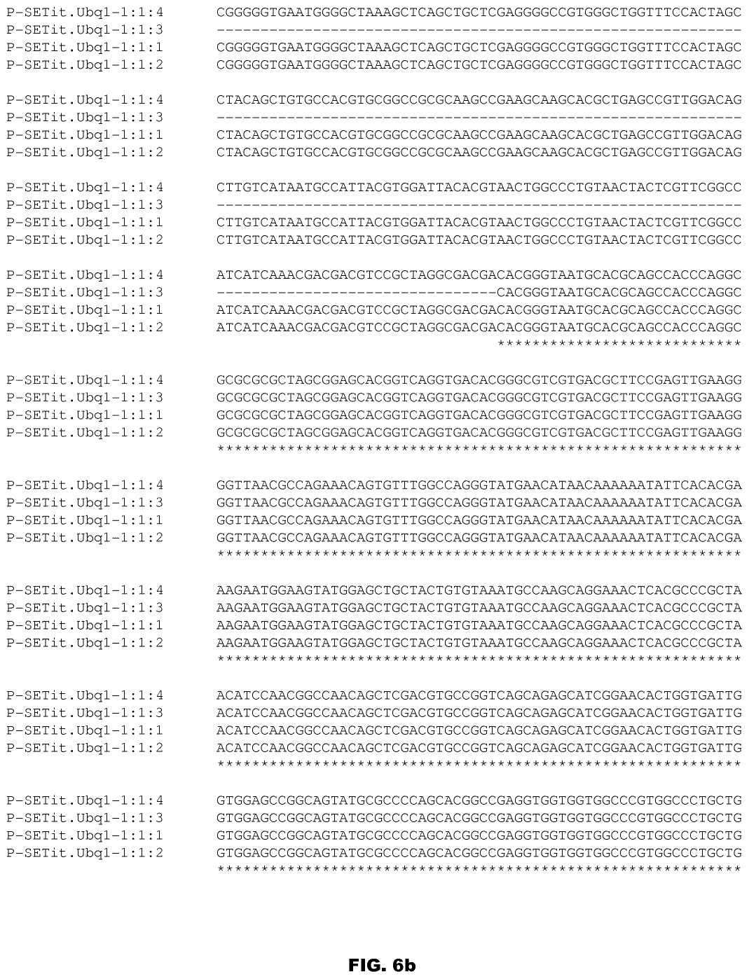

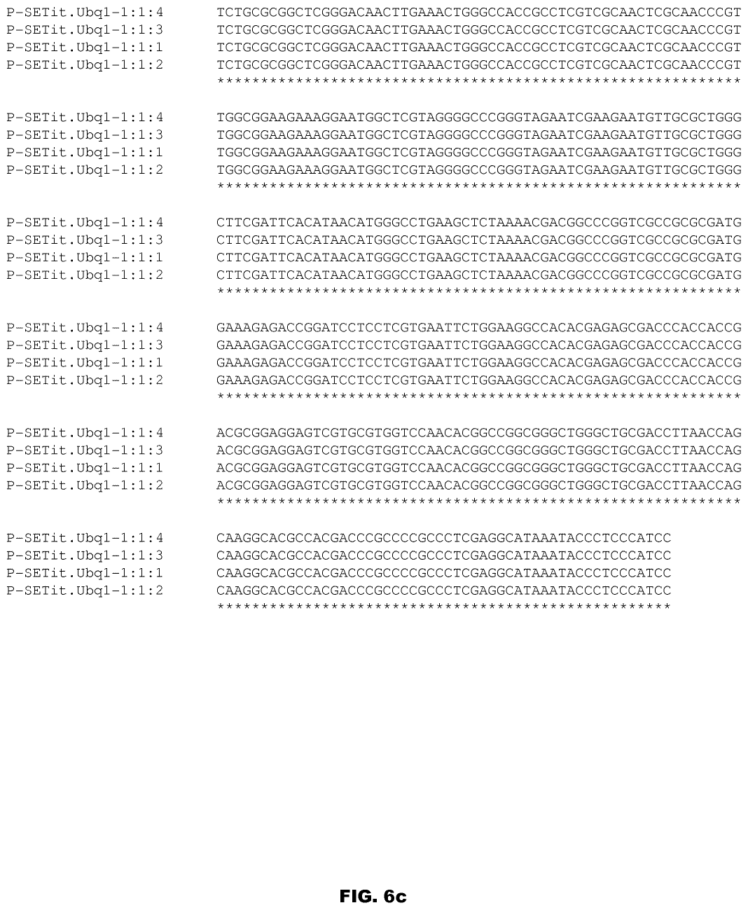

As shown in Table 1, the sequence listing and FIGS. 1-7, variants of promoter sequences from the species A. gerardii, E. ravennae, Z. mays subsp. mexicana, S. bicolor, C. lacryma-jobi, S. italica, and S. viridis were engineered which comprise shorter promoter fragments of, for instance, P-ANDge.Ubq1-1:1:11 (SEQ ID NO:2), P-ERIra.Ubq1-1:1:10 (SEQ ID NO:19) or other respective promoters from other species, and for instance resulting in P-ANDge.Ubq1-1:1:9 (SEQ ID NO: 6), P-ERIra.Ubq1-1:1:9 (SEQ ID NO: 23), P-Cl.Ubq1-1:1:10 (SEQ ID NO: 96), P-SETit.Ubq1-1:1:2 (SEQ ID NO: 76) and P-Sv.Ubq1-1:1:2 (SEQ ID NO: 38), as well as other promoter fragments. P-SETit.Ubq1-1:1:4 (SEQ ID NO: 74) comprises a single nucleotide change relative to P-SETit.Ubq1-1:1:1 (SEQ ID NO: 70). Likewise, P-Sv.Ubq1-1:1:3 (SEQ ID NO: 40) comprises a single nucleotide change relative to P-Sv.Ubq1-1:1:4 (SEQ ID NO: 135).

In some instances, variants of specific introns were created by altering the last 3′ nucleotides of each respective intron following the sequence 5′-AG-3′ of the 3′ intron splice junction. These intron variants are shown in Table 2 below.

TABLE 2
3′ end sequence of intron variants.

| | | Intron |
|---|---|---|
| | | 3′ end |
| | | nucleotides |
| | | immediately |
| | | following |
| | SEQ ID | 3′ splice |
| Annotation | NO: | site AG |
| I-Cl.Ubq1-1:1:7 | 92 | GTG |
| I-Cl.Ubq1-1:1:6 | 94 | GTC |
| I-Cl.Ubq1-1:1:8 | 101 | GCG |
| I-Cl.Ubq1-1:1:9 | 103 | GAC |
| I-Cl.Ubq1-1:1:10 | 105 | ACC |
| I-Cl.Ubq1-1:1:11 | 107 | GGG |
| I-Cl.Ubq1-1:1:12 | 109 | GGT |
| I-Cl.Ubq1-1:1:13 | 111 | CGT |
| I-Cl.Ubq1-1:1:14 | 113 | TGT |
| I-SETit.Ubq1-1:1:2 | 118 | GTG |
| I-SETit.Ubq1-1:1:3 | 120 | GGT |
| I-SETit.Ubq1-1:1:4 | 122 | ACC |
| I-SETit.Ubq1-1:1:5 | 127 | GGC |
| I-Sv.Ubq1-1:1:2 | 129 | GTG |
| I-Sv.Ubq1-1:1:3 | 131 | GGT |
| I-Zm.UbqM1-1:1:13 (Allele-1) | 138 | GTC |
| I-Zm.UbqM1-1:1:17 (Allele-1) | 140 | GGT |
| I-Zm.UbqM1-1:1:14 (Allele-2) | 142 | GTC |
| I-Zm.UbqM1-1:1:19 (Allele-2) | 144 | GGT |
| I-Zm.UbqM1-1:1:15 (Allele-3) | 146 | GTC |
| I-Zm.UbqM1-1:1:18 (Allele-3) | 148 | GGT |
| I-Sb.Ubq6-1:1:2 | 154 | GTG |
| I-Sb.Ubq6-1:1:3 | 156 | GGT |
| I-Zm.UbqM1-1:1:20 (Allele-3) | 182 | CGG |

Also listed in Table 1 are three allelic variants isolated using the same primer sets designed for amplification of genomic DNA from Z. mays subsp. mexicana. Allelic variants of the EXP sequences are comprised of sequence that shares some identity within various regions of other sequences, but insertions, deletions and nucleotide mismatches may also be apparent within each promoter, leader and/or intron of each of the EXP sequences. The EXP sequence designated EXP-Zm.UbgM1:1:1 (SEQ ID NO: 41) represents a first allele (Allele-1) of the Z. mays subsp. mexicana Ubq1 gene transcriptional regulatory expression element group. The EXP sequences designated EXP-Zm.UbgM1:1:6 (SEQ ID NO: 137) and EXP-Zm.UbgM1:1:10 (SEQ ID NO: 139) represent a first allele (Allele-1), with the only difference between the two EXPs occurring in the last 3′ nucleotides of each respective intron following the sequence 5′-AG-3′ of the 3′ intron splice junction. The EXP sequence designated EXP-Zm.UbgM1:1:4 (SEQ ID NO: 45) represents a second allele (Allele-2) of the Z. mays subsp. mexicana Ubq1 gene transcriptional regulatory expression element group. The EXP sequences designated EXP-Zm.UbgM1:1:7 (SEQ ID NO: 141) and EXP-Zm.UbgM1:1:12 (SEQ ID NO: 143) represent a second allele (Allele-2), with the only difference between the two EXPs occurring in the last 3′ nucleotides of each respective intron following the sequence 5′-AG-3′ of the 3′ intron splice junction. The EXP sequences EXP-Zm.UbgM1:1:2 (SEQ ID NO: 49) and EXP-Zm.UbgM1:1:5 (SEQ ID NO: 53) represents a third allel (Allele-3) of the Z. mays subsp. mexicana Ubq1 gene transcriptional regulatory expression element group and comprise a single nucleotide difference at position 1034 within their respective introns (G for I-Zm.UbgM1-1:1:11, SEQ ID NO: 52 and T for I-Zm.UbgM1-1:1:12, SEQ ID NO: 54). The EXP sequences designated EXP-Zm.UbgM1:1:8 (SEQ ID NO: 145), EXP-Zm.UbgM1:1:9 (SEQ ID NO: 147), EXP-Zm.UbgM1:1:11 (SEQ ID NO: 149) and EXP-Zm.UbgM1:1:13 (SEQ ID NO: 181) also represent a third allele (Allele-3). The intron of EXP-Zm.UbgM1:1:9, I-Zm.UbgM1-1:1:16 (SEQ ID NO: 148) comprises a thymine residue at position 1034, while the introns of EXP-Zm.UbgM1:1:8, EXP-Zm.UbgM1:1:11 and EXP-Zm.UbgM1:1:13 (I-Zm.UbgM1-1:1:15, SEQ ID NO: 146; I-Zm.UbgM1-1:1:18, SEQ ID NO: 11 and; I-Zm.UbgM1-1:1:20, SEQ ID NO: 182) each comprise a guanine residue at position 1034. In addition, the last 3, 3′ end nucleotides of EXP-Zm.UbgM1:1:8 (SEQ ID NO: 145) and EXP-Zm.UbgM1:1:9 (SEQ ID NO: 147) differ from those of EXP-Zm.UbgM1:1:11 (SEQ ID NO: 149) and EXP-Zm.UbgM1:1:13 (SEQ ID NO: 181).

### Example 2: Analysis of Regulatory Elements Driving GUS in Corn Protoplasts

Corn leaf protoplasts were transformed with plant expression vectors containing an EXP sequence driving expression of the β-glucuronidase (GUS) transgene and compared to GUS expression in leaf protoplasts in which expression of GUS is driven by known constitutive promoters.

Expression of a transgene driven by EXP-ANDge.Ubq1:1:7 (SEQ ID NO: 5), EXP-ANDge.Ubq1:1:6 (SEQ ID NO: 12), EXP-ERIra.Ubq1:1:9 (SEQ ID NO: 22) or EXP-ERIra.Ubq1:1:8 (SEQ ID NO: 27) was compared with expression from known constitutive promoters. The foregoing EXP sequences were cloned into plant expression vectors as shown in Table 3 below to yield vectors in which an EXP sequence is operably linked 5′ to a β-glucuronidase (GUS) reporter that contained a processable intron (referred to as GUS-2, SEQ ID NO: 160) derived from the potato light-inducible tissue-specific ST-LS1 gene (GenBank Accession: X04753) or a contiguous GUS coding sequence (GUS-1, SEQ ID NOS: 159), which was operably linked 5′ to a 3′ UTR derived from the A. tumefaciens Nopaline synthase gene (T-AGRtu.nos-1:1:13, SEQ ID NO: 161) or the wheat Hsp17 gene (T-Ta.Hsp17-1:1:1, SEQ ID NO: 162).

TABLE 3
GUS plant expression plasmid construct and corresponding EXP
sequence, GUS coding sequence and 3′ UTR used for
transformation of corn leaf protoplasts. “SEQ ID NO:”
refers to given EXP sequence.

| | | SEQ ID | | |
|---|---|---|---|---|
| Plasmid | EXP sequence | NO: | GUS | 3′ UTR |
| pMON19469 | EXP-CaMV.35S- | 170 | GUS-1 | T-AGRtu.nos-1:1:13 |
| | enh + Zm.DnaK:1:1 | | | |
| pMON65328 | EXP-CaMV.35S- | 163 | GUS-2 | T-Ta.Hsp17-1:1:1 |
| | enh + Ta.Lhcb1 + Os.Act1:1:1 | | | |
| pMON25455 | EXP-Os.Act1:1:9 | 179 | GUS-1 | T-AGRtu.nos-1:1:13 |
| pMON122605 | EXP-Os.TubA-3:1:1 | 165 | GUS-1 | T-AGRtu.nos-1:1:13 |
| pMON136264 | EXP-ANDge.Ubq1:1:7 | 5 | GUS-1 | T-AGRtu.nos-1:1:13 |
| pMON136259 | EXP-ANDge.Ubq1:1:6 | 12 | GUS-1 | T-AGRtu.nos-1:1:13 |
| pMON136263 | EXP-ERIra.Ubq1:1:9 | 22 | GUS-1 | T-AGRtu.nos-1:1:13 |
| pMON136258 | EXP-ERIra.Ubq1:1:8 | 27 | GUS-1 | T-AGRtu.nos-1:1:13 |

Control plasmids (pMON19469, pMON65328, pMON25455 and pMON122605) used for comparison were constructed as described above and contain a known EXP sequence: EXP-CaMV.35S-enh+Zm.DnaK:1:1 (SEQ ID NO: 170), EXP-CaMV.35S-enh+Ta.Lhcb1+Os.Act1:1:1 (SEQ ID NO: 163), EXP-Os.Act1:1:9 (SEQ ID NO: 179), or EXP-Os.TubA-3:1:1 (SEQ ID NO: 165), respectively, operably linked 5′ to a GUS coding sequence and 3′ UTR. Three additional controls were provided to assess background GUS and luciferase expression: a no DNA control, an empty vector which is not designed for transgene expression, and an expression vector used to express green fluorescent protein (GFP).

Two plasmids, for use in co-transformation and normalization of data, were also constructed using methods known in the art. Each plasmid contained a specific luciferase coding sequence that was driven by a constitutive EXP sequence. The plant vector pMON19437 comprises a transgene cassette with a constitutive promoter operably linked 5′ to an intron, (EXP-CaMV.35S-enh+Zm.DnaK:1:1, SEQ ID NO: 170), operably linked 5′ to a firefly (Photinus pyralis) luciferase coding sequence (LUCIFERASE:1:3, SEQ ID NO: 166), operably linked 5′ to a 3′ UTR from the Agrobacterium tumefaciens nopaline synthase gene (T-AGRtu.nos-1:1:13, SEQ ID NO: 161). The plant vector pMON63934 comprises a transgene cassette with a constitutive EXP sequence (EXP-CaMV.35S-enh-Lhcb1, SEQ ID NO: 168), operably linked 5′ to a sea pansy (Renilla reniformis) luciferase coding sequence (CR-Ren.hRenilla Lucife-0:0:1, SEQ ID NO: 167), operably linked 5′ to a 3′ UTR from the Agrobacterium tumefaciens nopaline synthase gene (T-AGRtu.nos-1:1:13, SEQ ID NO: 161).

Corn leaf protoplasts were transformed using a PEG-based transformation method, as is well known in the art. Protoplast cells were transformed with pMON19437 plasmid DNA, pMON63934 plasmid DNA, and an equimolar quantity of one of the plasmids presented in Table 3 and incubated overnight in total darkness. Measurements of both GUS and luciferase were conducted by placing aliquots of a lysed preparation of cells transformed as above into two different small-well trays. One tray was used for GUS measurements, and a second tray was used to perform a dual luciferase assay using the dual luciferase reporter assay system (Promega Corp., Madison, WI; see for example, Promega Notes Magazine, No: 57, 1996, p. 02). One or two transformations for each EXP sequence were performed and the mean expression values for each EXP sequence determined from several samples from each transformation experiment. Sample measurements were made using four replicates of each EXP sequence construct transformation, or alternatively, three replicates of each EXP sequence construct per one of two transformation experiments. The mean GUS and luciferase expression levels are provided in Table 4. In this table, the firefly luciferase values (e.g. from expression of pMON19437) are provided in the column labeled “FLuc” and the Renilla luciferase values are provided as in the column labeled “RLuc.”

TABLE 4
Mean GUS and Luciferase activity in transformed corn leaf protoplast cells.

| | | SEQ ID | | | |
|---|---|---|---|---|---|
| Plasmid | EXP sequence | NO: | Gus | RLuc | FLuc |
| pMON19469 | EXP-CaMV.35S- | 170 | 789147 | 298899 | 36568 |
| | enh + Zm.DnaK:1:1 | | | | |
| pMON65328 | EXP-CaMV.35S- | 163 | 508327 | 158227 | 17193 |
| | enh + Ta.Lhcb1 + Os.Act1:1:1 | | | | |
| pMON25455 | EXP-Os.Act1:1:9 | 179 | 460579 | 183955 | 53813 |
| pMON122605 | EXP-Os.TubA-3:1:1 | 165 | 25082 | 25821 | 21004 |
| pMON136264 | EXP-ANDge.Ubq1:1:7 | 5 | 926083 | 101213 | 23704 |
| pMON136259 | EXP-ANDge.Ubq1:1:6 | 12 | 845274 | 193153 | 51479 |
| pMON136263 | EXP-ERIra.Ubq1:1:9 | 22 | 901985 | 132765 | 41313 |
| pMON136258 | EXP-ERIra.Ubq1:1:8 | 27 | 1011447 | 210635 | 66803 |

To compare the relative activity of each EXP sequence, GUS values were expressed as a ratio of GUS to luciferase activity and normalized with respect to the expression levels observed for the EXP sequence EXP-Os.TubA-3:1:1 (SEQ ID NO: 165). Table 5 below shows the GUS/RLuc ratios of expression normalized with respect to EXP-Os.TubA-3:1:1 expression in corn protoplasts.

As can be seen in Table 5, GUS expression, driven by EXP-ANDge.Ubq1:1:7 (SEQ ID NO: 5), EXP-ANDge.Ubq1:1:6 (SEQ ID NO: 12), EXP-ERIra.Ubq1:1:9 (SEQ ID NO: 22) or EXP-ERIra.Ubq1:1:8 (SEQ ID NO: 27) was 4.51 to 9.42 fold higher than GUS expression driven by EXP-Os.TubA-3:1:1 (SEQ ID NO: 165). GUS expression driven by EXP-ANDge.Ubq1:1:7 (SEQ ID NO: 5), EXP-ANDge.Ubq1:1:6 (SEQ ID NO: 12), EXP-ERIra.Ubq1:1:9 (SEQ ID NO: 22) or EXP-ERIra.Ubq1:1:8 (SEQ ID NO: 27) was also higher than that of EXP-CaMV.35S-enh+Zm.DnaK:1:1 (SEQ ID NO: 170), EXP-CaMV.35S-enh+Ta.Lhcb1+Os.Act1:1:1 (SEQ ID NO: 163), or EXP-Os.Act1:1:9 (SEQ ID NO: 179).

TABLE 5
GUS/RLuc fold expression as relative to EXP-Os.TubA-3:1:1 expression in corn
leaf protoplast cells.

| | | | | Gus/RLuc |
|---|---|---|---|---|
| | | | | Normalized |
| | | SEQ | | with respect to |
| | | ID | | EXP-Os.TubA- |
| Plasmid | EXP sequence | NO: | Gus/RLuc | 3:1:1 |
| pMON19469 | EXP-CaMV.35S-enh + Zm.DnaK:1:1 | 170 | 2.640000 | 2.72 |
| pMON65328 | EXP-CaMV.35S- | 163 | 3.210000 | 3.31 |
| | enh + Ta.Lhcb1 + Os.Act1:1:1 | | | |
| pMON25455 | EXP-Os.Act1:1:9 | 179 | 2.500000 | 2.57 |
| pMON122605 | EXP-Os.TubA-3:1:1 | 165 | 0.971000 | 1.00 |
| pMON136264 | EXP-ANDge.Ubq1:1:7 | 5 | 9.150000 | 9.42 |
| pMON136259 | EXP-ANDge.Ubq1:1:6 | 12 | 4.380000 | 4.51 |
| pMON136263 | EXP-ERIra.Ubq1:1:9 | 22 | 6.790000 | 6.99 |
| pMON136258 | EXP-ERIra.Ubq1:1:8 | 27 | 4.800000 | 4.94 |

Table 6 below show GUS/FLuc ratios of expression normalized with respect to EXP-Os.TubA-3:1:1 expression in corn protoplasts.

TABLE 6
GUS/FLuc fold expression as relative to EXP-Os.TubA-3:1:1 expression in corn
leaf protoplast cells.

| | | | | Normalized |
|---|---|---|---|---|
| | | | | with respect |
| | | SEQ | | to EXP- |
| | | ID | | Os.TubA- |
| Plasmid | EXP sequence | NO: | Gus/FLuc | 3:1:1 |
| pMON19469 | EXP-CaMV.35S-enh + Zm.DnaK:1:1 | 170 | 21.600000 | 18.15 |
| pMON65328 | EXP-CaMV.35S- | 163 | 29.600000 | 24.87 |
| | enh + Ta.Lhcb1 + Os.Act1:1:1 | | | |
| pMON25455 | EXP-Os.Act1:1:9 | 179 | 8.560000 | 7.19 |
| pMON122605 | EXP-Os.TubA-3:1:1 | 165 | 1.190000 | 1.00 |
| pMON136264 | EXP-ANDge.Ubq1:1:7 | 5 | 39.100000 | 32.86 |
| pMON136259 | EXP-ANDge.Ubq1:1:6 | 12 | 16.400000 | 13.78 |
| pMON136263 | EXP-ERIra.Ubq1:1:9 | 22 | 21.800000 | 18.32 |
| pMON136258 | EXP-ERIra.Ubq1:1:8 | 27 | 15.100000 | 12.69 |

As can be seen in Table 6, GUS expression, driven by EXP-ANDge.Ubq1:1:7 (SEQ ID NO: 5), EXP-ANDge.Ubq1:1:6 (SEQ ID NO: 12), EXP-ERIra.Ubq1:1:9 (SEQ ID NO: 22) or EXP-ERIra.Ubq1:1:8 (SEQ ID NO: 27) demonstrated the same general trend when expressed as ratio of GUS/FLuc values and is normalized with respect to EXP-Os.TubA-3:1:1 (SEQ ID NO: 165). Expression was 12.69 to 32.86 fold higher than GUS expression driven by EXP-Os.TubA-3:1:1 (SEQ ID NO: 165). GUS expression driven by EXP-ANDge.Ubq1:1:7 (SEQ ID NO: 5), EXP-ANDge.Ubq1:1:6 (SEQ ID NO: 12), EXP-ERIra.Ubq1:1:9 (SEQ ID NO: 22) or EXP-ERIra.Ubq1:1:8 (SEQ ID NO: 27) was also higher in certain comparisons than that of EXP-CaMV.35S-enh+Zm.DnaK:1:1 (SEQ ID NO: 170), EXP-CaMV.35S-enh+Ta.Lhcb1+Os.Act1:1:1 (SEQ ID NO: 163), or EXP-Os.Act1:1:9 (SEQ ID NO: 179).

### Example 3: Analysis of Regulatory Elements Driving GUS in Corn Protoplasts Using GUS Transgene Cassette Amplicons

Corn leaf protoplasts were transformed with DNA amplicons derived from plant expression vectors containing an EXP sequence, driving expression of the β-glucuronidase (GUS) transgene, and compared to leaf protoplast in which expression of GUS is driven by known constitutive promoters in a series of experiments presented below.

In a first set of experiments, corn protoplast cells, derived from leaf tissue were transformed as above with amplicons produced from amplification of GUS transgene cassettes comprising plant expression vectors to compare expression of a transgene (GUS) driven by one of EXP-ANDge.Ubq1:1:7 (SEQ ID NO: 5), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-SETit.Ubq1:1:5 (SEQ ID NO: 117), EXP-SETit.Ubq1:1:7 (SEQ ID NO: 123), EXP-SETit.Ubq1:1:6 (SEQ ID NO: 124), EXP-Sv.Ubq1:1:7 (SEQ ID NO: 128), EXP-Sv.Ubq1:1:8 (SEQ ID NO: 132), EXP-Sv.Ubq1:1:10 (SEQ ID NO: 134), EXP-Zm.UbgM1:1:6 (SEQ ID NO: 137), EXP-Zm.UbgM1:1:7 (SEQ ID NO: 141), EXP-Sb.Ubg4:1:2 (SEQ ID NO: 151), EXP-Sb.Ubg6:1:2 (SEQ ID NO: 153) and EXP-Cl.Ubq1:1:10 (SEQ ID NO: 98) with that of known constitutive promoters. Each EXP sequence comprising the amplification template from which the transgene cassette amplicon is produced was cloned using methods known in the art into a plant expression vector shown in Table 7 below under the heading of “Amplicon Template.” The resulting plant expression vectors comprise a transgene cassette comprised of a EXP sequence, operably linked 5′ to a coding sequence for β-glucuronidase (GUS) that either contains a processable intron (“GUS-2” as discussed in Example 2 above), or a contiguous GUS coding sequence (“GUS-1”, as discussed above), operably linked 5′ to a 3′ UTR T-AGRtu.nos-1:1:13 or T-Ta.Hsp17-1:1:1, as also noted above. Amplicons were produced using methods known to those skilled in the art using the plasmid construct templates presented in Table 7 below. Briefly, a 5′ oligonucleotide primer was designed to anneal to the promoter sequence and a 3′ oligonucleotide primer, which anneals at the 3′ end of the 3′ UTR was used for amplification of each transgene cassette. Successive 5′ deletions were introduced into the promoter sequences comprising the transgene cassettes, giving rise to different EXP sequences, by the use of different oligonucleotide primers which were designed to anneal at different positions within the promoter sequence comprising each amplicon template.

TABLE 7
GUS plant expression amplicons and corresponding plasmid construct amplicon
templates, EXP sequence, GUS coding sequence and 3′ UTR used for transformation of
corn leaf protoplasts.

| | | | SEQ | GUS | |
|---|---|---|---|---|---|
| | Amplicon | | ID | Coding | |
| Amplicon ID | Template | EXP sequence | NO: | Sequence | 3′ UTR |
| PCR0145942 | pMON25455 | EXP-Os.Act1:1:9 | 179 | GUS-1 | T- |
| | | | | | AGRtu.nos- |
| | | | | | 1:1:13 |
| PCR0145941 | pMON33449 | P-CAMV.35S-ENH- | 169 | GUS-1 | T- |
| | | 1:1:102/L-CAMV.35S- | | | AGRtu.nos- |
| | | 1:1:2 | | | 1:1:13 |
| PCR0145943 | pMON65328 | EXP-CaMV.35S- | 163 | GUS-2 | T- |
| | | enh + Ta.Lhcb1 + Os.Act1:1:1 | | | Ta.Hsp17- |
| | | | | | 1:1:1 |
| PCR0145944 | pMON81552 | EXP-CaMV.35S- | 170 | GUS-1 | T- |
| | | enh + Zm.DnaK:1:1 | | | AGRtu.nos- |
| | | | | | 1:1:13 |
| PCR0145892 | pMON136264 | EXP-ANDge.Ubq1:1:7 | 5 | GUS-1 | T- |
| | | | | | AGRtu.nos- |
| | | | | | 1:1:13 |
| PCR0145815 | pMON136264 | EXP-ANDge.Ubq1:1:10 | 10 | GUS-1 | T- |
| | | | | | AGRtu.nos- |
| | | | | | 1:1:13 |
| PCR0145893 | pMON136259 | EXP-ANDge.Ubq1:1:6 | 12 | GUS-1 | T- |
| | | | | | AGRtu.nos- |
| | | | | | 1:1:13 |
| PCR0145817 | pMON136264 | EXP-ANDge.Ubq1:1:11 | 14 | GUS-1 | T- |
| | | | | | AGRtu.nos- |
| | | | | | 1:1:13 |
| PCR0145819 | pMON136264 | EXP-ANDge.Ubq1:1:12 | 16 | GUS-1 | T- |
| | | | | | AGRtu.nos- |
| | | | | | 1:1:13 |
| PCR0145896 | pMON136263 | EXP-ERIra.Ubq1:1:9 | 22 | GUS-1 | T- |
| | | | | | AGRtu.nos- |
| | | | | | 1:1:13 |
| PCR0145820 | pMON136263 | EXP-ERIra.Ubq1:1:10 | 25 | GUS-1 | T- |
| | | | | | AGRtu.nos- |
| | | | | | 1:1:13 |
| PCR0145897 | pMON136258 | EXP-ERIra.Ubq1:1:8 | 27 | GUS-1 | T- |
| | | | | | AGRtu.nos- |
| | | | | | 1:1:13 |
| PCR0145821 | pMON136263 | EXP-ERIra.Ubq1:1:11 | 29 | GUS-1 | T- |
| | | | | | AGRtu.nos- |
| | | | | | 1:1:13 |
| PCR0145822 | pMON136263 | EXP-ERIra.Ubq1:1:12 | 31 | GUS-1 | T- |
| | | | | | AGRtu.nos- |
| | | | | | 1:1:13 |
| PCR0145900 | pMON140877 | EXP-SETit.Ubq1:1:5 | 117 | GUS-1 | T- |
| | | | | | AGRtu.nos- |
| | | | | | 1:1:13 |
| PCR0145928 | pMON140877 | EXP-SETit.Ubq1:1:7 | 123 | GUS-1 | T- |
| | | | | | AGRtu.nos- |
| | | | | | 1:1:13 |
| PCR0145905 | pMON140877 | EXP-SETit.Ubq1:1:6 | 124 | GUS-1 | T- |
| | | | | | AGRtu.nos- |
| | | | | | 1:1:13 |
| PCR0145909 | pMON140878 | EXP-Sv.Ubq1:1:7 | 128 | GUS-1 | T- |
| | | | | | AGRtu.nos- |
| | | | | | 1:1:13 |
| PCR0145929 | pMON140878 | EXP-Sv.Ubq1:1:8 | 132 | GUS-1 | T- |
| | | | | | AGRtu.nos- |
| | | | | | 1:1:13 |
| PCR0145911 | pMON140878 | EXP-Sv.Ubq1:1:10 | 134 | GUS-1 | T- |
| | | | | | AGRtu.nos- |
| | | | | | 1:1:13 |
| PCR0145914 | pMON140881 | EXP-Zm.UbqM1:1:6 | 137 | GUS-1 | T- |
| | | | | | AGRtu.nos- |
| | | | | | 1:1:13 |
| PCR0145915 | pMON140882 | EXP-Zm.UbqM1:1:7 | 141 | GUS-1 | T- |
| | | | | | AGRtu.nos- |
| | | | | | 1:1:13 |
| PCR0145921 | pMON140886 | EXP-Sb.Ubq4:1:2 | 151 | GUS-1 | T- |
| | | | | | AGRtu.nos- |
| | | | | | 1:1:13 |
| PCR0145920 | pMON140887 | EXP-Sb.Ubq6:1:2 | 153 | GUS-1 | T- |
| | | | | | AGRtu.nos- |
| | | | | | 1:1:13 |
| PCR0145922 | pMON140889 | EXP-Cl.Ubq1:1:10 | 98 | GUS-1 | T- |
| | | | | | AGRtu.nos- |
| | | | | | 1:1:13 |

Plasmid constructs listed as amplicon templates in Table 7 served as templates for amplification of transgene expression cassettes comprising the listed EXP sequences of Table 7. Control plasmids used to generate GUS transgene amplicons for comparison were constructed as previously described with known constitutive EXP sequences described in Example 2. Negative controls for determination of GUS and luciferase background, a no DNA control, and a control sample in which the two luciferase plasmids are used in transformation along with a plasmid DNA that does not express a coding sequence were also used. Plasmids pMON19437 and pMON63934, as discussed in Example 2, were also employed for co-transformation and normalization of data.

Corn leaf protoplasts were transformed using a PEG-based transformation method as described in Example 2, above. Table 8 below shows the average GUS and luciferase expression values determined for each transgene cassette.

TABLE 8
Mean GUS and Luciferase activity in transformed
corn leaf protoplast cells.

| | SEQ ID | | | |
|---|---|---|---|---|
| EXP sequence | NO: | GUS | RLuc | FLuc |
| EXP-Os.Act1:1:9 | 179 | 1540.3 | 105416.8 | 2671.8 |
| P-CAMV.35S-ENH-1:1:102/ | 169 | 10426.3 | 344088.6 | 8604.1 |
| L-CAMV.35S-1:1:2 | | | | |
| EXP-CaMV.35S-enh + | 163 | 12530.8 | 137722.6 | 3067.1 |
| Ta.Lhcb1 + Os.Act1:1:1 | | | | |
| EXP-CaMV.35S-enh + | 170 | 61036.1 | 208125.3 | 5787.6 |
| Zm.DnaK:1:1 | | | | |
| EXP-ANDge.Ubq1:1:7 | 5 | 59447.4 | 84667.6 | 2578.4 |
| EXP-ANDge.Ubq1:1:10 | 10 | 40123.3 | 76753.8 | 2419.8 |
| EXP-ANDge.Ubq1:1:6 | 12 | 42621.0 | 121751.3 | 3974.8 |
| EXP-ANDge.Ubq1:1:11 | 14 | 44358.5 | 87105.8 | 2687.1 |
| EXP-ANDge.Ubq1:1:12 | 16 | 48219.0 | 107762.1 | 3279.6 |
| EXP-ERIra.Ubq1:1:9 | 22 | 31253.0 | 171684.1 | 6476.1 |
| EXP-ERIra.Ubq1:1:10 | 25 | 7905.8 | 21235.6 | 462.4 |
| EXP-ERIra.Ubq1:1:8 | 27 | 39935.8 | 173766.6 | 5320.3 |
| EXP-ERIra.Ubq1:1:11 | 29 | 34141.3 | 111626.8 | 3377.6 |
| EXP-ERIra.Ubq1:1:12 | 31 | 11540.3 | 42362.1 | 1045.3 |
| EXP-SETit.Ubq1:1:5 | 117 | 20496.5 | 88695.8 | 2358.8 |
| EXP-SETit.Ubq1:1:7 | 123 | 75728.5 | 185223.8 | 4723.1 |
| EXP-SETit.Ubq1:1:6 | 124 | 44148.3 | 161216.3 | 4962.1 |
| EXP-Sv.Ubq1:1:7 | 128 | 15043.8 | 74670.6 | 1888.3 |
| EXP-Sv.Ubq1:1:8 | 132 | 31997.8 | 113787.1 | 3219.8 |
| EXP-Sv.Ubq1:1:10 | 134 | 38952.8 | 220208.6 | 7011.3 |
| EXP-Zm.UbqM1:1:6 | 137 | 30528.3 | 90113.1 | 2453.6 |
| EXP-Zm.UbqM1:1:7 | 141 | 34986.3 | 105724.7 | 2553.8 |
| EXP-Sb.Ubq4:1:2 | 151 | 9982.3 | 72593.8 | 2171.6 |
| EXP-Sb.Ubq6:1:2 | 153 | 33689.0 | 114709.6 | 3879.6 |
| EXP-Cl.Ubq1:1:10 | 98 | 50622.3 | 107084.3 | 2621.3 |

To compare the relative activity of each EXP sequence GUS values were expressed as a ratio of GUS to luciferase activity and normalized with respect to the expression levels observed for EXP-Os.Act1:1:1 and EXP-CaMV.35S-enh+Ta.Lhcb1+Os.Act1:1:1. Table 9 below shows the GUS/RLuc ratios of expression normalized with respect to EXP-Os.Act1:1:1 and EXP-CaMV.35S-enh+Ta.Lhcb1+Os.Act1:1:1 driven expression in corn protoplasts. Table 10 below shows the GUS/FLuc ratios of expression normalized with respect to EXP-Os.Act1:1:1 and EXP-CaMV.35S-enh+Ta.Lhcb1+Os.Act1:1:1 driven expression in corn protoplasts.

TABLE 9
GUS/RLuc and GUS/FLuc ratios of expression normalized
with respect to EXP-CaMV.35S-enh + Ta.Lhcb1 +
Os.Act1:1:1 (SEQ ID NO: 163) in corn protoplasts.

| | | GUS/RLuc | GUS/FLuc |
|---|---|---|---|
| | | Relative | Relative |
| | | to EXP- | to EXP- |
| | | CaMV.35S-enh + | CaMV.35S-enh + |
| | SEQ ID | Ta.Lhcb1 + | Ta.Lhcb1 + |
| EXP sequence | NO: | Os.Act1:1:1 | Os.Act1:1:1 |
| EXP-Os.Act1:1:9 | 179 | 0.16 | 0.14 |
| P-CAMV.35S-ENH- | 169 | 0.33 | 0.30 |
| 1:1:102/L-CAMV.35S- | | | |
| 1:1:2 | | | |
| EXP-CaMV.35S- | 163 | 1.00 | 1.00 |
| enh + Ta.Lhcb1 + | | | |
| Os.Act1:1:1 | | | |
| EXP-CaMV.35S- | 170 | 3.22 | 2.58 |
| enh + Zm.DnaK:1:1 | | | |
| EXP-ANDge.Ubq1:1:7 | 5 | 7.72 | 5.64 |
| EXP-ANDge.Ubq1:1:10 | 10 | 5.75 | 4.06 |
| EXP-ANDge.Ubq1:1:6 | 12 | 3.85 | 2.62 |
| EXP-ANDge.Ubq1:1:11 | 14 | 5.60 | 4.04 |
| EXP-ANDge.Ubq1:1:12 | 16 | 4.92 | 3.60 |
| EXP-ERIra.Ubq1:1:9 | 22 | 2.00 | 1.18 |
| EXP-ERIra.Ubq1:1:10 | 25 | 4.09 | 4.18 |
| EXP-ERIra.Ubq1:1:8 | 27 | 2.53 | 1.84 |
| EXP-ERIra.Ubq1:1:11 | 29 | 3.36 | 2.47 |
| EXP-ERIra.Ubq1:1:12 | 31 | 2.99 | 2.70 |
| EXP-SETit.Ubq1:1:5 | 117 | 2.54 | 2.13 |
| EXP-SETit.Ubq1:1:7 | 123 | 4.49 | 3.92 |
| EXP-SETit.Ubq1:1:6 | 124 | 3.01 | 2.18 |
| EXP-Sv.Ubq1:1:7 | 128 | 2.21 | 1.95 |
| EXP-Sv.Ubq1:1:8 | 132 | 3.09 | 2.43 |
| EXP-Sv.Ubq1:1:10 | 134 | 1.94 | 1.36 |
| EXP-Zm.UbqM1:1:6 | 137 | 3.72 | 3.05 |
| EXP-Zm.UbqM1:1:7 | 141 | 3.64 | 3.35 |
| EXP-Sb.Ubq4:1:2 | 151 | 1.51 | 1.13 |
| EXP-Sb.Ubq6:1:2 | 153 | 3.23 | 2.13 |
| EXP-Cl.Ubq1:1:10 | 98 | 5.20 | 4.73 |

TABLE 10
GUS/RLuc and GUS/FLuc ratios of expression normalized
with respect to EXP-Os.Act1:1:9 (SEQ ID NO: 179) in
corn leaf protoplasts.

| | | GUS/RLuc | GUS/FLuc |
|---|---|---|---|
| | SEQ | Relative | Relative |
| | ID | to EXP- | to EXP- |
| EXP sequence | NO: | Os.Act1:1:9 | Os.Act1:1:9 |
| EXP-Os.Act1:1:9 | 179 | 1.00 | 1.00 |
| P-CAMV.35S-ENH-1:1:102/L- | 169 | 2.07 | 2.10 |
| CAMV.35S-1:1:2 | | | |
| EXP-CaMV.35S- | 163 | 6.23 | 7.09 |
| enh + Ta.Lhcb1 + Os.Act1:1:1 | | | |
| EXP-CaMV.35S-enh + Zm.DnaK:1:1 | 170 | 20.07 | 18.29 |
| EXP-ANDge.Ubq1:1:7 | 5 | 48.05 | 39.99 |
| EXP-ANDge.Ubq1:1:10 | 10 | 35.78 | 28.76 |
| EXP-ANDge.Ubq1:1:6 | 12 | 23.96 | 18.60 |
| EXP-ANDge.Ubq1:1:11 | 14 | 34.85 | 28.64 |
| EXP-ANDge.Ubq1:1:12 | 16 | 30.62 | 25.50 |
| EXP-ERIra.Ubq1:1:9 | 22 | 12.46 | 8.37 |
| EXP-ERIra.Ubq1:1:10 | 25 | 25.48 | 29.66 |
| EXP-ERIra.Ubq1:1:8 | 27 | 15.73 | 13.02 |
| EXP-ERIra.Ubq1:1:11 | 29 | 20.93 | 17.53 |
| EXP-ERIra.Ubq1:1:12 | 31 | 18.64 | 19.15 |
| EXP-SETit.Ubq1:1:5 | 117 | 15.82 | 15.07 |
| EXP-SETit.Ubq1:1:7 | 123 | 27.98 | 27.81 |
| EXP-SETit.Ubq1:1:6 | 124 | 18.74 | 15.43 |
| EXP-Sv.Ubq1:1:7 | 128 | 13.79 | 13.82 |
| EXP-Sv.Ubq1:1:8 | 132 | 19.25 | 17.24 |
| EXP-Sv.Ubq1:1:10 | 134 | 12.11 | 9.64 |
| EXP-Zm.UbqM1:1:6 | 137 | 23.19 | 21.58 |
| EXP-Zm.UbqM1:1:7 | 141 | 22.65 | 23.76 |
| EXP-Sb.Ubq4:1:2 | 151 | 9.41 | 7.97 |
| EXP-Sb.Ubq6:1:2 | 153 | 20.10 | 15.06 |
| EXP-Cl.Ubq1:1:10 | 98 | 32.35 | 33.50 |

As can be seen in Tables 9 and 10, nearly all of the EXP sequences were capable of driving GUS transgene expression in corn cells. Average GUS expression was higher for EXP-ANDge.Ubq1:1:7 (SEQ ID NO: 5), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-SETit.Ubq1:1:5 (SEQ ID NO: 117), EXP-SETit.Ubq1:1:7 (SEQ ID NO: 123), EXP-SETit.Ubq1:1:6 (SEQ ID NO: 124), EXP-Sv.Ubq1:1:7 (SEQ ID NO: 128), EXP-Sv.Ubq1:1:8 (SEQ ID NO: 132), EXP-Sv.Ubq1:1:10 (SEQ ID NO: 134), EXP-Zm.UbgM1:1:6 (SEQ ID NO: 137), EXP-Zm.UbgM1:1:7 (SEQ ID NO: 141), EXP-Sb.Ubg4:1:2 (SEQ ID NO: 151), EXP-Sb.Ubg6:1:2 (SEQ ID NO: 153) and EXP-Cl.Ubq1:1:10 (SEQ ID NO: 98) when compared to GUS expression driven by EXP-Os.Act1:1:1 or EXP-CaMV.35S-enh+Ta.Lhcb1+Os.Act1:1:1.

In a second set of experiments, a GUS cassette amplicon comprising the EXP sequence EXP-Zm.UbgM1:1:8 (SEQ ID NO: 145) was compared to the control amplicons, PCR0145942 (EXP-Os.Act1:1:9, SEQ ID NO: 179) and PCR0145944 (EXP-CaMV.35S-enh+Zm.DnaK:1:1, SEQ ID NO: 170) with respect to GUS expression. GUS expression driven by the EXP sequence EXP-Zm.UbgM1:1:8 was higher than that of the two controls. Table 11 below shows the mean GUS and luciferase values determined for each amplicon. Table 12 below shows the GUS/RLuc and GUS/FLuc ratios of expression normalized with respect to EXP-Os.Act1:1:9 and EXP-CaMV.35S-enh+Zm.DnaK:1:1 driven expression in corn protoplasts.

TABLE 11
Mean GUS and Luciferase activity in transformed
corn leaf protoplast cells.

| | | SEQ | | | |
|---|---|---|---|---|---|
| | | ID | | | |
| Amplicon | EXP sequence | NO: | GUS | RLuc | FLuc |
| PCR0145942 | EXP-Os.Act1:1:9 | 179 | 1512.25 | 190461 | 11333.8 |
| PCR0145944 | EXP-CaMV.35S- | 170 | 41176.5 | 330837 | 13885.8 |
| | enh + Zm.DnaK:1:1 | | | | |
| PCR0145916 | EXP-Zm.UbqM1:1:8 | 145 | 79581.5 | 330756 | 15262.5 |

TABLE 12
GUS/RLuc and GUS/FLuc ratios of expression normalized with respect to EXP-
Os.Act1:1:9 (SEQ ID NO: 179) and EXP-CaMV.35S-enh + Zm.DnaK:1:1
(SEQ ID NO: 170) in corn leaf protoplasts.

| | | | | GUS/RLuc | |
|---|---|---|---|---|---|
| | | | | relative to | GUS/FLuc |
| | | GUS/RLuc | GUS/FLuc | EXP- | relative to EXP- |
| | SEQ | relative to | relative to | CaMV.35S- | CaMV.35S- |
| | ID | EXP- | EXP- | enh + Zm.DnaK: | enh + Zm.DnaK: |
| EXP sequence | NO: | Os.Act1:1:9 | Os.Act1:1:9 | 1:1 | 1:1 |
| EXP-Os.Act1:1:9 | 179 | 1.00 | 1.00 | 0.06 | 0.04 |
| EXP-CaMV.35S- | 170 | 15.68 | 22.22 | 1.00 | 1.00 |
| enh + Zm.DnaK:1:1 | | | | | |
| EXP-Zm.UbqM1:1:8 | 145 | 30.30 | 39.08 | 1.93 | 1.76 |

In a third set of experiments, amplicon GUS transgene cassettes were made as described above and assayed for expression driven by the EXP sequences, EXP-ANDge.Ubq1:1:8 (SEQ ID NO: 8), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116). The amplicons were comprised of an EXP sequence operably linked to the GUS-1 coding sequence which was operably linked to the T-AGRtu.nos-1:1:13 3′ UTR. Expression was compared to the controls EXP-Os.Act1:1:9 (SEQ ID NO: 179) and EXP-CaMV.35S-enh+Zm.DnaK:1:1 (SEQ ID NO: 170). Table 13 below shows the mean GUS and luciferase values determined for each amplicon. Table 14 below shows the GUS/RLuc ratios of expression normalized with respect to EXP-Os.Act1:1:9 and EXP-CaMV.35S-enh+Zm.DnaK:1:1 driven expression in corn protoplasts.

TABLE 13
Mean GUS and Luciferase activity in
transformed corn leaf protoplast cells.

| | | SEQ | | |
|---|---|---|---|---|
| Amplicon ID | EXP sequence | ID NO: | GUS | RLuc |
| PCR0145942 | EXP-Os.Act1:1:9 | 179 | 9445.25 | 929755 |
| PCR0145944 | EXP-CaMV.35S-enh+ | 170 | 78591.25 | 445127 |
| | Zm.DnaK:1:1 | | | |
| PCR0146628 | EXP-ANDge.Ubq1:1:8 | 8 | 192056.75 | 972642 |
| PCR0145922 | EXP-Cl.Ubq1:1:10 | 98 | 175295.25 | 395563 |
| PCR0145945 | EXP-Cl.Ubq1:1:13 | 114 | 173674.5 | 402966 |
| PCR0145946 | EXP-Cl.Ubq1:1:14 | 115 | 185987.5 | 390052 |
| PCR0145947 | EXP-Cl.Ubq1:1:15 | 116 | 9435 | 320749 |

TABLE 14
GUS/RLuc and GUS/FLuc ratios of expression normalized
with respect to EXP-Os.Act1:1:9 (SEQ ID NO: 179) and
EXP-CaMV.35S-enh+Zm.DnaK:1:1 (SEQ ID NO: 170)
in corn leaf protoplasts.

| | | GUS/RLuc | GUS/RLuc |
|---|---|---|---|
| | | relative to | relative to EXP- |
| | SEQ ID | EXP- | CaMV.35S- |
| EXP sequence | NO: | Os.Act1:1:9 | enh + Zm.DnaK:1:1 |
| EXP-Os.Act1:1:9 | 179 | 1.00 | 0.06 |
| EXP-CaMV.35S- | 170 | 17.38 | 1.00 |
| enh+Zm.DnaK:1:1 | | | |
| EXP-ANDge.Ubq1:1:8 | 8 | 19.44 | 1.12 |
| EXP-Cl.Ubq1:1:10 | 98 | 43.62 | 2.51 |
| EXP-Cl.Ubq1:1:13 | 114 | 42.43 | 2.44 |
| EXP-Cl.Ubq1:1:14 | 115 | 46.94 | 2.70 |
| EXP-Cl.Ubq1:1:15 | 116 | 2.90 | 0.17 |

As can be seen in Table 14 above, the EXP sequences EXP-ANDge.Ubq1:1:8 (SEQ ID NO: 8), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116) are capable of driving transgene expression. Expression driven by EXP-ANDge.Ubq1:1:8 (SEQ ID NO: 8), EXP-Cl.Ubq1:1:10 (SEQ ID NO: 98), EXP-Cl.Ubq1:1:13 (SEQ ID NO: 114) and EXP-Cl.Ubq1:1:14 (SEQ ID NO: 115) was higher than that of both controls. Expression driven by EXP-Cl.Ubq1:1:15 (SEQ ID NO: 116) was lower than EXP-CaMV.35S-enh+Zm.DnaK:1:1 (SEQ ID NO: 170) but higher than the control, EXP-Os.Act1:1:9 (SEQ ID NO: 179).

In a fourth set of experiments, amplicon GUS transgene cassettes were made as described above and assayed for expression driven by the EXP sequences, EXP-Cl.Ubq1:1:10 (SEQ ID NO: 98), EXP-Cl.Ubq1:1:16 (SEQ ID NO: 93) and EXP-Cl.Ubq1:1:17 (SEQ ID NO: 97). Expression was compared to the controls EXP-Os.Act1:1:9 (SEQ ID NO: 179) and EXP-CaMV.35S-enh+Zm.DnaK:1:1 (SEQ ID NO: 170). Table 15 below shows the mean GUS and luciferase values determined for each amplicon. Table 16 below shows the GUS/RLuc and GUS/FLuc ratios of expression normalized with respect to EXP-Os.Act1:1:9 and EXP-CaMV.35S-enh+Zm.DnaK:1:1 driven expression in corn protoplasts.

TABLE 15
Mean GUS and Luciferase activity in transformed corn leaf protoplast cells.

| | | SEQ | | | |
|---|---|---|---|---|---|
| | | ID | | | |
| Amplicon ID | EXP sequence | NO: | GUS | RLuc | FLuc |
| PCR0145942 | EXP-Os.Act1:1:9 | 179 | 5333.5 | 171941.75 | 77817.88 |
| PCR0145944 | EXP-CaMV.35S-enh+Zm.DnaK:1:1 | 170 | 88517 | 177260.25 | 54207.38 |
| PCR0145922 | EXP-Cl.Ubq1:1:10 | 98 | 130125.75 | 194216 | 32055 |
| pMON146750 | EXP-Cl.Ubq1:1:16 | 93 | 134101.75 | 182317.5 | 32434.5 |
| pMON146751 | EXP-Cl.Ubq1:1:17 | 97 | 107122.5 | 151783.25 | 51354.38 |

TABLE 16
GUS/RLuc and GUS/FLuc ratios of expression normalized with respect to EXP-
Os.Act1:1:9 (SEQ ID NO: 179) and EXP-CaMV.35S-enh+Zm.DnaK:1:1 (SEQ ID NO: 170)
in corn leaf protoplasts.

| | | | | | GUS/RLuc | GUS/FLuc |
|---|---|---|---|---|---|---|
| | | | GUS/RLuc | GUS/FLuc | relative to | relative to |
| | | SEQ | relative to | relative to | EXP- | EXP- |
| | | ID | EXP- | EXP- | CaMV.35S- | CaMV.35S- |
| Amplicon ID | EXP sequence | NO: | Os.Act1:1:9 | Os.Act1:1:9 | enh+Zm.DnaK:1:1 | enh+Zm.DnaK:1:1 |
| PCR0145942 | EXP-Os.Act1:1:9 | 179 | 1.00 | 1.00 | 0.06 | 0.04 |
| PCR0145944 | EXP-CaMV.35S- | 170 | 16.10 | 23.83 | 1.00 | 1.00 |
| | enh+Zm.DnaK:1:1 | | | | | |
| PCR0145922 | EXP-Cl.Ubq1:1:10 | 98 | 21.60 | 59.23 | 1.34 | 2.49 |
| pMON146750 | EXP-Cl.Ubq1:1:16 | 93 | 23.71 | 60.32 | 1.47 | 2.53 |
| PMON146751 | EXP-Cl.Ubq1:1:17 | 97 | 22.75 | 30.43 | 1.41 | 1.28 |

As can be seen in Table 16, the EXP sequences EXP-Cl.Ubq1:1:10 (SEQ ID NO: 98), EXP-Cl.Ubq1:1:16 (SEQ ID NO: 93) and EXP-Cl.Ubq1:1:17 (SEQ ID NO: 97) were able to drive transgene expression. Expression driven by each of the EXP sequences was higher than that of both controls.

In a fifth set of experiments, amplicon GUS transgene cassettes were made as described above assay expression driven by the EXP sequences, EXP-Zm.UbqM1:1:11 (SEQ ID NO: 149) and EXP-Cl.Ubq1:1:23 (SEQ ID NO: 108). Expression was compared to the controls EXP-Os.Act1:1:9 (SEQ ID NO: 179) and EXP-CaMV.35S-enh+Ta.Lhcb1+Os.Act1:1:1 (SEQ ID NO: 163). Table 17 below shows the mean GUS and luciferase values determined for each amplicon. Table 18 below shows the GUS/RLuc ratios of expression normalized with respect to EXP-Os.Act1:1:9 and EXP-CaMV.35S-enh+Ta.Lhcb1+Os.Act1:1:1 driven expression in corn protoplasts.

TABLE 17
Mean GUS and Luciferase activity in transformed corn leaf protoplast cells.

| | | | SEQ | | |
|---|---|---|---|---|---|
| | | | ID | | |
| Template | Amplicon | EXP sequence | NO: | GUS | RLuc |
| PMON65328 | PCR0145943 | EXP-CaMV.35S- | 163 | 70352.00 | 79028.75 |
| | | enh+Ta.Lhcb1+ | | | |
| | | Os.Act1:1:1 | | | |
| pMON25455 | PCR0145942 | EXP-Os.Act1:1:9 | 179 | 33155.25 | 92337.00 |
| pMON131962 | pMON131962 | EXP-Zm.UbqM1:1:11 | 149 | 18814.75 | 33663.00 |
| pMON132047 | pMON132047 | EXP-Cl.Ubq1:1:23 | 108 | 15387.50 | 40995.50 |

TABLE 18
GUS/RLuc ratios of expression normalized with respect to EXP-Os.Act1:1:9
(SEQ ID NO: 179) and EXP-CaMV.35S-enh+Ta.Lhcb1+Os.Act1:1:1 (SEQ ID NO: 163) in
corn leaf protoplasts.

| | | SEQ | GUS/RLuc | GUS/RLuc relative |
|---|---|---|---|---|
| | | ID | relative to | to EXP-CaMV.35S- |
| Amplicon | EXP sequence | NO: | EXP-Os.Act1:1:9 | enh+Ta.Lhcb1+Os.Act1:1:1 |
| PCR0145943 | EXP-CaMV.35S- | 163 | 2.48 | 1.00 |
| | enh+Ta.Lhcb1+ | | | |
| | Os.Act1:1:1 | | | |
| PCR0145942 | EXP-Os.Act1:1:9 | 179 | 1.00 | 0.40 |
| pMON131962 | EXP-Zm.UbqM1:1:11 | 149 | 1.56 | 0.63 |
| pMON132047 | EXP-Cl.Ubq1:1:23 | 108 | 1.05 | 0.42 |

As can be seen in Table 18 above, the EXP sequences, EXP-Zm.UbqM1:1:11 (SEQ ID NO: 149) and EXP-Cl.Ubq1:1:23 (SEQ ID NO: 108) were able to drive GUS expression in corn leaf protoplasts. Expression was similar to that of the control, EXP-Os.Act1:1:9 (SEQ ID NO: 179) and lower than that of EXP-CaMV.35S-enh+Ta.Lhcb1+Os.Act1:1:1 (SEQ ID NO: 163).

The efficacy of regulatory elements driving GUS expression from amplicons can be similarly studied in sugarcane leaf protoplasts. For instance, sugarcane protoplasts may be transformed with DNA amplicons derived from plant expression vectors containing an EXP sequence, driving expression of the β-glucuronidase (GUS) transgene, and compared to leaf protoplast in which expression of GUS is driven by known constitutive promoters. Likewise, regulatory elements driving CP4 expression from amplicons in corn or wheat protoplasts may be similarly studied.

### Example 4: Analysis of Regulatory Elements Driving GUS in Wheat Protoplasts Using GUS Transgene Cassette Amplicons

Wheat leaf protoplasts were transformed with DNA amplicons derived from plant expression vectors containing an EXP sequence, driving expression of the β-glucuronidase (GUS) transgene, and compared to leaf protoplast in which expression of GUS was driven by known constitutive promoters.

Wheat protoplast cells derived from leaf tissue were transformed using methods known in the art with amplicons produced from amplification of GUS transgene cassettes comprising plant expression vectors to compare expression of a transgene (GUS) driven by the EXP sequences listed in Tables 10-11 with that of known constitutive promoters with methodology as described in a previous example (Example 3), using the same GUS cassette amplicons as that used for assay in Corn in Example 3 above. Control GUS cassette amplicons and Luciferase plasmids used for wheat protoplast transformation were also the same as those presented in the previous example and provided in Table 7 above in Example 3. Likewise, negative controls were used for the determination of GUS and luciferase background, as described above. Wheat leaf protoplasts were transformed using a PEG-based transformation method, as described in Example 3 above. Table 19 lists mean GUS and LUC activity seen in transformed wheat leaf protoplast cells, and Table 20 shows normalized GUS/RLuc ratios of expression in wheat protoplasts.

TABLE 19
Mean GUS and Luciferase activity in
transformed wheat leaf protoplast cells

| | SEQ | | | |
|---|---|---|---|---|
| EXP sequence | ID NO: | GUS | RLuc | GUS/RLuc |
| EXP-Os.Act1:1:9 | 179 | 2976.33 | 53334.8 | 0.0558047 |
| P-CaMV.35S-ENH- | 169 | 1431.33 | 55996.1 | 0.0255612 |
| 1:1:102/L-CaMV.35S- | | | | |
| 1:1:2 | | | | |
| EXP-CaMV.35S-enh+ | 163 | 29299.3 | 50717.4 | 0.5776973 |
| Ta.Lhcb1+Os.Act1:1:1 | | | | |
| EXP-CaMV.35S-enh+ | 170 | 34294.3 | 63307.9 | 0.5417066 |
| Zm.DnaK:1:1 | | | | |
| EXP-ANDge.Ubq1:1:7 | 5 | 68444.3 | 60329.1 | 1.1345158 |
| EXP-ANDge.Ubq1:1:10 | 10 | 60606.3 | 60659.4 | 0.9991245 |
| EXP-ANDge.Ubq1:1:6 | 12 | 33386.3 | 56712.1 | 0.5886984 |
| EXP-ANDge.Ubq1:1:11 | 14 | 43237.3 | 48263.4 | 0.8958609 |
| EXP-ANDge.Ubq1:1:12 | 16 | 51712.7 | 64702.8 | 0.7992341 |
| EXP-ERIra.Ubq1:1:9 | 22 | 20998.3 | 60273.4 | 0.3483845 |
| EXP-ERIra.Ubq1:1:10 | 25 | 17268.3 | 25465.4 | 0.6781084 |
| EXP-ERIra.Ubq1:1:8 | 27 | 34635.7 | 59467.1 | 0.5824341 |
| EXP-ERIra.Ubq1:1:11 | 29 | 28979 | 56153.8 | 0.516065 |
| EXP-ERIra.Ubq1:1:12 | 31 | 41409.7 | 55152.4 | 0.7508221 |
| EXP-SETit.Ubq1:1:5 | 117 | 39427.7 | 57463.1 | 0.6861388 |
| EXP-SETit.Ubq1:1:7 | 123 | 108091 | 49330.4 | 2.191169 |
| EXP-SETit.Ubq1:1:6 | 124 | 58703 | 46110.1 | 1.2731047 |
| EXP-Sv.Ubq1:1:7 | 128 | 29330 | 43367.1 | 0.676319 |
| EXP-Sv.Ubq1:1:8 | 132 | 53359 | 40076.4 | 1.3314306 |
| EXP-Sv.Ubq1:1:10 | 134 | 49122.7 | 53180.8 | 0.9236922 |
| EXP-Zm.UbqM1:1:6 | 137 | 37268 | 54088.1 | 0.6890239 |
| EXP-Zm.UbqM1:1:7 | 141 | 51408 | 47297.4 | 1.0869087 |
| EXP-Sb.Ubq4:1:2 | 151 | 35660.3 | 62591.1 | 0.5697347 |
| EXP-Sb.Ubq6:1:2 | 153 | 27543 | 57826.4 | 0.4763046 |
| EXP-Cl.Ubq1:1:10 | 98 | 54493.3 | 41964.1 | 1.2985699 |

TABLE 20
GUS/RLuc ratios of expression normalized with respect to EXP-Os.Act1:1:9
(SEQ ID NO: 179) and EXP-CaMV.35S-enh+Ta.Lhcb1+
Os.Act1:1:1 (SEQ ID NO: 163) in wheat leaf protoplasts.

| | | GUS/RLuc | GUS/RLuc Relative |
|---|---|---|---|
| | | Relative to | to EXP-CaMV.35S-enh+ |
| EXP sequence | SEQ ID NO: | EXP-Os.Act1:1:9 | Ta.Lhcb1+Os.Act1:1:1 |
| EXP-Os.Act1:1:9 | 179 | 1.00 | 0.10 |
| P-CaMV.35S-ENH- | 169 | 0.46 | 0.04 |
| 1:1:102/L-CaMV.35S-1:1:2 | | | |
| EXP-CaMV.35S- | 163 | 10.35 | 1.00 |
| enh+Ta.Lhcb1+ | | | |
| Os.Act1:1:1 | | | |
| EXP-CaMV.35S- | 170 | 9.71 | 0.94 |
| enh+Zm.DnaK:1:1 | | | |
| EXP-ANDge.Ubq1:1:7 | 5 | 20.33 | 1.96 |
| EXP-ANDge.Ubq1:1:10 | 10 | 17.90 | 1.73 |
| EXP-ANDge.Ubq1:1:6 | 12 | 10.55 | 1.02 |
| EXP-ANDge.Ubq1:1:11 | 14 | 16.05 | 1.55 |
| EXP-ANDge.Ubq1:1:12 | 16 | 14.32 | 1.38 |
| EXP-ERIra.Ubq1:1:9 | 22 | 6.24 | 0.60 |
| EXP-ERIra.Ubq1:1:10 | 25 | 12.15 | 1.17 |
| EXP-ERIra.Ubq1:1:8 | 27 | 10.44 | 1.01 |
| EXP-ERIra.Ubq1:1:11 | 29 | 9.25 | 0.89 |
| EXP-ERIra.Ubq1:1:12 | 31 | 13.45 | 1.30 |
| EXP-SETit.Ubq1:1:5 | 117 | 12.30 | 1.19 |
| EXP-SETit.Ubq1:1:7 | 123 | 39.26 | 3.79 |
| EXP-SETit.Ubq1:1:6 | 124 | 22.81 | 2.20 |
| EXP-Sv.Ubq1:1:7 | 128 | 12.12 | 1.17 |
| EXP-Sv.Ubq1:1:8 | 132 | 23.86 | 2.30 |
| EXP-Sv.Ubq1:1:10 | 134 | 16.55 | 1.60 |
| EXP-Zm.UbqM1:1:6 | 137 | 12.35 | 1.19 |
| EXP-Zm.UbqM1:1:7 | 141 | 19.48 | 1.88 |
| EXP-Sb.Ubq4:1:2 | 151 | 10.21 | 0.99 |
| EXP-Sb.Ubq6:1:2 | 153 | 8.54 | 0.82 |
| EXP-Cl.Ubq1:1:10 | 98 | 23.27 | 2.25 |

As can be seen in Table 20 above, nearly all of the EXP sequences were capable of driving GUS transgene expression in wheat cells. GUS transgene expression driven by EXP-ANDge.Ubq1:1:7 (SEQ ID NO: 5), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-SETit.Ubq1:1:5 (SEQ ID NO: 117), EXP-SETit.Ubq1:1:7 (SEQ ID NO: 123), EXP-SETit.Ubq1:1:6 (SEQ ID NO: 124), EXP-Sv.Ubq1:1:7 (SEQ ID NO: 128), EXP-Sv.Ubq1:1:8 (SEQ ID NO: 132), EXP-Sv.Ubq1:1:10 (SEQ ID NO: 134), EXP-Zm.UbgM1:1:6 (SEQ ID NO: 137), EXP-Zm.UbgM1:1:7 (SEQ ID NO: 141), EXP-Sb.Ubg4:1:2 (SEQ ID NO: 151), EXP-Sb.Ubg6:1:2 (SEQ ID NO: 153) and EXP-Cl.Ubq1:1:10 (SEQ ID NO: 98) was much higher than GUS expression driven by EXP-Os.Act1:1:9. GUS expression of the amplicons in wheat leaf protoplast cells relative to EXP-CaMV.35S-enh+Ta.Lhcb1+Os.Act1:1:1 was slightly different from the expression observed in corn protoplast cells. Each of EXP-ANDge.Ubq1:1:7 (SEQ ID NO: 5), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:10 (SEQ ID NO: 25), EXP-ERIra.Ubq1:1:8 (SEQ ID NO: 27), EXP-ERIra.Ubq1:1:12 (SEQ ID NO: 31), EXP-SETit.Ubq1:1:5 (SEQ ID NO: 117), EXP-SETit.Ubq1:1:7 (SEQ ID NO: 123), EXP-SETit.Ubq1:1:6 (SEQ ID NO: 124), EXP-Sv.Ubq1:1:7 (SEQ ID NO: 128), EXP-Sv.Ubq1:1:8 (SEQ ID NO: 132), EXP-Sv.Ubq1:1:10 (SEQ ID NO: 134), EXP-Zm.UbgM1:1:6 (SEQ ID NO: 137), EXP-Zm.UbgM1:1:7 (SEQ ID NO: 141) and EXP-Cl.Ubq1:1:10 (SEQ ID NO: 98) demonstrated higher levels of GUS expression relative to EXP-CaMV.35S-enh+Ta.Lhcb1+Os.Act1:1:1. The EXP sequences EXP-ERIra.Ubq1:1:9 (SEQ ID NO: 22), EXP-ERIra.Ubq1:1:11 (SEQ ID NO: 29), EXP-Sb.Ubg4:1:2 (SEQ ID NO: 151) and EXP-Sb.Ubg6:1:2 (SEQ ID NO: 153) demonstrated lower levels of GUS expression relative to EXP-CaMV.35S-enh+Ta.Lhcb1+Os.Act1:1:1.

In a second set of experiments, amplicon GUS transgene cassettes were made as described above and assayed for expression driven by the EXP sequences, EXP-ANDge.Ubq1:1:8 (SEQ ID NO: 8), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116). The amplicons were comprised of an EXP sequence operably linked to the GUS-1 coding sequence which was operably linked to the T-AGRtu.nos-1:1:13 3′ UTR. Expression was compared to the controls EXP-Os.Act1:1:9 (SEQ ID NO: 179) and EXP-CaMV.35S-enh+Zm.DnaK:1:1 (SEQ ID NO: 170). Table 21 below shows the mean GUS and luciferase values determined for each amplicon. Table 22 below shows the GUS/RLuc ratios of expression normalized with respect to EXP-Os.Act1:1:9 and EXP-CaMV.35S-enh+Zm.DnaK:1:1 driven expression in corn protoplasts.

TABLE 21
Mean GUS and Luciferase activity in
transformed wheat leaf protoplast cells.

| Amplicon | | SEQ ID | | |
|---|---|---|---|---|
| ID | EXP sequence | NO: | GUS | RLuc |
| PCR0145942 | EXP-Os.Act1:1:9 | 179 | 1234 | 176970.5 |
| PCR0145944 | EXP-CaMV.35S-enh + | 170 | 12883.5 | 119439 |
| | Zm.DnaK:1:1 | | | |
| PCR0146628 | EXP-ANDge.Ubq1:1:8 | 8 | 38353.3 | 171535.3 |
| PCR0145922 | EXP-Cl.Ubq1:1:10 | 98 | 34938 | 154245.8 |
| PCR0145945 | EXP-Cl.Ubq1:1:13 | 114 | 32121 | 122220.8 |
| PCR0145946 | EXP-Cl.Ubq1:1:14 | 115 | 56814 | 143318.3 |
| PCR0145947 | EXP-Cl.Ubq1:1:15 | 116 | 1890.5 | 167178.5 |

TABLE 22
GUS/RLuc and GUS/FLuc ratios of expression normalized with
respect to EXP-Os.Act1:1:9 (SEQ ID NO: 179) and EXP-CaMV.35S-
enh+Zm.DnaK:1:1 (SEQ ID NO: 170) in wheat leaf protoplasts.

| | | | GUS/RLuc |
|---|---|---|---|
| | | GUS/RLuc | relative to EXP- |
| | SEQ ID | relative to EXP- | CaMV.35S- |
| EXP sequence | NO: | Os.Act1:1:9 | enh+Zm.DnaK:1:1 |
| EXP-Os.Act1:1:9 | 179 | 1.00 | 0.06 |
| EXP-CaMV.35S-enh+ | 170 | 15.47 | 1.00 |
| Zm.DnaK:1:1 | | | |
| EXP-ANDge.Ubq1:1:8 | 8 | 32.07 | 2.07 |
| EXP-Cl.Ubq1:1:10 | 98 | 32.48 | 2.10 |
| EXP-Cl.Ubq1:1:13 | 114 | 37.69 | 2.44 |
| EXP-Cl.Ubq1:1:14 | 115 | 56.85 | 3.68 |
| EXP-Cl.Ubq1:1:15 | 116 | 1.62 | 0.10 |

As can be seen in Table 22 above, the EXP sequences EXP-ANDge.Ubq1:1:8 (SEQ ID NO: 8), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116) are capable of driving transgene expression. Expression driven by EXP-ANDge.Ubq1:1:8 (SEQ ID NO: 8), EXP-Cl.Ubq1:1:10 (SEQ ID NO: 98), EXP-Cl.Ubq1:1:13 (SEQ ID NO: 114) and EXP-Cl.Ubq1:1:14 (SEQ ID NO: 115) was higher than that of both controls. Expression driven by EXP-Cl.Ubq1:1:15 (SEQ ID NO: 116) was lower than EXP-CaMV.35S-enh+Zm.DnaK:1:1 (SEQ ID NO: 170) but higher than the control, EXP-Os.Act1:1:9 (SEQ ID NO: 179).

In a third set of experiments, amplicon GUS transgene cassettes were made as described above to assay expression driven by the EXP sequences, EXP-Cl.Ubq1:1:10 (SEQ ID NO: 98), EXP-Cl.Ubq1:1:16 (SEQ ID NO: 93) and EXP-Cl.Ubq1:1:17 (SEQ ID NO: 97). Expression was compared to the controls EXP-Os.Act1:1:9 (SEQ ID NO: 179) and EXP-CaMV.35S-enh+Zm.DnaK:1:1 (SEQ ID NO: 170). Table 23 below shows the mean GUS and luciferase values determined for each amplicon. Table 24 below shows the GUS/RLuc and GUS/FLuc ratios of expression normalized with respect to EXP-Os.Act1:1:9 and EXP-CaMV.35S-enh+Zm.DnaK:1:1 driven expression in corn protoplasts.

TABLE 23
Mean GUS and Luciferase activity in
transformed wheat leaf protoplast cells.

| | | SEQ | | | |
|---|---|---|---|---|---|
| | | ID | | | |
| Amplicon ID | EXP sequence | NO: | GUS | RLuc | FLuc |
| PCR0145942 | EXP-Os.Act1:1:9 | 179 | 478 | 46584.5 | 2709.75 |
| PCR0145944 | EXP-CaMV.35S- | 170 | 8178.5 | 43490.8 | 2927.25 |
| | enh+Zm.DnaK:1:1 | | | | |
| PCR0145922 | EXP-Cl.Ubq1:1:10 | 98 | 22068.3 | 47662.3 | 1289 |
| pMON146750 | EXP-Cl.Ubq1:1:16 | 93 | 34205 | 45064.5 | 1379.63 |
| pMON146751 | EXP-Cl.Ubq1:1:17 | 97 | 31758 | 45739.3 | 2820.75 |

TABLE 24
GUS/RLuc and GUS/FLuc ratios of expression normalized with respect to EXP-
Os.Act1:1:9 (SEQ ID NO: 179) and EXP-CaMV.35S-enh+Zm.DnaK:1:1 (SEQ ID NO: 170)
in wheat leaf protoplasts.

| | | | | | GUS/RLuc | GUS/FLuc |
|---|---|---|---|---|---|---|
| | | | GUS/RLuc | GUS/FLuc | relative to | relative to |
| | | SEQ | relative | relative | EXP- | EXP- |
| | | ID | to EXP- | to EXP- | CaMV.35S- | CaMV.35S- |
| Amplicon ID | EXP sequence | NO: | Os.Act1:1:9 | Os.Act1:1:9 | enh+Zm.DnaK:1:1 | enh+Zm.DnaK:1:1 |
| PCR0145942 | EXP-Os.Act1:1:9 | 179 | 1.00 | 1.00 | 0.05 | 0.06 |
| PCR0145944 | EXP-CaMV.35S- | 170 | 18.33 | 15.84 | 1.00 | 1.00 |
| | enh+Zm.DnaK:1:1 | | | | | |
| PCR0145922 | EXP-Cl.Ubq1:1:10 | 98 | 45.12 | 97.05 | 2.46 | 6.13 |
| pMON146750 | EXP-Cl.Ubq1:1:16 | 93 | 73.97 | 140.55 | 4.04 | 8.87 |
| pMON146751 | EXP-Cl.Ubq1:1:17 | 97 | 67.67 | 63.82 | 3.69 | 4.03 |

As can be seen in Table 24 above, the EXP sequences EXP-Cl.Ubq1:1:10 (SEQ ID NO: 98), EXP-Cl.Ubq1:1:16 (SEQ ID NO: 93) and EXP-Cl.Ubq1:1:17 (SEQ ID NO: 97) were able to drive transgene expression. Expression driven by each of the EXP sequences was higher than that of both controls.

In a fourth set of experiments, amplicon GUS transgene cassettes were made as described above to assay expression driven by the EXP sequences, EXP-Zm.UbqM1:1:11 (SEQ ID NO: 149) and EXP-Cl.Ubq1:1:23 (SEQ ID NO: 108). Expression was compared to the controls EXP-Os.Act1:1:9 (SEQ ID NO: 179) and EXP-CaMV.35S-enh+Ta.Lhcb1+Os.Act1:1:1 (SEQ ID NO: 163). Table 25 below shows the mean GUS and luciferase values determined for each amplicon. Table 26 below shows the GUS/RLuc ratios of expression normalized with respect to EXP-Os.Act1:1:9 and EXP-CaMV.35S-enh+Ta.Lhcb1+Os.Act1:1:1 driven expression in corn protoplasts.

TABLE 25
Mean GUS and Luciferase activity in
transformed wheat leaf protoplast cells.

| | | | SEQ ID | | |
|---|---|---|---|---|---|
| Template | Amplicon ID | EXP sequence | NO: | GUS | RLuc |
| pMON65328 | PCR0145943 | EXP-CaMV.35S- | 163 | 67459.13 | 11682.00 |
| | | enh+Ta.Lhcb1+ | | | |
| | | Os.Act1:1:1 | | | |
| PMON25455 | PCR0145942 | EXP-Os.Act1:1:9 | 179 | 56618.33 | 16654.83 |
| pMON131962 | pMON131962 | EXP-Zm.UbqM1:1:11 | 149 | 53862.13 | 10313.75 |
| pMON132047 | pMON132047 | EXP-Cl.Ubq1:1:23 | 108 | 38869.38 | 12279.00 |

TABLE 26
GUS/RLuc ratios of expression normalized with respect to EXP-Os.Act1:1:9
(SEQ ID NO: 179) and EXP-CaMV.35S-enh+Ta.Lhcb1+Os.Act1:1:1
(SEQ ID NO: 163) in wheat leaf protoplasts.

| | | | GUS/RLuc | |
|---|---|---|---|---|
| | | | relative to | GUS/RLuc relative to EXP- |
| | | SEQ ID | EXP- | CaMV.35S- |
| Amplicon ID | EXP sequence | NO: | Os.Act1:1:9 | enh+Ta.Lhcb1+Os.Act1:1:1 |
| PCR0145943 | EXP-CaMV.35S- | 163 | 1.70 | 1.00 |
| | enh+Ta.Lhcb1+ | | | |
| | Os.Act1:1:1 | | | |
| PCR0145942 | EXP-Os.Act1:1:9 | 179 | 1.00 | 0.59 |
| PMON131962 | EXP-Zm.UbqM1:1:11 | 149 | 1.54 | 0.90 |
| pMON132047 | EXP-Cl.Ubq1:1:23 | 108 | 0.93 | 0.55 |

As can be seen in Table 26 above, the EXP sequences, EXP-Zm.UbqM1:1:11 (SEQ ID NO: 149) and EXP-Cl.Ubq1:1:23 (SEQ ID NO: 108) were able to drive GUS expression in wheat leaf protoplasts. Expression was similar to that of the control, EXP-Os.Act1:1:9 (SEQ ID NO: 179) and lower than that of EXP-CaMV.35S-enh+Ta.Lhcb1+Os.Act1:1:1 (SEQ ID NO: 163).

### Example 5: Analysis of Regulatory Elements Driving GUS in Sugarcane Protoplasts Using GUS Transgene Cassette Amplicons

Sugarcane leaf protoplasts were transformed with DNA amplicons derived from plant expression vectors containing an EXP sequence, driving expression of the β-glucuronidase (GUS) transgene, and compared to leaf protoplast in which expression of GUS was driven by known constitutive promoters.

Sugarcane protoplast cells derived from leaf tissue were transformed using a PEG-based transformation method, as described in Example 3 above with amplicons produced from amplification of GUS transgene cassettes comprising plant expression vectors to compare expression of a transgene (GUS) driven by one of EXP-ANDge.Ubq1:1:7 (SEQ ID NO: 5), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116) and presented in Table 27 below, with that of known constitutive promoters.

TABLE 27
GUS plant expression amplicons and corresponding
plasmid construct amplicon template and EXP sequence.

| | | | SEQ |
|---|---|---|---|
| Amplicon | Amplicon | | ID |
| ID | Template | EXP sequence | NO: |
| PCR0145942 | pMON25455 | EXP-Os.Act1:1:9 | 179 |
| PCR0145944 | pMON81552 | EXP-CaMV.35S- | 170 |
| | | enh+Zm.DnaK:1:1 | |
| PCR0145892 | pMON136264 | EXP-ANDge.Ubq1:1:7 | 5 |
| PCR0145815 | pMON136264 | EXP-ANDge.Ubq1:1:10 | 10 |
| PCR0145893 | pMON136259 | EXP-ANDge.Ubq1:1:6 | 12 |
| PCR0145817 | pMON136264 | EXP-ANDge.Ubq1:1:11 | 14 |
| PCR0145819 | pMON136264 | EXP-ANDge.Ubq1:1:12 | 16 |
| PCR0145896 | pMON136263 | EXP-ERIra.Ubq1:1:9 | 22 |
| PCR0145820 | pMON136263 | EXP-ERIra.Ubq1:1:10 | 25 |
| PCR0145897 | pMON136258 | EXP-ERIra.Ubq1:1:8 | 27 |
| PCR0145821 | pMON136263 | EXP-ERIra.Ubq1:1:11 | 29 |
| PCR0145822 | pMON136263 | EXP-ERIra.Ubq1:1:12 | 31 |
| PCR0145922 | pMON140889 | EXP-Cl.Ubq1:1:10 | 98 |
| PCR0145945 | pMON140889 | EXP-Cl.Ubq1:1:13 | 114 |
| PCR0145946 | pMON140889 | EXP-Cl.Ubq1:1:14 | 115 |
| PCR0145947 | pMON140889 | EXP-Cl.Ubq1:1:15 | 116 |

Control GUS cassette amplicons and Luciferase plasmids used for sugarcane protoplast transformation were also the same as those presented in Examples 2 through 4 and provided in Table 7 above in Example 3. Likewise, negative controls were used for the determination of GUS and luciferase background, as described above. Table 28 lists mean GUS and Luc activity seen in transformed sugarcane leaf protoplast cells, and Table 29 shows normalized GUS/RLuc ratios of expression in sugarcane leaf protoplasts.

TABLE 28
Mean GUS and Luciferase activity in
transformed wheat leaf protoplast cells.

| | SEQ | | | |
|---|---|---|---|---|
| | ID | | | |
| EXP sequence | NO: | GUS | RLuc | FLuc |
| EXP-Os.Act1:1:9 | 179 | 6667.5 | 3024.5 | 1129.25 |
| EXP-CaMV.35S- | 170 | 14872.8 | 5171 | 2019.5 |
| enh+Zm.DnaK:1:1 | | | | |
| EXP-ANDge.Ubq1:1:7 | 5 | 15225 | 4618.25 | 1775.75 |
| EXP-ANDge.Ubq1:1:10 | 10 | 17275.3 | 4333 | 1678 |
| EXP-ANDge.Ubq1:1:6 | 12 | 17236 | 5633.25 | 2240 |
| EXP-ANDge.Ubq1:1:11 | 14 | 22487.8 | 6898.25 | 2878 |
| EXP-ANDge.Ubq1:1:12 | 16 | 22145.3 | 6240.25 | 2676.5 |
| EXP-ERIra.Ubq1:1:9 | 22 | 16796.5 | 7759.75 | 3179 |
| EXP-ERIra.Ubq1:1:10 | 25 | 16267.5 | 5632.75 | 2436.75 |
| EXP-ERIra.Ubq1:1:8 | 27 | 25351 | 9019.5 | 4313.5 |
| EXP-ERIra.Ubq1:1:11 | 29 | 16652.3 | 3672.25 | 1534 |
| EXP-ERIra.Ubq1:1:12 | 31 | 12654.5 | 3256.75 | 1261.5 |
| EXP-Cl.Ubq1:1:10 | 98 | 22383.8 | 7097.5 | 3109.25 |
| EXP-Cl.Ubq1:1:13 | 114 | 14532.3 | 2786.5 | 1198.25 |
| EXP-Cl.Ubq1:1:14 | 115 | 19244.5 | 3455.25 | 1475 |
| EXP-Cl.Ubq1:1:15 | 116 | 6676.5 | 3870.25 | 1497.75 |

TABLE 29
GUS/RLuc and GUS/FLuc ratios of expression normalized with respect to EXP-
Os.Act1:1:9 (SEQ ID NO: 179) and EXP-CaMV.35S-enh+Zm.DnaK:1:1
(SEQ ID NO: 170) in sugarcane leaf protoplasts.

| | | | | GUS/RLuc | GUS/FLuc |
|---|---|---|---|---|---|
| | | GUS/RLuc | GUS/FLuc | relative to | relative to |
| | SEQ | relative | relative | EXP- | EXP- |
| | ID | to EXP- | to EXP- | CaMV.35S- | CaMV.35S- |
| EXP sequence | NO: | Os.Act1:1:9 | Os.Act1:1:9 | enh+Zm.DnaK:1:1 | enh+Zm.DnaK:1:1 |
| EXP-Os.Act1:1:9 | 179 | 1.00 | 1.00 | 0.77 | 0.80 |
| EXP-CaMV.35S- | 170 | 1.30 | 1.25 | 1.00 | 1.00 |
| enh+Zm.DnaK:1:1 | | | | | |
| EXP-ANDge.Ubq1:1:7 | 5 | 1.50 | 1.45 | 1.15 | 1.16 |
| EXP-ANDge.Ubq1:1:10 | 10 | 1.81 | 1.74 | 1.39 | 1.40 |
| EXP-ANDge.Ubq1:1:6 | 12 | 1.39 | 1.30 | 1.06 | 1.04 |
| EXP-ANDge.Ubq1:1:11 | 14 | 1.48 | 1.32 | 1.13 | 1.06 |
| EXP-ANDge.Ubq1:1:12 | 16 | 1.61 | 1.40 | 1.23 | 1.12 |
| EXP-ERIra.Ubq1:1:9 | 22 | 0.98 | 0.89 | 0.75 | 0.72 |
| EXP-ERIra.Ubq1:1:10 | 25 | 1.31 | 1.13 | 1.00 | 0.91 |
| EXP-ERIra.Ubq1:1:8 | 27 | 1.27 | 1.00 | 0.98 | 0.80 |
| EXP-ERIra.Ubq1:1:11 | 29 | 2.06 | 1.84 | 1.58 | 1.47 |
| EXP-ERIra.Ubq1:1:12 | 31 | 1.76 | 1.70 | 1.35 | 1.36 |
| EXP-Cl.Ubq1:1:10 | 98 | 1.43 | 1.22 | 1.10 | 0.98 |
| EXP-Cl.Ubq1:1:13 | 114 | 2.37 | 2.05 | 1.81 | 1.65 |
| EXP-Cl.Ubq1:1:14 | 115 | 2.53 | 2.21 | 1.94 | 1.77 |
| EXP-Cl.Ubq1:1:15 | 116 | 0.78 | 0.75 | 0.60 | 0.61 |

As can be seen in Table 29 above, the EXP sequences EXP-ANDge.Ubq1:1:7 (SEQ ID NO: 5), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116) were all capable of driving transgene expression in sugarcane protoplasts. The EXP sequences, EXP-ANDge.Ubq1:1:7 (SEQ ID NO: 5), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:10 (SEQ ID NO: 25), EXP-ERIra.Ubq1:1:8 (SEQ ID NO: 27), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114) and EXP-Cl.Ubq1:1:14 (SEQ ID NO: 115) expressed GUS higher than EXP-CaMV.35S-enh+Zm.DnaK:1:1 (SEQ ID NO: 170) in this experiment.

### Example 6: Analysis of Regulatory Elements Driving CP4 in Corn Protoplasts

This example illustrates the ability of EXP-Sv.Ubq1:1:7 (SEQ ID NO: 128), EXP-Sv.Ubq1:1:8 (SEQ ID NO: 132), EXP-Sv.Ubq1:1:9 (SEQ ID NO: 133), EXP-Zm.UbgM1:1:6 (SEQ ID NO: 137), EXP-Zm.UbgM1:1:8 (SEQ ID NO: 145), EXP-Zm.UbgM1:1:7 (SEQ ID NO: 141), EXP-SETit.Ubq1:1:5 (SEQ ID NO: 117), EXP-SETit.Ubq1:1:7 (SEQ ID NO: 123), EXP-SETit.Ubq1:1:6 (SEQ ID NO: 124), EXP-Sb.Ubg4:1:2 (SEQ ID NO: 151) and EXP-Sb.Ubg6:1:2 (SEQ ID NO: 153) in driving expression of glyphosate tolerance gene CP4 in corn protoplasts. These EXP sequences were cloned into plant binary transformation plasmid constructs using methods known in the art. The resulting plant expression vectors contained a right border region from A. tumefaciens, an ubiquitin EXP sequence operably linked 5′ to a plastid targeted glyphosate tolerant EPSPS coding sequence (CP4, U.S. RE39247), operably linked 5′ to the T-AGRtu.nos-1:1:13 3′ UTR and a left border region from A. tumefaciens (B-AGRtu.left border). The resulting plasmid constructs were used to transform corn leaf protoplasts cells using methods known in the art.

Plasmid constructs listed in Table 30, with EXP sequences as defined in Table 1, were utilized. Three control plasmids (pMON30098, pMON42410, and pMON30167), with known constitutive regulatory elements driving either CP4 or GFP, were constructed and used to compare the relative CP4 expression levels driven by these EXP sequences with CP4 expression driven by known constitutive expression elements. Two other plasmids (pMON19437 and pMON63934) were also used as described above to evaluate transformation efficiency and viability. Each plasmid contains a specific luciferase coding sequence driven by a constitutive EXP sequence.

Corn leaf protoplasts were transformed using a PEG-based transformation method, as described in Example 2 above. Measurements of both CP4 and luciferase were conducted similarly to Example 2 above. The average levels of CP4 protein expression expressed as part per million (ppm) is shown in Table 30 below.

TABLE 30
Average CP4 protein expression in corn leaf protoplasts.

| | | SEQ | CP4 | CP4 |
|---|---|---|---|---|
| | | ID | Average | STDEV |
| Plasmid | EXP sequence | NO: | ppm | ppm |
| No DNA | No DNA | | 0 | 0 |
| pMON30098 | GFP | | 0 | 0 |
| pMON42410 | EXP-CaMV.35S- | 163 | 34.1 | 15.6 |
| | enh + Ta.Lhcb1 + | | | |
| | Os.Act1:1:1 | | | |
| pMON30167 | EXP-Os.Act1:1:1 | 164 | 40.4 | 11.6 |
| pMON129203 | EXP-Sv.Ubq1:1:7 | 128 | 45.2 | 6.2 |
| pMON129204 | EXP-Sv.Ubq1:1:8 | 132 | 101.9 | 13.8 |
| pMON129205 | EXP-Sv.Ubq1:1:9 | 133 | 71.1 | 8.7 |
| pMON129210 | EXP-Zm.UbqM1:1:6 | 137 | 137.1 | 14.8 |
| pMON129211 | EXP-Zm.UbqM1:1:8 | 145 | 136.5 | 12.3 |
| pMON129212 | EXP-Zm.UbqM1:1:7 | 141 | 170.2 | 18.1 |
| pMON129200 | EXP-SETit.Ubq1:1:5 | 117 | 44.3 | 9.5 |
| pMON129201 | EXP-SETit.Ubq1:1:7 | 123 | 105.1 | 8.4 |
| pMON129202 | EXP-SETit.Ubq1:1:6 | 124 | 124.9 | 33.7 |
| pMON129219 | EXP-Sb.Ubq4:1:2 | 151 | 14.3 | 1 |
| pMON129218 | EXP-Sb.Ubq6:1:2 | 153 | 75.7 | 8.9 |

As can be seen in Table 30, EXP-Sv.Ubq1:1:7 (SEQ ID NO: 128), EXP-Sv.Ubq1:1:8 (SEQ ID NO: 132), EXP-Sv.Ubq1:1:9 (SEQ ID NO: 133), EXP-Zm.UbgM1:1:6 (SEQ ID NO: 137), EXP-Zm.UbgM1:1:8 (SEQ ID NO: 145), EXP-Zm.UbgM1:1:7 (SEQ ID NO: 141), EXP-SETit.Ubq1:1:5 (SEQ ID NO: 117), EXP-SETit.Ubq1:1:7 (SEQ ID NO: 123), EXP-SETit.Ubq1:1:6 (SEQ ID NO: 124) and EXP-Sb.Ubg6:1:2 (SEQ ID NO: 153) drove expression of the CP4 transgene at levels close to or higher than CP4 expression levels driven by EXP-CaMV.35S-enh+Ta.Lhcb1+Os.Act1:1:1 and EXP-Os.Act1:1:1. The EXP sequence, EXP-Sb.Ubg4:1:2 (SEQ ID NO: 151) demonstrated the ability to drive expression of CP4, but the level of expression was lower than that of the constitutive controls.

Similar data to that above may also be obtained from plants stably transformed with plasmid constructs described above, for instance, plants of progeny generation(s) R₀, R₁ or F₁ or later. Likewise, expression from other plasmid constructs may be studied. For instance, pMON141619, comprises the EXP sequence EXP-ANDge.Ubq1:1:8, while pMON142862 is comprised of the EXP sequence EXP-ERIra.Ubq1:1:8. These and other constructs may be analyzed in this manner.

### Example 7: Analysis of Regulatory Elements Driving CP4 in Corn Protoplasts Using CP4 Transgene Cassette Amplicons

This example illustrates the ability of EXP-ANDge.Ubq1:1:7 (SEQ ID NO: 5), EXP-ANDge.Ubq1:1:8 (SEQ ID NO: 8), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115), EXP-Cl.Ubq1:1:15 (SEQ ID NO: 116), EXP-Cl.Ubq1:1:16 (SEQ ID NO: 93) and EXP-Cl.Ubq1:1:17 (SEQ ID NO: 97) in driving expression of glyphosate tolerance gene CP4 in corn protoplasts. These EXP sequences were cloned into plant binary transformation plasmid constructs. The resulting plant expression vectors were used as amplification templates to produce a transgene cassette amplicon comprised of an ubiquitin EXP sequence operably linked 5′ to a plastid targeted glyphosate tolerant EPSPS coding sequence (CP4, U.S. RE39247), operably linked 5′ to the T-AGRtu.nos-1:1:13 3′ UTR and a left border region from A. tumefaciens. The resulting amplicons were used to transform corn leaf protoplasts cells.

Corn leaf protoplasts were transformed using a PEG-based transformation method, as described in Example 2 above. Measurements of both CP4 were conducted using an ELISA-based assay. The average levels of CP4 protein expression expressed as part per million (ppm) is shown in Tables 31 and 32 below.

In a first series of experiments, expression of CP4 driven by amplicons comprised of the EXP sequences EXP-ANDge.Ubq1:1:7 (SEQ ID NO: 5), EXP-ANDge.Ubq1:1:8 (SEQ ID NO: 8), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116) were assayed in transformed corn leaf protoplasts and compared to CP4 expression levels driven by the constitutive controls, EXP-CaMV.35S-enh+Zm.DnaK:1:1 (SEQ ID NO: 170) and EXP-Os.Act1:1:1 (SEQ ID NO: 164). The average levels of CP4 protein expression expressed as part per million (ppm) is shown in Tables 31 below.

TABLE 31
Average CP4 protein expression in corn leaf protoplasts.

| | | | | CP4 | CP4 |
|---|---|---|---|---|---|
| | | | | ng/mg | ng/mg |
| | | | | total | total |
| Amplicon | Amplicon | | SEQ ID | protein | protein |
| Template | ID | EXP sequence | NO: | Average | STDEV |
| | | no DNA | | 0.0 | 0.0 |
| pMON30098 | | GFP (negative control) | | 0.0 | 0.0 |
| pMON19469 | PCR24 | EXP-CaMV.35S- | 170 | 605.5 | 27.6 |
| | | enh + Zm.DnaK:1:1 | | | |
| pMON30167 | PCR25 | EXP-Os.Act1:1:1 | 164 | 50.6 | 14.2 |
| pMON140896 | PCR41 | EXP-ANDge.Ubq1:1:7 | 5 | 459.0 | 60.9 |
| pMON140917 | PCR42 | EXP-ANDge.Ubq1:1:8 | 8 | 258.2 | 38.4 |
| pMON140897 | PCR43 | EXP-ANDge.Ubq1:1:10 | 10 | 324.8 | 21.6 |
| pMON140898 | PCR44 | EXP-ANDge.Ubq1:1:6 | 12 | 394.9 | 66.4 |
| pMON140899 | PCR45 | EXP-ANDge.Ubq1:1:11 | 14 | 508.7 | 89.6 |
| pMON140900 | PCR46 | EXP-ANDge.Ubq1:1:12 | 16 | 329.3 | 14.5 |
| pMON140904 | PCR50 | EXP-ERIra.Ubq1:1:9 | 22 | 148.6 | 24.4 |
| pMON140905 | PCR51 | EXP-ERIra.Ubq1:1:10 | 25 | 215.8 | 22.6 |
| pMON140906 | PCR52 | EXP-ERIra.Ubq1:1:8 | 27 | 376.6 | 44.1 |
| pMON140907 | PCR53 | EXP-ERIra.Ubq1:1:11 | 29 | 459.9 | 104.7 |
| pMON140908 | PCR54 | EXP-ERIra.Ubq1:1:12 | 31 | 221.6 | 15.9 |
| pMON140913 | PCR19 | EXP-Cl.Ubq1:1:10 | 98 | 287.8 | 50.9 |
| pMON140914 | PCR20 | EXP-Cl.Ubq1:1:13 | 114 | 585.8 | 47.9 |
| pMON140915 | PCR21 | EXP-Cl.Ubq1:1:14 | 115 | 557.5 | 76.6 |
| pMON140916 | PCR22 | EXP-Cl.Ubq1:1:15 | 116 | 33.2 | 9.5 |

As can be seen in Table 31 above, the EXP sequences EXP-ANDge.Ubq1:1:7 (SEQ ID NO: 5), EXP-ANDge.Ubq1:1:8 (SEQ ID NO: 8), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116) were able to drive CP4 expression. All of the EXP sequences with the exception of one EXP-Cl.Ubq1:1:15 (SEQ ID NO: 116) drove CP4 expression levels at a much higher level than the constitutive control, EXP-Os.Act1:1:1 (SEQ ID NO: 164). Expression levels were lower than that of EXP-CaMV.35S-enh+Zm.DnaK:1:1 (SEQ ID NO: 170).

In a second series of experiments, expression of CP4 driven by amplicons comprised of the EXP sequences EXP-Cl.Ubq1:1:10 (SEQ ID NO: 98), EXP-Cl.Ubq1:1:16 (SEQ ID NO: 93) and EXP-Cl.Ubq1:1:17 (SEQ ID NO: 97) were assayed in transformed corn leaf protoplasts and compared to CP4 expression levels driven by the constitutive control, EXP-Os.Act1:1:1 (SEQ ID NO: 164). The average levels of CP4 protein expression expressed as part per million (ppm) is shown in Tables 32 below.

TABLE 32
Average CP4 protein expression in corn leaf protoplasts.

| | | | | Maize | Maize |
|---|---|---|---|---|---|
| | | | | Leaf | Leaf |
| | | | SEQ | CP4 | CP4 |
| Amplicon | | | ID | mg/total | mg/total |
| Template | Amplicon ID | EXP sequence | NO: | protein Avg | protein StdDev |
| pMON30167 | PCR25 | EXP-Os.Act1:1:1 | 164 | 12.2 | 1.69 |
| pMON140913 | PCR19 | EXP-Cl.Ubq1:1:10 | 98 | 307.5 | 24.21 |
| pMON142748 | pMON142748 | EXP-Cl.Ubq1:1:16 | 93 | 245.95 | 30.14 |
| pMON142749 | pMON142749 | EXP-Cl.Ubq1:1:17 | 97 | 302.85 | 25.32 |

As can be seen in Table 32 above, the EXP sequences EXP-Cl.Ubq1:1:10 (SEQ ID NO: 98), EXP-Cl.Ubq1:1:16 (SEQ ID NO: 93) and EXP-Cl.Ubq1:1:17 (SEQ ID NO: 97) were able to drive CP4 expression. Expression levels driven by all three EXP sequences were higher than that of the constitutive control, EXP-Os.Act1:1:1 (SEQ ID NO: 164).

### Example 8: Analysis of Regulatory Elements Driving CP4 in Wheat Protoplasts

This example illustrates the ability of EXP-Sv.Ubq1:1:7 (SEQ ID NO: 128), EXP-Sv.Ubq1:1:8 (SEQ ID NO: 132), EXP-Sv.Ubq1:1:9 (SEQ ID NO: 133), EXP-Zm.UbgM1:1:6 (SEQ ID NO: 137), EXP-Zm.UbgM1:1:8 (SEQ ID NO: 145), EXP-Zm.UbgM1:1:7 (SEQ ID NO: 141), EXP-SETit.Ubq1:1:5 (SEQ ID NO: 117), EXP-SETit.Ubq1:1:7 (SEQ ID NO: 123), EXP-SETit.Ubq1:1:6 (SEQ ID NO: 124), EXP-Sb.Ubq4:1:2 (SEQ ID NO: 151) and EXP-Sb.Ubq6:1:2 (SEQ ID NO: 153) to drive CP4 expression in wheat leaf protoplasts. These EXP sequences were cloned into plant binary transformation plasmid constructs using methods known in the art, and as described in Examples 2 and 5 above.

Three control plasmids (pMON30098, pMON4241, as previously described, and pMON43647 comprising a right border region from Agrobacterium tumefaciens with EXP-Os.Act1+CaMV.35S.2xA1-B3+Cs.Act1:5:1 (SEQ ID NO: 138) operably linked 5′ to a plastid targeted glyphosate tolerance coding sequence (CP4, U.S. RE39247), operably linked 5′ to T-AGRtu.nos-1:1:13, and a left border region (B-AGRtu.left border) with known constitutive regulatory elements driving either CP4 or GFP were constructed as outlined in Example 5.

Wheat leaf protoplasts were transformed using a PEG-based transformation method as described in the previous examples with the exception that 1.5×10⁵ protoplast cells per assay were used. Assays of luciferase and CP4 transgene expression were performed as described in Example 6 above. The mean CP4 expression levels determined by CP4 ELISA are presented in Table 34 below.

TABLE 34
Mean CP4 Protein Expression in Wheat Leaf Protoplast Cells.

| | | SEQ | CP4 | CP4 |
|---|---|---|---|---|
| | | ID | Average | STDEV |
| Plasmid | EXP sequence | NO: | ppm | ppm |
| No DNA | No DNA | | 0 | 0 |
| pMON30098 | GFP | | 0 | 0 |
| pMON43647 | EXP-Os.Act1 + | 172 | 656.2 | 124.5 |
| | CaMV.35S.2xA1-B3 + | | | |
| | Os.Act1:1:1 | | | |
| pMON42410 | EXP-CaMV.35S-enh + | 163 | 438.3 | 78.9 |
| | Ta.Lhcb1 + Os.Act1:1:1 | | | |
| pMON30167 | EXP-Os.Act1:1:1 | 164 | 583 | 107.4 |
| pMON129203 | EXP-Sv.Ubq1:1:7 | 128 | 156.9 | 25.1 |
| pMON129204 | EXP-Sv.Ubq1:1:8 | 132 | 39.5 | 7 |
| pMON129205 | EXP-Sv.Ubq1:1:9 | 133 | 154.5 | 56.5 |
| pMON129210 | EXP-Zm.UbqM1:1:6 | 137 | 1500 | 0 |
| pMON129211 | EXP-Zm.UbqM1:1:8 | 145 | 199.7 | 64.9 |
| pMON129212 | EXP-Zm.UbqM1:1:7 | 141 | 234.6 | 66.9 |
| pMON129200 | EXP-SETit.Ubq1:1:5 | 117 | 725.7 | 149.7 |
| pMON129201 | EXP-SETit.Ubq1:1:7 | 123 | 64.9 | 14.5 |
| pMON129202 | EXP-SETit.Ubq1:1:6 | 124 | 122.9 | 48.7 |
| pMON129219 | EXP-Sb.Ubq4:1:2 | 151 | 113.1 | 32.8 |

The total amount of CP4 expression in wheat protoplasts driven by the EXP sequences and the known constitutive EXP sequence EXP-CaMV.35S-enh+Ta.Lhcb1+Os.Act1:1:1 demonstrated different levels of CP4 expression in wheat protoplasts when compared to corn protoplasts.

Several EXP sequences drove CP4 expression at lower levels in wheat protoplasts than the known constitutive EXP sequences EXP-Os.Act1+CaMV.35S.2xA1-B3+Os.Act1:1:1 and EXP-CaMV.35S-enh+Ta.Lhcb1+Os.Act1:1:1. Two EXP sequences, EXP-Zm.UbgM1:1:6 (SEQ ID NO: 137), and EXP-SETit.Ubq1:1:5 (SEQ ID NO: 117), provide higher levels of CP4 expression in wheat protoplasts than the known constitutive, EXP sequences in this assay. EXP-Zm.UbgM1:1:2 drove expression of CP4 at the highest level, with expression levels being 2.2 to 3.4 fold higher than EXP-Os.Act1+CaMV.35S.2xA1-B3+Os.Act1:1:1 and EXP-CaMV.35S-enh+Ta.Lhcb1+Os.Act1:1:1, respectively. All EXP sequences assayed demonstrated the capacity to drive expression of CP4 in wheat cells.

### Example 9: Analysis of Regulatory Elements Driving CP4 in Wheat Protoplasts Using CP4 Transgene Cassette Amplicons

This example illustrates the ability of EXP-ANDge.Ubq1:1:7 (SEQ ID NO: 5), EXP-ANDge.Ubq1:1:8 (SEQ ID NO: 8), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115), EXP-Cl.Ubq1:1:15 (SEQ ID NO: 116), EXP-Cl.Ubq1:1:16 (SEQ ID NO: 93) and EXP-Cl.Ubq1:1:17 (SEQ ID NO: 97) in driving expression of glyphosate tolerance gene CP4 in wheat protoplasts. These EXP sequences were cloned into plant binary transformation plasmid constructs. The resulting plant expression vectors were used as amplification templates to produce a transgene cassette amplicon comprised of an ubiquitin EXP sequence operably linked 5′ to a plastid targeted glyphosate tolerant EPSPS coding sequence (CP4, U.S. RE39247), operably linked 5′ to the T-AGRtu.nos-1:1:13 3′ UTR and a left border region from A. tumefaciens. The resulting amplicons were used to transform corn leaf protoplasts cells.

Wheat leaf protoplasts were transformed using a PEG-based transformation method, as described in Example 2 above. Measurements of both CP4 were conducted using an ELISA-based assay. The average levels of CP4 protein expression expressed as part per million (ppm) is shown in Tables 35 and 36 below.

In a first series of experiments, expression of CP4 driven by amplicons comprised of the EXP sequences EXP-ANDge.Ubq1:1:7 (SEQ ID NO: 5), EXP-ANDge.Ubq1:1:8 (SEQ ID NO: 8), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116) were assayed in transformed wheat leaf protoplasts and compared to CP4 expression levels driven by the constitutive controls, EXP-CaMV.35S-enh+Zm.DnaK:1:1 (SEQ ID NO: 170) and EXP-Os.Act1:1:1 (SEQ ID NO: 164). The average levels of CP4 protein expression expressed as part per million (ppm) is shown in Tables 35 below.

TABLE 35
Average CP4 protein expression in wheat leaf protoplasts.

| | | | | CP4 | CP4 |
|---|---|---|---|---|---|
| | | | SEQ | ng/mg | ng/mg |
| Amplicon | Amplicon | | ID | total | total |
| Template | ID | EXP sequence | NO: | protein Average | protein STDEV |
| | | no DNA | | 0.00 | 0.00 |
| pMON30098 | | GFP (negative control) | | 0.00 | 0.00 |
| pMON19469 | PCR24 | EXP-CaMV.35S- | 170 | 76.11 | 18.65 |
| | | enh + Zm.DnaK:1:1 | | | |
| pMON30167 | PCR25 | EXP-Os.Act1:1:1 | 164 | 3.83 | 0.73 |
| pMON140896 | PCR41 | EXP-ANDge.Ubq1:1:7 | 5 | 103.46 | 16.31 |
| pMON140917 | PCR42 | EXP-ANDge.Ubq1:1:8 | 8 | 61.48 | 1.99 |
| pMON140897 | PCR43 | EXP-ANDge.Ubq1:1:10 | 10 | 62.65 | 4.58 |
| pMON140898 | PCR44 | EXP-ANDge.Ubq1:1:6 | 12 | 48.74 | 3.09 |
| pMON140899 | PCR45 | EXP-ANDge.Ubq1:1:11 | 14 | 54.91 | 3.50 |
| pMON140900 | PCR46 | EXP-ANDge.Ubq1:1:12 | 16 | 42.81 | 5.97 |
| pMON140904 | PCR50 | EXP-ERIra.Ubq1:1:9 | 22 | 31.26 | 1.69 |
| pMON140905 | PCR51 | EXP-ERIra.Ubq1:1:10 | 25 | 49.82 | 5.96 |
| pMON140906 | PCR52 | EXP-ERIra.Ubq1:1:8 | 27 | 37.43 | 4.52 |
| pMON140907 | PCR53 | EXP-ERIra.Ubq1:1:11 | 29 | 27.17 | 0.96 |
| pMON140908 | PCR54 | EXP-ERIra.Ubq1:1:12 | 31 | 17.41 | 4.13 |
| pMON140913 | PCR19 | EXP-Cl.Ubq1:1:10 | 98 | 66.66 | 13.45 |
| pMON140914 | PCR20 | EXP-Cl.Ubq1:1:13 | 114 | 79.42 | 10.74 |
| pMON140915 | PCR21 | EXP-Cl.Ubq1:1:14 | 115 | 75.53 | 9.32 |
| pMON140916 | PCR22 | EXP-Cl.Ubq1:1:15 | 116 | 0.00 | 0.00 |

As can be seen in Table 31 above, the EXP sequences EXP-ANDge.Ubq1:1:7 (SEQ ID NO: 5), EXP-ANDge.Ubq1:1:8 (SEQ ID NO: 8), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116) were able to drive CP4 expression. All of the EXP sequences with the exception of one EXP-Cl.Ubq1:1:15 (SEQ ID NO: 116) drove CP4 expression levels at a much higher level than the constitutive control, EXP-Os.Act1:1:1 (SEQ ID NO: 164). Expression levels were around the same level or lower than that of EXP-CaMV.35S-enh+Zm.DnaK:1:1 (SEQ ID NO: 170) for most of the EXP sequences.

In a second series of experiments, expression of CP4 driven by amplicons comprised of the EXP sequences EXP-Cl.Ubq1:1:10 (SEQ ID NO: 98), EXP-Cl.Ubq1:1:16 (SEQ ID NO: 93) and EXP-Cl.Ubq1:1:17 (SEQ ID NO: 97) were assayed in transformed wheat leaf protoplasts and compared to CP4 expression levels driven by the constitutive control, EXP-Os.Act1:1:1 (SEQ ID NO: 164). The average levels of CP4 protein expression expressed as part per million (ppm) is shown in Tables 32 below.

TABLE 36
Average CP4 protein expression in wheat leaf protoplasts.

| | | | | Maize | Maize |
|---|---|---|---|---|---|
| | | | | Leaf | Leaf |
| | | | SEQ | CP4 | CP4 |
| Amplicon | | | ID | mg/total | mg/total |
| Template | Amplicon ID | EXP sequence | NO: | protein Avg | protein StdDev |
| pMON30167 | PCR25 | EXP-Os.Act1:1:1 | 164 | 15.84 | 2.12 |
| pMON140913 | PCR19 | EXP-Cl.Ubq1:1:10 | 98 | 736.32 | 79.56 |
| pMON142748 | pMON142748 | EXP-Cl.Ubq1:1:16 | 93 | 593.72 | 80.22 |
| pMON142749 | pMON142749 | EXP-Cl.Ubq1:1:17 | 97 | 763.95 | 86.94 |

As can be seen in Table 36 above, the EXP sequences EXP-Cl.Ubq1:1:10 (SEQ ID NO: 98), EXP-Cl.Ubq1:1:16 (SEQ ID NO: 93) and EXP-Cl.Ubq1:1:17 (SEQ ID NO: 97) were able to drive CP4 expression. Expression levels driven by all three EXP sequences were higher than that of the constitutive control, EXP-Os.Act1:1:1 (SEQ ID NO: 164).

### Example 10: Analysis of Regulatory Elements Driving CP4 in Sugarcane Protoplasts

This example illustrates the ability of EXP-Sv.Ubq1:1:7 (SEQ ID NO: 128), EXP-Sv.Ubq1:1:8 (SEQ ID NO: 132), EXP-Sv.Ubq1:1:9 (SEQ ID NO: 133), EXP-Zm.UbgM1:1:6 (SEQ ID NO: 137), EXP-Zm.UbgM1:1:8 (SEQ ID NO: 145), EXP-Zm.UbgM1:1:7 (SEQ ID NO: 141), EXP-SETit.Ubq1:1:5 (SEQ ID NO: 117), EXP-SETit.Ubq1:1:7 (SEQ ID NO: 123), EXP-SETit.Ubq1:1:6 (SEQ ID NO: 124), EXP-Sb.Ubg4:1:2 (SEQ ID NO: 151), EXP-Sb.Ubg6:1:2 (SEQ ID NO: 153) and EXP-Cl.Ubq1:1:10 (SEQ ID NO: 98) in driving expression of CP4 in sugar cane leaf protoplasts. The EXP sequences were cloned into plant binary transformation plasmid constructs. The resulting vectors contained a right border region from Agrobacterium tumefaciens, an ubiquitin EXP sequence operably linked 5′ to a plastid targeted glyphosate tolerant EPSPS coding sequence (CP4, U.S. RE39247), operably linked 5′ to the T-AGRtu.nos-1:1:13 (SEQ ID NO: 127) or T-CaMV.35S-1:1:1 (SEQ ID NO: 140) 3′ UTR and a left border region from A. tumefaciens (B-AGRtu.left border). The resulting plasmid constructs were used to transform sugarcane leaf protoplasts cells using PEG transformation method.

Plasmid constructs pMON129203, pMON12904, pMON12905, pMON129210, pMON129211, pMON129212, pMON129200, pMON129201, pMON129202, pMON129219, and pMON129218 are as described in Table 12 above.

Three control plasmids (pMON30167 described above; pMON130803 also comprising EXP-Os.Act1::M (SEQ ID NO: 164); and pMON132804 comprising EXP-P-CaMV.35S-enh-1:1:13/L-CaMV.35S-1:1:2/I-Os.Act1-1:1:19 (SEQ ID NO: 139), with known constitutive regulatory elements driving CP4 were constructed and used to compare the relative CP4 expression levels driven by the ubiquitin EXP sequences listed in Table 37 below.

Sugarcane leaf protoplasts were transformed using a PEG-based transformation method. The mean CP4 expression levels determined by CP4 ELISA are presented in Table 37 below.

TABLE 37
Mean CP4 Protein Expression in Sugarcane Leaf Protoplast Cells.

| | | | Experiment 1 | | Experiment 2 | |
|---|---|---|---|---|---|---|
| | | SEQ | CP4 | CP4 | CP4 | CP4 |
| Plasmid | | ID | Average | STDEV | Average | STDEV |
| Construct | EXP sequence | NO: | ppm | ppm | ppm | ppm |
| pMON132804 | EXP-P-CaMV.35S-enh- | 173 | 557.97 | 194.05 | 283.63 | 95.8 |
| | 1:1:13/L-CaMV.35S-1:1:2/I- | | | | | |
| | Os.Act1-1:1:19 | | | | | |
| pMON30167 | EXP-Os.Act1:1:1 | 164 | 57.15 | 20.99 | 18.36 | 5.41 |
| pMON130803 | EXP-Os.Act1:1:1 | 164 | 34.26 | 1.61 | 16.57 | 3.71 |
| pMON129203 | EXP-Sv.Ubq1:1:7 | 128 | 89.2 | 32.46 | 56.86 | 9.55 |
| pMON129204 | EXP-Sv.Ubq1:1:8 | 132 | 87.2 | 45.87 | 98.46 | 12.93 |
| pMON129205 | EXP-Sv.Ubq1:1:9 | 133 | 263.57 | 70.14 | 72.53 | 9.25 |
| pMON129210 | EXP-Zm.UbqM1:1:6 | 137 | 353.08 | 29.16 | 199.31 | 41.7 |
| pMON129211 | EXP-Zm.UbqM1:1:8 | 145 | 748.18 | 15.1 | 411.24 | 17.12 |
| pMON129212 | EXP-Zm.UbqM1:1:7 | 141 | 454.88 | 75.77 | 215.06 | 23.22 |
| pMON129200 | EXP-SETit.Ubq1:1:5 | 117 | 150.74 | 63.21 | 91.71 | 41.35 |
| pMON129201 | EXP-SETit.Ubq1:1:7 | 123 | 119.57 | 58.1 | 102.72 | 31.12 |
| pMON129202 | EXP-SETit.Ubq1:1:6 | 124 | 43.79 | 25.77 | 97.63 | 46.07 |
| pMON129219 | EXP-Sb.Ubq4:1:2 | 151 | 95.63 | 38.69 | | |
| pMON129218 | EXP-Sb.Ubq6:1:2 | 153 | 343.34 | 119.2 | 179.75 | 51.16 |
| pMON129221 | EXP-Cl.Ubq1:1:10 | 98 | 374.8 | 205.28 | 258.93 | 38.03 |

As can be seen in Table 37 above, the EXP sequences demonstrated the ability to drive expression CP4 expression in sugarcane protoplasts. The levels of expression were similar to or greater than that of CP4 expression driven by EXP-Os.Act1:1:1 (SEQ ID NO: 164). One EXP sequence, EXP-Zm.UbgM1:1:8 (SEQ ID NO: 145), demonstrated higher levels of expression when compared to EXP-P-CaMV.35S-enh-1:1:13/L-CaMV.35S-1:1:2/I-Os.Act1-1:1:19 (SEQ ID NO: 139) in sugarcane protoplasts.

### Example 11: Analysis of Regulatory Elements Driving CP4 in Sugarcane Protoplasts Using CP4 Transgene Cassette Amplicons

This example illustrates the ability of EXP-ANDge.Ubq1:1:7 (SEQ ID NO: 5), EXP-ANDge.Ubq1:1:8 (SEQ ID NO: 8), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116) in driving expression of the glyphosate tolerance gene CP4 in sugarcane protoplasts. These EXP sequences were cloned into plant binary transformation plasmid constructs. The resulting plant expression vectors were used as amplification templates to produce a transgene cassette amplicon comprised of an ubiquitin EXP sequence operably linked 5′ to a plastid targeted glyphosate tolerant EPSPS coding sequence (CP4, U.S. RE39247), operably linked 5′ to the T-AGRtu.nos-1:1:13 3′ UTR and a left border region from A. tumefaciens. The resulting amplicons were used to transform sugarcane leaf protoplasts cells.

Sugarcane leaf protoplasts were transformed using a PEG-based transformation method, as described in Example 2 above. Measurements of both CP4 were conducted using an ELISA-based assay.

Expression of CP4 driven by amplicons comprised of the EXP sequences EXP-ANDge.Ubq1:1:7 (SEQ ID NO: 5), EXP-ANDge.Ubq1:1:8 (SEQ ID NO: 8), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114), EXP-Cl.Ubq1:1:14 (SEQ ID NO: 115) and EXP-Cl.Ubq1:1:15 (SEQ ID NO: 116) were assayed in transformed wheat leaf protoplasts and compared to CP4 expression levels driven by the constitutive controls, EXP-CaMV.35S-enh+Zm.DnaK:1:1 (SEQ ID NO: 170) and EXP-Os.Act1:1:1 (SEQ ID NO: 164). The average levels of CP4 protein expression expressed as part per million (ppm) is shown in Table 38 below.

TABLE 38
Average CP4 protein expression in sugarcane leaf protoplasts.

| | | | | CP4 | CP4 |
|---|---|---|---|---|---|
| | | | SEQ | ng/mg | ng/mg |
| Amplicon | Amplicon | | ID | total | total |
| Template | ID | EXP sequence | NO: | protein Average | protein STDEV |
| pMON19469 | PCR24 | EXP-CaMV.35S- | 170 | 99.6 | 7.2 |
| | | enh + Zm.DnaK:1:1 | | | |
| pMON30167 | PCR25 | EXP-Os.Act1:1:1 | 164 | 0.0 | 0.0 |
| pMON140896 | PCR41 | EXP-ANDge.Ubq1:1:7 | 5 | 21.9 | 3.3 |
| pMON140917 | PCR42 | EXP-ANDge.Ubq1:1:8 | 8 | 15.4 | 1.9 |
| pMON140897 | PCR43 | EXP-ANDge.Ubq1:1:10 | 10 | 20.7 | 2.2 |
| pMON140898 | PCR44 | EXP-ANDge.Ubq1:1:6 | 12 | 21.8 | 2.8 |
| pMON140899 | PCR45 | EXP-ANDge.Ubq1:1:11 | 14 | 36.9 | 7.2 |
| pMON140900 | PCR46 | EXP-ANDge.Ubq1:1:12 | 16 | 51.7 | 5.6 |
| pMON140904 | PCR50 | EXP-ERIra.Ubq1:1:9 | 22 | 10.3 | 1.1 |
| pMON140905 | PCR51 | EXP-ERIra.Ubq1:1:10 | 25 | 25.3 | 4.7 |
| pMON140906 | PCR52 | EXP-ERIra.Ubq1:1:8 | 27 | 29.9 | 4.6 |
| pMON140907 | PCR53 | EXP-ERIra.Ubq1:1:11 | 29 | 44.0 | 7.1 |
| pMON140908 | PCR54 | EXP-ERIra.Ubq1:1:12 | 31 | 37.0 | 5.4 |
| pMON140913 | PCR19 | EXP-Cl.Ubq1:1:10 | 98 | 19.2 | 1.3 |
| pMON140914 | PCR20 | EXP-Cl.Ubq1:1:13 | 114 | 20.5 | 2.1 |
| pMON140915 | PCR21 | EXP-Cl.Ubq1:1:14 | 115 | 23.2 | 1.6 |
| pMON140916 | PCR22 | EXP-Cl.Ubq1:1:15 | 116 | 0.0 | 0.0 |

As can be seen in Table 38 above, the EXP sequences EXP-ANDge.Ubq1:1:7 (SEQ ID NO: 5), EXP-ANDge.Ubq1:1:8 (SEQ ID NO: 8), EXP-ANDge.Ubq1:1:10 (SEQ ID NO: 10), EXP-ANDge.Ubq1:1:6 (SEQ ID NO: 12), EXP-ANDge.Ubq1:1:11 (SEQ ID NO: 14), EXP-ANDge.Ubq1:1:12 (SEQ ID NO: 16), EXP-ERIra.Ubq1:1:9 (SEQ ID NO: 22), EXP-ERIra.Ubq1:1:10 (SEQ ID NO: 25), EXP-ERIra.Ubq1:1:8 (SEQ ID NO: 27), EXP-ERIra.Ubq1:1:11 (SEQ ID NO: 29), EXP-ERIra.Ubq1:1:12 (SEQ ID NO: 31), EXP-Cl.Ubq1:1:10 (SEQ ID NO: 98), EXP-Cl.Ubq1:1:13 (SEQ ID NO: 114) and EXP-Cl.Ubq1:1:14 (SEQ ID NO: 115) were able to drive CP4 expression. EXP-Cl.Ubq1:1:15 (SEQ ID NO: 116) did not appear to express CP4 expression in this assay.

### Example 12: Analysis of Regulatory Elements Driving GUS in Transgenic Corn

Corn plants were transformed with plant expression vectors containing a EXP sequences driving expression of the β-glucuronidase (GUS) transgene, and the resulting plants were analyzed for GUS protein expression. The ubiquitin EXP sequences were cloned into plant binary transformation plasmid constructs using methods known in the art.

The resulting plant expression vectors contain a right border region from A. tumefaciens, a first transgene cassette to assay the EXP sequence operably linked to a coding sequence for β-glucuronidase (GUS) that possesses the processable intron GUS-2, described above, operably linked 5′ to the 3′ UTR from the rice lipid transfer protein gene (T-Os.LTP-1:1:1, SEQ ID NO: 141); a second transgene selection cassette used for selection of transformed plant cells that confers resistance to the herbicide glyphosate (driven by the rice Actin 1 promoter), and a left border region from A. tumefaciens. The resulting plasmids were used to transform corn plants. Table 39 lists the plasmid designations, the EXP sequences and the SEQ ID NOs, which are also described in Table 1.

TABLE 39
Binary plant transformation plasmids and the associated EXP sequences.

| Plasmid Construct | EXP sequence | SEQ ID NO: | Data |
|---|---|---|---|
| pMON142865 | EXP-ANDge.Ubq1:1:8 | 8 | R₀ and R₁ |
| pMON142864 | EXP-ERIra.Ubq1:1:8 | 27 | R₀ and R₁ |
| pMON142729 | EXP-Cl.Ubq1:1:12 | 90 | R₀ |
| pMON142730 | EXP-Cl.Ubq1:1:11 | 95 | R₀ |
| pMON132047 | EXP-Cl.Ubq1:1:23 | 108 | R₀ |
| pMON132037 | EXP-SETit.Ubq1:1:10 | 119 | R₀ and F₁ |
| pMON131957 | EXP-SETit.Ubq1:1:11 | 125 | F1 |
| pMON131958 | EXP-Sv.Ubq1:1:11 | 130 | R₀ and F₁ |
| pMON131959 | EXP-Sv.Ubq1:1:12 | 136 | R₀ |
| pMON131961 | EXP-Zm.UbqM1:1:10 | 139 | R₀ |
| pMON131963 | EXP-Zm.UbqM1:1:12 | 143 | R₀ |
| pMON131962 | EXP-Zm.UbqM1:1:11 | 149 | R₀ |
| pMON132932 | EXP-Sb.Ubq4:1:2 | 151 | R₀ |
| pMON132931 | EXP-Sb.Ubq6:1:3 | 155 | R₀ |
| pMON132974 | EXP-Sb.Ubq7:1:2 | 157 | R₀ and F₁ |

Plants were transformed using Agrobacterium-mediated transformations, for instance as described in U.S. Patent Application Publication 20090138985.

Histochemical GUS analysis was used for qualitative expression analysis of transformed plants. Whole tissue sections were incubated with GUS staining solution X-Gluc (5-bromo-4-chloro-3-indolyl-b-glucuronide) (1 milligram/milliliter) for an appropriate length of time, rinsed, and visually inspected for blue coloration. GUS activity was qualitatively determined by direct visual inspection or inspection under a microscope using selected plant organs and tissues. The R₀ plants are inspected for expression in the roots and leaves as well as the anther, silk and developing seed and embryo 21 days after pollination (21 DAP).

For quantitative analysis, total protein was extracted from selected tissues of transformed corn plants. One microgram of total protein was used with the fluorogenic substrate 4-methyleumbelliferyl-β-D-glucuronide (MUG) in a total reaction volume of 50 microliters. The reaction product, 4-methlyumbelliferone (4-MU), is maximally fluorescent at high pH, where the hydroxyl group is ionized. Addition of a basic solution of sodium carbonate simultaneously stops the assay and adjusts the pH for quantifying the fluorescent product. Fluorescence was measured with excitation at 365 nm, emission at 445 nm using a Fluoromax-3 (Horiba; Kyoto, Japan) with Micromax Reader, with slit width set at excitation 2 nm and emission 3 nm.

The average R₀ GUS expression observed for each transformation is presented in Tables 40 and 41 below. The R₀ GUS assay performed on transformants transformed with pMON131957 (EXP-SETit.Ubq1:1:11, SEQ ID NO:125) did not pass quality standards. These transformants were assayed at F1 generation and are presented further below in this example.

TABLE 40
Average R₀ GUS expression in root and leaf tissue.

| | SEQ | | | | | | | | |
|---|---|---|---|---|---|---|---|---|---|
| | ID | V3 | V4 | V7 | VT | V3 | V4 | V7 | VT |
| EXP sequence | NO: | Root | Root | Root | Root | Leaf | Leaf | Leaf | Leaf |
| EXP-ANDge.Ubq1:1:8 | 8 | nd | 255 | 199 | 70 | nd | 638 | 168 | 130 |
| EXP-ERIra.Ubq1:1:8 | 27 | nd | 477 | 246 | 62 | nd | 888 | 305 | 242 |
| EXP-Cl.Ubq1:1:12 | 90 | nd | 27 | 147 | 52 | nd | 75 | 189 | 199 |
| EXP-Cl.Ubq1:1:11 | 95 | nd | 28 | 77 | 50 | nd | 101 | 177 | 223 |
| EXP-Cl.Ubq1:1:23 | 108 | 0 | nd | 75 | 34 | 201 | nd | 194 | 200 |
| EXP-SETit.Ubq1:1:10 | 119 | 0 | nd | 29 | 57 | 58 | nd | 37 | 46 |
| EXP-Sv.Ubq1:1:11 | 130 | nd | nd | nd | 9 | 20 | nd | 55 | 29 |
| EXP-Sv.Ubq1:1:12 | 136 | 63 | nd | 0 | 28 | 184 | nd | 27 | 16 |
| EXP-Zm.UbqM1:1:10 | 139 | 0 | nd | 237 | 18 | 221 | nd | 272 | 272 |
| EXP-Zm.UbqM1:1:12 | 143 | 0 | nd | 21 | 43 | 234 | nd | 231 | 196 |
| EXP-Zm.UbqM1:1:11 | 149 | 124 | nd | 103 | 112 | 311 | nd | 369 | 297 |
| EXP-Sb.Ubq4:1:2 | 151 | 125 | nd | 0 | 95 | 233 | nd | 150 | 88 |
| EXP-Sb.Ubq6:1:3 | 155 | 154 | nd | 13 | 128 | 53 | nd | 39 | 55 |
| EXP-Sb.Ubq7:1:2 | 157 | 37 | nd | 22 | 18 | 165 | nd | 89 | 177 |

TABLE 41
Average R₀ GUS expression in corn reproductive organs (anther, silk) and
developing seed (embryo and endosperm).

| | SEQ | | | | |
|---|---|---|---|---|---|
| | ID | VT | VT/R1 | 21 DAP | 21 DAP |
| EXP sequence | NO: | Anther | Silk | Embryo | Endosperm |
| EXP-ANDge.Ubq1:1:8 | 8 | 247 | 256 | 24 | 54 |
| EXP-ERIra.Ubq1:1:8 | 27 | 246 | 237 | 36 | 61 |
| EXP-Cl.Ubq1:1:12 | 90 | 420 | 121 | 26 | 220 |
| EXP-Cl.Ubq1:1:11 | 95 | 326 | 227 | 41 | 221 |
| EXP-Cl.Ubq1:1:23 | 108 | 598 | 416 | 212 | 234 |
| EXP-SETit.Ubq1:1:10 | 119 | 132 | 85 | 50 | 63 |
| EXP-Sv.Ubq1:1:11 | 130 | 217 | 3 | 45 | 92 |
| EXP-Sv.Ubq1:1:12 | 136 | 120 | 21 | 49 | 112 |
| EXP-Zm.UbqM1:1:10 | 139 | 261 | 506 | 403 | 376 |
| EXP-Zm.UbqM1:1:12 | 143 | 775 | 362 | 253 | 247 |
| EXP-Zm.UbqM1:1:11 | 149 | 551 | 452 | 234 | 302 |
| EXP-Sb.Ubq4:1:2 | 151 | 213 | 0 | 25 | 79 |
| EXP-Sb.Ubq6:1:3 | 155 | 295 | 87 | 51 | 61 |
| EXP-Sb.Ubq7:1:2 | 157 | 423 | 229 | 274 | 90 |

In R₀ corn plants, GUS expression levels in the leaf and root differed amongst the ubiquitin EXP sequences. While all of the EXP sequences demonstrated the ability to drive GUS transgene expression in stably transformed plants, each EXP sequence demonstrated a unique pattern of expression relative to the others. For example, high levels of GUS expression were observed in early stages of root development (V4 and V7) for EXP-ANDge.Ubq1:1:8 (SEQ ID NO: 8) and EXP-ERIra.Ubq1:1:8 (SEQ ID NO: 27) and declined by VT stage. Root expression driven by EXP-Zm.UbgM1:1:10 (SEQ ID NO: 139) demonstrated no expression at V3 but was high at V7 and then dropped by VT stage. Root expression driven by EXP-Zm.UbgM1:1:11 (SEQ ID NO: 149) was maintained to a similar level throughout development from stages V3, V7 through VT. Root expression was observed to increase from early development (V3/V4) to V7 stage and then drop from V7 to V8 stage in plants transformed with EXP-Cl.Ubq1:1:12 (SEQ ID NO: 90), EXP-Cl.Ubq1:1:11 (SEQ ID NO: 95) and EXP-Cl.Ubq1:1:23 (SEQ ID NO: 108). GUS expression levels showed dramatic differences in leaf tissue as well. The highest levels of leaf expression were conferred in early development (V3/V4) with EXP-ANDge.Ubq1:1:8 (SEQ ID NO: 8) and EXP-ERIra.Ubq1:1:8 (SEQ ID NO: 27) which decline at V7 through VT stage. GUS expression is retained from V3 through VT stage using EXP-Zm.UbgM1:1:10 (SEQ ID NO: 139), EXP-Zm.UbgM1:1:11 (SEQ ID NO: 149), EXP-Zm.UbgM1:1:12 (SEQ ID NO: 143) and EXP-Cl.Ubq1:1:23 (SEQ ID NO: 108); and to a lower extent using EXP-SETit.Ubq1:1:10 (SEQ ID NO: 119) and EXP-Sb.Ubg6:1:3 (SEQ ID NO: 155). Expression in the leaf increased from V3 to V7 to VT stage using EXP-Cl.Ubq1:1:12 (SEQ ID NO: 90), EXP-Cl.Ubq1:1:11 (SEQ ID NO: 95) and EXP-Cl.Ubq1:1:23 (SEQ ID NO: 108) while expression declined from V3 to VT stage using EXP-Sv.Ubq1:1:12 (SEQ ID NO: 136) and EXP-Sb.Ubg4:1:2 (SEQ ID NO: 151).

Likewise, with respect to reproductive tissue (anther and silk) and developing seed (21DAP embryo and endosperm) different patterns of expression were observed unique to each EXP sequence. For example, High levels of expression were observed in anther and silk as well as the developing seed using EXP-Zm.UbgM1:1:10 (SEQ ID NO: 139), EXP-Zm.UbgM1:1:11 (SEQ ID NO: 149), EXP-Zm.UbgM1:1:12 (SEQ ID NO: 143) and EXP-Cl.Ubq1:1:23 (SEQ ID NO: 108). Expression was high in the anther and silk but low in the developing seed using EXP-ANDge.Ubq1:1:8 (SEQ ID NO: 8) and EXP-ERIra.Ubq1:1:8 (SEQ ID NO: 27). Expression driven by EXP-Sb.Ubg7:1:2 (SEQ ID NO: 157) was high in reproductive tissue and high in the developing embryo but was lower in the developing endosperm. The EXP sequence, EXP-Sb.Ubg4:1:2 (SEQ ID NO: 151) only demonstrated expression in the anther but not in the silk and expressed much lower in the developing seed. EXP-Sv.Ubq1:1:11 (SEQ ID NO: 130) demonstrated a similar pattern as EXP-Sb.Ubg4:1:2 (SEQ ID NO: 151) with respect to reproductive tissue and developing seed, but whereas EXP-Sb.Ubg4:1:2 (SEQ ID NO: 151) showed expression in root and leaf tissues, EXP-Sv.Ubq1:1:11 (SEQ ID NO: 130) expressed much lower in these same tissues.

R₀ generation transformants, selected for single copy insertions were crossed with a non-transgenic LH244 line (resulting in F₁) or were self-pollinated (resulting in R₁) in order to produce an F₁ or R₁ population of seeds. In either case, heterozygous F₁ or R₁ plants were selected for study. GUS expression levels were measured in selected tissues over the course of development as previously described. The F₁ or R₁ tissues used for this study included: imbibed seed embryo, imbibed seed endosperm, root and coleoptide at 4 days after germination (DAG); leaf and root at V3 stage; root and mature leaf at V8 stage; root, mature leaves, VT stage (at tasseling, prior to reproduction) anther, pollen, leaf and senescing leaf; R1 cob, silk, root and internode; kernel 12 days after pollination (DAP) and; embryo and endosperm 21 and 38 DAP. Selected tissue samples were also analyzed for F₁ plants exposed to conditions of drought and cold stress for transformants comprising pMON132037 (EXP-SETit.Ubq1:1:10, SEQ ID NO: 119), pMON131957 (EXP-SETit.Ubq1:1:11, SEQ ID NO: 125), pMON131958 (EXP-Sv.Ubq1:1:11, SEQ ID NO: 130) and pMON132974 (EXP-Sb.Ubg7:1:2, SEQ ID NO: 157). V3 root and leaf tissue was sampled after cold and drought exposure.

Drought stress was induced in F₁, V3 plants transformed with pMON132037 (EXP-SETit.Ubq1:1:10, SEQ ID NO: 119), pMON131957 (EXP-SETit.Ubq1:1:11, SEQ ID NO: 125), pMON131958 (EXP-Sv.Ubq1:1:11, SEQ ID NO: 130) and pMON132974 (EXP-Sb.Ubg7:1:2, SEQ ID NO: 157) by withholding watering for 4 days allowing the water content to be reduced by at least 50% of the original water content of the fully watered plant. The drought protocol was comprised essentially of the following steps. V3 stage plants were deprived of water. As a corn plant experiences drought, the shape of the leaf will change from the usual healthy and unfolded appearance to a leaf demonstrating folding at the mid-rib vascular bundle and appearing V-shaped when viewed from the leaf tip to the stem. This change in morphology usually began to occur by about 2 days after the cessation of watering and was shown in earlier experiments to be associated with water loss of around 50% as measured by weight of pots prior to cessation of watering and weight of pots when the leaf curl morphology was observed in un-watered plants. Plants were considered to be under drought conditions, when the leaves showed wilting as evidenced by an inward curling (V-shape) of the leaf. This level of stress is considered to be a form of sub-lethal stress. Once each plant demonstrated drought induction as defined above, the plant was destroyed to acquire both root and leaf samples.

In addition to drought, F₁ V3 stage plants transformed with pMON132037 (EXP-SETit.Ubq1:1:10, SEQ ID NO: 119), pMON131957 (EXP-SETit.Ubq1:1:11, SEQ ID NO: 125), pMON131958 (EXP-Sv.Ubq1:1:11, SEQ ID NO: 130) and pMON132974 (EXP-Sb.Ubg7:1:2, SEQ ID NO: 157) were also exposed to conditions of cold to determine if the regulatory elements demonstrated cold-induced expression of GUS. Whole plants were assayed for induction of GUS expression under cold stress at V3 stage. V3 stage corn plants were exposed to a temperature of 12° C. in a growth chamber for 24 hours. Plants in the growth chamber were grown under a white light fluence of 800 micro moles per meter squared per second with a light cycle of ten hours of white light and fourteen hours of darkness. After cold exposure, leaf and root tissues were sampled for quantitative GUS expression.

GUS expression was measured as described above. The average F₁ GUS expression determined for each tissue sample is presented in Tables 42 and 43 below.

TABLE 42
Average F₁ GUS expression in plants transformed with pMON142864 and
pMON142865.

| Organ | pMON142864 | pMON142865 |
|---|---|---|
| V3 Leaf | 86 | 74 |
| V3 Root | 41 | 52 |
| V8 Leaf | 109 | 123 |
| V8 Root | 241 | 252 |
| VT Flower, anthers | 168 | 208 |
| VT Leaf | 158 | 104 |
| R1 Cob | 171 | 224 |
| R1 silk | 314 | 274 |
| R1 Root | 721 | 308 |
| R1 internode | 428 | 364 |
| R2 Seed-12DAP | 109 | 72 |
| R3 Seed-21DAP-Embryo | 45 | 32 |
| R3 Seed-21DAP- | 175 | 196 |
| Endosperm | | |
| R5 Seed-38DAP-Embryo | 163 | 58 |
| R5 Seed-38DAP- | 90 | 69 |
| Endosperm | | |

TABLE 43
Average F₁ GUS expression in plants transformed with pMON132037,
pMON131957, pMON131958 and pMON132974.

| Organ | pMON132037 | pMON131957 | pMON131958 | pMON132974 |
|---|---|---|---|---|
| Imbibed Seed | 536 | 285 | 288 | 1190 |
| Embryo | | | | |
| Imbibed Seed | 95 | 71 | 73 | 316 |
| Endosperm | | | | |
| Coleoptile-4 | 218 | 60 | 143 | 136 |
| DAG | | | | |
| Root-4 DAG | 74 | 33 | 101 | 48 |
| V3 Leaf | 104 | 120 | 66 | 52 |
| V3 Root | 74 | 71 | 81 | 194 |
| V3 Leaf-cold | 73 | 15 | 72 | N/A |
| V3 Root-cold | 113 | 44 | 89 | 49 |
| V3 Leaf- | 97 | 344 | 103 | 157 |
| drought | | | | |
| V3 Root- | 205 | 153 | 129 | 236 |
| drought | | | | |
| V8 Leaf | 185 | 142 | 77 | 282 |
| V8 Root | 33 | 16 | 61 | 28 |
| VT Flower- | 968 | 625 | 619 | 888 |
| anthers | | | | |
| VT Leaf | 138 | 89 | 132 | 268 |
| VT Leaf- | 121 | 100 | 156 | 345 |
| senescing | | | | |
| VT Pollen | 610 | 1119 | 332 | 4249 |
| R1 Cob | 291 | 70 | 168 | 127 |
| R1 silk | 164 | 124 | 167 | 101 |
| R1 Root | 36 | 39 | 39 | 21 |
| R1 internode | 255 | 89 | 232 | 141 |
| R2 Seed- | 138 | 170 | 165 | 169 |
| 12DAP | | | | |
| R3 Seed-21 | 94 | 97 | 489 | 389 |
| DAP-Embryo | | | | |
| R3 Seed-21 | 57 | 118 | 52 | 217 |
| DAP- | | | | |
| Endosperm | | | | |
| R5 Seed-38 | 600 | 147 | 377 | 527 |
| DAP-Embryo | | | | |
| R5 Seed-38 | 58 | 36 | 57 | 106 |
| DAP- | | | | |
| Endosperm | | | | |

In F₁ corn plants, GUS expression levels in the various tissues sampled differed amongst the ubiquitin EXP sequences. While all of the EXP sequences demonstrated the ability to drive GUS transgene expression in stably transformed F₁ corn plants, each EXP sequence demonstrated a unique pattern of expression relative to the others. For example, R1 root expression is about twice that for EXP-ERIra.Ubq1:1:8 (SEQ ID NO: 27) than

EXP-ANDge.Ubq1:1:8 (SEQ ID NO: 8). GUS expression in the developing seed embryo at 38 DAP is almost three fold higher for EXP-ERIra.Ubq1:1:8 (SEQ ID NO: 27) than EXP-ANDge.Ubq1:1:8 (SEQ ID NO: 8). In contrast leaf and root expression at V3 and V8 stage is about the same for EXP-ERIra.Ubq1:1:8 (SEQ ID NO: 27) than EXP-ANDge.Ubq1:1:8 (SEQ ID NO: 8).

The F1 GUS expression in imbided seeds (embryo and endosperm tissues) was much higher in plants transformed with EXP-Sb.Ubg7:1:2 (SEQ ID NO: 157) than in those transformed with EXP-SETit.Ubq1:1:10 (SEQ ID NO: 119), EXP-SETit.Ubq1:1:11 (SEQ ID NO: 125) and EXP-Sv.Ubq1:1:11 (SEQ ID NO: 130). Drought caused an increase in V3 root expression in plants transformed with EXP-SETit.Ubq1:1:10 (SEQ ID NO: 119), EXP-SETit.Ubq1:1:11 (SEQ ID NO: 125), EXP-Sv.Ubq1:1:11 (SEQ ID NO: 130) and EXP-Sb.Ubg7:1:2 (SEQ ID NO: 157), but only increased leaf expression in plants transformed with EXP-SETit.Ubq1:1:11 (SEQ ID NO: 125), EXP-Sv.Ubq1:1:11 (SEQ ID NO: 130) and EXP-Sb.Ubg7:1:2 (SEQ ID NO: 157). The drought enhanced V3 expression was greatest using EXP-SETit.Ubq1:1:11 (SEQ ID NO: 125). Pollen expression was also much higher in plants transformed with EXP-Sb.Ubg7:1:2 (SEQ ID NO: 157) than in those transformed with EXP-SETit.Ubq1:1:10 (SEQ ID NO: 119), EXP-SETit.Ubq1:1:11 (SEQ ID NO: 125) and EXP-Sv.Ubq1:1:11 (SEQ ID NO: 130). Expression in the R1 internode was greatest with EXP-SETit.Ubq1:1:10 (SEQ ID NO: 119) and EXP-Sv.Ubq1:1:11 (SEQ ID NO: 130) and least in plants transformed with EXP-SETit.Ubq1:1:11 (SEQ ID NO: 125).

Each EXP sequence demonstrated the ability to drive transgene expression in stably transformed corn plants. However, each EXP sequence had a pattern of expression for each tissue that was unique and offers an opportunity to select the EXP sequence which will best provide expression of a specific transgene depending upon the tissue expression strategy needed to achieve the desired results. This example demonstrates EXP sequences isolated from homologous genes do not necessarily behave equivalently in the transformed plant and that expression can only be determined through empirical investigation of the properties for each EXP sequence and cannot be predicted based upon the gene homology from which the promoter was derived.

### Example 13: Analysis of Regulatory Elements Driving CP4 in Transgenic Corn

Corn plants were transformed with plant expression vectors containing EXP sequences driving expression of the CP4 transgene, and the resulting plants were analyzed for CP4 protein expression.

The EXP sequences EXP-ANDge.Ubq1:1:8 (SEQ ID NO: 8), EXP-ERIra.Ubq1:1:8 (SEQ ID NO: 27), EXP-Cl.Ubq1:1:10 (SEQ ID NO: 98), EXP-Sv.Ubq1:1:9 (SEQ ID NO: 133) and EXP-Zm.UbgM1:1:7 (SEQ ID NO: 141) were cloned into plant binary transformation plasmid constructs. The resulting vectors contained a right border region from Agrobacterium tumefaciens, an ubiquitin EXP sequence operably linked 5′ to a plastid targeted glyphosate tolerant EPSPS coding sequence (CP4, U.S. RE39247), operably linked 5′ to the T-AGRtu.nos-1:1:13 (SEQ ID NO: 127) 3′ UTR and a left border region from A. tumefaciens. Table 44 below shows the plasmid constructs used to transform corn and the corresponding EXP sequences.

TABLE 44
CP4 plasmid constructs and corresponding EXP sequences
used to transform corn.

| Plasmid | | SEQ ID | |
|---|---|---|---|
| Construct | EXP sequence | NO: | Data |
| pMON141619 | EXP-ANDge.Ubq1:1:8 | 8 | R₀ and F₁ |
| pMON142862 | EXP-ERIra.Ubq1:1:8 | 27 | R₀ and F₁ |
| pMON129221 | EXP-Cl.Ubq1:1:10 | 98 | R₀ and F₁ |
| pMON129205 | EXP-Sv.Ubq1:1:9 | 133 | R₀ and F₁ |
| pMON129212 | EXP-Zm.UbqM1:1:7 | 141 | R₀ |

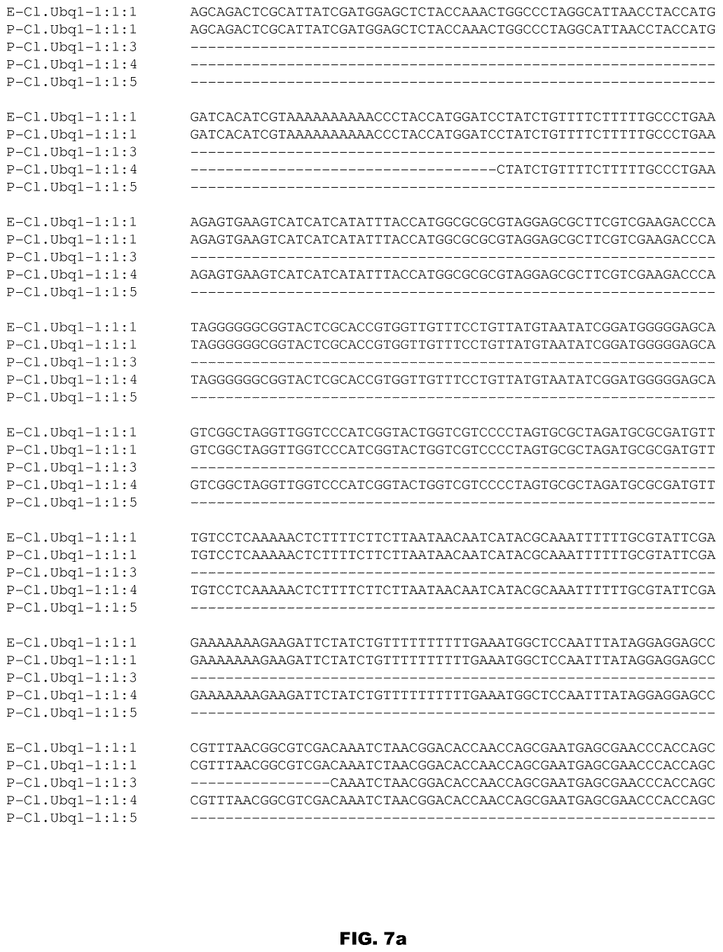

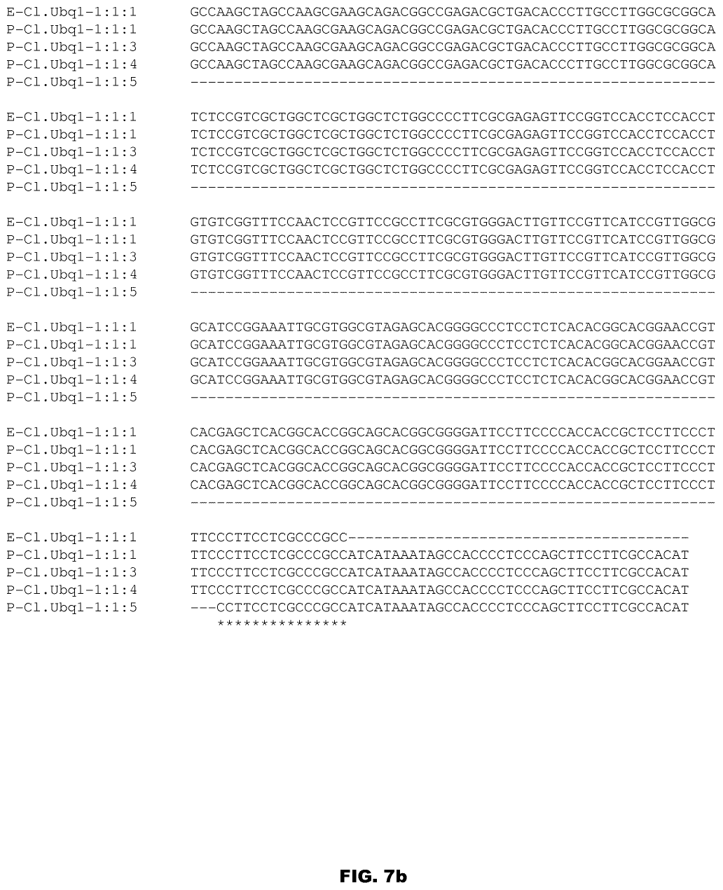

The resulting plasmids were used to transform corn plants. Transformed plants were selected for one or two copies of the inserted T-DNA and grown in the greenhouse. Selected tissues were sampled from the R₀ transformed plants at specific stages of development and CP4 protein levels were measured in those tissues using an CP4 ELISA assay. The average CP4 expression observed for each transformation is presented in Tables 45 and 46 below and graphically in FIG. 7.

TABLE 45
Average leaf and root CP4 expression in R₀ transformed corn plants.

| | SEQ | | | | | | |
|---|---|---|---|---|---|---|---|
| | ID | V4 | V7 | VT | V4 | V7 | VT |
| EXP sequence | NO: | Leaf | Leaf | Leaf | Root | Root | Root |
| EXP-ANDge.Ubq1:1:8 | 8 | 20.90 | 18.53 | 25.49 | 11.50 | 26.54 | 17.20 |
| EXP-ERIra.Ubq1:1:8 | 27 | 19.92 | 16.60 | 25.58 | 9.92 | 26.31 | 13.33 |
| EXP-Cl.Ubq1:1:10 | 98 | 10.70 | 12.49 | 17.42 | 7.56 | 13.95 | 6.68 |
| EXP-Sv.Ubq1:1:9 | 133 | 3.72 | 4.34 | 4.48 | 2.90 | 6.99 | 2.78 |
| EXP-Zm.UbqM1:1:7 | 141 | 13.42 | 21.89 | 38.78 | 9.56 | 16.69 | 11.15 |

TABLE 46
Average CP4 expression in reproductive tissue and developing seed in R₀
transformed corn plants.

| | SEQ | | | | |
|---|---|---|---|---|---|
| | ID | VT | R1 | R3 | R3 |
| EXP sequence | NO: | Tassel | Silk | Embryo | Endosperm |
| EXP-ANDge.Ubq1:1:8 | 8 | 24.14 | 5.55 | 7.29 | 4.91 |
| EXP-ERIra.Ubq1:1:8 | 27 | 19.20 | 10.27 | 12.60 | 4.70 |
| EXP-Cl.Ubq1:1:10 | 98 | 18.70 | 16.21 | 8.26 | 8.82 |
| EXP-Sv.Ubq1:1:9 | 133 | 7.10 | 4.72 | 3.13 | 1.74 |
| EXP-Zm.UbqM1:1:7 | 141 | 67.25 | 11.21 | 7.85 | 10.69 |

As can be seen in Tables 45 and 46, each of the EXP sequences EXP-ANDge.Ubq1:1:8 (SEQ ID NO: 8), EXP-ERIra.Ubq1:1:8 (SEQ ID NO: 27), EXP-Cl.Ubq1:1:10 (SEQ ID NO: 98), EXP-Sv.Ubq1:1:9 (SEQ ID NO: 133) and EXP-Zm.UbqM1:1:7 (SEQ ID NO: 141) was able to drive CP4 expression in all tissues sampled from the R₀ transformed plants. Higher expression of CP4 in the root and leaf of transformants comprising EXP-ANDge.Ubq1:1:8 (SEQ ID NO: 8) and EXP-ERIra.Ubq1:1:8 (SEQ ID NO: 27) driving CP4 than EXP-Cl.Ubq1:1:10 (SEQ ID NO: 98) driving CP4 may be related to the level of vegetative tolerance to glyphosate application as observed for these populations of transformants (see Example 14 below).

Each EXP sequence exhibited a unique expression pattern with respect to the level of expression for each tissue sampled. For example, while CP4 expression in leaf, root and tassel were similar for the EXP sequences, EXP-ANDge.Ubq1:1:8 (SEQ ID NO: 8) and EXP-ERIra.Ubq1:1:8 (SEQ ID NO: 27), expression in silk using EXP-ANDge.Ubq1:1:8 (SEQ ID NO: 8) was half that of expression driven by ERIra.Ubq1:1:8 (SEQ ID NO: 21). This might be advantageous for expression of transgenes in which constitutive expression is desired but less expression in silk tissue would be preferred. The EXP sequences demonstrate unique patterns of CP4 constitutive expression in R₀ transformed corn plants.

The R₀ transformed corn plants were crossed with a non-transgenic LH244 variety to produce F1 seed. The resulting F1 generation seed was analyzed for segregation of the transgene cassette and plants heterozygous for the CP4 cassette were selected for analysis of CP4 expression. Seed was grown in the greenhouse and two groups of plants were produced, one group was sprayed with glyphosate while the other was left unsprayed. Expression of CP4 was analyzed in selected tissues using a standard ELISA based assay. The average CP4 expression is shown in Tables 47 and 48 below.

TABLE 47
Average CP4 expression in F₁ transformed corn plants.

| Organ | pMON141619 | pMON142862 | pMON129221 |
|---|---|---|---|
| V4 Leaf | 11.50 | 13.51 | 7.68 |
| V4 Root | 12.48 | 12.60 | 10.29 |
| V7 Leaf | 16.59 | 20.21 | 12.01 |
| V7 Root | 11.00 | 13.62 | 8.15 |
| VT Leaf | 39.88 | 44.85 | 29.42 |
| VT Root | 17.43 | 21.83 | 13.43 |
| VT Flower, anthers | 52.74 | 55.72 | 53.62 |
| R1 Silk | 16.01 | 23.81 | 14.42 |
| R3 Seed-21 DAP- | 33.29 | 57.96 | 51.64 |
| Embryo | | | |
| R3 Seed-21 DAP- | 2.99 | 3.20 | 6.44 |
| Endosperm | | | |

As can be seen in Table 47 above, CP4 expression in leaf and root was higher in F₁ transformants transformed with pMON141619 (EXP-ANDge.Ubq1:1:8, SEQ ID NO: 5) and pMON142862 (EXP-ERIra.Ubq1:1:8, SEQ ID NO: 27) than in those transformed with pMON129221 (EXP-Cl.Ubq1:1:10, SEQ ID NO: 98). Expression in the anther tissue was similar for all three EXP sequences while expression in the silk was highest using EXP-ERIra.Ubq1:1:8 (SEQ ID NO: 27). Expression in the developing embryo (21 DAP) was highest in transformants comprising EXP-ERIra.Ubq1:1:8 (SEQ ID NO: 27) and EXP-Cl.Ubq1:1:10 (SEQ ID NO: 98) driving CP4. Expression in the developing endosperm was higher in transformants comprising EXP-Cl.Ubq1:1:10 (SEQ ID NO: 98) driving CP4.

TABLE 48
Average CP4 expression in F₁ transformed corn plants.

| Organ | pMON129205 |
|---|---|
| V4 Leaf | 1.73 |
| V4 Root | 2.44 |
| V7 Leaf | 2.84 |
| V7 Root | 1.51 |
| VT Leaf | 3.29 |
| VT Root | 2.63 |
| VT Flower, anthers | 7.52 |
| R1 Silk | 1.99 |
| R3 Seed-21 DAP-Embryo | 3.40 |
| R3 Seed-21 DAP-Endosperm | 1.79 |

As can be seen in Tables 47-48 above, CP4 expression was lower in all tissues of F₁ transformants transformed with pMON129205 (EXP-Sv.Ubq1:1:9, SEQ ID NO: 133) than those transformed with pMON141619 (EXP-ANDge.Ubq1:1:8, SEQ ID NO: 8), pMON142862 (EXP-ERIra.Ubq1:1:8, SEQ ID NO: 27) and pMON129221 (EXP-Cl.Ubq1:1:10, SEQ ID NO: 98).

The unique patterns of expression conferred by each of the EXP sequences assayed provide an opportunity to produce a transgenic plant in which expression can be fine-tuned to make small adjustments in transgene expression for optimal performance or effectiveness. In addition, empirical testing of these EXP sequences driving different transgene expression may produce results in which one particular EXP sequence is most suitable for expression of a specific transgene or class of transgenes while another EXP sequence is found to be best for a different transgene or class of transgenes.

### Example 14: Analysis of Vegetative Glyphosate Tolerance in R₀ Transgenic Corn Plants

Corn plants were transformed with plant expression vectors containing EXP sequences driving expression of the CP4 transgene, and the resulting plants were assessed for vegetative and reproductive tolerance to glyphosate application.

F₁ transformed corn plants described in Example 13 above transformed with pMON141619, pMON142862, pMON129221, pMON129205 and pMON129212 and comprised of the EXP sequences EXP-ANDge.Ubq1:1:8 (SEQ ID NO: 8), EXP-ERIra.Ubq1:1:8 (SEQ ID NO: 27), EXP-Cl.Ubq1:1:10 (SEQ ID NO: 98), EXP-Sv.Ubq1:1:9 (SEQ ID NO: 133) and EXP-Zm.UbgM1:1:7 (SEQ ID NO: 141), respectively driving CP4 were assessed for both vegetative and reproductive tolerance when sprayed with glyphosate. Ten F1 plants for each event were divided into two groups, the first group consisting of five plants that received glyphosate spray and V4 and V8 stage of development; and a second group of five plants that were left unsprayed (i.e. control). Glyphosate was applied by broadcast foliar spray application using Roundup WeatherMax® at an application rate of 1.5 a.e./acre (a.e. acid equivalent). After seven to ten days, the leaves of each plant were assessed for damage. Vegetative tolerance (Veg Tol in Table 49) was assessed comparing the unsprayed and sprayed plants for each event and a damage rating scale was used to provide a final rating for vegetative tolerance (T=tolerant, NT=not tolerant). In addition seed set was assayed for all of the plants in each event. Seed set measures between control plants and sprayed plants was compared and an assignment of reproductive tolerance (Repro Tol in Table 49) was given for each event based upon the percent seed set of sprayed plants relative to the controls (T=tolerant, NT=not tolerant). Table 49 below shows the vegetative and reproductive tolerance ratings for each event sprayed at V4 and V8 stage. The letter “T” denotes tolerant and “NT” denotes not tolerant.

TABLE 49
Leaf damage ratings of individual transformed corn events at V4 and V8 stage.

| | | | | | Veg | |
|---|---|---|---|---|---|---|
| Plasmid | | SEQ ID | | Veg Tol | Tol | Repro |
| Construct | EXP sequence | NO: | Event | V4 | V8 | Tol |
| pMON141619 | EXP- | 8 | Event 1 | T | T | NT |
| | ANDge.Ubq1:1:8 | | Event 2 | T | T | T |
| | | | Event 3 | T | T | NT |
| | | | Event 4 | T | T | NT |
| | | | Event 5 | T | T | T |
| | | | Event 6 | T | T | NT |
| | | | Event 7 | T | T | T |
| | | | Event 8 | T | T | T |
| | | | Event 9 | T | T | NT |
| pMON142862 | EXP-ERIra.Ubq1:1:8 | 27 | Event 1 | T | T | T |
| | | | Event 2 | T | T | NT |
| | | | Event 3 | T | T | T |
| | | | Event 4 | T | T | T |
| | | | Event 5 | T | T | NT |
| | | | Event 6 | T | T | T |
| | | | Event 7 | T | T | NT |
| | | | Event 8 | T | T | T |
| | | | Event 9 | T | T | T |
| pMON129221 | EXP-Cl.Ubq1:1:10 | 98 | Event 1 | T | T | NT |
| | | | Event 2 | T | T | NT |
| | | | Event 3 | NT | NT | T |
| | | | Event 4 | NT | NT | T |
| | | | Event 5 | T | T | NT |
| | | | Event 6 | NT | NT | T |
| | | | Event 7 | T | T | T |
| pMON129205 | EXP-Sv.Ubq1:1:9 | 133 | Event 1 | NT | NT | |
| | | | Event 2 | NT | NT | NT |
| | | | Event 3 | T | T | NT |
| | | | Event 4 | NT | NT | |
| | | | Event 5 | NT | NT | NT |
| | | | Event 6 | NT | NT | NT |
| | | | Event 7 | NT | NT | NT |
| pMON129212 | EXP-Zm.UbqM1:1:7 | 141 | Event 1 | T | T | |
| | | | Event 2 | T | T | |
| | | | Event 3 | T | T | |
| | | | Event 4 | T | T | |
| | | | Event 5 | T | T | |
| | | | Event 6 | T | T | |
| | | | Event 7 | T | T | |
| | | | Event 8 | T | T | |
| | | | Event 9 | T | T | |
| | | | Event 10 | T | T | |

From Table 49 above, all transformed events assayed comprising CP4 transgene cassettes comprising the EXP sequences EXP-ANDge.Ubq1:1:8 (SEQ ID NO: 8), EXP-ERIra.Ubq1:1:8 (SEQ ID NO: 27) and EXP-Zm.UbgM1:1:7 (SEQ ID NO: 141) demonstrated full vegetative tolerance based upon damage ratings that did not exceed a score of ten. Four events of nine comprising EXP-ANDge.Ubq1:1:8 (SEQ ID NO: 8) and six events of nine comprising EXP-ERIra.Ubq1:1:8 (SEQ ID NO: 27) were both vegetatively and reproductively tolerant to glyphosate application. In contrast, events comprising EXP-Cl.Ubq1:1:10 (SEQ ID NO: 98) were either vegetatively tolerant or reproductively tolerant but not both. Only one event comprising EXP-Sv.Ubq1:1:9 (SEQ ID NO: 133) demonstrated vegetative tolerance and none of the events tested were reproductive tolerant. All events comprising EXP-Zm.UbgM1:1:7 (SEQ ID NO: 141) demonstrated vegetative tolerance but and assessment of reproductive tolerance is still in progress.

### Example 15: Analysis of Expression Using Different 3′ End Intron/Exon Splice Junction Sequences

Corn and Wheat leaf protoplast cells were transformed with plant expression constructs comprising EXP sequences driving GUS expression that comprise the same promoter and leader but have different 3′ end nucleotides following the intron/exon splice junction sequence, 5′-AG-3′ to see if expression is affected by the slight change in sequence. Expression was also compared to that of two constitutive control plasmids.

Plant expression constructs are built comprising a GUS expression cassette. The resulting vectors are comprised of the Coix lacryma-jobi ubiquitin promoter, P-Cl.Ubq1-1:1:1 (SEQ ID NO: 80) operably linked 5′ to the leader sequence, L-Cl.Ubq1-1:1:1 (SEQ ID NO: 81), operably linked 5′ to an intron element shown in Table 50 below which each comprise different nucleotides at the very 3′ end just after the intron/exon splice junction 5′-AG-3′ sequence, operably linked 5′ to a GUS coding sequence which is operably linked 5′ to T-AGRtu.nos-1:1:13 (SEQ ID NO: 127) 3′ UTR. Table 50 below shows the plant expression constructs and the corresponding 3′ end sequence.

TABLE 50
Plant expression constructs, introns and 3′ end sequence following
the intron/exon splice junction sequence 5′-AG-3′.

| | | | | Intron 3′ end |
|---|---|---|---|---|
| | | | | nucleotides |
| | | SEQ | | immediately |
| Plasmid | | ID | | following 3′ |
| construct | EXP sequence | NO: | Intron Variant | splice site AG |
| pMON140889 | EXP-Cl.Ubq1:1:10 | 98 | I-Cl.Ubq1-1:1:6 | GTC |
| | | | (SEQ ID NO: 94) | |
| pMON146795 | EXP-Cl.Ubq1:1:18 | 99 | I-Cl.Ubq1-1:1:7 | GTG |
| | | | (SEQ ID NO: 92) | |
| pMON146796 | EXP-Cl.Ubq1:1:19 | 100 | I-Cl.Ubq1-1:1:8 | GCG |
| | | | (SEQ ID NO: 101) | |
| pMON146797 | EXP-Cl.Ubq1:1:20 | 102 | I-Cl.Ubq1-1:1:9 | GAC |
| | | | (SEQ ID NO: 103) | |
| pMON146798 | EXP-Cl.Ubq1:1:21 | 104 | I-Cl.Ubq1-1:1:10 | ACC |
| | | | (SEQ ID NO: 105) | |
| pMON146799 | EXP-Cl.Ubq1:1:22 | 106 | I-Cl.Ubq1-1:1:11 | GGG |
| | | | (SEQ ID NO: 107) | |
| pMON146800 | EXP-Cl.Ubq1:1:23 | 108 | I-Cl.Ubq1-1:1:12 | GGT |
| | | | (SEQ ID NO: 109) | |
| pMON146801 | EXP-Cl.Ubq1:1:24 | 110 | I-Cl.Ubq1-1:1:13 | CGT |
| | | | (SEQ ID NO: 111) | |
| pMON146802 | EXP-Cl.Ubq1:1:25 | 112 | I-Cl.Ubq1-1:1:14 | TGT |
| | | | (SEQ ID NO: 113) | |
| pMON25455 | EXP-Os.Act1:1:9 | 179 | | Constitutive |
| | | | | Control |
| pMON65328 | EXP-CaMV.35S-enh + | 163 | | Constitutive |
| | Ta.Lhcb1 + | | | Control |
| | Os.Act1:1:1 | | | |

Corn and Wheat protoplasts were transformed as previously described and assayed for GUS and luciferase expression. Table 51 below shows the average GUS and RLuc values for both corn and wheat protoplast expression.

TABLE 51
Average GUS and RLuc values for corn and wheat protoplast cells.

| | Intron 3′ | | | | | | |
|---|---|---|---|---|---|---|---|
| | end | | | | | | |
| | nucleotides | | | | | | |
| | immed. | | | | | | |
| | following 3′ | Corn | | | Wheat | | |
| | splice site | Average | Average | GUS/ | Ave. | Ave. | GUS/ |
| EXP sequence | AG | GUS | RLuc | RLuc | GUS | RLuc | RLuc |
| EXP- | GTC | 140343.0 | 93870.75 | 1.50 | 40906.25 | 17381.75 | 2.35 |
| Cl.Ubq1:1:10 | | | | | | | |
| EXP- | GTG | 143106.25 | 60565.25 | 2.36 | 56709.00 | 17898.75 | 3.17 |
| Cl.Ubq1:1:18 | | | | | | | |
| EXP- | GCG | 136326.83 | 88589.75 | 1.54 | 43211.00 | 17352.50 | 2.49 |
| Cl.Ubq1:1:19 | | | | | | | |
| EXP- | GAC | 138110.83 | 104751.42 | 1.32 | 31711.50 | 17953.75 | 1.77 |
| Cl.Ubq1:1:20 | | | | | | | |
| EXP- | ACC | 137906.75 | 72519.50 | 1.90 | 54164.17 | 17772.83 | 3.05 |
| Cl.Ubq1:1:21 | | | | | | | |
| EXP- | GGG | 137306.83 | 92643.42 | 1.48 | 55198.25 | 14476.75 | 3.81 |
| Cl.Ubq1:1:22 | | | | | | | |
| EXP- | GGT | 144085.50 | 64351.25 | 2.24 | 43008.83 | 13911.50 | 3.09 |
| Cl.Ubq1:1:23 | | | | | | | |
| EXP- | CGT | 142061.50 | 65884.00 | 2.16 | 51210.50 | 15041.00 | 3.40 |
| Cl.Ubq1:1:24 | | | | | | | |
| EXP- | TGT | 140353.00 | 61249.50 | 2.29 | 49577.75 | 15348.25 | 3.23 |
| Cl.Ubq1:1:25 | | | | | | | |
| EXP-Os.Act1:1:9 | Constitutive | 37665.25 | 65835.50 | 0.57 | 10830.25 | 17716.50 | 0.61 |
| | Control | | | | | | |
| EXP-CaMV.35S- | Constitutive | 49833.75 | 41268.75 | 1.21 | 15598.83 | 14877.50 | 1.05 |
| enh+Ta.Lhcb1+Os.Act1:1:1 | Control | | | | | | |

The GUS/RLuc values for each Coix lacryma-jobi ubiquitin EXP sequence from PP Table 46 above were used to normalize the expression relative to the two constitutive controls EXP-Os.Act1:1:9 (SEQ ID NO: 179) and EXP-CaMV.35S-enh+Ta.Lhcb1+Os.Act1:1:1 (SEQ ID NO: 163) and are presented in Table 52 below.

TABLE 52
Normalized expression values of the Coix lacryma-jobi ubiquitin EXP sequences relative to
EXP-Os.Act1:1:9 (SEQ ID NO: 179) and EXP-CaMV.35S-enh + Ta.Lhcb1 + Os.Act1:1:1 (SEQ ID NO: 163).

| | | Corn | | Wheat | |
|---|---|---|---|---|---|
| | | | GUS/RLuc | | GUS/Rluc |
| | Intron 3′ end | | Normalized | | Normalized |
| | nucleotides | GUS/RLuc | with respect to | GUS/Rluc | with respect to |
| | immediately | Normalized | EXP-CaMV.35S-enh + | Normalized | EXP-CaMV.35S-enh + |
| | following 3′ | with respect to | Ta.Lhcb1 + | with respect to | Ta.Lhcb1 + |
| EXP sequence | splice site AG | EXP-Os.Act1:1:9 | Os.Act1:1:1 | EXP-Os.Act1:1:9 | Os.Act1:1:1 |
| EXP-Cl.Ubq1:1:10 | GTC | 2.61 | 1.24 | 3.85 | 2.24 |
| EXP-Cl.Ubq1:1:18 | GTG | 4.13 | 1.96 | 5.18 | 3.02 |
| EXP-Cl.Ubq1:1:19 | GCG | 2.69 | 1.27 | 4.07 | 2.38 |
| EXP-Cl.Ubq1:1:20 | GAC | 2.30 | 1.09 | 2.89 | 1.68 |
| EXP-Cl.Ubq1:1:21 | ACC | 3.32 | 1.57 | 4.99 | 2.91 |
| EXP-Cl.Ubq1:1:22 | GGG | 2.59 | 1.23 | 6.24 | 3.64 |
| EXP-Cl.Ubq1:1:23 | GGT | 3.91 | 1.85 | 5.06 | 2.95 |
| EXP-Cl.Ubq1:1:24 | CGT | 3.77 | 1.79 | 5.57 | 3.25 |
| EXP-Cl.Ubq1:1:25 | TGT | 4.01 | 1.90 | 5.28 | 3.08 |
| EXP-Os.Act1:1:9 | Constitutive | 1.00 | 0.47 | 1.00 | 0.58 |
| | Control | | | | |
| EXP-CaMV.35S-enh + | Constitutive | 2.11 | 1.00 | 1.72 | 1.00 |
| Ta.Lhcb1 + | Control | | | | |
| Os.Act1:1:1 | | | | | |

As is shown in Table 52 above, each of the Coix lacryma-jobi ubiquitin EXP sequences provided expression that was greater than either constitutive control in both corn and wheat. Expression in corn protoplasts was relatively similar for all of the Coix ubiquitin EXP sequences. Expression in wheat was a little more variable. The use of different 3′ end nucleotides following the intron/exon splice junction sequence, 5′-AG-3′ did not appear to dramatically affect expression of GUS with the exception of GUS driven by EXP-Cl.Ubq1:1:20 (SEQ ID NO: 102). EXP-Cl.Ubq1:1:20 comprises the 3′ end nucleotide sequences, 5′-GAC-3′ following the intron/exon splice junction 5′-AG-3′ sequence and caused expression to drop slightly relative to the other Coix ubiquitin EXP sequences. Assessment of the resulting spliced messenger RNA showed that approximately 10% of the mRNA expressed using EXP-Cl.Ubq1:1:20 (SEQ ID NO: 102) to drive GUS expression was improperly spliced. The mRNA resulting from GUS expression using the other Coix ubiquitin EXP sequences appeared to process properly. This experiment provides evidence that any of the 3′ end nucleotides for any of the intron variants presented in Table 2 of Example 1 with the exception of the 3′ end sequence 5′-GAC-3′ which is found associated only with the intron element, I-Cl.Ubq1-1:1:9 (SEQ ID NO: 103) should be suitable for use in transgene expression cassettes without significant loss of activity and processing.

### Example 16: Enhancers Derived from the Regulatory Elements

Enhancers are derived from the promoter elements provided herein, such as those presented as SEQ ID NOS: 2, 6, 9, 11, 13, 15, 17, 19, 23, 26, 28, 30, 32, 34, 38, 40, 42, 46, 50, 56, 60, 64, 66, 70, 74, 76, 78, 80, 84, 86, 88, 91, 96 and 135. The enhancer element may be comprised of one or more cis regulatory elements that, when operably linked 5′ or 3′ to a promoter element, or operably linked 5′ or 3′ to additional enhancer elements that are operably linked to a promoter, can enhance or modulate expression of a transgene, or provide expression of a transgene in a specific cell type or plant organ or at a particular time point in development or circadian rhythm. Enhancers are made by removing the TATA box or functionally similar elements and any downstream sequence from the promoters that allow transcription to be initiated from the promoters provided herein as described above, including fragments thereof, in which the TATA box or functionally similar elements and sequence downstream of the TATA box are removed. The enhancer element, E-Cl.Ubq1-1:1:1 (SEQ ID NO: 89) which is derived from the promoter element, P-Cl.Ubq1-1:1:1 is provided herein to demonstrate enhancers derived from a promoter element.

Enhancer elements may be derived from the promoter elements provided herein and cloned using methods known in the art to be operably linked 5′ or 3′ to a promoter element, or operably linked 5′ or 3′ to additional enhancer elements that are operably linked to a promoter. Alternatively, enhancer elements are cloned, using methods known in the art, to be operably linked to one or more copies of the enhancer element which are operably linked 5′ or 3′ to a promoter element, or operably linked 5′ or 3′ to additional enhancer elements that are operably linked to a promoter. Enhancer elements can also be cloned to be operably linked 5′ or 3′ to a promoter element derived from a different genus organism, or operably linked 5′ or 3′ to additional enhancer elements derived from other genus organisms or the same genus organism that are operably linked to a promoter derived from either the same or different genus organism, resulting in a chimeric regulatory element. A GUS expression plant transformation vector is constructed using methods known in the art similar to the constructs described in the previous examples in which the resulting plant expression vectors contain a right border region from A. tumefaciens, a first transgene cassette to test the regulatory or a chimeric regulatory element comprised of, a regulatory or chimeric regulatory element, operably linked to an intron derived from the HSP70 heat shock protein of Z. mays (I-Zm.DnaK-1:1:1 SEQ ID NO: 144) or any of the introns presented herein or any other intron, operably linked to a coding sequence for β-glucuronidase (GUS) that either possesses a processable intron (GUS-2, SEQ ID NO: 160) or no intron (GUS-1, SEQ ID NO: 159), operably linked to the Nopaline synthase 3′ UTR from A. tumefaciens (T-AGRtu.nos-1:1:13, SEQ ID NO: 161) or the 3′ UTR from the rice lipid transfer protein gene (T-Os.LTP-1:1:1, SEQ ID NO: 175); a second transgene selection cassette used for selection of transformed plant cells that confers resistance to the herbicide glyphosate (driven by the rice Actin 1 promoter), or alternatively, the antibiotic kanamycin (driven by the rice Actin 1 promoter) and a left border region from A. tumefaciens. The resulting plasmids are used to transform corn plants or other genus plants by the methods described above or by other Agrobacterium-mediated or particle bombardment methods known in the art. Alternatively, protoplast cells derived from corn or other genus plants are transformed using methods known in the art to perform transient assays

GUS expression driven by the regulatory element comprising one or more enhancers is evaluated in stable or transient plant assays to determine the effects of the enhancer element on expression of a transgene. Modifications to one or more enhancer elements or duplication of one or more enhancer elements is performed based upon empirical experimentation and the resulting gene expression regulation that is observed using each regulatory element composition. Altering the relative positions of one or more enhancers in the resulting regulatory or chimeric regulatory element may affect the transcriptional activity or specificity of the regulatory or chimeric regulatory element and is determined empirically to identify the best enhancers for the desired transgene expression profile within the corn plant or other genus plant.

### Example 17: Analysis of Intron Enhancement of GUS Activity Using Plant Derived Protoplasts

An intron is selected based upon experimentation and comparison with an intronless expression vector control to empirically select an intron and configuration within the vector T-DNA element arrangement for optimal expression of a transgene. For example, in the expression of an herbicide resistance gene, such as CP4 which confers tolerance to glyphosate, it is desirable to have transgene expression within the reproductive tissues as well as the vegetative tissues, to prevent the loss of yield when applying the herbicide. An intron in this instance would be selected upon its ability when operably linked to a constitutive promoter, to enhance expression of the herbicide resistance conferring transgene, particularly within the reproductive cells and tissues of the transgenic plant and thus providing both vegetative and reproductive tolerance to the transgenic plant, when sprayed with the herbicide. In most ubiquitin genes, the 5′ UTR is comprised of a leader, which has an intron sequence embedded within it. The expression elements derived from such genes are therefore assayed using the entire 5′ UTR comprising the promoter, leader, and intron. To achieve different expression profiles or to modulate the level of transgene expression, the intron from such an expression element may be removed or substituted with a heterologous intron.

Introns presented herein as SEQ ID NOS: 4, 7, 21, 24, 36, 44, 48, 52, 54, 58, 62, 68, 72, 82, 92, 94, 101, 103, 105, 107, 109, 111, 113, 118, 120, 122, 127, 129, 131, 138, 140, 142, 144, 146, 148, 150, 152, 154, 156, 158 and 182 are identified using genomic DNA contigs in comparison to expressed sequence tag clusters or cDNA contigs to identify exon and intron sequences within the genomic DNA. In addition, 5′ UTR or leader sequences are also used to define the intron/exon splice junction of one or more introns under conditions when the gene sequence encodes a leader sequence that is interrupted by one or more introns. Introns are cloned using methods known in the art into a plant transformation vector to be operably linked 3′ to a transcriptional regulatory element and leader fragment and operably linked 5′ to either a second leader fragment or to coding sequences, for instance as depicted in the two transgene cassettes presented in FIG. 1.

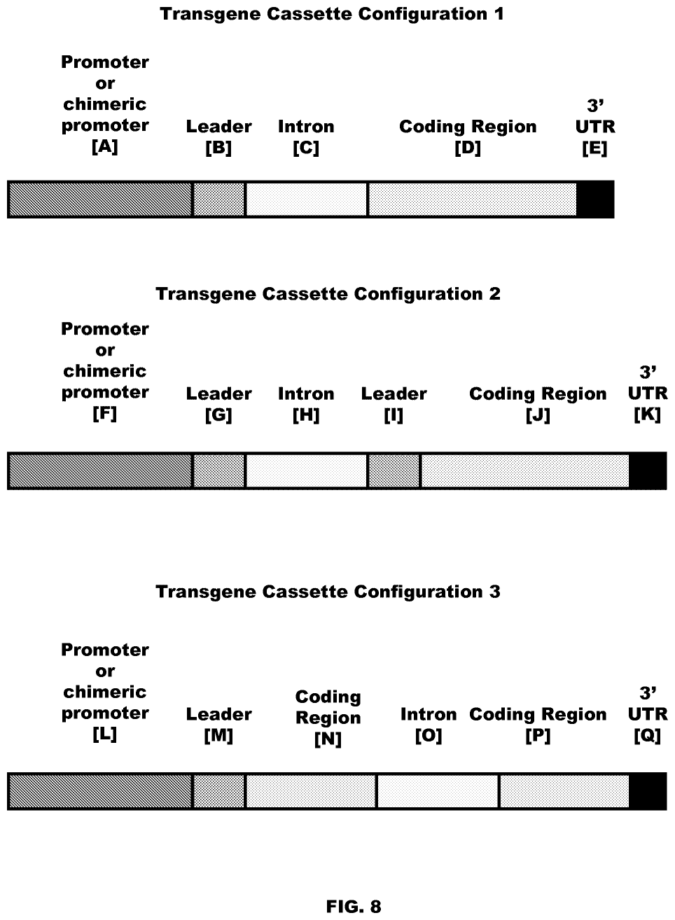

Thus, for instance, a first possible transgene cassette (Transgene Cassette Configuration 1 in FIG. 8) is comprised of a promoter or chimeric promoter element [A], operably linked 5′ to a leader element [B], operably linked 5′ to a test intron element [C], operably linked to a coding region [D], which is operably linked to a 3′ UTR element [E]. Alternatively, a second possible transgene cassette (Transgene Cassette Configuration 2 in FIG. 8) is comprised of a promoter or chimeric promoter element [F], operably linked 5′ to a first leader element or first leader element fragment [G], operably linked 5′ to a test intron element [H], operably linked 5′ to a second leader element or first leader element second fragment [I], operably linked to a coding region [J], which is operably linked to a 3′ UTR element [K]. Further, a third possible transgene cassette (Transgene Cassette Configuration 3 in FIG. 8) is comprised of a promoter or chimeric promoter element [L], operably linked 5′ to a leader element [M], operably linked 5′ to a first fragment of the coding sequence element [N], operably linked 5′ to an intron element [O] element, operably linked 5′ to a second fragment of the coding sequence element [P], which is operably linked to a 3′ UTR element [Q]. Transgene Cassette Configuration 3 is designed to allow splicing of the intron in such a manner as to produce a complete open reading frame without a frame shift between the first and second fragment of the coding sequence.

The first 6 nucleotides on the 5′ end and the last 6 nucleotides on the 3′ end of the introns presented as SEQ ID NOS: 4, 7, 21, 24, 36, 44, 48, 52, 54, 58, 62, 68, 72, 82, 92, 94, 101, 103, 105, 107, 109, 111, 113, 118, 120, 122, 127, 129, 131, 138, 140, 142, 144, 146, 148, 150, 152, 154, 156, 158 and 182 represent nucleotides before and after the intron/exon splice junction, respectively. These short 6 nucleotide sequences, for example, can be modified by having additional sequence appended (i.e. native or artificial) to facilitate cloning of the intron into a plant transformation vector, so long as the first and second nucleotides from the 5′ end (GT) and the fourth and fifth nucleotide from the 3′ end (AG) of SEQ ID NOS: 4, 7, 21, 24, 36, 44, 48, 52, 54, 58, 62, 68, 72, 82, 92, 94, 101, 103, 105, 107, 109, 111, 113, 118, 120, 122, 127, 129, 131, 138, 140, 142, 144, 146, 148, 150, 152, 154, 156, 158 and 182 are preserved, thus preserving the intron/exon splice junction of the intron. As discussed above, it may be preferable to avoid using the nucleotide sequence AT or the nucleotide A just prior to the 5′ end of the splice site (GT) and the nucleotide G or the nucleotide sequence TG, respectively just after 3′ end of the splice site (AG) to eliminate the potential of unwanted start codons from being formed during processing of the messenger RNA into the final transcript. The sequence around the 5′ or 3′ end splice junction sites of the intron can thus be modified.

The introns are assayed for an enhancement effect through the ability to enhance expression in transient assay or stable plant assay. For transient assay of intron enhancement, a base plant vector is constructed using methods known in the art. The intron is cloned into a base plant vector which comprises an expression cassette comprised of a constitutive promoter such as the Cauliflower mosaic virus promoter, P-CaMV.35S-enh-1:1:9 (SEQ ID NO: 176), operably linked 5′ to a leader element, L-CaMV.35S-1:1:15 (SEQ ID NO: 177 (acacgctg)), operably linked 5′ to a test intron element (e.g. one of SEQ ID NOS: 4, 7, 21, 24, 36, 44, 48, 52, 54, 58, 62, 68, 72, 82, 92, 94, 101, 103, 105, 107, 109, 111, 113, 118, 120, 122, 127, 129, 131, 138, 140, 142, 144, 146, 148, 150, 152, 154, 156, 158 and 182), operably linked to a coding sequence for β-glucuronidase (GUS) that either possesses a processable intron (GUS-2, SEQ ID NO: 160) or no intron (GUS-1, SEQ ID NO: 159), operably linked to the Nopaline synthase 3′ UTR from A. tumefaciens (T-AGRtu.nos-1:1:13, SEQ ID NO: 161). Protoplast cells derived from corn or other genus plant tissue are transformed with the base plant vector and luciferase control vectors as described previously in Example 2 above and assayed for activity. To compare the relative ability of the intron to enhance expression, GUS values are expressed as a ratio of GUS to luciferase activity and compared with those levels imparted by a construct comprising the constitutive promoter operably linked to a known intron standard such as that as the intron derived from the HSP70 heat shock protein of Zea mays, I-Zm.DnaK-1:1:1 (SEQ ID NO: 178) as well as a construct comprising the constitutive promoter but without an intron operably linked to the promoter.

For stable plant assay of the introns presented as SEQ ID NOS: 4, 7, 21, 24, 36, 44, 48, 52, 54, 58, 62, 68, 72, 82, 92, 94, 101, 103, 105, 107, 109, 111, 113, 118, 120, 122, 127, 129, 131, 138, 140, 142, 144, 146, 148, 150, 152, 154, 156, 158 and 182, a GUS expression plant transformation vector is constructed similar to the constructs described in the previous examples in which the resulting plant expression vectors contains a right border region from A. tumefaciens, a first transgene cassette to test the intron comprised of a constitutive promoter such as the Cauliflower mosaic virus promoter, P-CaMV.35S-enh-1:1:9 (SEQ ID NO: 176), operably linked 5′ to a leader element, L-CaMV.35S-1:1:15 (SEQ ID NO: 177 (acacgctg)), operably linked 5′ to a test intron element provided herein, operably linked to a coding sequence for β-glucuronidase (GUS) that either possesses a processable intron (GUS-2, SEQ ID NO: 160) or no intron (GUS-1, SEQ ID NO: 158), operably linked to the Nopaline synthase 3′ UTR from A. tumefaciens (T-AGRtu.nos-1:1:13, SEQ ID NO: 161); a second transgene selection cassette used for selection of transformed plant cells that confers resistance to glyphosate (driven by the rice Actin 1 promoter), or alternatively, the antibiotic kanamycin (driven by the rice Actin 1 promoter) and a left border region from A. tumefaciens. The resulting plasmids are used to transform corn plants or other genus plants by the methods described above or by Agrobacterium-mediated methods known in the art. Single-copy or low copy number transformants are selected for comparison to single-copy or low copy number transformed plants, transformed with a plant transformation vector identical to the test vector but without the test intron to determine if the test intron provides an intron mediated enhancement effect.

Any of the introns presented as SEQ ID NOS: 4, 7, 21, 24, 36, 44, 48, 52, 54, 58, 62, 68, 72, 82, 92, 94, 101, 103, 105, 107, 109, 111, 113, 118, 120, 122, 127, 129, 131, 138, 140, 142, 144, 146, 148, 150, 152, 154, 156, 158 and 182 can be modified in a number of ways, such as deleting fragments within the intron sequence, which may reduce expression or duplication of fragments with the intron that may enhance expression. In addition, sequences within the intron that may affect the specificity of expression to either particular cells types or tissues and organs can be duplicated or altered or deleted to affect expression and patterns of expression of the transgene. In addition, the introns provided herein can be modified to remove any potential start codons (ATG) that may cause unintentional transcripts from being expressed from improperly spliced introns as different, longer or truncated proteins. Once the intron has been empirically tested, or it has been altered based upon experimentation, the intron is used to enhance expression of a transgene in stably transformed plants that can be of any genus monocot or dicot plant, so long as the intron provides enhancement of the transgene. The intron can also be used to enhance expression in other organisms, such as algae, fungi or animal cells, so long as the intron provides enhancement or attenuation or specificity of expression of the transgene to which it is operably linked.

Having illustrated and described the principles of the present invention, it should be apparent to persons skilled in the art that the invention can be modified in arrangement and detail without departing from such principles. We claim all modifications that are within the spirit and scope of the claims. All publications and published patent documents cited herein are hereby incorporated by reference to the same extent as if each individual publication or patent application is specifically and individually indicated to be incorporated by reference.

What is claimed is:

1. A DNA molecule comprising a DNA sequence selected from the group consisting of:
a) a sequence with at least 85 percent sequence identity to any of SEQ ID NOs: 1-158 and 180-183;
b) a sequence comprising any of SEQ ID NOs: 1-158 and 180-183; and
c) a fragment of any of SEQ ID NOs: 1-158 and 180-183, wherein the fragment has gene-regulatory activity;
wherein said sequence is operably linked to a heterologous transcribable polynucleotide molecule.

2. The DNA molecule of claim 1, wherein said sequence has at least 90 percent sequence identity to the DNA sequence of any of SEQ ID NOs: 1-158 and 180-183.

3. The DNA molecule of claim 1, wherein said sequence has at least 95 percent sequence identity to the DNA sequence of any of SEQ ID NOs: 1-158 and 180-183.

4. The DNA molecule of claim 1, wherein the DNA sequence comprises gene regulatory activity.

5. The DNA molecule of claim 1, wherein the heterologous transcribable polynucleotide molecule comprises a gene of agronomic interest.

6. The DNA molecule of claim 5, wherein the gene of agronomic interest confers herbicide tolerance in plants.

7. The DNA molecule of claim 5, wherein the gene of agronomic interest confers pest resistance in plants.

8. A transgenic plant cell comprising a heterologous DNA molecule comprising a sequence selected from the group consisting of:
a) a sequence with at least 85 percent sequence identity to any of SEQ ID NOs: 1-158 and 180-183;
b) a sequence comprising any of SEQ ID NOs: 1-158 and 180-183; and
c) a fragment of any of SEQ ID NOs: 1-158 and 180-183, wherein the fragment has gene-regulatory activity;
wherein said sequence is operably linked to a heterologous transcribable polynucleotide molecule.

9. The transgenic plant cell of claim 8, wherein said transgenic plant cell is a monocotyledonous plant cell.

10. The transgenic plant cell of claim 8, wherein said transgenic plant cell is a dicotyledonous plant cell.

11. A transgenic plant, or part thereof, comprising the DNA molecule of claim 1.

12. A progeny plant of the transgenic plant of claim 11, or a part thereof, wherein the progeny plant or part thereof comprises said DNA molecule.

13. A transgenic seed, wherein the seed comprises the DNA molecule of claim 1.

14. A method of producing a commodity product comprising obtaining a transgenic plant or part thereof according to claim 11 and producing the commodity product therefrom.

15. The method of claim 14, wherein the commodity product is protein concentrate, protein isolate, grain, starch, seeds, meal, flour, biomass, or seed oil.

16. A commodity product comprising the DNA molecule of claim 1.

17. The commodity product of claim 16, wherein the commodity product is protein concentrate, protein isolate, grain, starch, seeds, meal, flour, biomass, or seed oil.

18. A method of expressing a transcribable polynucleotide molecule comprising obtaining a transgenic plant according to claim 11 and cultivating plant, wherein the transcribable polynucleotide is expressed.


SEQUENCE LISTING (183 sequences)

SEQ ID NO: 1 - DNA, 3741 bp, Andropogon gerardii
  source 1..3741: mol_type = other DNA
agcagactcgcattatcgatggaggggtgggtttagaaccctgaaaactggtactgtttcgaactgaaaaacactgtagcacttttcgtttgtttgtggtaaatattatcttactatggtctaactaggctcaaaagaatcgtctcgcaatgtacatctaaattatgcaattagttattttgtttacctgcatttcatactccgagcatgcgtcttttggtacatttaatgcttcgatgtgatgggaattttaaaaattttggagaaaagttggtttctaaacacccccgaggacgaaattggattcggtctttgacgcggatgcagcaactgcagtgcgcaggataccatcttagccgttgcgtcgaagttcgctttgctaacgttttgagaaaattaaaccagctttgaccaacgtgagacgagcgccttacgtggcagtgtaatggaaccgggcacggcaagtttgacgctgtagtgttagccggtctcgttacgtttggcacaacttagttgaatccggcttccggcaaactatatggcaagttagacccaagtgtgagccggccaccgcaagttattgggacattatacgtaggaagcaagtgtataataagaatatgagataatgtaagcagctatatgaatcatcacgtcatatttatgttaagatgaagaggatagaataaacggtatgtaaatttatagcgagtgatagacgggcacaaggcctcctagctatttccataaatcggattttgtaagaacaaaaaagaggacttattataagagaatgtggtaagtaagtatactctctccgtttcaaattataagttgttttgatttttttggtacatctattttactatgcattagatataataatgtgtctagatacataacaaaatggatgaatcaaaaaagtcaaagtgatttacaatttggaacggagagagtaagttcaagccgtcaaggcacttctatgcaaccacagtcaacttgaatgccgcttgagtgccttctcaagtttttttttcttgcaaaaatcatttcttttttttaaaaaaagtataatttggatcgtgcaaatttctctctaggtgtgtgtgtgactgtgtgagtaacaatttctctagttgtgcgcgactgctgcttactttggagattacaatatctttctaaaatgcttcgattacttatttataaaccgtctctaaggccaattgctcaagattcattcaacaattgaaacgtctcacatgattaaatcatataaagtttctaagtcttgtttgacaagatttttttagattttcatctaaattggatgaaactatcaaacactaattttaaaaaatataagagaagctccggagataaaaggtcgtctatgttattataagagtaaagtcgtctattctcttcgtcccaacatatataattctaagcatgaattgctttctttttggacaaaaggagcatgccacaacacaagaatgatgtcaccgtcatgcttggatccttttatggtaaagcttcaccttctataatctaacaatagagaaatcagggaaaaatcatgttttggttgtttttatttctaacctccacaataactttggtttaccattttttgtttgattttagttttagagaagcgtttataacaggacctaaaatcttttttcagtacacagtacaacgcagacgctcatacacgcacgcacactcacctctatgaacacacgtaagaaaaccctacaccttgagcaccttcgaaggactgagccggtaaatatagagattctcgaagtcactattagcgcctcgttgtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttccccaacctcatcttctctcgtgttgttcggcacaacccgatcgatccccaactccctcgtcgtctctcctcgcgagcctcgtcgatcccccgcttcaaggtacggcgatcgattatcttccctctctctaccttctctctcttatagggcctgctagctctgttcctgtttttccatggctgcgaggtacaatagatcggcgatccatggttagggcctgctagttgtgttcctgtttttccatggctgcgaggcacaatagatctgatggcgttatgatggttaacttgtcatactcttgcgatctatggtccctttaggagtttaggacatctatttaatttcggatagttcgagatctgtgatccatggttagtaccctaggcagtggggttagatccgtgctgttatggttcgtagatggattctgattgctcagtaactgggaatcctgggatggttctagctggttcgcagataagatcgatttcatgatatgctatatcttgtttggttgccgtggttccgttaaatctgtctgttatgatcttagtctttgataaggttcggtcgtgctagctacgtcctgtgcagcacttaattgtcaggtcataatttttagcatgccttttttttattggtttggttttgtctgactgggctgtagatagtttcaatctttgtctgactgggctgtagatagtttcaatctacctgtcggtttattttattaaatttggatctgtatgtgtgtcatatatcttcatcttttagatatatcgataggtttatatgttgctgtcggttttttactgttcctttatgagatatattcatgcttagatacatgaaacaacgtgctgttacagtttaatagttcttgtttatctaataaacaaataaggataggtatatgctgcagttagttttactggtactttttttgacatgaacctacggcttaataattagtcttcatcaaataaaaagcatattttttaattatttcgatatacttgaatgatgtcatatgcagcatctgtgtgaatttttggccctgtcttcatatgctgtttatttgtttgggactgtttctttggttgataactcatcctgttgtttggtgatccttttgcag

SEQ ID NO: 2 - DNA, 2603 bp, Andropogon gerardii
  source 1..2603: mol_type = other DNA
agcagactcgcattatcgatggaggggtgggtttagaaccctgaaaactggtactgtttcgaactgaaaaacactgtagcacttttcgtttgtttgtggtaaatattatcttactatggtctaactaggctcaaaagaatcgtctcgcaatgtacatctaaattatgcaattagttattttgtttacctgcatttcatactccgagcatgcgtcttttggtacatttaatgcttcgatgtgatgggaattttaaaaattttggagaaaagttggtttctaaacacccccgaggacgaaattggattcggtctttgacgcggatgcagcaactgcagtgcgcaggataccatcttagccgttgcgtcgaagttcgctttgctaacgttttgagaaaattaaaccagctttgaccaacgtgagacgagcgccttacgtggcagtgtaatggaaccgggcacggcaagtttgacgctgtagtgttagccggtctcgttacgtttggcacaacttagttgaatccggcttccggcaaactatatggcaagttagacccaagtgtgagccggccaccgcaagttattgggacattatacgtaggaagcaagtgtataataagaatatgagataatgtaagcagctatatgaatcatcacgtcatatttatgttaagatgaagaggatagaataaacggtatgtaaatttatagcgagtgatagacgggcacaaggcctcctagctatttccataaatcggattttgtaagaacaaaaaagaggacttattataagagaatgtggtaagtaagtatactctctccgtttcaaattataagttgttttgatttttttggtacatctattttactatgcattagatataataatgtgtctagatacataacaaaatggatgaatcaaaaaagtcaaagtgatttacaatttggaacggagagagtaagttcaagccgtcaaggcacttctatgcaaccacagtcaacttgaatgccgcttgagtgccttctcaagtttttttttcttgcaaaaatcatttcttttttttaaaaaaagtataatttggatcgtgcaaatttctctctaggtgtgtgtgtgactgtgtgagtaacaatttctctagttgtgcgcgactgctgcttactttggagattacaatatctttctaaaatgcttcgattacttatttataaaccgtctctaaggccaattgctcaagattcattcaacaattgaaacgtctcacatgattaaatcatataaagtttctaagtcttgtttgacaagatttttttagattttcatctaaattggatgaaactatcaaacactaattttaaaaaatataagagaagctccggagataaaaggtcgtctatgttattataagagtaaagtcgtctattctcttcgtcccaacatatataattctaagcatgaattgctttctttttggacaaaaggagcatgccacaacacaagaatgatgtcaccgtcatgcttggatccttttatggtaaagcttcaccttctataatctaacaatagagaaatcagggaaaaatcatgttttggttgtttttatttctaacctccacaataactttggtttaccattttttgtttgattttagttttagagaagcgtttataacaggacctaaaatcttttttcagtacacagtacaacgcagacgctcatacacgcacgcacactcacctctatgaacacacgtaagaaaaccctacaccttgagcaccttcgaaggactgagccggtaaatatagagattctcgaagtcactattagcgcctcgttgtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttc

SEQ ID NO: 3 - DNA, 99 bp, Andropogon gerardii
  source 1..99: mol_type = other DNA
cccaacctcatcttctctcgtgttgttcggcacaacccgatcgatccccaactccctcgtcgtctctcctcgcgagcctcgtcgatcccccgcttcaag

SEQ ID NO: 4 - DNA, 1039 bp, Andropogon gerardii
  source 1..1039: mol_type = other DNA
gtacggcgatcgattatcttccctctctctaccttctctctcttatagggcctgctagctctgttcctgtttttccatggctgcgaggtacaatagatcggcgatccatggttagggcctgctagttgtgttcctgtttttccatggctgcgaggcacaatagatctgatggcgttatgatggttaacttgtcatactcttgcgatctatggtccctttaggagtttaggacatctatttaatttcggatagttcgagatctgtgatccatggttagtaccctaggcagtggggttagatccgtgctgttatggttcgtagatggattctgattgctcagtaactgggaatcctgggatggttctagctggttcgcagataagatcgatttcatgatatgctatatcttgtttggttgccgtggttccgttaaatctgtctgttatgatcttagtctttgataaggttcggtcgtgctagctacgtcctgtgcagcacttaattgtcaggtcataatttttagcatgccttttttttattggtttggttttgtctgactgggctgtagatagtttcaatctttgtctgactgggctgtagatagtttcaatctacctgtcggtttattttattaaatttggatctgtatgtgtgtcatatatcttcatcttttagatatatcgataggtttatatgttgctgtcggttttttactgttcctttatgagatatattcatgcttagatacatgaaacaacgtgctgttacagtttaatagttcttgtttatctaataaacaaataaggataggtatatgctgcagttagttttactggtactttttttgacatgaacctacggcttaataattagtcttcatcaaataaaaagcatattttttaattatttcgatatacttgaatgatgtcatatgcagcatctgtgtgaatttttggccctgtcttcatatgctgtttatttgtttgggactgtttctttggttgataactcatcctgttgtttggtgatccttttgcag

SEQ ID NO: 5 - DNA, 3255 bp, Andropogon gerardii
  source 1..3255: mol_type = other DNA
ctcgttacgtttggcacaacttagttgaatccggcttccggcaaactatatggcaagttagacccaagtgtgagccggccaccgcaagttattgggacattatacgtaggaagcaagtgtataataagaatatgagataatgtaagcagctatatgaatcatcacgtcatatttatgttaagatgaagaggatagaataaacggtatgtaaatttatagcgagtgatagacgggcacaaggcctcctagctatttccataaatcggattttgtaagaacaaaaaagaggacttattataagagaatgtggtaagtaagtatactctctccgtttcaaattataagttgttttgatttttttggtacatctattttactatgcattagatataataatgtgtctagatacataacaaaatggatgaatcaaaaaagtcaaagtgatttacaatttggaacggagagagtaagttcaagccgtcaaggcacttctatgcaaccacagtcaacttgaatgccgcttgagtgccttctcaagtttttttttcttgcaaaaatcatttcttttttttaaaaaaagtataatttggatcgtgcaaatttctctctaggtgtgtgtgtgactgtgtgagtaacaatttctctagttgtgcgcgactgctgcttactttggagattacaatatctttctaaaatgcttcgattacttatttataaaccgtctctaaggccaattgctcaagattcattcaacaattgaaacgtctcacatgattaaatcatataaagtttctaagtcttgtttgacaagatttttttagattttcatctaaattggatgaaactatcaaacactaattttaaaaaatataagagaagctccggagataaaaggtcgtctatgttattataagagtaaagtcgtctattctcttcgtcccaacatatataattctaagcatgaattgctttctttttggacaaaaggagcatgccacaacacaagaatgatgtcaccgtcatgcttggatccttttatggtaaagcttcaccttctataatctaacaatagagaaatcagggaaaaatcatgttttggttgtttttatttctaacctccacaataactttggtttaccattttttgtttgattttagttttagagaagcgtttataacaggacctaaaatcttttttcagtacacagtacaacgcagacgctcatacacgcacgcacactcacctctatgaacacacgtaagaaaaccctacaccttgagcaccttcgaaggactgagccggtaaatatagagattctcgaagtcactattagcgcctcgttgtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttccccaacctcatcttctctcgtgttgttcggcacaacccgatcgatccccaactccctcgtcgtctctcctcgcgagcctcgtcgatcccccgcttcaaggtacggcgatcgattatcttccctctctctaccttctctctcttatagggcctgctagctctgttcctgtttttccatggctgcgaggtacaatagatcggcgatccatggttagggcctgctagttgtgttcctgtttttccatggctgcgaggcacaatagatctgatggcgttatgatggttaacttgtcatactcttgcgatctatggtccctttaggagtttaggacatctatttaatttcggatagttcgagatctgtgatccatggttagtaccctaggcagtggggttagatccgtgctgttatggttcgtagatggattctgattgctcagtaactgggaatcctgggatggttctagctggttcgcagataagatcgatttcatgatatgctatatcttgtttggttgccgtggttccgttaaatctgtctgttatgatcttagtctttgataaggttcggtcgtgctagctacgtcctgtgcagcacttaattgtcaggtcataatttttagcatgccttttttttattggtttggttttgtctgactgggctgtagatagtttcaatctttgtctgactgggctgtagatagtttcaatctacctgtcggtttattttattaaatttggatctgtatgtgtgtcatatatcttcatcttttagatatatcgataggtttatatgttgctgtcggttttttactgttcctttatgagatatattcatgcttagatacatgaaacaacgtgctgttacagtttaatagttcttgtttatctaataaacaaataaggataggtatatgctgcagttagttttactggtactttttttgacatgaacctacggcttaataattagtcttcatcaaataaaaagcatattttttaattatttcgatatacttgaatgatgtcatatgcagcatctgtgtgaatttttggccctgtcttcatatgctgtttatttgtttgggactgtttctttggttgataactcatcctgttgtttggtgatccttttgcaggtg

SEQ ID NO: 6 - DNA, 2114 bp, Andropogon gerardii
  source 1..2114: mol_type = other DNA
ctcgttacgtttggcacaacttagttgaatccggcttccggcaaactatatggcaagttagacccaagtgtgagccggccaccgcaagttattgggacattatacgtaggaagcaagtgtataataagaatatgagataatgtaagcagctatatgaatcatcacgtcatatttatgttaagatgaagaggatagaataaacggtatgtaaatttatagcgagtgatagacgggcacaaggcctcctagctatttccataaatcggattttgtaagaacaaaaaagaggacttattataagagaatgtggtaagtaagtatactctctccgtttcaaattataagttgttttgatttttttggtacatctattttactatgcattagatataataatgtgtctagatacataacaaaatggatgaatcaaaaaagtcaaagtgatttacaatttggaacggagagagtaagttcaagccgtcaaggcacttctatgcaaccacagtcaacttgaatgccgcttgagtgccttctcaagtttttttttcttgcaaaaatcatttcttttttttaaaaaaagtataatttggatcgtgcaaatttctctctaggtgtgtgtgtgactgtgtgagtaacaatttctctagttgtgcgcgactgctgcttactttggagattacaatatctttctaaaatgcttcgattacttatttataaaccgtctctaaggccaattgctcaagattcattcaacaattgaaacgtctcacatgattaaatcatataaagtttctaagtcttgtttgacaagatttttttagattttcatctaaattggatgaaactatcaaacactaattttaaaaaatataagagaagctccggagataaaaggtcgtctatgttattataagagtaaagtcgtctattctcttcgtcccaacatatataattctaagcatgaattgctttctttttggacaaaaggagcatgccacaacacaagaatgatgtcaccgtcatgcttggatccttttatggtaaagcttcaccttctataatctaacaatagagaaatcagggaaaaatcatgttttggttgtttttatttctaacctccacaataactttggtttaccattttttgtttgattttagttttagagaagcgtttataacaggacctaaaatcttttttcagtacacagtacaacgcagacgctcatacacgcacgcacactcacctctatgaacacacgtaagaaaaccctacaccttgagcaccttcgaaggactgagccggtaaatatagagattctcgaagtcactattagcgcctcgttgtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttc

SEQ ID NO: 7 - DNA, 1042 bp, Andropogon gerardii
  source 1..1042: mol_type = other DNA
gtacggcgatcgattatcttccctctctctaccttctctctcttatagggcctgctagctctgttcctgtttttccatggctgcgaggtacaatagatcggcgatccatggttagggcctgctagttgtgttcctgtttttccatggctgcgaggcacaatagatctgatggcgttatgatggttaacttgtcatactcttgcgatctatggtccctttaggagtttaggacatctatttaatttcggatagttcgagatctgtgatccatggttagtaccctaggcagtggggttagatccgtgctgttatggttcgtagatggattctgattgctcagtaactgggaatcctgggatggttctagctggttcgcagataagatcgatttcatgatatgctatatcttgtttggttgccgtggttccgttaaatctgtctgttatgatcttagtctttgataaggttcggtcgtgctagctacgtcctgtgcagcacttaattgtcaggtcataatttttagcatgccttttttttattggtttggttttgtctgactgggctgtagatagtttcaatctttgtctgactgggctgtagatagtttcaatctacctgtcggtttattttattaaatttggatctgtatgtgtgtcatatatcttcatcttttagatatatcgataggtttatatgttgctgtcggttttttactgttcctttatgagatatattcatgcttagatacatgaaacaacgtgctgttacagtttaatagttcttgtttatctaataaacaaataaggataggtatatgctgcagttagttttactggtactttttttgacatgaacctacggcttaataattagtcttcatcaaataaaaagcatattttttaattatttcgatatacttgaatgatgtcatatgcagcatctgtgtgaatttttggccctgtcttcatatgctgtttatttgtttgggactgtttctttggttgataactcatcctgttgtttggtgatccttttgcaggtg

SEQ ID NO: 8 - DNA, 2785 bp, Andropogon gerardii
  source 1..2785: mol_type = other DNA
gttcaagccgtcaaggcacttctatgcaaccacagtcaacttgaatgccgcttgagtgccttctcaagtttttttttcttgcaaaaatcatttcttttttttaaaaaaagtataatttggatcgtgcaaatttctctctaggtgtgtgtgtgactgtgtgagtaacaatttctctagttgtgcgcgactgctgcttactttggagattacaatatctttctaaaatgcttcgattacttatttataaaccgtctctaaggccaattgctcaagattcattcaacaattgaaacgtctcacatgattaaatcatataaagtttctaagtcttgtttgacaagatttttttagattttcatctaaattggatgaaactatcaaacactaattttaaaaaatataagagaagctccggagataaaaggtcgtctatgttattataagagtaaagtcgtctattctcttcgtcccaacatatataattctaagcatgaattgctttctttttggacaaaaggagcatgccacaacacaagaatgatgtcaccgtcatgcttggatccttttatggtaaagcttcaccttctataatctaacaatagagaaatcagggaaaaatcatgttttggttgtttttatttctaacctccacaataactttggtttaccattttttgtttgattttagttttagagaagcgtttataacaggacctaaaatcttttttcagtacacagtacaacgcagacgctcatacacgcacgcacactcacctctatgaacacacgtaagaaaaccctacaccttgagcaccttcgaaggactgagccggtaaatatagagattctcgaagtcactattagcgcctcgttgtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttccccaacctcatcttctctcgtgttgttcggcacaacccgatcgatccccaactccctcgtcgtctctcctcgcgagcctcgtcgatcccccgcttcaaggtacggcgatcgattatcttccctctctctaccttctctctcttatagggcctgctagctctgttcctgtttttccatggctgcgaggtacaatagatcggcgatccatggttagggcctgctagttgtgttcctgtttttccatggctgcgaggcacaatagatctgatggcgttatgatggttaacttgtcatactcttgcgatctatggtccctttaggagtttaggacatctatttaatttcggatagttcgagatctgtgatccatggttagtaccctaggcagtggggttagatccgtgctgttatggttcgtagatggattctgattgctcagtaactgggaatcctgggatggttctagctggttcgcagataagatcgatttcatgatatgctatatcttgtttggttgccgtggttccgttaaatctgtctgttatgatcttagtctttgataaggttcggtcgtgctagctacgtcctgtgcagcacttaattgtcaggtcataatttttagcatgccttttttttattggtttggttttgtctgactgggctgtagatagtttcaatctttgtctgactgggctgtagatagtttcaatctacctgtcggtttattttattaaatttggatctgtatgtgtgtcatatatcttcatcttttagatatatcgataggtttatatgttgctgtcggttttttactgttcctttatgagatatattcatgcttagatacatgaaacaacgtgctgttacagtttaatagttcttgtttatctaataaacaaataaggataggtatatgctgcagttagttttactggtactttttttgacatgaacctacggcttaataattagtcttcatcaaataaaaagcatattttttaattatttcgatatacttgaatgatgtcatatgcagcatctgtgtgaatttttggccctgtcttcatatgctgtttatttgtttgggactgtttctttggttgataactcatcctgttgtttggtgatccttttgcaggtg

SEQ ID NO: 9 - DNA, 1644 bp, Andropogon gerardii
  source 1..1644: mol_type = other DNA
gttcaagccgtcaaggcacttctatgcaaccacagtcaacttgaatgccgcttgagtgccttctcaagtttttttttcttgcaaaaatcatttcttttttttaaaaaaagtataatttggatcgtgcaaatttctctctaggtgtgtgtgtgactgtgtgagtaacaatttctctagttgtgcgcgactgctgcttactttggagattacaatatctttctaaaatgcttcgattacttatttataaaccgtctctaaggccaattgctcaagattcattcaacaattgaaacgtctcacatgattaaatcatataaagtttctaagtcttgtttgacaagatttttttagattttcatctaaattggatgaaactatcaaacactaattttaaaaaatataagagaagctccggagataaaaggtcgtctatgttattataagagtaaagtcgtctattctcttcgtcccaacatatataattctaagcatgaattgctttctttttggacaaaaggagcatgccacaacacaagaatgatgtcaccgtcatgcttggatccttttatggtaaagcttcaccttctataatctaacaatagagaaatcagggaaaaatcatgttttggttgtttttatttctaacctccacaataactttggtttaccattttttgtttgattttagttttagagaagcgtttataacaggacctaaaatcttttttcagtacacagtacaacgcagacgctcatacacgcacgcacactcacctctatgaacacacgtaagaaaaccctacaccttgagcaccttcgaaggactgagccggtaaatatagagattctcgaagtcactattagcgcctcgttgtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttc

SEQ ID NO: 10 - DNA, 2613 bp, Andropogon gerardii
  source 1..2613: mol_type = other DNA
tctagttgtgcgcgactgctgcttactttggagattacaatatctttctaaaatgcttcgattacttatttataaaccgtctctaaggccaattgctcaagattcattcaacaattgaaacgtctcacatgattaaatcatataaagtttctaagtcttgtttgacaagatttttttagattttcatctaaattggatgaaactatcaaacactaattttaaaaaatataagagaagctccggagataaaaggtcgtctatgttattataagagtaaagtcgtctattctcttcgtcccaacatatataattctaagcatgaattgctttctttttggacaaaaggagcatgccacaacacaagaatgatgtcaccgtcatgcttggatccttttatggtaaagcttcaccttctataatctaacaatagagaaatcagggaaaaatcatgttttggttgtttttatttctaacctccacaataactttggtttaccattttttgtttgattttagttttagagaagcgtttataacaggacctaaaatcttttttcagtacacagtacaacgcagacgctcatacacgcacgcacactcacctctatgaacacacgtaagaaaaccctacaccttgagcaccttcgaaggactgagccggtaaatatagagattctcgaagtcactattagcgcctcgttgtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttccccaacctcatcttctctcgtgttgttcggcacaacccgatcgatccccaactccctcgtcgtctctcctcgcgagcctcgtcgatcccccgcttcaaggtacggcgatcgattatcttccctctctctaccttctctctcttatagggcctgctagctctgttcctgtttttccatggctgcgaggtacaatagatcggcgatccatggttagggcctgctagttgtgttcctgtttttccatggctgcgaggcacaatagatctgatggcgttatgatggttaacttgtcatactcttgcgatctatggtccctttaggagtttaggacatctatttaatttcggatagttcgagatctgtgatccatggttagtaccctaggcagtggggttagatccgtgctgttatggttcgtagatggattctgattgctcagtaactgggaatcctgggatggttctagctggttcgcagataagatcgatttcatgatatgctatatcttgtttggttgccgtggttccgttaaatctgtctgttatgatcttagtctttgataaggttcggtcgtgctagctacgtcctgtgcagcacttaattgtcaggtcataatttttagcatgccttttttttattggtttggttttgtctgactgggctgtagatagtttcaatctttgtctgactgggctgtagatagtttcaatctacctgtcggtttattttattaaatttggatctgtatgtgtgtcatatatcttcatcttttagatatatcgataggtttatatgttgctgtcggttttttactgttcctttatgagatatattcatgcttagatacatgaaacaacgtgctgttacagtttaatagttcttgtttatctaataaacaaataaggataggtatatgctgcagttagttttactggtactttttttgacatgaacctacggcttaataattagtcttcatcaaataaaaagcatattttttaattatttcgatatacttgaatgatgtcatatgcagcatctgtgtgaatttttggccctgtcttcatatgctgtttatttgtttgggactgtttctttggttgataactcatcctgttgtttggtgatccttttgcaggtg

SEQ ID NO: 11 - DNA, 1472 bp, Andropogon gerardii
  source 1..1472: mol_type = other DNA
tctagttgtgcgcgactgctgcttactttggagattacaatatctttctaaaatgcttcgattacttatttataaaccgtctctaaggccaattgctcaagattcattcaacaattgaaacgtctcacatgattaaatcatataaagtttctaagtcttgtttgacaagatttttttagattttcatctaaattggatgaaactatcaaacactaattttaaaaaatataagagaagctccggagataaaaggtcgtctatgttattataagagtaaagtcgtctattctcttcgtcccaacatatataattctaagcatgaattgctttctttttggacaaaaggagcatgccacaacacaagaatgatgtcaccgtcatgcttggatccttttatggtaaagcttcaccttctataatctaacaatagagaaatcagggaaaaatcatgttttggttgtttttatttctaacctccacaataactttggtttaccattttttgtttgattttagttttagagaagcgtttataacaggacctaaaatcttttttcagtacacagtacaacgcagacgctcatacacgcacgcacactcacctctatgaacacacgtaagaaaaccctacaccttgagcaccttcgaaggactgagccggtaaatatagagattctcgaagtcactattagcgcctcgttgtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttc

SEQ ID NO: 12 - DNA, 2255 bp, Andropogon gerardii
  source 1..2255: mol_type = other DNA
cacaagaatgatgtcaccgtcatgcttggatccttttatggtaaagcttcaccttctataatctaacaatagagaaatcagggaaaaatcatgttttggttgtttttatttctaacctccacaataactttggtttaccattttttgtttgattttagttttagagaagcgtttataacaggacctaaaatcttttttcagtacacagtacaacgcagacgctcatacacgcacgcacactcacctctatgaacacacgtaagaaaaccctacaccttgagcaccttcgaaggactgagccggtaaatatagagattctcgaagtcactattagcgcctcgttgtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttccccaacctcatcttctctcgtgttgttcggcacaacccgatcgatccccaactccctcgtcgtctctcctcgcgagcctcgtcgatcccccgcttcaaggtacggcgatcgattatcttccctctctctaccttctctctcttatagggcctgctagctctgttcctgtttttccatggctgcgaggtacaatagatcggcgatccatggttagggcctgctagttgtgttcctgtttttccatggctgcgaggcacaatagatctgatggcgttatgatggttaacttgtcatactcttgcgatctatggtccctttaggagtttaggacatctatttaatttcggatagttcgagatctgtgatccatggttagtaccctaggcagtggggttagatccgtgctgttatggttcgtagatggattctgattgctcagtaactgggaatcctgggatggttctagctggttcgcagataagatcgatttcatgatatgctatatcttgtttggttgccgtggttccgttaaatctgtctgttatgatcttagtctttgataaggttcggtcgtgctagctacgtcctgtgcagcacttaattgtcaggtcataatttttagcatgccttttttttattggtttggttttgtctgactgggctgtagatagtttcaatctttgtctgactgggctgtagatagtttcaatctacctgtcggtttattttattaaatttggatctgtatgtgtgtcatatatcttcatcttttagatatatcgataggtttatatgttgctgtcggttttttactgttcctttatgagatatattcatgcttagatacatgaaacaacgtgctgttacagtttaatagttcttgtttatctaataaacaaataaggataggtatatgctgcagttagttttactggtactttttttgacatgaacctacggcttaataattagtcttcatcaaataaaaagcatattttttaattatttcgatatacttgaatgatgtcatatgcagcatctgtgtgaatttttggccctgtcttcatatgctgtttatttgtttgggactgtttctttggttgataactcatcctgttgtttggtgatccttttgcaggtg

SEQ ID NO: 13 - DNA, 1114 bp, Andropogon gerardii
  source 1..1114: mol_type = other DNA
cacaagaatgatgtcaccgtcatgcttggatccttttatggtaaagcttcaccttctataatctaacaatagagaaatcagggaaaaatcatgttttggttgtttttatttctaacctccacaataactttggtttaccattttttgtttgattttagttttagagaagcgtttataacaggacctaaaatcttttttcagtacacagtacaacgcagacgctcatacacgcacgcacactcacctctatgaacacacgtaagaaaaccctacaccttgagcaccttcgaaggactgagccggtaaatatagagattctcgaagtcactattagcgcctcgttgtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttc

SEQ ID NO: 14 - DNA, 1912 bp, Andropogon gerardii
  source 1..1912: mol_type = other DNA
gtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttccccaacctcatcttctctcgtgttgttcggcacaacccgatcgatccccaactccctcgtcgtctctcctcgcgagcctcgtcgatcccccgcttcaaggtacggcgatcgattatcttccctctctctaccttctctctcttatagggcctgctagctctgttcctgtttttccatggctgcgaggtacaatagatcggcgatccatggttagggcctgctagttgtgttcctgtttttccatggctgcgaggcacaatagatctgatggcgttatgatggttaacttgtcatactcttgcgatctatggtccctttaggagtttaggacatctatttaatttcggatagttcgagatctgtgatccatggttagtaccctaggcagtggggttagatccgtgctgttatggttcgtagatggattctgattgctcagtaactgggaatcctgggatggttctagctggttcgcagataagatcgatttcatgatatgctatatcttgtttggttgccgtggttccgttaaatctgtctgttatgatcttagtctttgataaggttcggtcgtgctagctacgtcctgtgcagcacttaattgtcaggtcataatttttagcatgccttttttttattggtttggttttgtctgactgggctgtagatagtttcaatctttgtctgactgggctgtagatagtttcaatctacctgtcggtttattttattaaatttggatctgtatgtgtgtcatatatcttcatcttttagatatatcgataggtttatatgttgctgtcggttttttactgttcctttatgagatatattcatgcttagatacatgaaacaacgtgctgttacagtttaatagttcttgtttatctaataaacaaataaggataggtatatgctgcagttagttttactggtactttttttgacatgaacctacggcttaataattagtcttcatcaaataaaaagcatattttttaattatttcgatatacttgaatgatgtcatatgcagcatctgtgtgaatttttggccctgtcttcatatgctgtttatttgtttgggactgtttctttggttgataactcatcctgttgtttggtgatccttttgcaggtg

SEQ ID NO: 15 - DNA, 771 bp, Andropogon gerardii
  source 1..771: mol_type = other DNA
gtcaacgggaatgtcgcttaccacttaaagcataacgccgagaaatcccgtaataaatccagtaaaatacgagcacccgtgccaagttgaatatttgaacccgagtgggtagattccaccgcaaaggacctaaccagatcatttcgcaaacaggaactaaaatcggtagagagcccagacaaaagcctttcctaagagccactccagtggaagcccctactttaggtataaaatgcaatactagtggggctcctaaataaacttctatttttcatggccttctaaaattcactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttc

SEQ ID NO: 16 - DNA, 1623 bp, Andropogon gerardii
  source 1..1623: mol_type = other DNA
cactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttccccaacctcatcttctctcgtgttgttcggcacaacccgatcgatccccaactccctcgtcgtctctcctcgcgagcctcgtcgatcccccgcttcaaggtacggcgatcgattatcttccctctctctaccttctctctcttatagggcctgctagctctgttcctgtttttccatggctgcgaggtacaatagatcggcgatccatggttagggcctgctagttgtgttcctgtttttccatggctgcgaggcacaatagatctgatggcgttatgatggttaacttgtcatactcttgcgatctatggtccctttaggagtttaggacatctatttaatttcggatagttcgagatctgtgatccatggttagtaccctaggcagtggggttagatccgtgctgttatggttcgtagatggattctgattgctcagtaactgggaatcctgggatggttctagctggttcgcagataagatcgatttcatgatatgctatatcttgtttggttgccgtggttccgttaaatctgtctgttatgatcttagtctttgataaggttcggtcgtgctagctacgtcctgtgcagcacttaattgtcaggtcataatttttagcatgccttttttttattggtttggttttgtctgactgggctgtagatagtttcaatctttgtctgactgggctgtagatagtttcaatctacctgtcggtttattttattaaatttggatctgtatgtgtgtcatatatcttcatcttttagatatatcgataggtttatatgttgctgtcggttttttactgttcctttatgagatatattcatgcttagatacatgaaacaacgtgctgttacagtttaatagttcttgtttatctaataaacaaataaggataggtatatgctgcagttagttttactggtactttttttgacatgaacctacggcttaataattagtcttcatcaaataaaaagcatattttttaattatttcgatatacttgaatgatgtcatatgcagcatctgtgtgaatttttggccctgtcttcatatgctgtttatttgtttgggactgtttctttggttgataactcatcctgttgtttggtgatccttttgcaggtg

SEQ ID NO: 17 - DNA, 482 bp, Andropogon gerardii
  source 1..482: mol_type = other DNA
cactcccaaacccctagctatagaagtctcttatccatcctctaaataaaaatgggagtctattttatttcaccagagttgatcgtaaatttagtctctcaaattttataagttgagggtagaggatgactggagttgctctaaacggacctatcttcaagtgacctcagtgagcccgtttaacggcgtcgacaagtttaatctaacggacaccaaccagagaagagaaccaccgccagcgccgagccaagcgacgttgacatcttggcgcggcacggcatctccctggcgtctggccccctctcgagacttccgctccacctcccaccggtggcggtttccaagtccgttccgcctcctctcacacggcacgaaaccgtgacgggcaccggcagcacggggggattcctttcccaccgctccttccctttcccttcctctcccgccgctataaatagccagccccatccccagcttctttc

SEQ ID NO: 18 - DNA, 3483 bp, Tripidium ravennae
  source 1..3483: mol_type = other DNA; sub_species = ravennae
gtggccagcttttgttctagttcaacggccccggccttccgggcacctaataccctaattaatctattgcagctaacctcaaaagaaatgcatttgcagttgtctgtcccaatcaatctactagcagacttacattatagatggaggaaattaaattcagcctttgacgtggatgcaacaactgcactgcacaggataccatcttagccgttgtgtcaaagtttgctttgctaaacgttttgagaaaaccagctttgaccaacgcgagatgagcgccttacgtttggcacaatgtaatgtaatccggcacggcaagttagactctgtagtgttagccggcctctttacgtttggcatagtttaattgaatccggcatggcaagttagaccgtagtgtgagccggccaacgcaagttattatgacatatgtataagagcaagtgtattgtcacgtgatatttatgttgagatgaagaagagaaaataaacagcctgcaaatttatagcgagtgatagatgggcacaaggcttcctatttcttaaatcagactttgtaagaacaaaaaaaggacttataagagaatgggataaaccatatatcaatggtgtagtatgttagtatgcattaagatctgactattatatgagtgagttgttaaattcattttaggtgacatggcccggttaaattattagccataccctaacagctctaaaaaagatatattcgttgaggcacttttatgcaaccacatagtcaacttgaatgccgcttgagtgcgttctcaagttttttttcttgcaaattacgcttttttaagaaagtataatttggatcgtgcgattttttttctctaggtgtgcgtgactgtgtgagtaacaattttggatctcagaaaggtaataaaagaataatactgctgcctactttgaggattacaatatctttctctaaaatgttttggtttgttatttaaaccgtctttaaggccaattgctcaagattcattcaacaattgaaacgtctcacatgattaaatcatataaggttgctaaggtcttgtttgacaaggttttttttgtggaaatttcatctaaatttttgagtgaaactatcaaatactaatttaaaaaaggcaaattttgctggaggacactgcagaaacgtgtaattggccggcacaaaccgccaaacggagaatttgcccagtaccattataaattcatgataaattcatggttgtttgccagtggggctagggttcctcgcgtatggtgcggaatgtggtttggttcgaccaactcgaactcaatccgatccaaaggggcatcaatagtcattttagaaagtttctctctcccgagcagtggaaatgattattctatttggcgcgatgtccaccggcaaacaaccacgaatttgtaatggtactaggcaaattctccgtttggcggtgtgtgccggccaattacacgtttttgcggtgtcctccgacaaaatttgccttttaaaaacaattttataagagaagctccggagataaaaggccgtcaatgttacaagagtgaagtcgtctactccctccatcccaaaaaatgtaattctaagtatgagttgtattattatttttggacaaaaggagtataccacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctccccgtacggcgatcatcctccctttctctaccttctcttctctagactaggtcggcgatccatggttagggcctgctagttctgttcctgtttttccgtggctgcgaggtacaatagatctgatggcgttatgatggttaacttgtcatactcctgcggtgtgcggtctatagtgcttttaggacatcaatttgacctggctcgttcgagatcggcgatccatggttaggaccctaggcggtggagtcgggttagatccgcgctgtttgtgttagtagatggatgcgacctttacttcagacacgttctgattgttaacttgtcagcacctgggagtcctgggatggttctagctggttcgcagatgagatcgatttcatgatctgctgtatcttgtttcgttaggttccttttaatctatccgtggtattatgctaacctatgatatggttcgatcgtgctagctacgtcctgtgtcataatttttagcatgcccttttttgtttggttttgtctgattgggctgtagatcagagtatactgtttcaaactacctactggatatatttattaaatttgaatctgtatgtgtgtcacatatatcttcataattaaaatggatggaaagatatatggataggtacatgtgttgctgtgggttttactggtactttgttagatatacatgcttagatacatgaagcaacatgatgttacagttcaataattcttgtttacctaataaacaaataaggataggtgtatgttgctgtgggttttgctggtactttgttagatatatatgcttagatatatgaagcaacatcctgctacggtttaataattattgtttatatctaatagacaagcctgctttttaattattttgatatacttggatgatggcatacagcagctatgtgtggatttttaaatacccagcatcatgagcatgcatgaccctgccttagtatgctgtttatttgcttgagacttctttttttgttggtactcaccttttgtagtttggtgactcttctgcag

SEQ ID NO: 19 - DNA, 2536 bp, Tripidium ravennae
  source 1..2536: mol_type = other DNA; sub_species = ravennae
gtggccagcttttgttctagttcaacggccccggccttccgggcacctaataccctaattaatctattgcagctaacctcaaaagaaatgcatttgcagttgtctgtcccaatcaatctactagcagacttacattatagatggaggaaattaaattcagcctttgacgtggatgcaacaactgcactgcacaggataccatcttagccgttgtgtcaaagtttgctttgctaaacgttttgagaaaaccagctttgaccaacgcgagatgagcgccttacgtttggcacaatgtaatgtaatccggcacggcaagttagactctgtagtgttagccggcctctttacgtttggcatagtttaattgaatccggcatggcaagttagaccgtagtgtgagccggccaacgcaagttattatgacatatgtataagagcaagtgtattgtcacgtgatatttatgttgagatgaagaagagaaaataaacagcctgcaaatttatagcgagtgatagatgggcacaaggcttcctatttcttaaatcagactttgtaagaacaaaaaaaggacttataagagaatgggataaaccatatatcaatggtgtagtatgttagtatgcattaagatctgactattatatgagtgagttgttaaattcattttaggtgacatggcccggttaaattattagccataccctaacagctctaaaaaagatatattcgttgaggcacttttatgcaaccacatagtcaacttgaatgccgcttgagtgcgttctcaagttttttttcttgcaaattacgcttttttaagaaagtataatttggatcgtgcgattttttttctctaggtgtgcgtgactgtgtgagtaacaattttggatctcagaaaggtaataaaagaataatactgctgcctactttgaggattacaatatctttctctaaaatgttttggtttgttatttaaaccgtctttaaggccaattgctcaagattcattcaacaattgaaacgtctcacatgattaaatcatataaggttgctaaggtcttgtttgacaaggttttttttgtggaaatttcatctaaatttttgagtgaaactatcaaatactaatttaaaaaaggcaaattttgctggaggacactgcagaaacgtgtaattggccggcacaaaccgccaaacggagaatttgcccagtaccattataaattcatgataaattcatggttgtttgccagtggggctagggttcctcgcgtatggtgcggaatgtggtttggttcgaccaactcgaactcaatccgatccaaaggggcatcaatagtcattttagaaagtttctctctcccgagcagtggaaatgattattctatttggcgcgatgtccaccggcaaacaaccacgaatttgtaatggtactaggcaaattctccgtttggcggtgtgtgccggccaattacacgtttttgcggtgtcctccgacaaaatttgccttttaaaaacaattttataagagaagctccggagataaaaggccgtcaatgttacaagagtgaagtcgtctactccctccatcccaaaaaatgtaattctaagtatgagttgtattattatttttggacaaaaggagtataccacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctccccaacctcagcttctctcgttgttcggagcgcacacacaacccgatccccaatcccctcgtctctcctcgcgagcctcgtcgatccccgcttcaag

SEQ ID NO: 20 - DNA, 94 bp, Tripidium ravennae
  source 1..94: mol_type = other DNA; sub_species = ravennae
aacctcagcttctctcgttgttcggagcgcacacacaacccgatccccaatcccctcgtctctcctcgcgagcctcgtcgatccccgcttcaag

SEQ ID NO: 21 - DNA, 1041 bp, Tripidium ravennae
  source 1..1041: mol_type = other DNA; sub_species = ravennae
gtacggcgatcatcctccctttctctaccttctcttctctagactaggtcggcgatccatggttagggcctgctagttctgttcctgtttttccgtggctgcgaggtacaatagatctgatggcgttatgatggttaacttgtcatactcctgcggtgtgcggtctatagtgcttttaggacatcaatttgacctggctcgttcgagatcggcgatccatggttaggaccctaggcggtggagtcgggttagatccgcgctgtttgtgttagtagatggatgcgacctttacttcagacacgttctgattgttaacttgtcagcacctgggagtcctgggatggttctagctggttcgcagatgagatcgatttcatgatctgctgtatcttgtttcgttaggttccttttaatctatccgtggtattatgctaacctatgatatggttcgatcgtgctagctacgtcctgtgtcataatttttagcatgcccttttttgtttggttttgtctgattgggctgtagatcagagtatactgtttcaaactacctactggatatatttattaaatttgaatctgtatgtgtgtcacatatatcttcataattaaaatggatggaaagatatatggataggtacatgtgttgctgtgggttttactggtactttgttagatatacatgcttagatacatgaagcaacatgatgttacagttcaataattcttgtttacctaataaacaaataaggataggtgtatgttgctgtgggttttgctggtactttgttagatatatatgcttagatatatgaagcaacatcctgctacggtttaataattattgtttatatctaatagacaagcctgctttttaattattttgatatacttggatgatggcatacagcagctatgtgtggatttttaaatacccagcatcatgagcatgcatgaccctgccttagtatgctgtttatttgcttgagacttctttttttgttggtactcaccttttgtagtttggtgactcttctgcag

SEQ ID NO: 22 - DNA, 3152 bp, Tripidium ravennae
  source 1..3152: mol_type = other DNA; sub_species = ravennae
gtataagagcaagtgtattgtcacgtgatatttatgttgagatgaagaagagaaaataaacagcctgcaaatttatagcgagtgatagatgggcacaaggcttcctatttcttaaatcagactttgtaagaacaaaaaaaggacttataagagaatgggataaaccatatatcaatggtgtagtatgttagtatgcattaagatctgactattatatgagtgagttgttaaattcattttaggtgacatggcccggttaaattattagccataccctaacagctctaaaaaagatatattcgttgaggcacttttatgcaaccacatagtcaacttgaatgccgcttgagtgcgttctcaagttttttttcttgcaaattacgcttttttaagaaagtataatttggatcgtgcgattttttttctctaggtgtgcgtgactgtgtgagtaacaattttggatctcagaaaggtaataaaagaataatactgctgcctactttgaggattacaatatctttctctaaaatgttttggtttgttatttaaaccgtctttaaggccaattgctcaagattcattcaacaattgaaacgtctcacatgattaaatcatataaggttgctaaggtcttgtttgacaaggttttttttgtggaaatttcatctaaatttttgagtgaaactatcaaatactaatttaaaaaaggcaaattttgctggaggacactgcagaaacgtgtaattggccggcacaaaccgccaaacggagaatttgcccagtaccattataaattcatgataaattcatggttgtttgccagtggggctagggttcctcgcgtatggtgcggaatgtggtttggttcgaccaactcgaactcaatccgatccaaaggggcatcaatagtcattttagaaagtttctctctcccgagcagtggaaatgattattctatttggcgcgatgtccaccggcaaacaaccacgaatttgtaatggtactaggcaaattctccgtttggcggtgtgtgccggccaattacacgtttttgcggtgtcctccgacaaaatttgccttttaaaaacaattttataagagaagctccggagataaaaggccgtcaatgttacaagagtgaagtcgtctactccctccatcccaaaaaatgtaattctaagtatgagttgtattattatttttggacaaaaggagtataccacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctccccaacctcagcttctctcgttgttcggagcgcacacacaacccgatccccaatcccctcgtctctcctcgcgagcctcgtcgatccccgcttcaaggtacggcgatcatcctccctttctctaccttctcttctctagactaggtcggcgatccatggttagggcctgctagttctgttcctgtttttccgtggctgcgaggtacaatagatctgatggcgttatgatggttaacttgtcatactcctgcggtgtgcggtctatagtgcttttaggacatcaatttgacctggctcgttcgagatcggcgatccatggttaggaccctaggcggtggagtcgggttagatccgcgctgtttgtgttagtagatggatgcgacctttacttcagacacgttctgattgttaacttgtcagcacctgggagtcctgggatggttctagctggttcgcagatgagatcgatttcatgatctgctgtatcttgtttcgttaggttccttttaatctatccgtggtattatgctaacctatgatatggttcgatcgtgctagctacgtcctgtgtcataatttttagcatgcccttttttgtttggttttgtctgattgggctgtagatcagagtatactgtttcaaactacctactggatatatttattaaatttgaatctgtatgtgtgtcacatatatcttcataattaaaatggatggaaagatatatggataggtacatgtgttgctgtgggttttactggtactttgttagatatacatgcttagatacatgaagcaacatgatgttacagttcaataattcttgtttacctaataaacaaataaggataggtgtatgttgctgtgggttttgctggtactttgttagatatatatgcttagatatatgaagcaacatcctgctacggtttaataattattgtttatatctaatagacaagcctgctttttaattattttgatatacttggatgatggcatacagcagctatgtgtggatttttaaatacccagcatcatgagcatgcatgaccctgccttagtatgctgtttatttgcttgagacttctttttttgttggtactcaccttttgtagtttggtgactcttctgcaggtg

SEQ ID NO: 23 - DNA, 2014 bp, Tripidium ravennae
  source 1..2014: mol_type = other DNA; sub_species = ravennae
gtataagagcaagtgtattgtcacgtgatatttatgttgagatgaagaagagaaaataaacagcctgcaaatttatagcgagtgatagatgggcacaaggcttcctatttcttaaatcagactttgtaagaacaaaaaaaggacttataagagaatgggataaaccatatatcaatggtgtagtatgttagtatgcattaagatctgactattatatgagtgagttgttaaattcattttaggtgacatggcccggttaaattattagccataccctaacagctctaaaaaagatatattcgttgaggcacttttatgcaaccacatagtcaacttgaatgccgcttgagtgcgttctcaagttttttttcttgcaaattacgcttttttaagaaagtataatttggatcgtgcgattttttttctctaggtgtgcgtgactgtgtgagtaacaattttggatctcagaaaggtaataaaagaataatactgctgcctactttgaggattacaatatctttctctaaaatgttttggtttgttatttaaaccgtctttaaggccaattgctcaagattcattcaacaattgaaacgtctcacatgattaaatcatataaggttgctaaggtcttgtttgacaaggttttttttgtggaaatttcatctaaatttttgagtgaaactatcaaatactaatttaaaaaaggcaaattttgctggaggacactgcagaaacgtgtaattggccggcacaaaccgccaaacggagaatttgcccagtaccattataaattcatgataaattcatggttgtttgccagtggggctagggttcctcgcgtatggtgcggaatgtggtttggttcgaccaactcgaactcaatccgatccaaaggggcatcaatagtcattttagaaagtttctctctcccgagcagtggaaatgattattctatttggcgcgatgtccaccggcaaacaaccacgaatttgtaatggtactaggcaaattctccgtttggcggtgtgtgccggccaattacacgtttttgcggtgtcctccgacaaaatttgccttttaaaaacaattttataagagaagctccggagataaaaggccgtcaatgttacaagagtgaagtcgtctactccctccatcccaaaaaatgtaattctaagtatgagttgtattattatttttggacaaaaggagtataccacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctcccc

SEQ ID NO: 24 - DNA, 1044 bp, Tripidium ravennae
  source 1..1044: mol_type = other DNA; sub_species = ravennae
gtacggcgatcatcctccctttctctaccttctcttctctagactaggtcggcgatccatggttagggcctgctagttctgttcctgtttttccgtggctgcgaggtacaatagatctgatggcgttatgatggttaacttgtcatactcctgcggtgtgcggtctatagtgcttttaggacatcaatttgacctggctcgttcgagatcggcgatccatggttaggaccctaggcggtggagtcgggttagatccgcgctgtttgtgttagtagatggatgcgacctttacttcagacacgttctgattgttaacttgtcagcacctgggagtcctgggatggttctagctggttcgcagatgagatcgatttcatgatctgctgtatcttgtttcgttaggttccttttaatctatccgtggtattatgctaacctatgatatggttcgatcgtgctagctacgtcctgtgtcataatttttagcatgcccttttttgtttggttttgtctgattgggctgtagatcagagtatactgtttcaaactacctactggatatatttattaaatttgaatctgtatgtgtgtcacatatatcttcataattaaaatggatggaaagatatatggataggtacatgtgttgctgtgggttttactggtactttgttagatatacatgcttagatacatgaagcaacatgatgttacagttcaataattcttgtttacctaataaacaaataaggataggtgtatgttgctgtgggttttgctggtactttgttagatatatatgcttagatatatgaagcaacatcctgctacggtttaataattattgtttatatctaatagacaagcctgctttttaattattttgatatacttggatgatggcatacagcagctatgtgtggatttttaaatacccagcatcatgagcatgcatgaccctgccttagtatgctgtttatttgcttgagacttctttttttgttggtactcaccttttgtagtttggtgactcttctgcaggtg

SEQ ID NO: 25 - DNA, 2663 bp, Tripidium ravennae
  source 1..2663: mol_type = other DNA; sub_species = ravennae
ctgctgcctactttgaggattacaatatctttctctaaaatgttttggtttgttatttaaaccgtctttaaggccaattgctcaagattcattcaacaattgaaacgtctcacatgattaaatcatataaggttgctaaggtcttgtttgacaaggttttttttgtggaaatttcatctaaatttttgagtgaaactatcaaatactaatttaaaaaaggcaaattttgctggaggacactgcagaaacgtgtaattggccggcacaaaccgccaaacggagaatttgcccagtaccattataaattcatgataaattcatggttgtttgccagtggggctagggttcctcgcgtatggtgcggaatgtggtttggttcgaccaactcgaactcaatccgatccaaaggggcatcaatagtcattttagaaagtttctctctcccgagcagtggaaatgattattctatttggcgcgatgtccaccggcaaacaaccacgaatttgtaatggtactaggcaaattctccgtttggcggtgtgtgccggccaattacacgtttttgcggtgtcctccgacaaaatttgccttttaaaaacaattttataagagaagctccggagataaaaggccgtcaatgttacaagagtgaagtcgtctactccctccatcccaaaaaatgtaattctaagtatgagttgtattattatttttggacaaaaggagtataccacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctccccaacctcagcttctctcgttgttcggagcgcacacacaacccgatccccaatcccctcgtctctcctcgcgagcctcgtcgatccccgcttcaaggtacggcgatcatcctccctttctctaccttctcttctctagactaggtcggcgatccatggttagggcctgctagttctgttcctgtttttccgtggctgcgaggtacaatagatctgatggcgttatgatggttaacttgtcatactcctgcggtgtgcggtctatagtgcttttaggacatcaatttgacctggctcgttcgagatcggcgatccatggttaggaccctaggcggtggagtcgggttagatccgcgctgtttgtgttagtagatggatgcgacctttacttcagacacgttctgattgttaacttgtcagcacctgggagtcctgggatggttctagctggttcgcagatgagatcgatttcatgatctgctgtatcttgtttcgttaggttccttttaatctatccgtggtattatgctaacctatgatatggttcgatcgtgctagctacgtcctgtgtcataatttttagcatgcccttttttgtttggttttgtctgattgggctgtagatcagagtatactgtttcaaactacctactggatatatttattaaatttgaatctgtatgtgtgtcacatatatcttcataattaaaatggatggaaagatatatggataggtacatgtgttgctgtgggttttactggtactttgttagatatacatgcttagatacatgaagcaacatgatgttacagttcaataattcttgtttacctaataaacaaataaggataggtgtatgttgctgtgggttttgctggtactttgttagatatatatgcttagatatatgaagcaacatcctgctacggtttaataattattgtttatatctaatagacaagcctgctttttaattattttgatatacttggatgatggcatacagcagctatgtgtggatttttaaatacccagcatcatgagcatgcatgaccctgccttagtatgctgtttatttgcttgagacttctttttttgttggtactcaccttttgtagtttggtgactcttctgcaggtg

SEQ ID NO: 26 - DNA, 1525 bp, Tripidium ravennae
  source 1..1525: mol_type = other DNA; sub_species = ravennae
ctgctgcctactttgaggattacaatatctttctctaaaatgttttggtttgttatttaaaccgtctttaaggccaattgctcaagattcattcaacaattgaaacgtctcacatgattaaatcatataaggttgctaaggtcttgtttgacaaggttttttttgtggaaatttcatctaaatttttgagtgaaactatcaaatactaatttaaaaaaggcaaattttgctggaggacactgcagaaacgtgtaattggccggcacaaaccgccaaacggagaatttgcccagtaccattataaattcatgataaattcatggttgtttgccagtggggctagggttcctcgcgtatggtgcggaatgtggtttggttcgaccaactcgaactcaatccgatccaaaggggcatcaatagtcattttagaaagtttctctctcccgagcagtggaaatgattattctatttggcgcgatgtccaccggcaaacaaccacgaatttgtaatggtactaggcaaattctccgtttggcggtgtgtgccggccaattacacgtttttgcggtgtcctccgacaaaatttgccttttaaaaacaattttataagagaagctccggagataaaaggccgtcaatgttacaagagtgaagtcgtctactccctccatcccaaaaaatgtaattctaagtatgagttgtattattatttttggacaaaaggagtataccacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctcccc

SEQ ID NO: 27 - DNA, 2182 bp, Tripidium ravennae
  source 1..2182: mol_type = other DNA; sub_species = ravennae
ccaccggcaaacaaccacgaatttgtaatggtactaggcaaattctccgtttggcggtgtgtgccggccaattacacgtttttgcggtgtcctccgacaaaatttgccttttaaaaacaattttataagagaagctccggagataaaaggccgtcaatgttacaagagtgaagtcgtctactccctccatcccaaaaaatgtaattctaagtatgagttgtattattatttttggacaaaaggagtataccacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctccccaacctcagcttctctcgttgttcggagcgcacacacaacccgatccccaatcccctcgtctctcctcgcgagcctcgtcgatccccgcttcaaggtacggcgatcatcctccctttctctaccttctcttctctagactaggtcggcgatccatggttagggcctgctagttctgttcctgtttttccgtggctgcgaggtacaatagatctgatggcgttatgatggttaacttgtcatactcctgcggtgtgcggtctatagtgcttttaggacatcaatttgacctggctcgttcgagatcggcgatccatggttaggaccctaggcggtggagtcgggttagatccgcgctgtttgtgttagtagatggatgcgacctttacttcagacacgttctgattgttaacttgtcagcacctgggagtcctgggatggttctagctggttcgcagatgagatcgatttcatgatctgctgtatcttgtttcgttaggttccttttaatctatccgtggtattatgctaacctatgatatggttcgatcgtgctagctacgtcctgtgtcataatttttagcatgcccttttttgtttggttttgtctgattgggctgtagatcagagtatactgtttcaaactacctactggatatatttattaaatttgaatctgtatgtgtgtcacatatatcttcataattaaaatggatggaaagatatatggataggtacatgtgttgctgtgggttttactggtactttgttagatatacatgcttagatacatgaagcaacatgatgttacagttcaataattcttgtttacctaataaacaaataaggataggtgtatgttgctgtgggttttgctggtactttgttagatatatatgcttagatatatgaagcaacatcctgctacggtttaataattattgtttatatctaatagacaagcctgctttttaattattttgatatacttggatgatggcatacagcagctatgtgtggatttttaaatacccagcatcatgagcatgcatgaccctgccttagtatgctgtttatttgcttgagacttctttttttgttggtactcaccttttgtagtttggtgactcttctgcaggtg

SEQ ID NO: 28 - DNA, 1044 bp, Tripidium ravennae
  source 1..1044: mol_type = other DNA; sub_species = ravennae
ccaccggcaaacaaccacgaatttgtaatggtactaggcaaattctccgtttggcggtgtgtgccggccaattacacgtttttgcggtgtcctccgacaaaatttgccttttaaaaacaattttataagagaagctccggagataaaaggccgtcaatgttacaagagtgaagtcgtctactccctccatcccaaaaaatgtaattctaagtatgagttgtattattatttttggacaaaaggagtataccacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctcccc

SEQ ID NO: 29 - DNA, 1934 bp, Tripidium ravennae
  source 1..1934: mol_type = other DNA; sub_species = ravennae
accacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctccccaacctcagcttctctcgttgttcggagcgcacacacaacccgatccccaatcccctcgtctctcctcgcgagcctcgtcgatccccgcttcaaggtacggcgatcatcctccctttctctaccttctcttctctagactaggtcggcgatccatggttagggcctgctagttctgttcctgtttttccgtggctgcgaggtacaatagatctgatggcgttatgatggttaacttgtcatactcctgcggtgtgcggtctatagtgcttttaggacatcaatttgacctggctcgttcgagatcggcgatccatggttaggaccctaggcggtggagtcgggttagatccgcgctgtttgtgttagtagatggatgcgacctttacttcagacacgttctgattgttaacttgtcagcacctgggagtcctgggatggttctagctggttcgcagatgagatcgatttcatgatctgctgtatcttgtttcgttaggttccttttaatctatccgtggtattatgctaacctatgatatggttcgatcgtgctagctacgtcctgtgtcataatttttagcatgcccttttttgtttggttttgtctgattgggctgtagatcagagtatactgtttcaaactacctactggatatatttattaaatttgaatctgtatgtgtgtcacatatatcttcataattaaaatggatggaaagatatatggataggtacatgtgttgctgtgggttttactggtactttgttagatatacatgcttagatacatgaagcaacatgatgttacagttcaataattcttgtttacctaataaacaaataaggataggtgtatgttgctgtgggttttgctggtactttgttagatatatatgcttagatatatgaagcaacatcctgctacggtttaataattattgtttatatctaatagacaagcctgctttttaattattttgatatacttggatgatggcatacagcagctatgtgtggatttttaaatacccagcatcatgagcatgcatgaccctgccttagtatgctgtttatttgcttgagacttctttttttgttggtactcaccttttgtagtttggtgactcttctgcaggtg

SEQ ID NO: 30 - DNA, 796 bp, Tripidium ravennae
  source 1..796: mol_type = other DNA; sub_species = ravennae
accacaagaatgatatcatcgtcatgcttagatcctttttagtaaagcttgagcttctctaaaagtagagaaattagaaaaaaatcacgtttttgtggtcttgatttctagcctccacaaaatctttggttttacattttttgtttgattttggtttcagaagtccttatttatatgtgctagtttggcagcacttaaaatcgttagagagagcctaaacaaaagccttttcaaaacgaccttgagccagattggttgatggccaaaatttgattgtcaaaacttaggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctcccc

SEQ ID NO: 31 - DNA, 1649 bp, Tripidium ravennae
  source 1..1649: mol_type = other DNA; sub_species = ravennae
aggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctccccaacctcagcttctctcgttgttcggagcgcacacacaacccgatccccaatcccctcgtctctcctcgcgagcctcgtcgatccccgcttcaaggtacggcgatcatcctccctttctctaccttctcttctctagactaggtcggcgatccatggttagggcctgctagttctgttcctgtttttccgtggctgcgaggtacaatagatctgatggcgttatgatggttaacttgtcatactcctgcggtgtgcggtctatagtgcttttaggacatcaatttgacctggctcgttcgagatcggcgatccatggttaggaccctaggcggtggagtcgggttagatccgcgctgtttgtgttagtagatggatgcgacctttacttcagacacgttctgattgttaacttgtcagcacctgggagtcctgggatggttctagctggttcgcagatgagatcgatttcatgatctgctgtatcttgtttcgttaggttccttttaatctatccgtggtattatgctaacctatgatatggttcgatcgtgctagctacgtcctgtgtcataatttttagcatgcccttttttgtttggttttgtctgattgggctgtagatcagagtatactgtttcaaactacctactggatatatttattaaatttgaatctgtatgtgtgtcacatatatcttcataattaaaatggatggaaagatatatggataggtacatgtgttgctgtgggttttactggtactttgttagatatacatgcttagatacatgaagcaacatgatgttacagttcaataattcttgtttacctaataaacaaataaggataggtgtatgttgctgtgggttttgctggtactttgttagatatatatgcttagatatatgaagcaacatcctgctacggtttaataattattgtttatatctaatagacaagcctgctttttaattattttgatatacttggatgatggcatacagcagctatgtgtggatttttaaatacccagcatcatgagcatgcatgaccctgccttagtatgctgtttatttgcttgagacttctttttttgttggtactcaccttttgtagtttggtgactcttctgcaggtg

SEQ ID NO: 32 - DNA, 511 bp, Tripidium ravennae
  source 1..511: mol_type = other DNA; sub_species = ravennae
aggcaagccaagattttagcagctatttggtttggtaccaaaatttgccaatgatctgttcttttgccttttcaaccggtttatcagccgtacttcagcttattctctctcacagaacactattgaatcagccgaaaagccaccgcagaacaggaccagtatctcacaaatggcatgccaaatatactcaccgtcagtgagcccgtttaacggcgtcgacaagtctaacggccaccaaccagcgaaccaccagcgtcaagctagccaagcgaagcagacggccgagacgttgacaccttggcgcgggcatctctctggccccctctcgagagttccgctccacctccactggtggcggtttccaagtccgttccgcctcctgctcctcctcacacggcacgaaaccgtcacggcaccggcagcacgggggattcctttcccaccgctccttccctttcccttcctcgcccgccgttttaaatagccagccccatccccagcttctctcccc

SEQ ID NO: 33 - DNA, 2631 bp, Setaria viridis
  source 1..2631: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacaggtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacag

SEQ ID NO: 34 - DNA, 1493 bp, Setaria viridis
  source 1..1493: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacaggtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcc

SEQ ID NO: 35 - DNA, 127 bp, Setaria viridis
  source 1..127: mol_type = other DNA
cgttgccgcaagactcagatcagattccgatccccagttcttccccaatcaccttgtggtctctcgtgtcgcggttcccagggacgcctccggctcgtcgctcgacagcgatctccgccccagcaag

SEQ ID NO: 36 - DNA, 1011 bp, Setaria viridis
  source 1..1011: mol_type = other DNA
gtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacag

SEQ ID NO: 37 - DNA, 2173 bp, Setaria viridis
  source 1..2173: mol_type = other DNA
gccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacaggtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacag

SEQ ID NO: 38 - DNA, 1035 bp, Setaria viridis
  source 1..1035: mol_type = other DNA
gccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacaggtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcc

SEQ ID NO: 39 - DNA, 1819 bp, Setaria viridis
  source 1..1819: mol_type = other DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctccttgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacag

SEQ ID NO: 40 - DNA, 681 bp, Setaria viridis
  source 1..681: mol_type = other DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctccttgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcc

SEQ ID NO: 41 - DNA, 1922 bp, Zea mays
  source 1..1922: mol_type = other DNA; sub_species = Mexicana
gtcgtgcccctctctagagataatgagcattgcatgtctaagttataaaaaattaccacatatttttttttgtcacacttgtgtttgaagtgcagtttatctatctctatacatatatttaaacttcactatatgaataatatagtctatagtattaaaataatatcaatgttttagatgattatataactgaactgctagacatggtctaaaggacaaccgagtattttgacaacatgactctacagttttatctttttagtgtgcatgtgttctttttacttttgcaaatagcttcacctatataatacttcatccattttattagtacatccatttactaaatttttagtacatctattttattctattttagcctctaaattaagaaaacttaaactctattttagttttttatttaataatttagatataaaatagaataaaataaagtgactaaaaaataactaaataccttttaagaaataaaaaaactaaggaaccatttttcttgttccgagtagataatgacagcctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgagccggcacggcaggcggcctcctctcacggcaccggcagctacgggggattcctttcccaccgctccttcgctttcccttcctcgcccgccgtaataaatagaccccctccacaccctctttccccaacctcgtgttcgttcggagcgcgcacacacacaaccagatctcccccaaatccacccgtcggcacctccgcttcaaggtacgccgctcatcctcctcccccccctctctctaccttctctagatcggcgtttcggtccatggttagggcccggtagttctacttctgttcatgtttgtgttagatccgtgtttgtgttagatccgtgctgctagatttcgtacacggatgcgacctgtacatcagacatgttctgattgctaacttgccagtgtttctctttggggaatcctgggatggctctagccgttccgcagacgggatcgatttcatgaattttttttgtttcgttgcatagggtttggtttgcccttttcctttatttcaatatatgccgtgcacttgtttgtcgggtcatcttttcatgttttttttggcttggttgtgatgatgtggtctggttgggcggtcgttctagatcggagtagaatactgtttcaaactacctggtggatttattaaaggatctgtatgtatgtgccatacatcttcatagttacgagtttaagatgatggatggaaatatcgatctaggataggtatacatgttgatgcgggttttactgatgcatatacagagatgcttttttttcgcttggttgtgatgatgtggtctggtcgggcggtcgttctagatcggagtagaatactgtttcaaactacctggtggatttattaattttggatctgtatgtgtgtcatacatcttcatagttacgagtttaagatcgatggaaatatcgatctaggataggtatacatgttgatgtgggttttactgatgcatatacatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcag

SEQ ID NO: 42 - DNA, 850 bp, Zea mays
  source 1..850: mol_type = other DNA; sub_species = Mexicana
gtcgtgcccctctctagagataatgagcattgcatgtctaagttataaaaaattaccacatatttttttttgtcacacttgtgtttgaagtgcagtttatctatctctatacatatatttaaacttcactatatgaataatatagtctatagtattaaaataatatcaatgttttagatgattatataactgaactgctagacatggtctaaaggacaaccgagtattttgacaacatgactctacagttttatctttttagtgtgcatgtgttctttttacttttgcaaatagcttcacctatataatacttcatccattttattagtacatccatttactaaatttttagtacatctattttattctattttagcctctaaattaagaaaacttaaactctattttagttttttatttaataatttagatataaaatagaataaaataaagtgactaaaaaataactaaataccttttaagaaataaaaaaactaaggaaccatttttcttgttccgagtagataatgacagcctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgagccggcacggcaggcggcctcctctcacggcaccggcagctacgggggattcctttcccaccgctccttcgctttcccttcctcgcccgccgtaataaatagaccccctccacaccctctttcccc

SEQ ID NO: 43 - DNA, 78 bp, Zea mays
  source 1..78: mol_type = other DNA; sub_species = Mexicana
aacctcgtgttcgttcggagcgcgcacacacacaaccagatctcccccaaatccacccgtcggcacctccgcttcaag

SEQ ID NO: 44 - DNA, 994 bp, Zea mays
  source 1..994: mol_type = other DNA; sub_species = Mexicana
gtacgccgctcatcctcctcccccccctctctctaccttctctagatcggcgtttcggtccatggttagggcccggtagttctacttctgttcatgtttgtgttagatccgtgtttgtgttagatccgtgctgctagatttcgtacacggatgcgacctgtacatcagacatgttctgattgctaacttgccagtgtttctctttggggaatcctgggatggctctagccgttccgcagacgggatcgatttcatgaattttttttgtttcgttgcatagggtttggtttgcccttttcctttatttcaatatatgccgtgcacttgtttgtcgggtcatcttttcatgttttttttggcttggttgtgatgatgtggtctggttgggcggtcgttctagatcggagtagaatactgtttcaaactacctggtggatttattaaaggatctgtatgtatgtgccatacatcttcatagttacgagtttaagatgatggatggaaatatcgatctaggataggtatacatgttgatgcgggttttactgatgcatatacagagatgcttttttttcgcttggttgtgatgatgtggtctggtcgggcggtcgttctagatcggagtagaatactgtttcaaactacctggtggatttattaattttggatctgtatgtgtgtcatacatcttcatagttacgagtttaagatcgatggaaatatcgatctaggataggtatacatgttgatgtgggttttactgatgcatatacatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcag

SEQ ID NO: 45 - DNA, 1971 bp, Zea mays
  source 1..1971: mol_type = other DNA; sub_species = Mexicana
gtcgtgcccctctctagagataaagagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctccttttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccttctttccccaacctcgtgttgttcggagcgcacacacacacaaccagatctcccccaaatccacccgtcggcacctccgcttcaaggtacgccgctcatcctcccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagaagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtctggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcag

SEQ ID NO: 46 - DNA, 887 bp, Zea mays
  source 1..887: mol_type = other DNA; sub_species = Mexicana
gtcgtgcccctctctagagataaagagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctccttttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccttctttcccc

SEQ ID NO: 47 - DNA, 77 bp, Zea mays
  source 1..77: mol_type = other DNA; sub_species = Mexicana
aacctcgtgttgttcggagcgcacacacacacaaccagatctcccccaaatccacccgtcggcacctccgcttcaag

SEQ ID NO: 48 - DNA, 1007 bp, Zea mays
  source 1..1007: mol_type = other DNA; sub_species = Mexicana
gtacgccgctcatcctcccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagaagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtctggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcag

SEQ ID NO: 49 - DNA, 2005 bp, Zea mays
  source 1..2005: mol_type = other DNA; sub_species = Mexicana
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcag

SEQ ID NO: 50 - DNA, 877 bp, Zea mays
  source 1..877: mol_type = other DNA; sub_species = Mexicana
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttcccc

SEQ ID NO: 51 - DNA, 78 bp, Zea mays
  source 1..78: mol_type = other DNA; sub_species = Mexicana
aacctcgtgttcgttcggagcgcacacacacgcaaccagatctcccccaaatccagccgtcggcacctccgcttcaag

SEQ ID NO: 52 - DNA, 1050 bp, Zea mays
  source 1..1050: mol_type = other DNA; sub_species = Mexicana
gtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcag

SEQ ID NO: 53 - DNA, 2005 bp, Zea mays
  source 1..2005: mol_type = other DNA; sub_species = Mexicana
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcag

SEQ ID NO: 54 - DNA, 1050 bp, Zea mays
  source 1..1050: mol_type = other DNA; sub_species = Mexicana
gtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcag

SEQ ID NO: 55 - DNA, 1632 bp, Sorghum bicolor
  source 1..1632: mol_type = other DNA
ccaagtccaaatgtcaattcccttgaagatgatctatttttatcttttgcattttgttatggaagtttgcaaatagcaacaaatgctaagtcaatttgccaaagtctttggagatgctcttagtctataattgaacaatatttgtaaaatacaaaaaaaaatagtactatttttattttaaaaaatttttggaagtaaacaaggccgaggatggggaaacggaagtccaacacgtcgttttctaagttgggctcaaaagcccatcacggaactgacctgctatgggtcggaggagagcgcgtccagatggttccagaggctggtggtggtgggccaaacgcggaactccgccaccgccacggcctcgtgcgcaagcgcagcgcgttgccgtgagccgtgacgtaaccctccgttgcccacgataaaagctccacccccgaccccggccccccgatttcccctacggaccagtctccccccgatcgcaatcgcgaattcgtcgcaccatcggcacgcagacgaacgaagcaaggctctccccatcggctcgtcaaggtatgcgttccctagatttgttcccttcctctctcggtttgtctatatatatgcatgtatggtcgattcccgatctcgtcgattctcggtttcgccttccgtacgaagattcgtttagattgttcatatgttctgttgtgttaccagattgatcggatcaacttgatccagttatcttcgctcctccgattagatccgtttctatttcagtatatatatactagtatagtatctagggttcacactgttgaccgactggttacttggaattgatccgtgctgagttcagttgttgccgtccataaaggcccgtgctattgtctgttctgaaacgaaatcctgtagatttcttagggttagtgttcaattcatcaaaaggttgattagtgaattatcaaatttgagagggttaaatcattctcatcatgttgtctcgaatgtaatcccaaagatattatagactgtgtttcgatttgatggattgatttgtgtatcatctaaatcaacaaggctaagtcatcagttcatagaatcatgtttaggtttccgttcaatagactagttttatcaatatataaaattataagaagggtagggtaaatcacgttgcctcaaatgccatcctgtatggtttggtttcaattcaattagtttggttgattagggtatgctctggattaagatggttaaatcttccctagcatcttccctgcctatccttacttgatccgtttcggatatgttggaagtacagcgagcttatttcatgttgatagtgacccctttcagattatactattgaatattgtatgtttgccacttctgtatgttgaattatcctgctaaattagcaatggaattagcatattggcaattggtatgcatggacctaatcaggacggatgtggttatgttagtttcaattcattgtcaattcattgttcacctgcgttagatatatatgatgatttttacgtgtagttcatagttcttgagttttggatctttcttatctgatatatgctttcctgtgcctgtgctttattgtgtcttaccatgcgatttttgtctatgcag

SEQ ID NO: 56 - DNA, 401 bp, Sorghum bicolor
  source 1..401: mol_type = other DNA
ccaagtccaaatgtcaattcccttgaagatgatctatttttatcttttgcattttgttatggaagtttgcaaatagcaacaaatgctaagtcaatttgccaaagtctttggagatgctcttagtctataattgaacaatatttgtaaaatacaaaaaaaaatagtactatttttattttaaaaaatttttggaagtaaacaaggccgaggatggggaaacggaagtccaacacgtcgttttctaagttgggctcaaaagcccatcacggaactgacctgctatgggtcggaggagagcgcgtccagatggttccagaggctggtggtggtgggccaaacgcggaactccgccaccgccacggcctcgtgcgcaagcgcagcgcgttgccgtgagccgtgac

SEQ ID NO: 57 - DNA, 154 bp, Sorghum bicolor
  source 1..154: mol_type = other DNA
gtaaccctccgttgcccacgataaaagctccacccccgaccccggccccccgatttcccctacggaccagtctccccccgatcgcaatcgcgaattcgtcgcaccatcggcacgcagacgaacgaagcaaggctctccccatcggctcgtcaag

SEQ ID NO: 58 - DNA, 1077 bp, Sorghum bicolor
  source 1..1077: mol_type = other DNA
gtatgcgttccctagatttgttcccttcctctctcggtttgtctatatatatgcatgtatggtcgattcccgatctcgtcgattctcggtttcgccttccgtacgaagattcgtttagattgttcatatgttctgttgtgttaccagattgatcggatcaacttgatccagttatcttcgctcctccgattagatccgtttctatttcagtatatatatactagtatagtatctagggttcacactgttgaccgactggttacttggaattgatccgtgctgagttcagttgttgccgtccataaaggcccgtgctattgtctgttctgaaacgaaatcctgtagatttcttagggttagtgttcaattcatcaaaaggttgattagtgaattatcaaatttgagagggttaaatcattctcatcatgttgtctcgaatgtaatcccaaagatattatagactgtgtttcgatttgatggattgatttgtgtatcatctaaatcaacaaggctaagtcatcagttcatagaatcatgtttaggtttccgttcaatagactagttttatcaatatataaaattataagaagggtagggtaaatcacgttgcctcaaatgccatcctgtatggtttggtttcaattcaattagtttggttgattagggtatgctctggattaagatggttaaatcttccctagcatcttccctgcctatccttacttgatccgtttcggatatgttggaagtacagcgagcttatttcatgttgatagtgacccctttcagattatactattgaatattgtatgtttgccacttctgtatgttgaattatcctgctaaattagcaatggaattagcatattggcaattggtatgcatggacctaatcaggacggatgtggttatgttagtttcaattcattgtcaattcattgttcacctgcgttagatatatatgatgatttttacgtgtagttcatagttcttgagttttggatctttcttatctgatatatgctttcctgtgcctgtgctttattgtgtcttaccatgcgatttttgtctatgcag

SEQ ID NO: 59 - DNA, 2000 bp, Sorghum bicolor
  source 1..2000: mol_type = other DNA
cactagctgcgcatgataaagccacaagccaaaattaattattatgggtgagaataaatacgtaccagcaccggccatagaaaaagtacattattaaaggtctaatttggaaacagtctgaaaacgacgtgcgctgcagaggtaaatgtaattttcggcactaaaaccattatcaactaattcattcaataacagttatttagaaaatgtatagctcgctctaaaaaaacagtttagaaaaacagtcaaaataattcgaccaacaaacagttaataaggttcattaaatatataatgcacggtgctatttgatcttttaaaggaaaaagaggaatagtcgtgggcgccaggcgggaattggggcgcgggagtctgccggacgacgcgttccgtccgaacggccggacccgacgaggcccccccgccgccccacgtcgcagaaccgtccgtgggtggtaatctggccgggtacaccagccgtccccttgggcggcctcacagcactgggctcacacgtgagttttgttctgggcttcggatcgcaccatatgggcctcggcatcagaaagacggggcccgtctgggatagaagagacaggaacctcctcgtggattccagaagccagccacgagcgaccaccgacgcggaggatactcgtcgtccaagtccaacacggcgggcgggcgggcggacgcgtgggctgggctaactgcctaaccttaacctccaaggcacgccaaggcccgcttctcccacccgacataaatatccccccatccaggcaaggcgcagagcctcagaccagattccgatcaatcacccataagctccccccaaatctgttcctcgtctcccgtctcgcggtttcctacttccctcggacgcctccggcaagtcgctcgaccgcgcgattccgcccgctcaaggtatcaactcggttcaccactccaatctacgtctgatttagatgttacttccatctatgtctaatttagatgttactccgatgcgattggattatgtttatgcggtttgcactgctctggaaactggaatctagggtttcgagtgatttgatcgatcgcgatctgtgatttcgttgcgccttgtgtatgcttggagtgatctaggcttgtatatgcggcatcgcgatctgacgcggttgctttgtagaggctgggggtctaggctgtgattttagaatcaaataaagctgttccttaccgtagatgtttcctacatgttctgtccagtactccagtgctatattcacattgtttgaggcttgagttttgtcgatcagtggtcatgagaaaaatatatctcatgattttagaggcacctattgggaaaggtagatggttccgttttacatgttttatagaccttgtggcatggctcctttgttctatgggtgctttattttcctgaataacagtaatgcgagactggtctatgggtgctttgaccagtaatgcgagactagttatttgatcatggtgcagttcctagtgattacgaacaacaatttggtagctcagttcattcagcattggtttctacgatccttatcattttacttctgaatgaatttatttatttaagatattacagtgcaataaactgctgtataatatcagtaacaaactgctattactagtaaatgcctagattcataataattcattattctacttgaaaatgatcttaggcctttttatgcggtcctacgcatccttccacaggacttgctgtttgtttgttttttgtaatccctcgctgggacgcagaatggttcatctgtgctaataatttttttgcatatataagtttatagttctcattattcatgtggctatggtagcctgtaaaatctattgtaataacatattagtcagccatacatctgttccaacttgctcaattgcaaatcatatctccacttaaagcacatgtttgcaagctttctgacaagtttctttgtgtttgattgaaacag

SEQ ID NO: 60 - DNA, 791 bp, Sorghum bicolor
  source 1..791: mol_type = other DNA
cactagctgcgcatgataaagccacaagccaaaattaattattatgggtgagaataaatacgtaccagcaccggccatagaaaaagtacattattaaaggtctaatttggaaacagtctgaaaacgacgtgcgctgcagaggtaaatgtaattttcggcactaaaaccattatcaactaattcattcaataacagttatttagaaaatgtatagctcgctctaaaaaaacagtttagaaaaacagtcaaaataattcgaccaacaaacagttaataaggttcattaaatatataatgcacggtgctatttgatcttttaaaggaaaaagaggaatagtcgtgggcgccaggcgggaattggggcgcgggagtctgccggacgacgcgttccgtccgaacggccggacccgacgaggcccccccgccgccccacgtcgcagaaccgtccgtgggtggtaatctggccgggtacaccagccgtccccttgggcggcctcacagcactgggctcacacgtgagttttgttctgggcttcggatcgcaccatatgggcctcggcatcagaaagacggggcccgtctgggatagaagagacaggaacctcctcgtggattccagaagccagccacgagcgaccaccgacgcggaggatactcgtcgtccaagtccaacacggcgggcgggcgggcggacgcgtgggctgggctaactgcctaaccttaacctccaaggcacgccaaggcccgcttctcccacccgacataaatatccccccatccaggcaaggcgc

SEQ ID NO: 61 - DNA, 136 bp, Sorghum bicolor
  source 1..136: mol_type = other DNA
agagcctcagaccagattccgatcaatcacccataagctccccccaaatctgttcctcgtctcccgtctcgcggtttcctacttccctcggacgcctccggcaagtcgctcgaccgcgcgattccgcccgctcaag

SEQ ID NO: 62 - DNA, 1073 bp, Sorghum bicolor
  source 1..1073: mol_type = other DNA
gtatcaactcggttcaccactccaatctacgtctgatttagatgttacttccatctatgtctaatttagatgttactccgatgcgattggattatgtttatgcggtttgcactgctctggaaactggaatctagggtttcgagtgatttgatcgatcgcgatctgtgatttcgttgcgccttgtgtatgcttggagtgatctaggcttgtatatgcggcatcgcgatctgacgcggttgctttgtagaggctgggggtctaggctgtgattttagaatcaaataaagctgttccttaccgtagatgtttcctacatgttctgtccagtactccagtgctatattcacattgtttgaggcttgagttttgtcgatcagtggtcatgagaaaaatatatctcatgattttagaggcacctattgggaaaggtagatggttccgttttacatgttttatagaccttgtggcatggctcctttgttctatgggtgctttattttcctgaataacagtaatgcgagactggtctatgggtgctttgaccagtaatgcgagactagttatttgatcatggtgcagttcctagtgattacgaacaacaatttggtagctcagttcattcagcattggtttctacgatccttatcattttacttctgaatgaatttatttatttaagatattacagtgcaataaactgctgtataatatcagtaacaaactgctattactagtaaatgcctagattcataataattcattattctacttgaaaatgatcttaggcctttttatgcggtcctacgcatccttccacaggacttgctgtttgtttgttttttgtaatccctcgctgggacgcagaatggttcatctgtgctaataatttttttgcatatataagtttatagttctcattattcatgtggctatggtagcctgtaaaatctattgtaataacatattagtcagccatacatctgttccaacttgctcaattgcaaatcatatctccacttaaagcacatgtttgcaagctttctgacaagtttctttgtgtttgattgaaacag

SEQ ID NO: 63 - DNA, 2064 bp, Sorghum bicolor
  source 1..2064: mol_type = other DNA
cattaaaagtcattatgtgcatgcgtcgtaactaacatggatatgttgctgcactatctcctcgcactagctgcgcatgataaagccacaagccaaaattaattattatgggtgagaataaatacgtaccagcaccggccatagaaaaagtacattattaaaggtctaatttggaaacagtctgaaaacgacgtgcgctgcagaggtaaatgtaattttcggcactaaaaccattatcaactaattcattcaataacagttatttagaaaatgtatagctcgctctaaaaaaacagtttagaaaaacagtcaaaataattcgaccaacaaacagttaataaggttcattaaatatataatgcacggtgctatttgatcttttaaaggaaaaagaggaatagtcgtgggcgccaggcgggaattggggcgcgggagtctgccggacgacgcgttccgtccgaacggccggacccgacgaggcccccccgccgccccacgtcgcagaaccgtccgtgggtggtaatctggccgggtacaccagccgtccccttgggcggcctcacagcactgggctcacacgtgagttttgttctgggcttcggatcgcaccatatgggcctcggcatcagaaagacggggcccgtctgggatagaagagacaggaacctcctcgtggattccagaagccagccacgagcgaccaccgacgcggaggatactcgtcgtccaagtccaacacggcgggcgggcgggcggacgcgtgggctgggctaactgcctaaccttaacctccaaggcacgccaaggcccgcttctcccacccgacataaatatccccccatccaggcaaggcgcagagcctcagaccagattccgatcaatcacccataagctccccccaaatctgttcctcgtctcccgtctcgcggtttcctacttccctcggacgcctccggcaagtcgctcgaccgcgcgattccgcccgctcaaggtatcaactcggttcaccactccaatctacgtctgatttagatgttacttccatctatgtctaatttagatgttactccgatgcgattggattatgtttatgcggtttgcactgctctggaaactggaatctagggtttcgagtgatttgatcgatcgcgatctgtgatttcgttgcgccttgtgtatgcttggagtgatctaggcttgtatatgcggcatcgcgatctgacgcggttgctttgtagaggctgggggtctaggctgtgattttagaatcaaataaagctgttccttaccgtagatgtttcctacatgttctgtccagtactccagtgctatattcacattgtttgaggcttgagttttgtcgatcagtggtcatgagaaaaatatatctcatgattttagaggcacctattgggaaaggtagatggttccgttttacatgttttatagaccttgtggcatggctcctttgttctatgggtgctttattttcctgaataacagtaatgcgagactggtctatgggtgctttgaccagtaatgcgagactagttatttgatcatggtgcagttcctagtgattacgaacaacaatttggtagctcagttcattcagcattggtttctacgatccttatcattttacttctgaatgaatttatttatttaagatattacagtgcaataaactgctgtataatatcagtaacaaactgctattactagtaaatgcctagattcataataattcattattctacttgaaaatgatcttaggcctttttatgcggtcctacgcatccttccacaggacttgctgtttgtttgttttttgtaatccctcgctgggacgcagaatggttcatctgtgctaataatttttttgcatatataagtttatagttctcattattcatgtggctatggtagcctgtaaaatctattgtaataacatattagtcagccatacatctgttccaacttgctcaattgcaaatcatatctccacttaaagcacatgtttgcaagctttctgacaagtttctttgtgtttgattgaaacag

SEQ ID NO: 64 - DNA, 855 bp, Sorghum bicolor
  source 1..855: mol_type = other DNA
cattaaaagtcattatgtgcatgcgtcgtaactaacatggatatgttgctgcactatctcctcgcactagctgcgcatgataaagccacaagccaaaattaattattatgggtgagaataaatacgtaccagcaccggccatagaaaaagtacattattaaaggtctaatttggaaacagtctgaaaacgacgtgcgctgcagaggtaaatgtaattttcggcactaaaaccattatcaactaattcattcaataacagttatttagaaaatgtatagctcgctctaaaaaaacagtttagaaaaacagtcaaaataattcgaccaacaaacagttaataaggttcattaaatatataatgcacggtgctatttgatcttttaaaggaaaaagaggaatagtcgtgggcgccaggcgggaattggggcgcgggagtctgccggacgacgcgttccgtccgaacggccggacccgacgaggcccccccgccgccccacgtcgcagaaccgtccgtgggtggtaatctggccgggtacaccagccgtccccttgggcggcctcacagcactgggctcacacgtgagttttgttctgggcttcggatcgcaccatatgggcctcggcatcagaaagacggggcccgtctgggatagaagagacaggaacctcctcgtggattccagaagccagccacgagcgaccaccgacgcggaggatactcgtcgtccaagtccaacacggcgggcgggcgggcggacgcgtgggctgggctaactgcctaaccttaacctccaaggcacgccaaggcccgcttctcccacccgacataaatatccccccatccaggcaaggcgc

SEQ ID NO: 65 - DNA, 2000 bp, Sorghum bicolor
  source 1..2000: mol_type = other DNA
agaagtaaaaaaaaagttcgtttcagaatcataaaggtaagttaaaaaaagaccatacaaaaaagaggtatttaatgataaactataatccagaatttgttaggatagtatataagaataagaccttgtttagtttcaaaaaaatttgcaaaattttccagattcctcgtcacatcaaatctttagaggtatgcatggagtattaaatatagacaagacctaaataagaaaacatgaaatgttcacgaaaaaaatcaagccaatgcatgatcgaagcaaacggtatagtaacggtgttaacctgatccattgatctttgtaatctttaacggccacctaccgcgggcagcaaacggcgtccccctcctcgatatctccgcggcggcctctggctttttccgcggaattgcgcggtggggacggattccacgagaccgcaacgcaaccgcctctcgccgctgggccccacaccgctcggtgccgtagcccgtagcctcacgggattctttctccctcctcccccgtgtataaattggcttcatcccctccctgcctcatccatccaaatcccactccccaatcccatcccgtcggagaaattcatcgaagcgaagcgaagcgaatcctcccgatcctctcaaggtacgcgagttttcgaatcccctccagacccctcgtatgctttccctgttcgttttcgtcgtagcgtttgattaggtatgctttccctgttcgtgttcgtcgtagggttcgattaggtcgtgtgaggccatggcctgctgtgataaatttatttgttgttatatcggatctgtagtcgatttgggggtcgtggtgtagatccgcgggctgtgatgaagttatttggtgtgattgtgctcgcgtgattctgcgcgttgagctcgagtagatctgatggttggacgaccgattggttcgttggctggctgcgctaaggttgggctgggctcatgttgcgttcgctgttgcgcgtgattccgcggatggacttgcgcttgattgccgccagatcacgttacgattatgtgatttcgtttggaactttttagatttgtagcttctgcttattatatgacagatgcgcctactgctcatatgcctgtggtaaataatggatggctgtgggtcaaactagttgattgtcgagtcatgtatcatatacaggtgtatagacttgcgtctaattgtttgcatgttgcagttatatgatttgttttagattgtttgttccactcatctaggctgtaaaagggacactacttattagcttgttgtttaatctttttattagtagattatattggtaatgttttactaattattattatgttatatgtgacttctgctcatgcctgattataatcatagatcactgtagttgattgttgaatcatgtgtcaaatacccgtatacataacactacacatttgcttagttgtttccttaactcatgcaaattgaacaccatgtatgatttgcatggtgctgtaatgttaaatactacagtcctgttggtacttgtttagtaagaatctgcttcatacaactatatgctatgcctgatgataatcatatatctttgtgtaattaataattagttgactgttgaataatgtatcgagtacataccatggcacaattgcttagtcacttccttaaccatgcatattgaactgaccccttcatgttctgctgaattgttctattctgattagaccatacatcatgtattgcaatctttatttgcaattgtaatgtaatggttcggttctcaaatgttaaatgctatagttgtgctactttctaatgttaaatgctatagctgtgctacttgtaagatctgcttcatagtttagttaaattaggatgatgagctttgatgctgtaactttgtttgattatgttcatagttgatcagtttttgttagactcacagtaacttatggtctcactcttcttctggtctttgatgtttgcag

SEQ ID NO: 66 - DNA, 565 bp, Sorghum bicolor
  source 1..565: mol_type = other DNA
agaagtaaaaaaaaagttcgtttcagaatcataaaggtaagttaaaaaaagaccatacaaaaaagaggtatttaatgataaactataatccagaatttgttaggatagtatataagaataagaccttgtttagtttcaaaaaaatttgcaaaattttccagattcctcgtcacatcaaatctttagaggtatgcatggagtattaaatatagacaagacctaaataagaaaacatgaaatgttcacgaaaaaaatcaagccaatgcatgatcgaagcaaacggtatagtaacggtgttaacctgatccattgatctttgtaatctttaacggccacctaccgcgggcagcaaacggcgtccccctcctcgatatctccgcggcggcctctggctttttccgcggaattgcgcggtggggacggattccacgagaccgcaacgcaaccgcctctcgccgctgggccccacaccgctcggtgccgtagcccgtagcctcacgggattctttctccctcctcccccgtgtataaattggcttcatcccctccctgcctcatccatcca

SEQ ID NO: 67 - DNA, 77 bp, Sorghum bicolor
  source 1..77: mol_type = other DNA
aatcccactccccaatcccatcccgtcggagaaattcatcgaagcgaagcgaagcgaatcctcccgatcctctcaag

SEQ ID NO: 68 - DNA, 1358 bp, Sorghum bicolor
  source 1..1358: mol_type = other DNA
gtacgcgagttttcgaatcccctccagacccctcgtatgctttccctgttcgttttcgtcgtagcgtttgattaggtatgctttccctgttcgtgttcgtcgtagggttcgattaggtcgtgtgaggccatggcctgctgtgataaatttatttgttgttatatcggatctgtagtcgatttgggggtcgtggtgtagatccgcgggctgtgatgaagttatttggtgtgattgtgctcgcgtgattctgcgcgttgagctcgagtagatctgatggttggacgaccgattggttcgttggctggctgcgctaaggttgggctgggctcatgttgcgttcgctgttgcgcgtgattccgcggatggacttgcgcttgattgccgccagatcacgttacgattatgtgatttcgtttggaactttttagatttgtagcttctgcttattatatgacagatgcgcctactgctcatatgcctgtggtaaataatggatggctgtgggtcaaactagttgattgtcgagtcatgtatcatatacaggtgtatagacttgcgtctaattgtttgcatgttgcagttatatgatttgttttagattgtttgttccactcatctaggctgtaaaagggacactacttattagcttgttgtttaatctttttattagtagattatattggtaatgttttactaattattattatgttatatgtgacttctgctcatgcctgattataatcatagatcactgtagttgattgttgaatcatgtgtcaaatacccgtatacataacactacacatttgcttagttgtttccttaactcatgcaaattgaacaccatgtatgatttgcatggtgctgtaatgttaaatactacagtcctgttggtacttgtttagtaagaatctgcttcatacaactatatgctatgcctgatgataatcatatatctttgtgtaattaataattagttgactgttgaataatgtatcgagtacataccatggcacaattgcttagtcacttccttaaccatgcatattgaactgaccccttcatgttctgctgaattgttctattctgattagaccatacatcatgtattgcaatctttatttgcaattgtaatgtaatggttcggttctcaaatgttaaatgctatagttgtgctactttctaatgttaaatgctatagctgtgctacttgtaagatctgcttcatagtttagttaaattaggatgatgagctttgatgctgtaactttgtttgattatgttcatagttgatcagtttttgttagactcacagtaacttatggtctcactcttcttctggtctttgatgtttgcag

SEQ ID NO: 69 - DNA, 2622 bp, Setaria italica
  source 1..2622: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacacgtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacag

SEQ ID NO: 70 - DNA, 1492 bp, Setaria italica
  source 1..1492: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacacgtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcc

SEQ ID NO: 71 - DNA, 127 bp, Setaria italica; identical to SEQ ID NO: 35
  source 1..127: mol_type = other DNA

SEQ ID NO: 72 - DNA, 1003 bp, Setaria italica
  source 1..1003: mol_type = other DNA
gtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacag

SEQ ID NO: 73 - DNA, 2622 bp, Setaria italica
  source 1..2622: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacccgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacacgtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacag

SEQ ID NO: 74 - DNA, 1492 bp, Setaria italica
  source 1..1492: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacccgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacacgtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcc

SEQ ID NO: 75 - DNA, 2164 bp, Setaria italica
  source 1..2164: mol_type = other DNA
gccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacacgtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacag

SEQ ID NO: 76 - DNA, 1034 bp, Setaria italica
  source 1..1034: mol_type = other DNA
gccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacacgtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcc

SEQ ID NO: 77 - DNA, 1810 bp, Setaria italica
  source 1..1810: mol_type = other DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacag

SEQ ID NO: 78 - DNA, 680 bp, Setaria italica
  source 1..680: mol_type = other DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcc

SEQ ID NO: 79 - DNA, 1940 bp, Coix lacryma-jobi
  source 1..1940: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcag

SEQ ID NO: 80 - DNA, 837 bp, Coix lacryma-jobi
  source 1..837: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacat

SEQ ID NO: 81 - DNA, 86 bp, Coix lacryma-jobi
  source 1..86: mol_type = other DNA
cctctcatcatcttctctcgtgtagcacgcgcagcccgatccccaatcccctctcctcgcgagcctcgtcgatccctcgcttcaag

SEQ ID NO: 82 - DNA, 1017 bp, Coix lacryma-jobi
  source 1..1017: mol_type = other DNA
gtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcag

SEQ ID NO: 83 - DNA, 1845 bp, Coix lacryma-jobi
  source 1..1845: mol_type = other DNA
ctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcag

SEQ ID NO: 84 - DNA, 742 bp, Coix lacryma-jobi
  source 1..742: mol_type = other DNA
ctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacat

SEQ ID NO: 85 - DNA, 1504 bp, Coix lacryma-jobi
  source 1..1504: mol_type = other DNA
caaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcag

SEQ ID NO: 86 - DNA, 401 bp, Coix lacryma-jobi
  source 1..401: mol_type = other DNA
caaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacat

SEQ ID NO: 87 - DNA, 1157 bp, Coix lacryma-jobi
  source 1..1157: mol_type = other DNA
ccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcag

SEQ ID NO: 88 - DNA, 54 bp, Coix lacryma-jobi
  source 1..54: mol_type = other DNA
ccttcctcgcccgccatcataaatagccacccctcccagcttccttcgccacat

SEQ ID NO: 89 - DNA, 798 bp, Coix lacryma-jobi
  source 1..798: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgcc

SEQ ID NO: 90 - DNA, 3393 bp, Coix lacryma-jobi
  source 1..3393: mol_type = other DNA
ggttctatacaacaccacacactgtgtgagtgtgtgaccagtggccaacttttgttcagttcaacgatcctggcctttccgggcacccaatacactaattaatctattgcagctaacctcaaaagaaatgcatttgcagttgtctgtcctaatcaatctactagcagactcacattattgatgtaggaaataaaattcagcctgtgacgtggatgcaacaactgcactgcacaggataccatcttagccgttgtgtcacaatttgctttgctaatgttttgagaaacccagctttgacaaacgtaagatcgatgagggccttacgtttggcacaatatgtattgtaatccggcacggcaagttagactcggtagtgtttagccggcatctttatgtttggcacaatttaatttaattcggcatggtaggttagactgcagcgtgagccggtcattgcaagttattatgacatgttagagcatctccaacaagttggaaaaaatgacttggtatatcatggtatatcatgagttttagcaacttattaattcatttgacaagtaaaaaaaagatccctcttcaacaatttgctattccaactcgctaaaataaaaaaaaattaggctcacctaggccgatctgcgttgccgcgggagaggagggtaaaagattttgcgctaggagaggtggaggaacagggcgcgggagccggccacggtgaaatcacgggatagcaacctcacccgcgcgcgcaaatttacgcgtgtggcatggaggaatagaaagttggaaaagatagcaagttcatttagggagttgttggagaagaatatttgtgcttttaccaaatttataagaatagcaagtgagaatagagagttgttggagatgctcaacaaatatacacaataaagtggtataataagcggcaagttattatgacatatataagagcaagtatacaataaggtgaactgttatatcgatcgatttttttttgagcacatatcgatcgaatttattgtaagatagaaaagagaagatataaaaacttatagtgatgaacaataataatataaagattatttttaaactatgaaaacaataaccgaactactcgctctcttctaattagtaaagtaaaggcttctcattgtatatatataaaaaaattcgttctgatttcttatattcaagacggggagagtgctgagtgctaacttactagtctacgagagaagcttcaaatcaaacagtgtactatagggcttacacaatttttctgagggaagcgattgtctgaaatgaactaaaaggctgagagctggaaaaagtagcttattctgattctgtgaagtgattctccatgctgattttaaaagtttatgataaaaaatcaaagagaataactttcagccacagaatcacttctctcagagaatcaacttatatggagaatcagaatcagatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtg

SEQ ID NO: 91 - DNA, 2287 bp, Coix lacryma-jobi
  source 1..2287: mol_type = other DNA
ggttctatacaacaccacacactgtgtgagtgtgtgaccagtggccaacttttgttcagttcaacgatcctggcctttccgggcacccaatacactaattaatctattgcagctaacctcaaaagaaatgcatttgcagttgtctgtcctaatcaatctactagcagactcacattattgatgtaggaaataaaattcagcctgtgacgtggatgcaacaactgcactgcacaggataccatcttagccgttgtgtcacaatttgctttgctaatgttttgagaaacccagctttgacaaacgtaagatcgatgagggccttacgtttggcacaatatgtattgtaatccggcacggcaagttagactcggtagtgtttagccggcatctttatgtttggcacaatttaatttaattcggcatggtaggttagactgcagcgtgagccggtcattgcaagttattatgacatgttagagcatctccaacaagttggaaaaaatgacttggtatatcatggtatatcatgagttttagcaacttattaattcatttgacaagtaaaaaaaagatccctcttcaacaatttgctattccaactcgctaaaataaaaaaaaattaggctcacctaggccgatctgcgttgccgcgggagaggagggtaaaagattttgcgctaggagaggtggaggaacagggcgcgggagccggccacggtgaaatcacgggatagcaacctcacccgcgcgcgcaaatttacgcgtgtggcatggaggaatagaaagttggaaaagatagcaagttcatttagggagttgttggagaagaatatttgtgcttttaccaaatttataagaatagcaagtgagaatagagagttgttggagatgctcaacaaatatacacaataaagtggtataataagcggcaagttattatgacatatataagagcaagtatacaataaggtgaactgttatatcgatcgatttttttttgagcacatatcgatcgaatttattgtaagatagaaaagagaagatataaaaacttatagtgatgaacaataataatataaagattatttttaaactatgaaaacaataaccgaactactcgctctcttctaattagtaaagtaaaggcttctcattgtatatatataaaaaaattcgttctgatttcttatattcaagacggggagagtgctgagtgctaacttactagtctacgagagaagcttcaaatcaaacagtgtactatagggcttacacaatttttctgagggaagcgattgtctgaaatgaactaaaaggctgagagctggaaaaagtagcttattctgattctgtgaagtgattctccatgctgattttaaaagtttatgataaaaaatcaaagagaataactttcagccacagaatcacttctctcagagaatcaacttatatggagaatcagaatcagatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacat

SEQ ID NO: 92 - DNA, 1020 bp, Coix lacryma-jobi
  source 1..1020: mol_type = other DNA
gtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtg

SEQ ID NO: 93 - DNA, 3393 bp, Coix lacryma-jobi
  source 1..3393: mol_type = other DNA
ggttctatacaacaccacacactgtgtgagtgtgtgaccagtggccaacttttgttcagttcaacgatcctggcctttccgggcacccaatacactaattaatctattgcagctaacctcaaaagaaatgcatttgcagttgtctgtcctaatcaatctactagcagactcacattattgatgtaggaaataaaattcagcctgtgacgtggatgcaacaactgcactgcacaggataccatcttagccgttgtgtcacaatttgctttgctaatgttttgagaaacccagctttgacaaacgtaagatcgatgagggccttacgtttggcacaatatgtattgtaatccggcacggcaagttagactcggtagtgtttagccggcatctttatgtttggcacaatttaatttaattcggcatggtaggttagactgcagcgtgagccggtcattgcaagttattatgacatgttagagcatctccaacaagttggaaaaaatgacttggtatatcatggtatatcatgagttttagcaacttattaattcatttgacaagtaaaaaaaagatccctcttcaacaatttgctattccaactcgctaaaataaaaaaaaattaggctcacctaggccgatctgcgttgccgcgggagaggagggtaaaagattttgcgctaggagaggtggaggaacagggcgcgggagccggccacggtgaaatcacgggatagcaacctcacccgcgcgcgcaaatttacgcgtgtggcatggaggaatagaaagttggaaaagatagcaagttcatttagggagttgttggagaagaatatttgtgcttttaccaaatttataagaatagcaagtgagaatagagagttgttggagatgctcaacaaatatacacaataaagtggtataataagcggcaagttattatgacatatataagagcaagtatacaataaggtgaactgttatatcgatcgatttttttttgagcacatatcgatcgaatttattgtaagatagaaaagagaagatataaaaacttatagtgatgaacaataataatataaagattatttttaaactatgaaaacaataaccgaactactcgctctcttctaattagtaaagtaaaggcttctcattgtatatatataaaaaaattcgttctgatttcttatattcaagacggggagagtgctgagtgctaacttactagtctacgagagaagcttcaaatcaaacagtgtactatagggcttacacaatttttctgagggaagcgattgtctgaaatgaactaaaaggctgagagctggaaaaagtagcttattctgattctgtgaagtgattctccatgctgattttaaaagtttatgataaaaaatcaaagagaataactttcagccacagaatcacttctctcagagaatcaacttatatggagaatcagaatcagatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtc

SEQ ID NO: 94 - DNA, 1020 bp, Coix lacryma-jobi
  source 1..1020: mol_type = other DNA
gtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtc

SEQ ID NO: 95 - DNA, 2166 bp, Coix lacryma-jobi
  source 1..2166: mol_type = other DNA
gtctacgagagaagcttcaaatcaaacagtgtactatagggcttacacaatttttctgagggaagcgattgtctgaaatgaactaaaaggctgagagctggaaaaagtagcttattctgattctgtgaagtgattctccatgctgattttaaaagtttatgataaaaaatcaaagagaataactttcagccacagaatcacttctctcagagaatcaacttatatggagaatcagaatcagatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtg

SEQ ID NO: 96 - DNA, 1060 bp, Coix lacryma-jobi
  source 1..1060: mol_type = other DNA
gtctacgagagaagcttcaaatcaaacagtgtactatagggcttacacaatttttctgagggaagcgattgtctgaaatgaactaaaaggctgagagctggaaaaagtagcttattctgattctgtgaagtgattctccatgctgattttaaaagtttatgataaaaaatcaaagagaataactttcagccacagaatcacttctctcagagaatcaacttatatggagaatcagaatcagatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacat

SEQ ID NO: 97 - DNA, 2166 bp, Coix lacryma-jobi
  source 1..2166: mol_type = other DNA
gtctacgagagaagcttcaaatcaaacagtgtactatagggcttacacaatttttctgagggaagcgattgtctgaaatgaactaaaaggctgagagctggaaaaagtagcttattctgattctgtgaagtgattctccatgctgattttaaaagtttatgataaaaaatcaaagagaataactttcagccacagaatcacttctctcagagaatcaacttatatggagaatcagaatcagatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtc

SEQ ID NO: 98 - DNA, 1943 bp, Coix lacryma-jobi
  source 1..1943: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtc

SEQ ID NO: 99 - DNA, 1943 bp, Coix lacryma-jobi
  source 1..1943: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtg

SEQ ID NO: 100 - DNA, 1943 bp, Coix lacryma-jobi
  source 1..1943: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggcg

SEQ ID NO: 101 - DNA, 1020 bp, Coix lacryma-jobi
  source 1..1020: mol_type = other DNA
gtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggcg

SEQ ID NO: 102 - DNA, 1943 bp, Coix lacryma-jobi
  source 1..1943: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggac

SEQ ID NO: 103 - DNA, 1020 bp, Coix lacryma-jobi
  source 1..1020: mol_type = other DNA
gtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggac

SEQ ID NO: 104 - DNA, 1943 bp, Coix lacryma-jobi
  source 1..1943: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcagacc

SEQ ID NO: 105 - DNA, 1020 bp, Coix lacryma-jobi
  source 1..1020: mol_type = other DNA
gtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcagacc

SEQ ID NO: 106 - DNA, 1943 bp, Coix lacryma-jobi
  source 1..1943: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcagggg

SEQ ID NO: 107 - DNA, 1020 bp, Coix lacryma-jobi
  source 1..1020: mol_type = other DNA
gtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcagggg

SEQ ID NO: 108 - DNA, 1943 bp, Coix lacryma-jobi
  source 1..1943: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcagggt

SEQ ID NO: 109 - DNA, 1020 bp, Coix lacryma-jobi
  source 1..1020: mol_type = other DNA
gtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcagggt

SEQ ID NO: 110 - DNA, 1943 bp, Coix lacryma-jobi
  source 1..1943: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcagcgt

SEQ ID NO: 111 - DNA, 1020 bp, Coix lacryma-jobi
  source 1..1020: mol_type = other DNA
gtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcagcgt

SEQ ID NO: 112 - DNA, 1943 bp, Coix lacryma-jobi
  source 1..1943: mol_type = other DNA
agcagactcgcattatcgatggagctctaccaaactggccctaggcattaacctaccatggatcacatcgtaaaaaaaaaaccctaccatggatcctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcagtgt

SEQ ID NO: 113 - DNA, 1020 bp, Coix lacryma-jobi
  source 1..1020: mol_type = other DNA
gtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcagtgt

SEQ ID NO: 114 - DNA, 1848 bp, Coix lacryma-jobi
  source 1..1848: mol_type = other DNA
ctatctgttttctttttgccctgaaagagtgaagtcatcatcatatttaccatggcgcgcgtaggagcgcttcgtcgaagacccataggggggcggtactcgcaccgtggttgtttcctgttatgtaatatcggatgggggagcagtcggctaggttggtcccatcggtactggtcgtcccctagtgcgctagatgcgcgatgtttgtcctcaaaaactcttttcttcttaataacaatcatacgcaaattttttgcgtattcgagaaaaaaagaagattctatctgttttttttttgaaatggctccaatttataggaggagcccgtttaacggcgtcgacaaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtc

SEQ ID NO: 115 - DNA, 1507 bp, Coix lacryma-jobi
  source 1..1507: mol_type = other DNA
caaatctaacggacaccaaccagcgaatgagcgaacccaccagcgccaagctagccaagcgaagcagacggccgagacgctgacacccttgccttggcgcggcatctccgtcgctggctcgctggctctggccccttcgcgagagttccggtccacctccacctgtgtcggtttccaactccgttccgccttcgcgtgggacttgttccgttcatccgttggcggcatccggaaattgcgtggcgtagagcacggggccctcctctcacacggcacggaaccgtcacgagctcacggcaccggcagcacggcggggattccttccccaccaccgctccttccctttcccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtc

SEQ ID NO: 116 - DNA, 1160 bp, Coix lacryma-jobi
  source 1..1160: mol_type = other DNA
ccttcctcgcccgccatcataaatagccacccctcccagcttccttcgccacatcctctcatcatcttctctcgtgtagcacgcgcagcccgatccccaatcccctctcctcgcgagcctcgtcgatccctcgcttcaaggtatggctatcgtccttcctctctctctctttaccttatctagatcggcgatccatggttagggcctgctagttctccgttcgtgtttgtcgatggctgtgaggcacaatagatccgtcggcgttatgatggttagcctgtcatgctcttgcgatctgtggttcctttaggaaaggcattaatttaatccctgatggttcgagatcggtgatccatggttagtaccctaagctgtggagtcgggtttagatccgcgctgttcgtaggcgatctgttctgattgttaacttgtcagtacctgcgaatcctcggtggttctagctggttcggagatcagatcgattccattatctgctatacatcttgtttcgttgcctaggctccgtttaatctatccatcgtatgatgttagcctttgatatgattcgatcgtgctagctatgtcctgtggacttaattgtcaggtcctaatttttaggaagactgttccaaaccatctgctggatttattaaatttggatctggatgtgtcacatacaccttcataattaaaatggatggaaatatctcttatcttttagatatggataggcatttatatgatgctgtgagttttactagtactttcttagaatatatgtacttttttagacggaatattgatatgtatacatgtgtagatacatgaagcaacatgctgctgtagtctaataattcctgttcatctaataatcaagtatgtatatgttctgtgtgttttattggtatttgattagatatatacatgcttagatacatacatgaagcagcatgctgctacagtttaatcattattgtttatccaataaacaaacatgctttttaatttatcttgatatgcttggatgacggaatatgcagagattttaagtacccagcatcatgagcatgcatgaccctgcgttagtatgctgtttatttgcttgagactctttcttttgtagatactcaccctgttttctggtgatcctactgcaggtc

SEQ ID NO: 117 - DNA, 2625 bp, Setaria italica
  source 1..2625: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacacgtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 118 - DNA, 1006 bp, Setaria italica
  source 1..1006: mol_type = other DNA
gtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 119 - DNA, 2625 bp, Setaria italica
  source 1..2625: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacccgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacacgtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacagggt

SEQ ID NO: 120 - DNA, 1006 bp, Setaria italica
  source 1..1006: mol_type = other DNA
gtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacagggt

SEQ ID NO: 121 - DNA, 2625 bp, Setaria italica
  source 1..2625: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacccgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacacgtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacagacc

SEQ ID NO: 122 - DNA, 1006 bp, Setaria italica
  source 1..1006: mol_type = other DNA
gtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacagacc

SEQ ID NO: 123 - DNA, 2167 bp, Setaria italica
  source 1..2167: mol_type = other DNA
gccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacacgtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 124 - DNA, 1813 bp, Setaria italica
  source 1..1813: mol_type = other DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 125 - DNA, 1813 bp, Setaria italica
  source 1..1813: mol_type = other DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacagggt

SEQ ID NO: 126 - DNA, 1813 bp, Setaria italica
  source 1..1813: mol_type = other DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacagggc

SEQ ID NO: 127 - DNA, 1006 bp, Setaria italica
  source 1..1006: mol_type = other DNA
gtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacagggc

SEQ ID NO: 128 - DNA, 2634 bp, Setaria viridis
  source 1..2634: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacaggtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 129 - DNA, 1014 bp, Setaria viridis
  source 1..1014: mol_type = other DNA
gtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 130 - DNA, 2634 bp, Setaria viridis
  source 1..2634: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacaggtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacagggt

SEQ ID NO: 131 - DNA, 1014 bp, Setaria viridis
  source 1..1014: mol_type = other DNA
gtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacagggt

SEQ ID NO: 132 - DNA, 2176 bp, Setaria viridis
  source 1..2176: mol_type = other DNA
gccgtttttgaagtatccaggattagaagcttctactgcgcttttatattatagctgtggacctgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacaggtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 133 - DNA, 1822 bp, Setaria viridis
  source 1..1822: mol_type = other DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctccttgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 134 - DNA, 1822 bp, Setaria viridis
  source 1..1822: mol_type = other DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 135 - DNA, 681 bp, Setaria viridis
  source 1..681: mol_type = other DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcc

SEQ ID NO: 136 - DNA, 1822 bp, Setaria viridis
  source 1..1822: mol_type = other DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagacatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatccaagaatgttgcgctgggcttcgattcacataacatgggcctgaagctctaaaacgacggcccggtcaccgggcgatggaaagagaccggatcctccttgtgaattctggaaggccacacgagagcgacccaccaccgacgcggaggagtcgtgcgtggtccaacacggccggcgggctgggctgcgaccttaaccagcaaggcacgccacgacccgcctcgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttgtttgcaatttgcgatttgctcgtttgttgcgcatcgtagtttatgtttggagtaatcgaggatttgtatgcggcgtcggcgctacctgcttaatcacgccatgtgacgcggttacttgcagaggctgggttagtgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggttaagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttatgatgtagctggctttgtattctaaaggctgctattattcatccatcgatttcacctatatgtaatccagagctttcgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacagggt

SEQ ID NO: 137 - DNA, 1925 bp, Zea mays
  source 1..1925: mol_type = other DNA; sub_species = Mexicana
gtcgtgcccctctctagagataatgagcattgcatgtctaagttataaaaaattaccacatatttttttttgtcacacttgtgtttgaagtgcagtttatctatctctatacatatatttaaacttcactatatgaataatatagtctatagtattaaaataatatcaatgttttagatgattatataactgaactgctagacatggtctaaaggacaaccgagtattttgacaacatgactctacagttttatctttttagtgtgcatgtgttctttttacttttgcaaatagcttcacctatataatacttcatccattttattagtacatccatttactaaatttttagtacatctattttattctattttagcctctaaattaagaaaacttaaactctattttagttttttatttaataatttagatataaaatagaataaaataaagtgactaaaaaataactaaataccttttaagaaataaaaaaactaaggaaccatttttcttgttccgagtagataatgacagcctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgagccggcacggcaggcggcctcctctcacggcaccggcagctacgggggattcctttcccaccgctccttcgctttcccttcctcgcccgccgtaataaatagaccccctccacaccctctttccccaacctcgtgttcgttcggagcgcgcacacacacaaccagatctcccccaaatccacccgtcggcacctccgcttcaaggtacgccgctcatcctcctcccccccctctctctaccttctctagatcggcgtttcggtccatggttagggcccggtagttctacttctgttcatgtttgtgttagatccgtgtttgtgttagatccgtgctgctagatttcgtacacggatgcgacctgtacatcagacatgttctgattgctaacttgccagtgtttctctttggggaatcctgggatggctctagccgttccgcagacgggatcgatttcatgaattttttttgtttcgttgcatagggtttggtttgcccttttcctttatttcaatatatgccgtgcacttgtttgtcgggtcatcttttcatgttttttttggcttggttgtgatgatgtggtctggttgggcggtcgttctagatcggagtagaatactgtttcaaactacctggtggatttattaaaggatctgtatgtatgtgccatacatcttcatagttacgagtttaagatgatggatggaaatatcgatctaggataggtatacatgttgatgcgggttttactgatgcatatacagagatgcttttttttcgcttggttgtgatgatgtggtctggtcgggcggtcgttctagatcggagtagaatactgtttcaaactacctggtggatttattaattttggatctgtatgtgtgtcatacatcttcatagttacgagtttaagatcgatggaaatatcgatctaggataggtatacatgttgatgtgggttttactgatgcatatacatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcaggtc

SEQ ID NO: 138 - DNA, 997 bp, Zea mays
  source 1..997: mol_type = other DNA; sub_species = Mexicana
gtacgccgctcatcctcctcccccccctctctctaccttctctagatcggcgtttcggtccatggttagggcccggtagttctacttctgttcatgtttgtgttagatccgtgtttgtgttagatccgtgctgctagatttcgtacacggatgcgacctgtacatcagacatgttctgattgctaacttgccagtgtttctctttggggaatcctgggatggctctagccgttccgcagacgggatcgatttcatgaattttttttgtttcgttgcatagggtttggtttgcccttttcctttatttcaatatatgccgtgcacttgtttgtcgggtcatcttttcatgttttttttggcttggttgtgatgatgtggtctggttgggcggtcgttctagatcggagtagaatactgtttcaaactacctggtggatttattaaaggatctgtatgtatgtgccatacatcttcatagttacgagtttaagatgatggatggaaatatcgatctaggataggtatacatgttgatgcgggttttactgatgcatatacagagatgcttttttttcgcttggttgtgatgatgtggtctggtcgggcggtcgttctagatcggagtagaatactgtttcaaactacctggtggatttattaattttggatctgtatgtgtgtcatacatcttcatagttacgagtttaagatcgatggaaatatcgatctaggataggtatacatgttgatgtgggttttactgatgcatatacatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcaggtc

SEQ ID NO: 139 - DNA, 1925 bp, Zea mays
  source 1..1925: mol_type = other DNA; sub_species = Mexicana
gtcgtgcccctctctagagataatgagcattgcatgtctaagttataaaaaattaccacatatttttttttgtcacacttgtgtttgaagtgcagtttatctatctctatacatatatttaaacttcactatatgaataatatagtctatagtattaaaataatatcaatgttttagatgattatataactgaactgctagacatggtctaaaggacaaccgagtattttgacaacatgactctacagttttatctttttagtgtgcatgtgttctttttacttttgcaaatagcttcacctatataatacttcatccattttattagtacatccatttactaaatttttagtacatctattttattctattttagcctctaaattaagaaaacttaaactctattttagttttttatttaataatttagatataaaatagaataaaataaagtgactaaaaaataactaaataccttttaagaaataaaaaaactaaggaaccatttttcttgttccgagtagataatgacagcctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgagccggcacggcaggcggcctcctctcacggcaccggcagctacgggggattcctttcccaccgctccttcgctttcccttcctcgcccgccgtaataaatagaccccctccacaccctctttccccaacctcgtgttcgttcggagcgcgcacacacacaaccagatctcccccaaatccacccgtcggcacctccgcttcaaggtacgccgctcatcctcctcccccccctctctctaccttctctagatcggcgtttcggtccatggttagggcccggtagttctacttctgttcatgtttgtgttagatccgtgtttgtgttagatccgtgctgctagatttcgtacacggatgcgacctgtacatcagacatgttctgattgctaacttgccagtgtttctctttggggaatcctgggatggctctagccgttccgcagacgggatcgatttcatgaattttttttgtttcgttgcatagggtttggtttgcccttttcctttatttcaatatatgccgtgcacttgtttgtcgggtcatcttttcatgttttttttggcttggttgtgatgatgtggtctggttgggcggtcgttctagatcggagtagaatactgtttcaaactacctggtggatttattaaaggatctgtatgtatgtgccatacatcttcatagttacgagtttaagatgatggatggaaatatcgatctaggataggtatacatgttgatgcgggttttactgatgcatatacagagatgcttttttttcgcttggttgtgatgatgtggtctggtcgggcggtcgttctagatcggagtagaatactgtttcaaactacctggtggatttattaattttggatctgtatgtgtgtcatacatcttcatagttacgagtttaagatcgatggaaatatcgatctaggataggtatacatgttgatgtgggttttactgatgcatatacatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcagggt

SEQ ID NO: 140 - DNA, 997 bp, Zea mays
  source 1..997: mol_type = other DNA; sub_species = Mexicana
gtacgccgctcatcctcctcccccccctctctctaccttctctagatcggcgtttcggtccatggttagggcccggtagttctacttctgttcatgtttgtgttagatccgtgtttgtgttagatccgtgctgctagatttcgtacacggatgcgacctgtacatcagacatgttctgattgctaacttgccagtgtttctctttggggaatcctgggatggctctagccgttccgcagacgggatcgatttcatgaattttttttgtttcgttgcatagggtttggtttgcccttttcctttatttcaatatatgccgtgcacttgtttgtcgggtcatcttttcatgttttttttggcttggttgtgatgatgtggtctggttgggcggtcgttctagatcggagtagaatactgtttcaaactacctggtggatttattaaaggatctgtatgtatgtgccatacatcttcatagttacgagtttaagatgatggatggaaatatcgatctaggataggtatacatgttgatgcgggttttactgatgcatatacagagatgcttttttttcgcttggttgtgatgatgtggtctggtcgggcggtcgttctagatcggagtagaatactgtttcaaactacctggtggatttattaattttggatctgtatgtgtgtcatacatcttcatagttacgagtttaagatcgatggaaatatcgatctaggataggtatacatgttgatgtgggttttactgatgcatatacatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcagggt

SEQ ID NO: 141 - DNA, 1974 bp, Zea mays
  source 1..1974: mol_type = other DNA; sub_species = Mexicana
gtcgtgcccctctctagagataaagagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctccttttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccttctttccccaacctcgtgttgttcggagcgcacacacacacaaccagatctcccccaaatccacccgtcggcacctccgcttcaaggtacgccgctcatcctcccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagaagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtctggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcaggtc

SEQ ID NO: 142 - DNA, 1010 bp, Zea mays
  source 1..1010: mol_type = other DNA; sub_species = Mexicana
gtacgccgctcatcctcccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagaagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtctggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcaggtc

SEQ ID NO: 143 - DNA, 1974 bp, Zea mays
  source 1..1974: mol_type = other DNA; sub_species = Mexicana
gtcgtgcccctctctagagataaagagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctccttttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccttctttccccaacctcgtgttgttcggagcgcacacacacacaaccagatctcccccaaatccacccgtcggcacctccgcttcaaggtacgccgctcatcctcccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagaagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtctggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcagggt

SEQ ID NO: 144 - DNA, 1010 bp, Zea mays
  source 1..1010: mol_type = other DNA; sub_species = Mexicana
gtacgccgctcatcctcccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagaagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtctggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgatacttctgcagggt

SEQ ID NO: 145 - DNA, 2008 bp, Zea mays
  source 1..2008: mol_type = other DNA; sub_species = Mexicana
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcaggtc

SEQ ID NO: 146 - DNA, 1053 bp, Zea mays
  source 1..1053: mol_type = other DNA; sub_species = Mexicana
gtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcaggtc

SEQ ID NO: 147 - DNA, 2008 bp, Zea mays
  source 1..2008: mol_type = other DNA; sub_species = Mexicana
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtc

SEQ ID NO: 148 - DNA, 1053 bp, Zea mays
  source 1..1053: mol_type = other DNA; sub_species = Mexicana
gtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtc

SEQ ID NO: 149 - DNA, 2008 bp, Zea mays
  source 1..2008: mol_type = other DNA; sub_species = Mexicana
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcagggt

SEQ ID NO: 150 - DNA, 1053 bp, Zea mays
  source 1..1053: mol_type = other DNA; sub_species = Mexicana
gtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcagggt

SEQ ID NO: 151 - DNA, 1635 bp, Sorghum bicolor
  source 1..1635: mol_type = other DNA
ccaagtccaaatgtcaattcccttgaagatgatctatttttatcttttgcattttgttatggaagtttgcaaatagcaacaaatgctaagtcaatttgccaaagtctttggagatgctcttagtctataattgaacaatatttgtaaaatacaaaaaaaaatagtactatttttattttaaaaaatttttggaagtaaacaaggccgaggatggggaaacggaagtccaacacgtcgttttctaagttgggctcaaaagcccatcacggaactgacctgctatgggtcggaggagagcgcgtccagatggttccagaggctggtggtggtgggccaaacgcggaactccgccaccgccacggcctcgtgcgcaagcgcagcgcgttgccgtgagccgtgacgtaaccctccgttgcccacgataaaagctccacccccgaccccggccccccgatttcccctacggaccagtctccccccgatcgcaatcgcgaattcgtcgcaccatcggcacgcagacgaacgaagcaaggctctccccatcggctcgtcaaggtatgcgttccctagatttgttcccttcctctctcggtttgtctatatatatgcatgtatggtcgattcccgatctcgtcgattctcggtttcgccttccgtacgaagattcgtttagattgttcatatgttctgttgtgttaccagattgatcggatcaacttgatccagttatcttcgctcctccgattagatccgtttctatttcagtatatatatactagtatagtatctagggttcacactgttgaccgactggttacttggaattgatccgtgctgagttcagttgttgccgtccataaaggcccgtgctattgtctgttctgaaacgaaatcctgtagatttcttagggttagtgttcaattcatcaaaaggttgattagtgaattatcaaatttgagagggttaaatcattctcatcatgttgtctcgaatgtaatcccaaagatattatagactgtgtttcgatttgatggattgatttgtgtatcatctaaatcaacaaggctaagtcatcagttcatagaatcatgtttaggtttccgttcaatagactagttttatcaatatataaaattataagaagggtagggtaaatcacgttgcctcaaatgccatcctgtatggtttggtttcaattcaattagtttggttgattagggtatgctctggattaagatggttaaatcttccctagcatcttccctgcctatccttacttgatccgtttcggatatgttggaagtacagcgagcttatttcatgttgatagtgacccctttcagattatactattgaatattgtatgtttgccacttctgtatgttgaattatcctgctaaattagcaatggaattagcatattggcaattggtatgcatggacctaatcaggacggatgtggttatgttagtttcaattcattgtcaattcattgttcacctgcgttagatatatatgatgatttttacgtgtagttcatagttcttgagttttggatctttcttatctgatatatgctttcctgtgcctgtgctttattgtgtcttaccatgcgatttttgtctatgcaggtc

SEQ ID NO: 152 - DNA, 1080 bp, Sorghum bicolor
  source 1..1080: mol_type = other DNA
gtatgcgttccctagatttgttcccttcctctctcggtttgtctatatatatgcatgtatggtcgattcccgatctcgtcgattctcggtttcgccttccgtacgaagattcgtttagattgttcatatgttctgttgtgttaccagattgatcggatcaacttgatccagttatcttcgctcctccgattagatccgtttctatttcagtatatatatactagtatagtatctagggttcacactgttgaccgactggttacttggaattgatccgtgctgagttcagttgttgccgtccataaaggcccgtgctattgtctgttctgaaacgaaatcctgtagatttcttagggttagtgttcaattcatcaaaaggttgattagtgaattatcaaatttgagagggttaaatcattctcatcatgttgtctcgaatgtaatcccaaagatattatagactgtgtttcgatttgatggattgatttgtgtatcatctaaatcaacaaggctaagtcatcagttcatagaatcatgtttaggtttccgttcaatagactagttttatcaatatataaaattataagaagggtagggtaaatcacgttgcctcaaatgccatcctgtatggtttggtttcaattcaattagtttggttgattagggtatgctctggattaagatggttaaatcttccctagcatcttccctgcctatccttacttgatccgtttcggatatgttggaagtacagcgagcttatttcatgttgatagtgacccctttcagattatactattgaatattgtatgtttgccacttctgtatgttgaattatcctgctaaattagcaatggaattagcatattggcaattggtatgcatggacctaatcaggacggatgtggttatgttagtttcaattcattgtcaattcattgttcacctgcgttagatatatatgatgatttttacgtgtagttcatagttcttgagttttggatctttcttatctgatatatgctttcctgtgcctgtgctttattgtgtcttaccatgcgatttttgtctatgcaggtc

SEQ ID NO: 153 - DNA, 2067 bp, Sorghum bicolor
  source 1..2067: mol_type = other DNA
cattaaaagtcattatgtgcatgcgtcgtaactaacatggatatgttgctgcactatctcctcgcactagctgcgcatgataaagccacaagccaaaattaattattatgggtgagaataaatacgtaccagcaccggccatagaaaaagtacattattaaaggtctaatttggaaacagtctgaaaacgacgtgcgctgcagaggtaaatgtaattttcggcactaaaaccattatcaactaattcattcaataacagttatttagaaaatgtatagctcgctctaaaaaaacagtttagaaaaacagtcaaaataattcgaccaacaaacagttaataaggttcattaaatatataatgcacggtgctatttgatcttttaaaggaaaaagaggaatagtcgtgggcgccaggcgggaattggggcgcgggagtctgccggacgacgcgttccgtccgaacggccggacccgacgaggcccccccgccgccccacgtcgcagaaccgtccgtgggtggtaatctggccgggtacaccagccgtccccttgggcggcctcacagcactgggctcacacgtgagttttgttctgggcttcggatcgcaccatatgggcctcggcatcagaaagacggggcccgtctgggatagaagagacaggaacctcctcgtggattccagaagccagccacgagcgaccaccgacgcggaggatactcgtcgtccaagtccaacacggcgggcgggcgggcggacgcgtgggctgggctaactgcctaaccttaacctccaaggcacgccaaggcccgcttctcccacccgacataaatatccccccatccaggcaaggcgcagagcctcagaccagattccgatcaatcacccataagctccccccaaatctgttcctcgtctcccgtctcgcggtttcctacttccctcggacgcctccggcaagtcgctcgaccgcgcgattccgcccgctcaaggtatcaactcggttcaccactccaatctacgtctgatttagatgttacttccatctatgtctaatttagatgttactccgatgcgattggattatgtttatgcggtttgcactgctctggaaactggaatctagggtttcgagtgatttgatcgatcgcgatctgtgatttcgttgcgccttgtgtatgcttggagtgatctaggcttgtatatgcggcatcgcgatctgacgcggttgctttgtagaggctgggggtctaggctgtgattttagaatcaaataaagctgttccttaccgtagatgtttcctacatgttctgtccagtactccagtgctatattcacattgtttgaggcttgagttttgtcgatcagtggtcatgagaaaaatatatctcatgattttagaggcacctattgggaaaggtagatggttccgttttacatgttttatagaccttgtggcatggctcctttgttctatgggtgctttattttcctgaataacagtaatgcgagactggtctatgggtgctttgaccagtaatgcgagactagttatttgatcatggtgcagttcctagtgattacgaacaacaatttggtagctcagttcattcagcattggtttctacgatccttatcattttacttctgaatgaatttatttatttaagatattacagtgcaataaactgctgtataatatcagtaacaaactgctattactagtaaatgcctagattcataataattcattattctacttgaaaatgatcttaggcctttttatgcggtcctacgcatccttccacaggacttgctgtttgtttgttttttgtaatccctcgctgggacgcagaatggttcatctgtgctaataatttttttgcatatataagtttatagttctcattattcatgtggctatggtagcctgtaaaatctattgtaataacatattagtcagccatacatctgttccaacttgctcaattgcaaatcatatctccacttaaagcacatgtttgcaagctttctgacaagtttctttgtgtttgattgaaacaggtg

SEQ ID NO: 154 - DNA, 1076 bp, Sorghum bicolor
  source 1..1076: mol_type = other DNA
gtatcaactcggttcaccactccaatctacgtctgatttagatgttacttccatctatgtctaatttagatgttactccgatgcgattggattatgtttatgcggtttgcactgctctggaaactggaatctagggtttcgagtgatttgatcgatcgcgatctgtgatttcgttgcgccttgtgtatgcttggagtgatctaggcttgtatatgcggcatcgcgatctgacgcggttgctttgtagaggctgggggtctaggctgtgattttagaatcaaataaagctgttccttaccgtagatgtttcctacatgttctgtccagtactccagtgctatattcacattgtttgaggcttgagttttgtcgatcagtggtcatgagaaaaatatatctcatgattttagaggcacctattgggaaaggtagatggttccgttttacatgttttatagaccttgtggcatggctcctttgttctatgggtgctttattttcctgaataacagtaatgcgagactggtctatgggtgctttgaccagtaatgcgagactagttatttgatcatggtgcagttcctagtgattacgaacaacaatttggtagctcagttcattcagcattggtttctacgatccttatcattttacttctgaatgaatttatttatttaagatattacagtgcaataaactgctgtataatatcagtaacaaactgctattactagtaaatgcctagattcataataattcattattctacttgaaaatgatcttaggcctttttatgcggtcctacgcatccttccacaggacttgctgtttgtttgttttttgtaatccctcgctgggacgcagaatggttcatctgtgctaataatttttttgcatatataagtttatagttctcattattcatgtggctatggtagcctgtaaaatctattgtaataacatattagtcagccatacatctgttccaacttgctcaattgcaaatcatatctccacttaaagcacatgtttgcaagctttctgacaagtttctttgtgtttgattgaaacaggtg

SEQ ID NO: 155 - DNA, 2067 bp, Sorghum bicolor
  source 1..2067: mol_type = other DNA
cattaaaagtcattatgtgcatgcgtcgtaactaacatggatatgttgctgcactatctcctcgcactagctgcgcatgataaagccacaagccaaaattaattattatgggtgagaataaatacgtaccagcaccggccatagaaaaagtacattattaaaggtctaatttggaaacagtctgaaaacgacgtgcgctgcagaggtaaatgtaattttcggcactaaaaccattatcaactaattcattcaataacagttatttagaaaatgtatagctcgctctaaaaaaacagtttagaaaaacagtcaaaataattcgaccaacaaacagttaataaggttcattaaatatataatgcacggtgctatttgatcttttaaaggaaaaagaggaatagtcgtgggcgccaggcgggaattggggcgcgggagtctgccggacgacgcgttccgtccgaacggccggacccgacgaggcccccccgccgccccacgtcgcagaaccgtccgtgggtggtaatctggccgggtacaccagccgtccccttgggcggcctcacagcactgggctcacacgtgagttttgttctgggcttcggatcgcaccatatgggcctcggcatcagaaagacggggcccgtctgggatagaagagacaggaacctcctcgtggattccagaagccagccacgagcgaccaccgacgcggaggatactcgtcgtccaagtccaacacggcgggcgggcgggcggacgcgtgggctgggctaactgcctaaccttaacctccaaggcacgccaaggcccgcttctcccacccgacataaatatccccccatccaggcaaggcgcagagcctcagaccagattccgatcaatcacccataagctccccccaaatctgttcctcgtctcccgtctcgcggtttcctacttccctcggacgcctccggcaagtcgctcgaccgcgcgattccgcccgctcaaggtatcaactcggttcaccactccaatctacgtctgatttagatgttacttccatctatgtctaatttagatgttactccgatgcgattggattatgtttatgcggtttgcactgctctggaaactggaatctagggtttcgagtgatttgatcgatcgcgatctgtgatttcgttgcgccttgtgtatgcttggagtgatctaggcttgtatatgcggcatcgcgatctgacgcggttgctttgtagaggctgggggtctaggctgtgattttagaatcaaataaagctgttccttaccgtagatgtttcctacatgttctgtccagtactccagtgctatattcacattgtttgaggcttgagttttgtcgatcagtggtcatgagaaaaatatatctcatgattttagaggcacctattgggaaaggtagatggttccgttttacatgttttatagaccttgtggcatggctcctttgttctatgggtgctttattttcctgaataacagtaatgcgagactggtctatgggtgctttgaccagtaatgcgagactagttatttgatcatggtgcagttcctagtgattacgaacaacaatttggtagctcagttcattcagcattggtttctacgatccttatcattttacttctgaatgaatttatttatttaagatattacagtgcaataaactgctgtataatatcagtaacaaactgctattactagtaaatgcctagattcataataattcattattctacttgaaaatgatcttaggcctttttatgcggtcctacgcatccttccacaggacttgctgtttgtttgttttttgtaatccctcgctgggacgcagaatggttcatctgtgctaataatttttttgcatatataagtttatagttctcattattcatgtggctatggtagcctgtaaaatctattgtaataacatattagtcagccatacatctgttccaacttgctcaattgcaaatcatatctccacttaaagcacatgtttgcaagctttctgacaagtttctttgtgtttgattgaaacagggt

SEQ ID NO: 156 - DNA, 1076 bp, Sorghum bicolor
  source 1..1076: mol_type = other DNA
gtatcaactcggttcaccactccaatctacgtctgatttagatgttacttccatctatgtctaatttagatgttactccgatgcgattggattatgtttatgcggtttgcactgctctggaaactggaatctagggtttcgagtgatttgatcgatcgcgatctgtgatttcgttgcgccttgtgtatgcttggagtgatctaggcttgtatatgcggcatcgcgatctgacgcggttgctttgtagaggctgggggtctaggctgtgattttagaatcaaataaagctgttccttaccgtagatgtttcctacatgttctgtccagtactccagtgctatattcacattgtttgaggcttgagttttgtcgatcagtggtcatgagaaaaatatatctcatgattttagaggcacctattgggaaaggtagatggttccgttttacatgttttatagaccttgtggcatggctcctttgttctatgggtgctttattttcctgaataacagtaatgcgagactggtctatgggtgctttgaccagtaatgcgagactagttatttgatcatggtgcagttcctagtgattacgaacaacaatttggtagctcagttcattcagcattggtttctacgatccttatcattttacttctgaatgaatttatttatttaagatattacagtgcaataaactgctgtataatatcagtaacaaactgctattactagtaaatgcctagattcataataattcattattctacttgaaaatgatcttaggcctttttatgcggtcctacgcatccttccacaggacttgctgtttgtttgttttttgtaatccctcgctgggacgcagaatggttcatctgtgctaataatttttttgcatatataagtttatagttctcattattcatgtggctatggtagcctgtaaaatctattgtaataacatattagtcagccatacatctgttccaacttgctcaattgcaaatcatatctccacttaaagcacatgtttgcaagctttctgacaagtttctttgtgtttgattgaaacagggt

SEQ ID NO: 157 - DNA, 2003 bp, Sorghum bicolor
  source 1..2003: mol_type = other DNA
agaagtaaaaaaaaagttcgtttcagaatcataaaggtaagttaaaaaaagaccatacaaaaaagaggtatttaatgataaactataatccagaatttgttaggatagtatataagaataagaccttgtttagtttcaaaaaaatttgcaaaattttccagattcctcgtcacatcaaatctttagaggtatgcatggagtattaaatatagacaagacctaaataagaaaacatgaaatgttcacgaaaaaaatcaagccaatgcatgatcgaagcaaacggtatagtaacggtgttaacctgatccattgatctttgtaatctttaacggccacctaccgcgggcagcaaacggcgtccccctcctcgatatctccgcggcggcctctggctttttccgcggaattgcgcggtggggacggattccacgagaccgcaacgcaaccgcctctcgccgctgggccccacaccgctcggtgccgtagcccgtagcctcacgggattctttctccctcctcccccgtgtataaattggcttcatcccctccctgcctcatccatccaaatcccactccccaatcccatcccgtcggagaaattcatcgaagcgaagcgaagcgaatcctcccgatcctctcaaggtacgcgagttttcgaatcccctccagacccctcgtatgctttccctgttcgttttcgtcgtagcgtttgattaggtatgctttccctgttcgtgttcgtcgtagggttcgattaggtcgtgtgaggccatggcctgctgtgataaatttatttgttgttatatcggatctgtagtcgatttgggggtcgtggtgtagatccgcgggctgtgatgaagttatttggtgtgattgtgctcgcgtgattctgcgcgttgagctcgagtagatctgatggttggacgaccgattggttcgttggctggctgcgctaaggttgggctgggctcatgttgcgttcgctgttgcgcgtgattccgcggatggacttgcgcttgattgccgccagatcacgttacgattatgtgatttcgtttggaactttttagatttgtagcttctgcttattatatgacagatgcgcctactgctcatatgcctgtggtaaataatggatggctgtgggtcaaactagttgattgtcgagtcatgtatcatatacaggtgtatagacttgcgtctaattgtttgcatgttgcagttatatgatttgttttagattgtttgttccactcatctaggctgtaaaagggacactacttattagcttgttgtttaatctttttattagtagattatattggtaatgttttactaattattattatgttatatgtgacttctgctcatgcctgattataatcatagatcactgtagttgattgttgaatcatgtgtcaaatacccgtatacataacactacacatttgcttagttgtttccttaactcatgcaaattgaacaccatgtatgatttgcatggtgctgtaatgttaaatactacagtcctgttggtacttgtttagtaagaatctgcttcatacaactatatgctatgcctgatgataatcatatatctttgtgtaattaataattagttgactgttgaataatgtatcgagtacataccatggcacaattgcttagtcacttccttaaccatgcatattgaactgaccccttcatgttctgctgaattgttctattctgattagaccatacatcatgtattgcaatctttatttgcaattgtaatgtaatggttcggttctcaaatgttaaatgctatagttgtgctactttctaatgttaaatgctatagctgtgctacttgtaagatctgcttcatagtttagttaaattaggatgatgagctttgatgctgtaactttgtttgattatgttcatagttgatcagtttttgttagactcacagtaacttatggtctcactcttcttctggtctttgatgtttgcagcgg

SEQ ID NO: 158 - DNA, 1361 bp, Sorghum bicolor
  source 1..1361: mol_type = other DNA
gtacgcgagttttcgaatcccctccagacccctcgtatgctttccctgttcgttttcgtcgtagcgtttgattaggtatgctttccctgttcgtgttcgtcgtagggttcgattaggtcgtgtgaggccatggcctgctgtgataaatttatttgttgttatatcggatctgtagtcgatttgggggtcgtggtgtagatccgcgggctgtgatgaagttatttggtgtgattgtgctcgcgtgattctgcgcgttgagctcgagtagatctgatggttggacgaccgattggttcgttggctggctgcgctaaggttgggctgggctcatgttgcgttcgctgttgcgcgtgattccgcggatggacttgcgcttgattgccgccagatcacgttacgattatgtgatttcgtttggaactttttagatttgtagcttctgcttattatatgacagatgcgcctactgctcatatgcctgtggtaaataatggatggctgtgggtcaaactagttgattgtcgagtcatgtatcatatacaggtgtatagacttgcgtctaattgtttgcatgttgcagttatatgatttgttttagattgtttgttccactcatctaggctgtaaaagggacactacttattagcttgttgtttaatctttttattagtagattatattggtaatgttttactaattattattatgttatatgtgacttctgctcatgcctgattataatcatagatcactgtagttgattgttgaatcatgtgtcaaatacccgtatacataacactacacatttgcttagttgtttccttaactcatgcaaattgaacaccatgtatgatttgcatggtgctgtaatgttaaatactacagtcctgttggtacttgtttagtaagaatctgcttcatacaactatatgctatgcctgatgataatcatatatctttgtgtaattaataattagttgactgttgaataatgtatcgagtacataccatggcacaattgcttagtcacttccttaaccatgcatattgaactgaccccttcatgttctgctgaattgttctattctgattagaccatacatcatgtattgcaatctttatttgcaattgtaatgtaatggttcggttctcaaatgttaaatgctatagttgtgctactttctaatgttaaatgctatagctgtgctacttgtaagatctgcttcatagtttagttaaattaggatgatgagctttgatgctgtaactttgtttgattatgttcatagttgatcagtttttgttagactcacagtaacttatggtctcactcttcttctggtctttgatgtttgcagcgg

SEQ ID NO: 159 - DNA, 1812 bp, synthetic construct
  source 1..1812: mol_type = other DNA
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 160 - DNA, 2001 bp, synthetic construct
  source 1..2001: mol_type = other DNA
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 161 - DNA, 253 bp, Agrobacterium tumefaciens
  source 1..253: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 162 - DNA, 210 bp, Triticum aestivum
  source 1..210: mol_type = other DNA
ctgcatgcgtttggacgtatgctcattcaggttggagccaatttggttgatgtgtgtgcgagttcttgcgagtctgatgagacatctctgtattgtgtttctttccccagtgttttctgtacttgtgtaatcggctaatcgccaacagattcggcgatgaataaatgagaaataaattgttctgattttgagtgcaaaaaaaaaggaatt

SEQ ID NO: 163 - DNA, 1204 bp, synthetic construct
  source 1..1204: mol_type = other DNA
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatcctctagaaccatcttccacacactcaagccacactattggagaacacacagggacaacacaccataagatccaagggaggcctccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaag

SEQ ID NO: 164 - DNA, 1399 bp, Oryza sativa
  source 1..1399: mol_type = other DNA
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaag

SEQ ID NO: 165 - DNA, 2181 bp, Oryza sativa
  source 1..2181: mol_type = other DNA
gacaacaacatgcttctcatcaacatggagggaagagggagggagaaagtgtcgcctggtcacctccattgtcacactagccactggccagctctcccacaccaccaatgccaggggcgagctttagcacagccaccgcttcacctccaccaccgcactaccctagcttcgcccaacagccaccgtcaacgcctcctctccgtcaacataagagagagagagaagaggagagtagccatgtggggaggaggaatagtacatggggcctaccgtttggcaagttattttgggttgccaagttaggccaataaggggagggatttggccatccggttggaaaggttattggggtagtatctttttactagaattgtcaaaaaaaaatagtttgagagccatttggagaggatgttgcctgttagaggtgctcttaggacatcaaattccataaaaacatcagaaaaattctctcgatgaagatttataaccactaaaactgccctcaattcgaagggagttcaaaacaattaaaatcatgttcgaattgagtttcaatttcactttaacccctttgaaatctcaatggtaaaacatcaacccgtcaggtagcatggttctttttattcctttcaaaaagagttaattacaaacagaatcaaaactaacagttaggcccaaggcccatccgagcaaacaatagatcatgggccaggcctgccaccaccctccccctcctggctcccgctcttgaatttcaaaatccaaaaatatcggcacgactggccgccgacggagcgggcggaaaatgacggaacaacccctcgaattctaccccaactacgcccaccaacccacacgccactgacaatccggtcccacccttgtgggcccacctacaagcgagacgtcagtcgctcgcagcaaccagtgggcccacctcccagtgagcggcgggtagatctggactcttacccacccacactaaacaaaacggcatgaatattttgcactaaaaccctcagaaaaattccgatattccaaaccagtacagttcctgaccgttggaggagccaaagtggagcggagtgtaaaattgggaaacttaatcgagggggttaaacgcaaaaacgccgaggcgcctcccgctctatagaaaggggaggagtgggaggtggaaaccctaccacaccgcagagaaaggcgtcttcgtactcgcctctctccgcgccctcctccgccgccgctcgccgccgttcgtctccgccgccaccggctagccatccaggtaaaacaaacaaaaacggatctgatgcttccattcctccgtttctcgtagtagcgcgcttcgatctgtgggtggatctgggtgatcctggggtgtggttcgttctgtttgatagatctgtcggtggatctggccttctgtggttgtcgatgtccggatctgcgttttgatcagtggtagttcgtggatctggcgaaatgttttggatctggcagtgagacgctaagaatcgggaaatgatgcaatattaggggggtttcggatggggatccactgaattagtctgtctccctgctgataatctgttcctttttggtagatctggttagtgtatgtttgtttcggatagatctgatcaatgcttgtttgttttttcaaattttctacctaggttgtataggaatggcatgcggatctggttggattgccatgatccgtgctgaaatgcccctttggttgatggatcttgatattttactgctgttcacctagatttgtactcccgtttatacttaatttgttgcttattatgaatagatctgtaacttaggcacatgtatggacggagtatgtggatctgtagtatgtacattgctgcgagctaagaactatttcagagcaagcacagaaaaaaatatttagacagattgggcaactatttgatggtctttggtatcatgctttgtagtgctcgtttctgcgtagtaatcttttgatctgatctgaagataggtgctattatattcttaaaggtcattagaacgctatctgaaaggctgtattatgtggattggttcacctgtgactccctgttcgtcttgtcttgataaatcctgtgataaaaaaaattcttaaggcgtaatttgttgaaatcttgttttgtcctatgcagcctg

SEQ ID NO: 166 - DNA, 1653 bp, synthetic construct
  source 1..1653: mol_type = other DNA
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 167 - DNA, 936 bp, synthetic construct
  source 1..936: mol_type = other DNA
atggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaa

SEQ ID NO: 168 - DNA, 675 bp, synthetic construct
  source 1..675: mol_type = other DNA
ggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaaccatcttccacacactcaagccacactattggagaacacacagggacaacacaccataa

SEQ ID NO: 169 - DNA, 622 bp, Cauliflower mosaic virus
  source 1..622: mol_type = other DNA
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatctagacgcaagacccttcctctatataaggaagttcatttcatttggagaggacacgctga

SEQ ID NO: 170 - DNA, 1446 bp, synthetic construct
  source 1..1446: mol_type = other DNA
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatctaccgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcgg

SEQ ID NO: 171 - DNA, 1165 bp, synthetic construct
  source 1..1165: mol_type = other DNA
ggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacaagctgactctagcagatcctctagaaccatcttccacacactcaagccacactattggagaacacacagggacaacacaccataagatccaagggaggcctccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggggaatggggctctcggatgtagatctgcgatccgccgttgttgggggagatgatggggggtttaaaatttgcgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaag

SEQ ID NO: 172 - DNA, 1751 bp, synthetic construct
  source 1..1751: mol_type = other DNA
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattagcttaggcctcatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgaggcctcatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgaagctaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaag

SEQ ID NO: 173 - DNA, 1101 bp, synthetic construct
  source 1..1101: mol_type = other DNA
ggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaccgccgccgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaag

SEQ ID NO: 174 - DNA, 200 bp, Cauliflower mosaic virus
  source 1..200: mol_type = other DNA
aaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagt

SEQ ID NO: 175 - DNA, 300 bp, Oryza sativa
  source 1..300: mol_type = other DNA
attaatcgatcctccgatcccttaattaccataccattacaccatgcatcaatatccatatatatataaaccctttcgcacgtacttatactatgttttgtcatacatatatatgtgtcgaacgatcgatctatcactgatatgatatgattgatccatcagcctgatctctgtatcttgttatttgtataccgtcaaataaaagtttcttccacttgtgttaataattagctactctcatctcatgaaccctatatataactagtttaatttgctgtcaattgaacatgatgatcgatg

SEQ ID NO: 176 - DNA, 623 bp, Cauliflower mosaic virus
  source 1..623: mol_type = other DNA
ggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatggtccgatgtgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctga

SEQ ID NO: 177 - skipped

SEQ ID NO: 178 - DNA, 804 bp, Zea mays
  source 1..804: mol_type = other DNA
accgtcttcggtacgcgctcactccgccctctgcctttgttactgccacgtttctctgaatgctctcttgtgtggtgattgctgagagtggtttagctggatctagaattacactctgaaatcgtgttctgcctgtgctgattacttgccgtcctttgtagcagcaaaatatagggacatggtagtacgaaacgaagatagaacctacacagcaatacgagaaatgtgtaatttggtgcttagcggtatttatttaagcacatgttggtgttatagggcacttggattcagaagtttgctgttaatttaggcacaggcttcatactacatgggtcaatagtatagggattcatattataggcgatactataataatttgttcgtctgcagagcttattatttgccaaaattagatattcctattctgtttttgtttgtgtgctgttaaattgttaacgcctgaaggaataaatataaatgacgaaattttgatgtttatctctgctcctttattgtgaccataagtcaagatcagatgcacttgttttaaatattgttgtctgaagaaataagtactgacagtattttgatgcattgatctgcttgtttgttgtaacaaaatttaaaaataaagagtttcctttttgttgctctccttacctcctgatggtatctagtatctaccaactgacactatattgcttctctttacatacgtatcttgctcgatgccttctccctagtgttgaccagtgttactcacatagtctttgctcatttcattgtaatgcagataccaagcgg

SEQ ID NO: 179 - DNA, 1396 bp, Oryza sativa
  source 1..1396: mol_type = other DNA
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccagcccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgtttttttttccgtctcggtctcgatctttggccttggtagtttgggtgggcgagaggcggcttcgtgccgcccagatcggtgcgcgggaggggcgggatctcgcggctggctctcgcccccgtggatccggcccggatctcgcggggaatggggctctcggatgtagatctgcgatccgccgttgttggggccgatgatggggcccttaaaatttccgccgtgctaaacaagatcaggaagaggggaaaagggcactatggtttatatttttatatatttctgctgcttcgtcaggcttagatgtgctagatctttctttcttctttttgtgggtagaatttaatccctcagcattgttcatcggtagtttttcttttcatgattcgtgacaaatgcagcctcgtgcggacgtttttttgtaggtagaag

SEQ ID NO: 180 - DNA, 2625 bp, Setaria italica
  source 1..2625: mol_type = other DNA
actgccgcgacacgcctcactggcgggagggctccgagcgctctctccccggcggccggcggagcagcgatctggattggagagaatagaggaaagagagggaaaaggagagagatagcgcaaagagctgaaaagataaggttgtgcgggctgtggtgattagaggaccactaatccctccatctcctaatgacgcggtgcccaagaccagtgccgcggcacaccagcgtctaagtgaacttccgctaaccttccggtcattgcgcctgaaagatgtcatgtggcgaggcccccctctcagtagattgccaactgcctaccgtgccactcttccatgcatgattgctcccgtctatcccgtttctcacaacagatagacaacagtaagcatcactaaagcaagcatgtgtagaaccttaaaaaaaggcttatactaccagtatactatcaaccagcatgccgtttttgaagtatccaggattagaagcttctactgcgcttttatattatagctgtggacccgtggtaacctttctcttttggcgcttgcttaatctcggccgtgctggtccatgcttaggcactaggcagagatagagccgggggtgaatggggctaaagctcagctgctcgaggggccgtgggctggtttccactagcctacagctgtgccacgtgcggccgcgcaagccgaagcaagcacgctgagccgttggacagcttgtcataatgccattacgtggattacacgtaactggccctgtaactactcgttcggccatcatcaaacgacgacgtccgctaggcgacgacacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 181 - DNA, 2008 bp, Zea mays
  source 1..2008: mol_type = other DNA; sub_species = Mexicana
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcagcgg

SEQ ID NO: 182 - DNA, 1053 bp, Zea mays
  source 1..1053: mol_type = other DNA; sub_species = Mexicana
gtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgttgggtgatacttctgcagcgg

SEQ ID NO: 183 - DNA, 2625 bp, Setaria italica; identical to SEQ ID NO: 180
  source 1..2625: mol_type = other DNA